(19) United States
(12) Patent Application Publication
(10) Pub. No.: US 2025/0326851 A1
(43) Pub. Date: Oct. 23, 2025

(54) MEDICAL USE OF CCR8 ANTIBODIES AND DOSING SCHEDULE

(71) Applicant: BAYER AKTIENGESELLSCHAFT, Leverkusen (DE)

(72) Inventors: Gökben KOCA, Berlin (DE); Joanna GRUDZINSKA-GOEBEL, Berlin (DE); Markus KRAUSS, Köln (DE); Marc KUNZE, Berlin (DE); Mark TRAUTWEIN, Wülfrath (DE); Matyas GORJANACZ, Berlin (DE); Pascale BUCHMANN, Leverkusen (DE); Dominik MUMBERG, Glienicke (DE); Stephan MENZ, Berlin (DE); Christian BERTLING, Wuppertal (DE); Helge ROIDER, Hohen Neuendorf (DE); Chirag PATEL, Boston, MA (US); Patricia COLE, East Hannover, NJ (US); Anita Natalie KREMER, Pfaffhausen (CH)

(21) Appl. No.: 19/110,032

(22) PCT Filed: Sep. 8, 2023
(86) PCT No.: PCT/EP2023/074698

§ 371 Date: Mar. 7, 2025

Related U.S. Application Data
(60) Provisional application No. 63/375,124, filed on Sep. 9, 2022.

(51) Int. Cl. C07K 16/28; A61K 9/00; A61P 35/00

(52) U.S. Cl. CPC C07K 16/2866; A61K 9/0019; A61P 35/00; C07K 2317/569

(57) ABSTRACT

The present invention relates to medical uses comprising the administration of anti-human CCR8 antibodies in specifically defined dosage regimens in monotherapy or combination therapy with an anti-PD-(L)1 antibody. The dosing schemes were developed for anti-human/cynomolgus CCR8 antibody TPP-23411, but they can also be used for other antibodies having similar properties as TPP-23411. The medical uses or dosage regimens may comprise a stratification step to select patients with an increased probability of treatment success. Suggested biomarkers are a) Tumor Proportion Score or Combined Positive Score as a measure for PD-(L)1 expression, b) analysing in a blood, plasma or serum sample inflammatory cytokines and c) previous treatment of the cancer for at least 6 months with an anti-PD-(L)1 antibody. Furthermore, provided are anti-human CCR8 antibody-based medical uses and treatment methods comprising the administration of a Zr-89-labeled anti-CD8 minibody to determine the abundance and/or distribution of CD8 cells by means of a PET scan for stratification or for monitoring treatment success or disease progression. Also provided is a method to reliably determine an anti-anti-CCR8 antibody in cynomolgus or human plasma. Finally, an anti-murine CCR8 surrogate antibody is disclosed that mimics the unusual half-life of TPP-23411.

### TECHNICAL FIELD

The present invention relates to medical uses comprising the administration of anti-human CCR8 antibodies in specifically defined dosage regimens in monotherapy or in combination therapy with an anti-PD-(L)1 antibody. The dosing schemes were developed for anti-human/cynomolgus CCR8 antibody TPP-23411, but they can also be used for other antibodies having similar properties as TPP-23411. In some embodiments the anti-human CCR8 antibody-based medical uses or methods of treatment comprise a stratification step to select patients with an increased probability of treatment success. Suggested biomarkers are
a. Tumor Proportion Score or Combined Positive Score as a measure for PD-(L)1 expression,
b. Analysing in a blood, plasma or serum sample inflammatory cytokines selected from the group of IFN-γ, IL-1β, IL-2, IL-4, IL-6, IL-8, IL-10, IL 12p70, IL-13, and TNF-α, and
c. previous treatment of the cancer for at least 6 months with an anti-PD-(L)1 antibody.

Furthermore, according to the present invention there are provided anti-human CCR8 antibody-based medical uses and treatment methods comprising the administration of a Zr-89-labeled anti-CD8 minibody to determine the abundance and/or distribution of CD8 cells by means of a PET scan for stratification or for monitoring treatment success or disease progression. According to the current invention there is also provided a method to reliably determine an anti-anti-CCR8 antibody in cynomolgus or human plasma. Finally, an anti-murine CCR8 surrogate antibody is disclosed that mimics the unusual half-life of TPP-23411.

### BACKGROUND

Targeting regulatory T cells (T regs) is an attractive approach to enhance anti-tumor immune responses in monotherapy settings or in combination with immune checkpoint inhibitors (ICIs), because Tregs suppress the anti-tumor immune functions of cytotoxic T cells and contribute to an immunosuppressive tumor microenvironment (TME). However, peripheral Tregs are physiologically indispensable for maintaining immune tolerance. Therefore, systemic depletion of Tregs may not only enhance anti-tumor immune responses but also elicit strong and undesirable autoimmunity. In essence, a key issue for tailoring Treg targeting cancer immunotherapy resides in ensuring specific depletion of tumor infiltrating Tregs without affecting peripheral Tregs.

Several Treg-depleting approaches have demonstrated a reduction in tumor burden and augmentation of anti-tumor immune responses in preclinical models. However, most of these approaches addressed surface receptors that are not specific for tumor infiltrating Tregs, e.g. CD25 or CCR4 and have therefore been associated with substantial side effects. In consequence, there is a high need for safe and effective medical uses to deplete tumor infiltrating Tregs while sparing both peripheral Tregs and effector T cells.

C C motif chemokine receptor 8 (CCR8) was identified as one of the most differentially and specifically expressed receptors on tumor infiltrating Tregs in comparison to peripheral Tregs. CCR8 has 4 natural ligands: CCL1, CCL8, CCL16, and CCL18; with CCL1 binding exclusively to CCR8. Neither genetic knock-out nor functional blockade of CCR8 significantly impacted tumor infiltration, activation, or suppressive capacity of CCR8+ Tregs (Campbell, Joseph R., et al. “Fc-optimized Anti-CCR8 antibody depletes regulatory T cells in human tumor models.” Cancer Research 81.11 (2021): 2983-2994). This suggests that CCR8 plays a redundant role with other chemokine receptors in tumor-homing of activated Tregs. Therefore, depletion of tumor-infiltrating CCR8+ Tregs, rather than blocking the function of CCR8, is key for specific immunotherapy with pan-tumor potential (Whiteside, Sarah K., et al. “CCR8 marks highly suppressive Treg cells within tumours but is dispensable for their accumulation and suppressive function.” Immunology 163.4 (2021): 512-520). TPP-23411 is a novel Treg depleting antibody that, due to the highly tumor specific expression profile of its target CCR8, specifically depletes tumor infiltrating Tregs while sparing both peripheral Tregs and effector T cells. It was first described in U.S. application Ser. No. 17/358,841 filed on Jun. 25, 2021, PCT Appln No. PCT/EP2021/067504, PCT Appln. No. PCT/EP2021/067578, PCT Appln. No. PCT/EP2021/067574, PCT Appln. No. PCT/EP2021/067579 and PCT Appln. No. PCT/EP2021/067580. Each of these documents is incorporated herein in its entirety, in particular for the description of the specific properties of TPP-23411 and the techniques used to analyze these properties.

TPP-23411 is a fully human IgG antibody and was generated with a phage display approach using chemically synthesized peptides comprising the sulfated N-term of human or cynomolgus CCR8 as epitopes. The respective sequences characterizing TPP-23411 are provided as SEQ ID NO:1 to SEQ ID NO:18, see also section “Brief description of the sequence IDs” herein.

TPP-23411 showed highly specific binding to both human and cynomolgus monkey CCR8 expressed by CHO cells, with a respective affinity in the same order of magnitude, e.g. low digit nanomolar range. TPP-23411 does not bind to CCR4, the closest paralogue of CCR8.

TPP-23411 is a low/non-internalizing antibody as demonstrated for human cells expressing endogenous CCR8. It is assumed that this property prolongs the presentation of TPP-23411 to the effector cells and could therefore improve the efficacy of ADCC and ADCP based Treg depletion.

TPP-23411 is characterized by a comparably high clearance rate in cynomolgus monkeys and human, see e.g. Examples 12 or 15 herein.

An antibody having similar properties as TPP-23411 is an antibody
a. characterized by a KD for binding CHO cells transfected with human CCR8 which is in the same order of magnitude as the KD of TPP-23411 for binding CHO cells transfected with human CCR8,
b. wherein the antibody induces ADCC and ADCP
   preferably wherein the antibody binds to human Fc gamma receptor IIIA variant V176 (CD16a) with a dissociation constant (KD) which is in the same order of magnitude as the KD of TPP-23411 for binding human Fc gamma receptor IIIA variant V176 (CD16a) and
   preferably wherein the antibody binds to human Fc gamma RIIA (CD32a) with a dissociation constant (KD) which is in the same order of magnitude as the KD of TPP-23411 for binding human Fc gamma RIIA (CD32a);
   preferably wherein the antibody is afucosylated;
c. preferably wherein the antibody is characterized by a half-life of <14 days, preferably <10 days, most preferably <7 days in human.

TPP-23411 is preferably afucosylated and induces both ADCC and ADCP. In consequence, after binding the Tregs, TPP-23411 recruits the respective effector cells via FC Receptor (FcR) interaction (NK cells for ADCC and macrophages for ADCP), such that these effector cells can deplete the CCR8-expressing Tregs. Indeed, TPP-23411 triggers potent and dose dependent depletion of human primary CCR8+ Tregs or ectopic human CCR8 expressing HEK293 target cells by engaging either human NK92V cells or human primary M2c macrophages as effector cells.

TPP-23411 does not block or neutralize CCL1-induced β3-arrestin signaling.

In pre-clinical experiments it was found that TPP-23411 surrogate antibodies show remarkable efficacy in syngeneic tumor models, either alone or in combination with PD-(L)1 inhibitors.

CCR8 antibodies can be combined with PD-(L)1 inhibitors or other checkpoint inhibitors.

Pembrolizumab (KEYTRUDA) is a potent humanized IgG4 mAb with high specificity of binding to PD-1 receptor, thus inhibiting its interaction with PD-L1 and PD-L2. Based on preclinical in vitro data, pembrolizumab has high affinity and potent receptor blocking activity for PD-1. Pembrolizumab has an acceptable preclinical safety profile and is in clinical development as an intravenous (IV) immunotherapy for advanced malignancies. Pembrolizumab is indicated for the treatment of patients across a number of cancer indications. Dosage forms and strength of pembrolizumab are solutions for injection provided in a single-dose vial with 100 mg/4 mL (25 mg/mL) solution. Pembrolizumab can be administered e.g. at a dose of 200 mg once every three weeks, or 400 mg once every 6 weeks. Therapeutic studies in mouse models have shown that administration of antibodies blocking PD-1/PD-L1 interaction enhances infiltration of tumor-specific CD8+ T cells and ultimately leads to tumor rejection, either as a monotherapy or in combination with other treatment modalities.

Nivolumab (OPDIVO) is another PD-1 blocking antibody indicated for the treatment of patients across a number of cancer indications. Dosage forms and strength are solutions for injection provided in a single-dose vial with 10 mg/mL (4 mL, 10 mL). Nivolumab can be administered by intravenous infusion after dilution, e.g. at a dose of 240 mg every two weeks, 360 mg every 3 weeks or 480 mg every 4 weeks.

Atezolizumab (TECENTRIQ) is a further PD-L1 blocking antibody and is likewise indicated for the treatment of patients across a number of cancer indications. Dosage forms and strength are solutions for injection provided in a single-dose vial with 840 mg/14 mL (60 mg/mL) or 1200 mg/20 mL (60 mg/mL). Atezolizumab can be administered by intravenous infusion after dilution, e.g. at a dose of 840 mg every two weeks, 1200 mg every 3 weeks, or 1680 mg every 4 weeks.

Zimberelimab (Arcus Biosciences) is a monoclonal antibody that binds PD-1 restoring the antitumor activity of T cells. Zimberelimab is in clinical studies for various cancer indications, e.g. for the treatment of first-line metastatic non-small cell lung cancer, e.g. in combination with domvanalimab, an anti-TIGIT monoclonal antibody, and etrumadenant, a dual A2a/A2b adenosine receptor antagonist. Zimberelimab can be administered by intravenous infusion after dilution, e.g. at a dose of 360 mg every 3 weeks.

Toripalimab, a recombinant, humanized PD-1 monoclonal antibody that binds to PD-1 and prevents binding of PD-1 with PD-L1 and PD-L2, is being developed by Shanghai Junshi Bioscience Co., Ltd for the treatment of various cancers. The approved dosage of toripalimab is 3 mg/kg every two weeks as an intravenous (IV) infusion.

Durvalumab (IMFINZI) is a PD-L1 blocking antibody indicated for various cancer types. Dosage forms and strength are solutions for injection provided in a single-dose vial with 500 mg/10 mL or 120 mg/2.4 mL (each 50 mg/mL). Durvalumab can be administered by intravenous infusion after dilution, e.g. at a dose of 10 mg/kg every two weeks or 1500 mg every 3 weeks as part of a combination scheme.

Further PD-(L)1 inhibitors and their dosing regimens (approved or in clinical studies) are known in the art and may be useful in the provided medical uses and treatment methods.

### Technical Problem

Coming up with an appropriate dosing regime is challenging if an antibody cannot be properly tested in the relevant model species. TPP-23411 is cross reactive to cynomolgus monkey but not to mouse CCR8 orthologue. Established models to find an optimal dose regimen could therefore not be applied to find an appropriate solution for the dosing in human patients for monotherapy and combination therapy.

Furthermore, while characterizing TPP-23411, an unusual PK/PD behavior and an increased clearance was observed by the inventors for this antibody in cynomolgus monkeys. Based upon the preliminary translational pharmacokinetic estimations described herein, TPP-23411 is characterized by a half-life of approximately ˜4 days (typical antibodies have a half-life of 21 days). This clearance behavior thus deviated not only from the predicted properties of TPP-23411 but deviated also from the common half-life of other antibodies for medical use and complicated the identification of a safe and efficient dosing scheme for the treatment of patients.

In order to find an appropriate solution for the dosing in human patients, the inventors had to come up with an anti-mouse CCR8 surrogate antibody that could be used to model the short half-life of TPP-23411. This anti-mouse CCR8 surrogate antibody is TPP-29338, which is provided herein.

Furthermore, there was a need to determine and quantify anti-anti-CCR8 antibodies in cynomolgus or human plasma or serum, in order to come up with a suitable dose regimen and for quality control. The medical use of TPP-23411 as provided herein is characterized by a particular dose regimen, which ensures a superior efficacy while fulfilling the necessary safety requirements. The successful mode of action is demonstrated for the inventive dosing regimen in Example 24. Furthermore, the dosing regimen according to the current invention also provides convenience of handling and dosing, thereby reducing dosing errors while improving patient quality of life and compliance.

Furthermore, the administration of a Treg depleting agent may come with substantial side effects or may not be effective in certain patient populations. While testing the dosing regimens according to the current invention, the inventors came up with certain (pre)medication schemes that were found to prevent adverse reactions observed upon intravenous administration of the anti-CCR8 antibody, see Example 23. In order to identify those patients who will likely profit from an anti-CCR8 antibody treatment and in order to identify those patients for whom the potential side-effects are acceptable after benefit risk assessment, stratification steps are provided herein.

Finally, the inventors found that IHC staining to determine the amount of T cells in tumor biopsies as a biomarker for stratification of monitoring can lack robustness, e.g. if the T cell distribution is not normally distributed. Therefore, the inventors suggest herein to apply a specific PET-based method to track the recruitment of T cells after administering an anti-human CCR8 antibody.

### BACKGROUND

Several companies have started or announced their plans to start clinical studies to administer compounds targeting CCR8. Each of the provided dosing regimens deviates from the dosing scheme of the inventive treatment method/medical use described herein at least in that the compound targeting CCR8 deviates from TPP-23411, but also in various other aspects.

Jounce and Gilead have developed the anti-CCR8 antibody JTX-1811/GS-1811 (see WO2021/163064 A1) and Gilead has announced the start of “A Phase 1 Study to Evaluate the Safety and Tolerability of GS-1811, an Afucosylated Anti-CCR8 Monoclonal Antibody, as Monotherapy and in Combination With Pembrolizumab in Adults With Advanced Solid Tumors” (NCT05007782). During dose escalation participants receive escalating dose levels of GS-1811 for up to 12 months to determine maximum tolerated dose (MTD) and/or the recommended phase 2 dose.

Shionogi has developed the anti-CCR8 antibody S-531011 (see WO2020/138489 A1) and has started “A Phase 1b/2, Multicenter, Open-label Study of 5-531011 as Monotherapy and in Combination With an Immune Checkpoint Inhibitor in Participants With Locally Advanced or Metastatic Solid Tumors” (NCT05101070). Participants will receive escalating doses of S-531011 by intravenous infusion for up to approximately 12 months. For the combination arm, participants will receive escalating doses of S-531011 in combination with pembrolizumab by intravenous infusion for up to approximately 12 months.

BMS has developed the anti-CCR8 antibody BMS-986340 (see WO2021/194942 A1) and has started “A Phase 1/2 Study of BMS-986340 as Monotherapy and in Combination With Nivolumab in Participants With Advanced Solid Tumors” in May 2021 (NCT04895709). Primary completion is expected for March 2024. In the dose escalation stage 4A19 is administered intravenously to subjects at a flat dose of 0.3, 1, 3, 10, 30, 100, 300 and 800 mg, once every two weeks (Q2W). In Part IB, 4A19 is administered intravenously (IV) to subjects at the same flat doses in combination with nivolumab administered IV at the FDA-approved flat dose of 480 mg once every 4 weeks (Q4W).

International phase application WO2022/00443 A1, entitled “METHODS AND COMPOSITIONS FOR TARGETING TREGS USING CCR8 INHIBITORS,” was filed 2020 Jul. 3 by Nanjing Immunophage Biotech Co., Ltd and discloses small molecule CCR8 inhibitors blocking the CCR8/CCL1 axis as demonstrated in a Calcium mobilization assay. In Nov. 15, 2021 Nanjing Immunophage has started “A Phase 1 Study to Evaluate the Safety, Tolerability, Pharmacokinetics, and Preliminary Anti-tumor Activity of IPG7236 Administered Orally as a Single Agent in Patients With Advanced Solid Tumors.” (see NCT05142592). The IPG7236 drug product is supplied as oral tablet dosage form, containing two strengths: 25 mg and 100 mg, respectively.

LM-108 is a humanized monoclonal anti-CCR8 antibody that was developed by LaNova Medicines. LaNova Medicines has announced the start of “A Phase I/II, Open-Label, Dose-Escalation and Dose-expansion Clinical Study to Evaluate the Safety, Tolerability, Pharmacokinetics and Preliminary Efficacy of LM-108 as a Single Agent or in Combination With Toripalimab in Advanced Solid Tumours” for August 2022 (NCT05518045).

U.S. application Ser. No. 17/280,137 discloses a Zr-89-labeled anti-CD8 minibody for PET scan but does not disclose the use of this minibody as part of the medical use of an anti-CCR8 antibody, where this specific method is superior to a conventional histopathological approach in order to reliably track the recruitment of T cells as a biomarker after administering an anti-human CCR8 antibody.

Descriptions of anti-drug antibody assays are known in the art, see e.g. EP3105592B1 or Seaman, Michael S., et al. “Optimization and qualification of a functional anti-drug antibody assay for HIV-1 bnAbs.” Journal of immunological methods 479 (2020): 112736, but the inventors are not aware of a specific method for detecting and quantifying anti-anti-CCR8 antibodies.

Various anti-CCR8 mouse surrogate antibodies exist, such as those previously described by the inventors in U.S. application Ser. No. 17/358,841 and PCT Appln Nos. PCT/EP2021/067504, PCT/EP2021/067578, PCT/EP2021/067574, PCT/EP2021/067579 and PCT Appln. No. PCT/EP2021/067580, but the inventors are not aware that any of these anti-CCR8 mouse surrogate antibodies might be suitable to model the fast clearance rate of TPP-23411.

### Solution to Problem

Based on various experimental data (see Examples 1 to 16), the inventors could successfully identify a medical use of an anti-CCR8 antibody comprising a particular administration scheme, which is further described inter alia in Example 17:

Provided is an anti-human CCR8 antibody having ADCC activity and ADCP activity for use in a method of treatment, comprising administering intravenously to a patient in need thereof the anti-CCR8 antibody in a total amount of
a. Approximately 1, 2.5, 3, 10, 30, 50, 100, 125, or 250 mg once every week, or
b. Approximately 16, 450, 500, 750, 1000 or 1500 mg once every three weeks.

Optionally the medical use may further comprise administering intravenously to the patient in need thereof an anti-PD-(L)1 antibody in a total amount of
i. Approximately 200 mg once every three weeks, preferably wherein the anti-PD-(L)1 antibody is pembrolizumab, or
ii. Approximately 400 mg once every six weeks, preferably wherein the anti-PD-(L)1 antibody is pembrolizumab, or
iii. Approximately 240 mg once every two weeks, preferably wherein the anti-PD-(L)1 antibody is nivolumab, or
iv. Approximately 360 mg once every three weeks, preferably wherein the anti-PD-(L)1 antibody is nivolumab, or
v. Approximately 480 mg once every four weeks, preferably wherein the anti-PD-(L)1 antibody is nivolumab, or
vi. Approximately 840 mg every two weeks, preferably wherein the anti-PD-(L)1 antibody is atezolizumab, or
vii. Approximately 1200 mg every three weeks, preferably wherein the anti-PD-(L)1 antibody is atezolizumab, or
viii. Approximately 1680 mg every four weeks, preferably wherein the anti-PD-(L)1 antibody is atezolizumab, or
ix. Approximately 360 mg every three weeks, preferably wherein the anti-PD-(L)1 antibody is Zimberelimab, or
x. Approximately 3 mg/kg every two weeks, preferably wherein the anti-PD-(L)1 antibody is Toripalimab, or
xi. Approximately 10 mg/kg every two weeks, preferably wherein the anti-PD-(L)1 antibody is Durvalumab, or
xii. Approximately 1500 mg every 3 weeks, preferably wherein the anti-PD-(L)1 antibody is Durvalumab.

Provided is also an anti-human CCR8 antibody having ADCC activity and/or ADCP activity for use in a method of treatment
a. comprising administering intravenously to a patient in need thereof the anti-CCR8 antibody in a total amount of 2.7 mg to 75 mg once every week,
b. preferably further comprising administering intravenously to the patient an anti-PD-(L)1 antibody in a total amount of
   i. Approximately 200 mg once every three weeks, preferably wherein the anti-PD-(L)1 antibody is pembrolizumab, or
   ii. Approximately 400 mg once every six weeks, preferably wherein the anti-PD-(L)1 antibody is pembrolizumab, or
   iii. Approximately 240 mg once every two weeks, preferably wherein the anti-PD-(L)1 antibody is nivolumab, or
   iv. Approximately 360 mg once every three weeks, preferably wherein the anti-PD-(L)1 antibody is nivolumab, or
   v. Approximately 480 mg once every four weeks, preferably wherein the anti-PD-(L)1 antibody is nivolumab, or
   vi. Approximately 840 mg every two weeks, preferably wherein the anti-PD-(L)1 antibody is atezolizumab, or
   vii. Approximately 1200 mg every three weeks, preferably wherein the anti-PD-(L)1 antibody is atezolizumab, or
   viii. Approximately 1680 mg every four weeks, preferably wherein the anti-PD-(L)1 antibody is atezolizumab, or
   ix. Approximately 360 mg every three weeks, preferably wherein the anti-PD-(L)1 antibody is Zimberelimab, or
   x. Approximately 3 mg/kg every two weeks, preferably wherein the anti-PD-(L)1 antibody is Toripalimab, or
   xi. Approximately 10 mg/kg every two weeks, preferably wherein the anti-PD-(L)1 antibody is Durvalumab, or
   xii. Approximately 1500 mg every 3 weeks, preferably wherein the anti-PD-(L)1 antibody is Durvalumab.

Provided is furthermore an anti-human CCR8 antibody having ADCC activity and ADCP activity for use in a method of treatment
a. comprising administering intravenously to a patient in need thereof the anti-CCR8 antibody in a total amount of 16 mg to 450 mg once every three weeks,
b. preferably further comprising administering intravenously to the patient an anti-PD-(L)1 antibody in a total amount of
   i. Approximately 200 mg once every three weeks, preferably wherein the anti-PD-(L)1 antibody is pembrolizumab, or
   ii. Approximately 400 mg once every six weeks, preferably wherein the anti-PD-(L)1 antibody is pembrolizumab, or
   iii. Approximately 240 mg once every two weeks, preferably wherein the anti-PD-(L)1 antibody is nivolumab, or
   iv. Approximately 360 mg once every three weeks, preferably wherein the anti-PD-(L)1 antibody is nivolumab, or
   v. Approximately 480 mg once every four weeks, preferably wherein the anti-PD-(L)1 antibody is nivolumab, or
   vi. Approximately 840 mg every two weeks, preferably wherein the anti-PD-(L)1 antibody is atezolizumab, or
   vii. Approximately 1200 mg every three weeks, preferably wherein the anti-PD-(L)1 antibody is atezolizumab, or
   viii. Approximately 1680 mg every four weeks, preferably wherein the anti-PD-(L)1 antibody is atezolizumab, or
   ix. Approximately 360 mg every three weeks, preferably wherein the anti-PD-(L)1 antibody is Zimberelimab, or
   x. Approximately 3 mg/kg every two weeks, preferably wherein the anti-PD-(L)1 antibody is Toripalimab, or
   xi. Approximately 10 mg/kg every two weeks, preferably wherein the anti-PD-(L)1 antibody is Durvalumab, or
   xii. Approximately 1500 mg every 3 weeks, preferably wherein the anti-PD-(L)1 antibody is Durvalumab.

The particular administration schemes provided herein avoid unacceptable adverse effects but maintain a sufficient dose of the anti-CCR8 antibody in the blood for optimal efficacy.

In some embodiments the anti-human CCR8 antibody-based medical uses or methods of treatment comprise a stratification step to select patients with an increased probability of treatment success and/or an improved benefit risk ratio. Suggested biomarkers are
a. Tumor Proportion Score or the Combined Positive Score as a measure for PD-(L)1 expression,
b. Analysing in a blood, plasma or serum sample inflammatory cytokines selected from the group of IFN-γ, IL-1β, IL-2, IL-4, IL-6, IL-8, IL-10, IL 12p70, IL-13, and TNF-α, and
c. Previous treatment of the cancer for at least 6 months with an anti-PD-(L)1 antibody.

Furthermore, according to the present invention there are provided anti-human CCR8 antibody-based medical uses and treatment methods comprising the administration of a Zr-89-labeled anti-CD8 minibody to determine the abundance and/or distribution of CD8 cells by means of a PET scan for stratification or for monitoring treatment success or disease progression.

According to the current invention there is also provided a method to reliably determine an anti-anti-CCR8 antibody in cynomolgus or human plasma using an anti-CCR8 antibody-based bridging ELISA method.

### BRIEF DESCRIPTION OF THE DRAWINGS

FIG. 1: Overview of the dose escalation part of the study schema.

FIG. 2: Dose-normalized plasma concentrations of TPP-23411 after intravenous and subcutaneous administration.

FIG. 3: Predicted human plasma c/t profile after single intravenous infusion of 1 mg/kg TPP-23411 over one hour.

FIG. 4: Simulations of c/t profiles after multiple intravenous infusion of 1 mg/kg TPP-23411 over one hour for QW, Q2W and Q3W administration.

FIG. 5: Correlation between responses to anti-CCR8 antibody treatment and anti-PD-L1 antibody (A) or anti-PD-1 (B) treatment in vivo. (A)-(B) The in vivo efficacy of antibody treatments was assessed in various syngeneic murine carcinoma models. The solid and dashed lines indicate the regression line and associated 95% confidence intervals, respectively.

FIG. 6: Correlation between the response to anti-mouse CCR8 antibody treatment and PD-L1 mRNA expression (A), and IFN-γ mRNA expression (B) in early, untreated tumors. The correlation was assessed between the in vivo efficacy of anti-CCR8 antibody treatments and (A) the baseline PD-L1 expression and (B) the baseline IFN-γ expression, across various syngeneic murine carcinoma models. The solid and dashed lines indicate the regression line and associated 95% confidence intervals, respectively.

FIG. 7: Increase of activated proliferating CD8+ T cells relative to the total number of CD3+ T cells starting ˜3 days after the first administration of 10 mg of anti-CCR8 antibody to a human patient. A continuous increase was observed in particular starting from the second week of treatment, presumably by induction of immune activation in the tumor. End of treatment is ˜3 weeks after the last infusion. The ratio of the two cell types at the timepoint of screening was set to 1.

FIG. 8: Decrease of the ratio of activated Tregs relative to the total number of CD3+ T cells starting ˜1 day after the first anti-CCR8 antibody administration (dosis of 10 mg). The ratio of the two cell types at the timepoint of screening was set to 1.

FIG. 9: Ratio of activated Tregs relative to the number of activated proliferating CD8+ T cells. The ratio of the two cell types at the timepoint of screening was set to 1.

FIG. 10: Reduction of Tregs in patient blood samples upon treatment with 1 mg, 3 mg, 10 mg or 30 mg of anti-CCR8 antibody. For each patient the ratios (activated Tregs/total CD3+ T cells) obtained for the timepoints C1D2 to C2D15 were divided by the ratios (activated Tregs/total CD3+ T cells) obtained upon screening (Scr) for the same patient, i.e. before administration of anti-CCR8 antibody. To obtain the data for one box, all patients treated at the indicated dose were grouped together. Even the lowest 1 mg cohort shows a median reduction of the activated Tregs to ˜53% of the screening value.

FIG. 11: Boxplots for TNF-alpha levels measured in pg/μl in blood or serum samples that were collected 4 hours after administration of 1 mg, 3 mg, 10 mg or 30 mg anti-CCR8 antibody to human patients.

FIG. 12: Boxplots for IFN-gamma levels measured in pg/μl in blood or serum samples that were collected 4 hours after administration of 1 mg, 3 mg, 10 mg or 30 mg anti-CCR8 antibody from human patients.

FIG. 13: Boxplots for IP10 (CXCL10) levels measured in pg/μl in blood or serum samples that were collected 4 hours after administration of 1 mg, 3 mg, 10 mg or 30 mg anti-CCR8 antibody to human patients.

FIG. 14: Boxplots for IL8 levels measured in pg/μl in blood or serum samples that were collected 4 hours after administration of 1 mg, 3 mg, 10 mg or 30 mg anti-CCR8 antibody to human patients.

FIG. 15: Boxplots for IL6 levels measured in pg/μl in blood or serum samples that were collected 4 hours after administration of 1 mg, 3 mg, 10 mg or 30 mg anti-CCR8 antibody to human patients.

FIG. 16: Boxplots for IL10 levels measured in pg/μl in blood or serum samples that were collected 4 hours after administration of 1 mg, 3 mg, 10 mg or 30 mg anti-CCR8 antibody to human patients.

### BRIEF DESCRIPTION OF THE SEQUENCE IDS

The Sequence Listing associated with this application is hereby incorporated by reference into the specification in its entirety. The name of the text file containing the Sequence Listing is BHC221019_WO_ST26_20230903.xml. The size of the text file is 92 kilobytes, and the text file was created on 03.09.2023.

| Int. | | | | |
|---|---|---|---|---|
| Compound | | Sequence | Seq | |
| Reference | Sequence Name | Region | Type | SEQ ID |
| TPP-23411 | 21360-hIgG1wtlambda | VH | PRT | SEQ ID NO: 1 |
| TPP-23411 | 21360-hIgG1wtlambda | HCDR1 | PRT | SEQ ID NO: 2 |
| TPP-23411 | 21360-hIgG1wtlambda | HCDR2 | PRT | SEQ ID NO: 3 |
| TPP-23411 | 21360-hIgG1wtlambda | HCDR3 | PRT | SEQ ID NO: 4 |
| TPP-23411 | 21360-hIgG1wtlambda | VL | PRT | SEQ ID NO: 5 |
| TPP-23411 | 21360-hIgG1wtlambda | LCDR1 | PRT | SEQ ID NO: 6 |
| TPP-23411 | 21360-hIgG1wtlambda | LCDR2 | PRT | SEQ ID NO: 7 |
| TPP-23411 | 21360-hIgG1wtlambda | LCDR3 | PRT | SEQ ID NO: 8 |
| TPP-23411 | 21360-hIgG1wtlambda | VH | DNA | SEQ ID NO: 9 |
| TPP-23411 | 21360-hIgG1wtlambda | HCDR1 | DNA | SEQ ID NO: 10 |
| TPP-23411 | 21360-hIgG1wtlambda | HCDR2 | DNA | SEQ ID NO: 11 |
| TPP-23411 | 21360-hIgG1wtlambda | HCDR3 | DNA | SEQ ID NO: 12 |
| TPP-23411 | 21360-hIgG1wtlambda | VL | DNA | SEQ ID NO: 13 |
| TPP-23411 | 21360-hIgG1wtlambda | LCDR1 | DNA | SEQ ID NO: 14 |
| TPP-23411 | 21360-hIgG1wtlambda | LCDR2 | DNA | SEQ ID NO: 15 |
| TPP-23411 | 21360-hIgG1wtlambda | LCDR3 | DNA | SEQ ID NO: 16 |
| TPP-23411 | 21360-hIgG1wtlambda | HC | PRT | SEQ ID NO: 17 |
| TPP-23411 | 21360-hIgG1wtlambda | LC | PRT | SEQ ID NO: 18 |
| TPP-29338 | 15285-mIgG2a-HQ-310/HN-330-Lambda | VH | PRT | SEQ ID NO: 19 |
| TPP-29338 | 15285-mIgG2a-HQ-310/HN-330-Lambda | HCDR1 | PRT | SEQ ID NO: 20 |
| TPP-29338 | 15285-mIgG2a-HQ-310/HN-330-Lambda | HCDR2 | PRT | SEQ ID NO: 21 |
| TPP-29338 | 15285-mIgG2a-HQ-310/HN-330-Lambda | HCDR3 | PRT | SEQ ID NO: 22 |
| TPP-29338 | 15285-mIgG2a-HQ-310/HN-330-Lambda | VL | PRT | SEQ ID NO: 23 |
| TPP-29338 | 15285-mIgG2a-HQ-310/HN-330-Lambda | LCDR1 | PRT | SEQ ID NO: 24 |
| TPP-29338 | 15285-mIgG2a-HQ-310/HN-330-Lambda | LCDR2 | PRT | SEQ ID NO: 25 |
| TPP-29338 | 15285-mIgG2a-HQ-310/HN-330-Lambda | LCDR3 | PRT | SEQ ID NO: 26 |
| TPP-29338 | 15285-mIgG2a-HQ-310/HN-330-Lambda | VH | DNA | SEQ ID NO: 27 |
| TPP-29338 | 15285-mIgG2a-HQ-310/HN-330-Lambda | HCDR1 | DNA | SEQ ID NO: 28 |
| TPP-29338 | 15285-mIgG2a-HQ-310/HN-330-Lambda | HCDR2 | DNA | SEQ ID NO: 29 |
| TPP-29338 | 15285-mIgG2a-HQ-310/HN-330-Lambda | HCDR3 | DNA | SEQ ID NO: 30 |
| TPP-29338 | 15285-mIgG2a-HQ-310/HN-330-Lambda | VL | DNA | SEQ ID NO: 31 |
| TPP-29338 | 15285-mIgG2a-HQ-310/HN-330-Lambda | LCDR1 | DNA | SEQ ID NO: 32 |
| TPP-29338 | 15285-mIgG2a-HQ-310/HN-330-Lambda | LCDR2 | DNA | SEQ ID NO: 33 |
| TPP-29338 | 15285-mIgG2a-HQ-310/HN-330-Lambda | LCDR3 | DNA | SEQ ID NO: 34 |
| TPP-29338 | 15285-mIgG2a-HQ-310/HN-330-Lambda | HC | PRT | SEQ ID NO: 35 |
| TPP-29338 | 15285-mIgG2a-HQ-310/HN-330-Lambda | LC | PRT | SEQ ID NO: 36 |
| TPP-27454 | novel-antiCCR8-AB-seqID41, 59-hIgG1Kappa | VH | PRT | SEQ ID NO: 37 |
| TPP-27454 | novel-antiCCR8-AB-seqID41, 59-hIgG1Kappa | HCDR1 | PRT | SEQ ID NO: 38 |
| TPP-27454 | novel-antiCCR8-AB-seqID41, 59-hIgG1Kappa | HCDR2 | PRT | SEQ ID NO: 39 |
| TPP-27454 | novel-antiCCR8-AB-seqID41, 59-hIgG1Kappa | HCDR3 | PRT | SEQ ID NO: 40 |
| TPP-27454 | novel-antiCCR8-AB-seqID41, 59-hIgG1Kappa | VL | PRT | SEQ ID NO: 41 |
| TPP-27454 | novel-antiCCR8-AB-seqID41, 59-hIgG1Kappa | LCDR1 | PRT | SEQ ID NO: 42 |
| TPP-27454 | novel-antiCCR8-AB-seqID41, 59-hIgG1Kappa | LCDR2 | PRT | SEQ ID NO: 43 |
| TPP-27454 | novel-antiCCR8-AB-seqID41, 59-hIgG1Kappa | LCDR3 | PRT | SEQ ID NO: 44 |
| TPP-27454 | novel-antiCCR8-AB-seqID41, 59-hIgG1Kappa | VH | DNA | SEQ ID NO: 45 |
| TPP-27454 | novel-antiCCR8-AB-seqID41, 59-hIgG1Kappa | VL | DNA | SEQ ID NO: 46 |
| TPP-27454 | novel-antiCCR8-AB-seqID41, 59-hIgG1Kappa | Heavy Chain | PRT | SEQ ID NO: 47 |
| TPP-27454 | novel-antiCCR8-AB-seqID41, 59-hIgG1Kappa | Light Chain | PRT | SEQ ID NO: 48 |
| TPP-31741 | Surface-Oncology-CCR8-1-hIgG1Lambda | VH | PRT | SEQ ID NO: 49 |
| TPP-31741 | Surface-Oncology-CCR8-1-hIgG1Lambda | HCDR1 | PRT | SEQ ID NO: 50 |
| TPP-31741 | Surface-Oncology-CCR8-1-hIgG1Lambda | HCDR2 | PRT | SEQ ID NO: 51 |
| TPP-31741 | Surface-Oncology-CCR8-1-hIgG1Lambda | HCDR3 | PRT | SEQ ID NO: 52 |
| TPP-31741 | Surface-Oncology-CCR8-1-hIgG1Lambda | VL | PRT | SEQ ID NO: 53 |
| TPP-31741 | Surface-Oncology-CCR8-1-hIgG1Lambda | LCDR1 | PRT | SEQ ID NO: 54 |
| TPP-31741 | Surface-Oncology-CCR8-1-hIgG1Lambda | LCDR2 | PRT | SEQ ID NO: 55 |
| TPP-31741 | Surface-Oncology-CCR8-1-hIgG1Lambda | LCDR3 | PRT | SEQ ID NO: 56 |
| TPP-31741 | Surface-Oncology-CCR8-1-hIgG1Lambda | VH | DNA | SEQ ID NO: 57 |
| TPP-31741 | Surface-Oncology-CCR8-1-hIgG1Lambda | VL | DNA | SEQ ID NO: 58 |
| TPP-31741 | Surface-Oncology-CCR8-1-hIgG1Lambda | Heavy Chain | PRT | SEQ ID NO: 59 |
| TPP-31741 | Surface-Oncology-CCR8-1-hIgG1Lambda | Light Chain | PRT | SEQ ID NO: 60 |
| TPP-31742 | Surface-Oncology-CCR8-2-hIgG1Lambda | VH | PRT | SEQ ID NO: 61 |
| TPP-31742 | Surface-Oncology-CCR8-2-hIgG1Lambda | HCDR1 | PRT | SEQ ID NO: 62 |
| TPP-31742 | Surface-Oncology-CCR8-2-hIgG1Lambda | HCDR2 | PRT | SEQ ID NO: 63 |
| TPP-31742 | Surface-Oncology-CCR8-2-hIgG1Lambda | HCDR3 | PRT | SEQ ID NO: 64 |
| TPP-31742 | Surface-Oncology-CCR8-2-hIgG1Lambda | VL | PRT | SEQ ID NO: 65 |
| TPP-31742 | Surface-Oncology-CCR8-2-hIgG1Lambda | LCDR1 | PRT | SEQ ID NO: 66 |
| TPP-31742 | Surface-Oncology-CCR8-2-hIgG1Lambda | LCDR2 | PRT | SEQ ID NO: 67 |
| TPP-31742 | Surface-Oncology-CCR8-2-hIgG1Lambda | LCDR3 | PRT | SEQ ID NO: 68 |
| TPP-31742 | Surface-Oncology-CCR8-2-hIgG1Lambda | VH | DNA | SEQ ID NO: 69 |
| TPP-31742 | Surface-Oncology-CCR8-2-hIgG1Lambda | VL | DNA | SEQ ID NO: 70 |
| TPP-31742 | Surface-Oncology-CCR8-2-hIgG1Lambda | Heavy Chain | PRT | SEQ ID NO: 71 |
| TPP-31742 | Surface-Oncology-CCR8-2-hIgG1Lambda | Light Chain | PRT | SEQ ID NO: 72 |
| TPP-31743 | BMS-4A19-hIgG1Kappa | VH | PRT | SEQ ID NO: 73 |
| TPP-31743 | BMS-4A19-hIgG1Kappa | HCDR1 | PRT | SEQ ID NO: 74 |
| TPP-31743 | BMS-4A19-hIgG1Kappa | HCDR2 | PRT | SEQ ID NO: 75 |
| TPP-31743 | BMS-4A19-hIgG1Kappa | HCDR3 | PRT | SEQ ID NO: 76 |
| TPP-31743 | BMS-4A19-hIgG1Kappa | VL | PRT | SEQ ID NO: 77 |
| TPP-31743 | BMS-4A19-hIgG1Kappa | LCDR1 | PRT | SEQ ID NO: 78 |
| TPP-31743 | BMS-4A19-hIgG1Kappa | LCDR2 | PRT | SEQ ID NO: 79 |
| TPP-31743 | BMS-4A19-hIgG1Kappa | LCDR3 | PRT | SEQ ID NO: 80 |
| TPP-31743 | BMS-4A19-hIgG1Kappa | VH | DNA | SEQ ID NO: 81 |
| TPP-31743 | BMS-4A19-hIgG1Kappa | VL | DNA | SEQ ID NO: 82 |
| TPP-31743 | BMS-4A19-hIgG1Kappa | Heavy Chain | PRT | SEQ ID NO: 83 |
| TPP-31743 | BMS-4A19-hIgG1Kappa | Light Chain | PRT | SEQ ID NO: 84 |
| TPP-31744 | Jounce-7-B16.001S83-hIgG1Kappa | VH | PRT | SEQ ID NO: 85 |
| TPP-31744 | Jounce-7-B16.001S83-hIgG1Kappa | HCDR1 | PRT | SEQ ID NO: 86 |
| TPP-31744 | Jounce-7-B16.001S83-hIgG1Kappa | HCDR2 | PRT | SEQ ID NO: 87 |
| TPP-31744 | Jounce-7-B16.001S83-hIgG1Kappa | HCDR3 | PRT | SEQ ID NO: 88 |
| TPP-31744 | Jounce-7-B16.001S83-hIgG1Kappa | VL | PRT | SEQ ID NO: 89 |
| TPP-31744 | Jounce-7-B16.001S83-hIgG1Kappa | LCDR1 | PRT | SEQ ID NO: 90 |
| TPP-31744 | Jounce-7-B16.001S83-hIgG1Kappa | LCDR2 | PRT | SEQ ID NO: 91 |
| TPP-31744 | Jounce-7-B16.001S83-hIgG1Kappa | LCDR3 | PRT | SEQ ID NO: 92 |
| TPP-31744 | Jounce-7-B16.001S83-hIgG1Kappa | VH | DNA | SEQ ID NO: 93 |
| TPP-31744 | Jounce-7-B16.001S83-hIgG1Kappa | VL | DNA | SEQ ID NO: 94 |
| TPP-31744 | Jounce-7-B16.001S83-hIgG1Kappa | Heavy Chain | PRT | SEQ ID NO: 95 |
| TPP-31744 | Jounce-7-B16.001S83-hIgG1Kappa | Light Chain | PRT | SEQ ID NO: 96 |

### Definitions

Unless otherwise defined, all scientific and technical terms used in the description, figures and claims have their ordinary meaning as commonly understood by one of ordinary skill in the art. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. Where reference to a database is made, the effective data shall be the version number applicable 06.05.2022, if not indicated otherwise. The materials, methods, and examples are illustrative only and are not intended to be limiting. Unless stated otherwise, the following terms used in this document, including the description and claims, have the definitions given below.

The expression “about” or “˜” as used herein refers to a value being within an acceptable error range for the particular value as determined by one of ordinary skill in the art, which will depend in part on how the value is measured or determined, i.e., on the limitations of the measurement system. For example, “about” can mean within 1 or more than 1 standard deviation, per the practice in the art.

The term “about” is also used to indicate that the amount or value in question may be the value designated or some other value that is approximately the same. The phrase is intended to convey that similar values promote equivalent results or effects as described herein. In this context “about” may refer to a range above and/or below of up to 10%. Wherever the term “about” is specified for a certain assay or embodiment, that definition prevails for the particular context.

If not defined otherwise, the term “approximately” means the provided value +/−10%.

The terms “comprising”, “including”, “containing”, “having” etc. shall be read expansively or open-ended and without limitation. The term comprising when used in the specification includes “consisting of”.

Singular forms such as “a”, “an” or “the” include plural references unless the context clearly indicates otherwise. Thus, for example, reference to “a monoclonal antibody” includes a single monoclonal antibody as well as a plurality of monoclonal antibodies, either the same or different. Likewise reference to “cell” includes a single cell as well as a plurality of cells.

Unless otherwise indicated, the term “at least” preceding a series of elements is to be understood to refer to every element in the series. The terms “at least one” and “at least one of” include for example, one, two, three, four, five or more elements.

It is furthermore understood that slight variations above and below a stated range can be used to achieve substantially the same results as a value within the range. Also, unless indicated otherwise, the disclosure of ranges is intended as a continuous range including every value between the minimum and maximum values.

The term “amino acid” or “amino acid residue” as used herein typically refers to a naturally-occurring amino acid. The one letter code is used herein to refer to the respective amino acid. As used herein, a “charged amino acid” is an amino acid which is negatively charged or positively charged. “Negatively charged amino acids” are aspartic acid (D) and glutamic acid (E). “Positively charged amino acids” are arginine (R) lysine (K) and histidine (H). “Polar amino acids” are all amino acids that form hydrogen bonds as donors or acceptors. These are all charged amino acids and asparagine (N), glutamine (Q), serine (S), threonine (T), tyrosine (Y) and cysteine (C). “Polar uncharged amino acids” are asparagine (N), glutamine (Q), serine (S), threonine (T), tyrosine (Y) and cysteine (C). “Amphiphatic amino acids” are tryptophan (W), tyrosine (Y) and methionine (M). “Aromatic amino acids” are phenylalanine (F), tyrosine (Y), and tryptophan (W). “Hydrophobic amino acids” are glycine (G), alanine (A), valine (V), leucine (L), isoleucine (I), proline (P), phenylalanine (F), methionine (M) and cysteine. “Small amino acids” are glycine (G), alanine (A), serine (S), proline (P), threonine (T), aspartic acid (D) and asparagine (N).

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

Where generic reference is made to a gene or protein from a certain species such as mouse, the analogue from human shall likewise be meant, if not stated otherwise or obviously incompatible.

This holds in particular in the context of biomarkers.

The term “isolated” when applied to a nucleic acid, polypeptide, protein or antibody, denotes that the nucleic acid, polypeptide, protein or antibody is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high-performance liquid chromatography. A protein, polypeptide or antibody that is the predominant species present in a preparation is substantially purified. In particular, an isolated gene is separated from open reading frames that flank the gene and encode a protein other than the gene of interest. An isolated polypeptide may however be immobilized, e.g. on beads or particles, e.g. via a suitable linker.

The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

As used herein, the term “synthetic”, with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

A “sulfation” is a posttranslational modification where a sulfate group is added to an amino acid such as a tyrosine residue of a polypeptide or protein. Tyrosine sulfation occurs in all multicellular organisms. Under physiological conditions it is catalyzed by tyrosylprotein sulfotransferases (TPSTs) 1 and 2, Golgi-resident enzymes which transfer sulfate from the cofactor PAPS (3′-phosphoadenosine 5′-phosphosulfate) to a context-dependent tyrosine in a protein substrate. Synthetic sulfation of tyrosine may be performed with a technique known in the art, e.g. as described in Bunschoten, Anton, et al. “A general sequence independent solid phase method for the site specific synthesis of multiple sulfated-tyrosine containing peptides.” Chemical Communications 21 (2009): 2999-3001. A sulfated polypeptide is a polypeptide comprising at least one sulfation. A non-sulfated polypeptide is a polypeptide comprising no sulfation.

The term “N terminus” or “N term” of a chemokine receptor as used herein refers to the N terminal amino acids of the chemokine receptor comprising at least the TRD. Where a polypeptide or protein comprises a signal peptide, the N terminus may also refer to the N terminal sequence behind the natural cleavage site of the polypeptide or protein. According to some preferred embodiments, the N terminus comprises the LID domain and the TRD domain of a chemokine receptor but does not comprise the natural cysteine between these two domains. Instead, the cysteine can be removed or can be replaced by a different amino acid.

“Sequence identity” or “percent identity” is a number that describes how similar a query sequence is to a target sequence, more precisely how many characters in each sequence are identical after alignment. The most popular tool to calculate sequence identity is BLAST (basic local alignment search tool, https://blast.ncbi.nlm.nih.gov/), which performs comparisons between pairs of sequences, searching for regions of local similarity. Suitable alignment methods are known in the art, e.g. Needleman-Wunsch algorithm for global-global alignment, using BLOSUM62 matrix, with gap opening penalty of 11 and a gap extension penalty of 1. Afterwards, the pairs of aligned identical residues can be counted and then divided by the total length of the alignment (including gaps, internal as well as external) to arrive at the percent identity value.

For “percent similarity” or “sequence similarity” values, the same approach as for percent identity values can be used, except that what is counted, instead of pairs of identical residues, is the aligned residue pairs with BLOSUM62 values that are not negative (i.e., ≥0).

“CC chemokine receptors” (CCR, also beta chemokine receptors) are integral membrane proteins that specifically bind and respond to cytokines of the CC chemokine family. They represent one subfamily of chemokine receptors, a large family of G protein-linked receptors that are known as seven transmembrane (7-TM) proteins since they span the cell membrane seven times. The subfamily of the CC chemokine receptors comprises CCR1, CCR2, CCR3, CCR4, CCR5, CCR6, CCR7, CCR8, CCR9 and CCR10.

The term “CCR8” refers to the C-C chemokine receptor type 8. The CCR8 protein is encoded by the gene CCR8 (NCBI gene ID 1237). Synonyms for CCR8 are inter alia CC-CKR-8, CCR-8, CDw198, CKRL1, CMKBR8, CMKBRL2, GPRCY6, CY6, TER1. The CCR8 protein comprises human, murine, rat, rhesus macaque and further mammalian and non-mammalian homologues. Sequence(s) for human CCR8 are accessible via UniProt Identifier P51685 (CCR8_HUMAN), for instance human isoform P51685-1 or P51685-2 (UniProt, Nov. 29, 2019). Sequence(s) for murine CCR8 are accessible via UniProt Identifier P56484 (CCR8_MOUSE). Sequence(s) for Rhesus macaque CCR8 are accessible via UniProt Identifier O97665 (CCR8_MACMU). Different isoforms and variants may exist for the different species and are all comprised by the term CCR8. Also comprised are CCR8 molecules before and after maturation, i.e., independent of cleavage of one or more pro-domains. In addition, synthetic variants of the CCR8 protein may be generated and are comprised by the term CCR8. The protein CCR8 may furthermore be subject to various modifications, e.g, synthetic or naturally occurring modifications, such as post translational modifications. Recombinant human CCR8 is commercially available or can be manufactured as known in the art. CCR8 is a receptor for the chemokine CCL1/SCYA1/I-309. Barington et al. have reported the importance of conserved extracellular disulfide bridges and aromatic residues in extracellular loop 2 (ECL-2) for ligand binding and activation in the chemokine receptor CCR8 (Barington, Line, et al. “Role of conserved disulfide bridges and aromatic residues in extracellular loop 2 of chemokine receptor CCR8 for chemokine and small molecule binding.” Journal of Biological Chemistry 291.31 (2016): 16208-16220.). Furthermore, they found that two distinct aromatic residues in ECL-2, Tyr184 (Cys+1) and Tyr187 (Cys+4), were crucial for binding of the CC chemokines CCL1 (agonist) and MC148 (antagonist), respectively, but not for small molecule binding.

“Programmed Death-1 (PD-1)” refers to an immunoinhibitory receptor belonging to the CD28 family. PD-1 is expressed predominantly on previously activated T cells in vivo and binds to two ligands, PD-L1 and PD-L2. The term “PD-1” as used herein includes without limitation human PD-1 (hPD-1), variants, isoforms, and species homologs of hPD-1, and analogs having at least one common epitope with hPD-1. The complete hPD-1 sequence can be found under GenBank Accession No. U64863 (Nov. 29, 2019).

“Programmed Death Ligand-1 (PD-L1)” is one of two cell surface glycoprotein ligands for PD-1 (the other being PD-L2) that down regulate T cell activation and cytokine secretion upon binding to PD-1. The term “PD-L1” as used herein includes without limitation human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7 (Nov. 29, 2019).

The term “PD-(L)1” refers to PD-1 and/or PD-L1.

“Tumor Proportion Score” (TPS) is the percentage of viable tumor cells showing partial or complete membrane staining at any intensity. For example, a specimen should be considered to have PD-L1 expression if TPS 1% and high PD-L1 expression if TPS 50%. For example, PD-L1 protein expression in NSCLC is usually determined by using Tumor Proportion Score (TPS).

A “historic Tumor Proportion Score” is a Tumor Proportion Score that has been obtained in the preparation or during monitoring of a previous (cancer) therapy or medical analysis, i.e. not using a sample obtained from a fresh biopsy in the preparation of an anti-CCR8 antibody therapy.

“Combined Positive Score” (CPS) is the number of staining cells (tumor cells, lymphocytes, macrophages) divided by the total number of viable tumor cells, multiplied by 100. For example, a specimen should be considered to have PD-L1 expression if CPS≥1 and to have high PD-L1 expression if CPS≥10. The FDA has approved the use of the PD-L1 IHC 22C3 pharmDx assay and the use of the VENTANA PD-L1 (SP263) Assay to determine a patient's eligibility for therapeutic antibody pembrolizumab.

A “historic Combined Positive Score” is a Combined Positive Score that has been obtained in the preparation or during monitoring of a previous (cancer) therapy or medical analysis, i.e. not using a sample obtained from a fresh biopsy in the preparation of an anti-CCR8 antibody therapy.

The “PD-L1 IHC 22C3 pharmDx assay” is a qualitative immunohistochemical assay using monoclonal mouse anti-PD-L1, Clone 22C3 intended for use in the detection of PD-L1 protein in formalin-fixed, paraffin-embedded (FFPE) non-small cell lung cancer (NSCLC) tissue using EnVision FLEX visualization system on Autostainer Link 48 (see e.g. https://www.accessdata.fda.gov/cdrh_docs/pdf15/p150013s001c.pdf).

The “VENTANA PD-L1 (SP263) Assay” is another qualitative immunohistochemical assay using rabbit monoclonal anti-PD-L1, Clone SP142 and can be used in FFPE tissues stained e.g. with OptiView DAB IHC Detection Kit and OptiView Amplification Kit on a BenchMark ULTRA instrument (see e.g. https://www.accessdata.fda.gov/cdrh_docs/pdf16/p160046c.pdf).

The term “modulation” refers to any alteration of an existing process or behavior, such as blocking (antagonism) and induction (agonism). For example, modulation of G protein independent signaling refers to any significant alteration of G-protein independent signaling.

The term “internalization” of an antibody, fragment or conjugate refers to the uptake of the antibody, fragment or conjugate into a cell. Preferably, internalization is determined for a cell line with endogenous target expression, e.g. for human or murine CCR8. Preferably, internalization is determined by measuring total internalized fluorescence intensity per cell and is quantified relative to an isotype control. In brief, the antibody, fragment or conjugate and a matching isotype control are labeled with a dye and internalized fluorescence is determined and quantified for the antibody, fragment or conjugate relative to the isotype control.

A “non-internalizing antibody” is defined as an antibody showing substantially the same internalization as a corresponding isotype control.

A “low internalizing antibody” is defined as an antibody showing an internalization which is equal to or lower than the 10-fold of the internalization of the isotype control, preferably lower than the 9-, 8-, 7-, 6-, 5-, 4-, 3-, 2-, 1.5-, 1.4-, 1.3-, 1.2-, or 1.1-fold of the internalization of the isotype control.

An “isotype control” is an antibody or fragment that does not bind a target but has the same class and type as the reference antibody or fragment recognizing the target.

An antibody or fragment is termed “cross-reactive” or “cross reactive” if the antibody or fragment binds an antigen from two or more different species, e.g. with a KD value of 10−7 M or less, more preferably of less than 10−8 M, even more preferably in the range from 10−9 M to 10−11 M.

By the term “specifically binds” as used herein with respect to an antibody, is meant an antibody which recognizes a specific antigen, but does not substantially recognize or bind other molecules in a sample: An antibody characterized by substantial unspecific binding would lack therapeutic applicability, such that these embodiments are excluded. However, as known in the art, specific binding of an antibody or binder does not necessarily exclude an antibody or binder binding to further antigens/target molecules. An antibody that specifically binds to an antigen from one species may also bind to that antigen from one or more further species. Such cross-species reactivity does not itself alter the classification of an antibody as specific.

In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In case of doubt, specific binding of an antibody or binder preferably describes binding of an antibody, antibody fragment or binder to its antigen/target with an affinity of at least 10⁻⁷ M (as KD value; i.e. preferably those with KD values smaller than 10⁻⁷ M), with the antibody or binder having an at least two times lower affinity for a non-specific antigen which is not the predetermined antigen/target molecule or a closely related antigen/target molecule.

The term “affinity” is a term of the art and describes the strength of binding between a binder, antibody or antibody fragment and a target. The “affinity” of antibodies and fragments thereof for a target can be determined using techniques well known in the art or described herein, for example by ELISA, isothermal titration calorimetry (ITC), surface plasmon resonance (SPR), flow cytometry or fluorescent polarization assays. Preferably the affinity is provided as dissociation constant KD.

The “dissociation constant” (KD) has molar units (M) and corresponds to the concentration of the binder/antibody at which half of the target proteins are occupied at equilibrium. The smaller the dissociation constant is, the higher is the affinity between the binder or antibody and its target. According to the current invention, the antibodies preferably have a target affinity of at least 10⁻⁷ M (as KD value), more preferably of at least 10⁻⁸ M, even more preferably in the range from 10⁻⁹ M to 10⁻¹¹ M. The KD values can be preferably determined by means of surface plasmon resonance spectroscopy, e.g. as described elsewhere herein. Where assay conditions were found to influence the determined KD, the assay setup with the least standard deviation shall be used.

“Half maximal effective concentration” (EC50) refers to the concentration of a drug, antibody, fragment, conjugate or molecule which induces a response halfway between the baseline and maximum after a specified incubation time. In the context of antibody binding, the EC50 thus reflects the antibody concentration needed for half-maximal binding. An EC50 can be determined if an inflection point can be determined by mathematical modeling (e.g., non-linear regression) of the dose-response curve describing the relationship between applied drug, antibody, fragment, conjugate or molecule concentration and signal. For example, if the dose-response curve follows a sigmoidal curve, an EC50 can be determined. Where the response is an inhibition, the EC50 is termed half maximal inhibitory concentration (IC50). EC80 can be determined mutatis mutandis.

The (effective) “half-life” of an antibody is the time it takes from its maximum concentration (Cmax) to half of its maximum concentration in the blood plasma. On average, the serum half-life of IgG subclasses (IgG1, IgG2, and IgG4) is ˜23 days as compared to 2-6 days for IgG3 and other Ig classes. Various methods are known in the art to analyze the half-life of an antibody, for example mass spectrometric methods or ELISA-based approaches.

The term “antibody” (Ab) refers to an immunoglobulin molecule (e.g. without limitation human IgG1, IgG2, IgG3, IgG4, IgM, IgD, IgE, IgA1, IgA2, mouse IgG1, IgG2a, IgG2b, IgG2c, IgG3, IgA, IgD, IgE or IgM, rat IgG1, IgG2a, IgG2b, IgG2c, IgA, IgD, IgE or IgM, rabbit IgA1, IgA2, IgA3, IgE, IgG, IgM, goat IgA, IgE, IgG1, IgG2, IgE, IgM or chicken IgY) that specifically binds to, or is immunologically reactive with, a particular antigen. Antibodies or antibody fragments comprise complementarity determining regions (CDRs), also known as hypervariable regions, in both the light chain and heavy chain variable domains. The more highly conserved portions of the variable domains are called the framework (FR). As is known in the art, the amino acid position/boundary delineating a hypervariable region of an antibody can vary, depending on the context and the various definitions known in the art. As used herein, numbering of immunoglobulin amino acid residues is done according to the immunoglobulin amino acid residue numbering system of Kabat et al., The variable domains of native heavy and light chains each comprise four FR regions. The three CDRs in each chain are held together in close proximity by the FR regions and, with the CDRs from the other chain, contribute to the formation of the antigen binding site of antibodies, see Kabat, E. A., et al. “Sequences of Proteins of Immunological Interest (Natl. Inst. Health, Bethesda, MD), GPO Publ.” No 165-462 (1987). The term antibody as used herein also refers to antibody fragments, except where explicitly stated otherwise. Depending on the respective context, the term antibody may also refer to any proteinaceous binding molecule with immunoglobulin-like function.

The term “CDR” refers to the complementary determining region of the antibody. As known in the art complementarity-determining regions (CDRs) are part of the variable chains in antibodies and T cell receptors. A set of CDRs constitutes a paratope. CDRs are crucial to the diversity of antigen specificities. There are three CDRs (CDR1, CDR2 and CDR3), arranged non-consecutively on the amino acid sequence of a variable domain of an antigen receptor. Since the antigen receptors are typically composed of two variable domains (on two different polypeptide chains, heavy and light chain), there are usually six CDRs for each antigen receptor that can collectively come into contact with the antigen. The CDRs of the light chain are LCDR1, LCDR2 and LCDR3. The CDRs of the heavy chain are termed HCDR1, HCDR2 and HCDR3. HCDR3 is the most variable complementary determining region (see, e.g., Chothia, Cyrus, and Arthur M. Lesk. “Canonical structures for the hypervariable regions of immunoglobulins.” Journal of molecular biology 196.4 (1987): 901-917.; Kabat, E. A., et al. “Sequences of proteins of immunological interest. Bethesda, MD: US Department of Health and Human Services.” Public Health Service, National Institutes of Health (1991): 103-511.).

The “constant region” refers to the portion of the antibody molecule that confers effector functions. The heavy chain constant region can be selected from any of the five isotypes: alpha (α), delta (δ), epsilon (ε), gamma (g), or mu (μ).

The term “Fc domain”, “Fc region” or “Fc part” as used herein refers to a C-terminal region of an antibody heavy chain that contains at least a portion of the constant region. The term includes native sequence Fc regions and variant Fc regions. For example, a human IgG heavy chain Fc region may extend from Cys226, or from Pro230, to the carboxyl-terminus of the heavy chain.

Antibodies or binding fragments according to the current invention may have been modified to alter at least one constant region-mediated biological effector function. For example, in some embodiments, an antibody may be modified to reduce or enhance at least one constant region-mediated biological effector function relative to the unmodified antibody, e.g., reduced or improved binding to the Fc receptor (FcγR). FcγR binding may be reduced, e.g. by mutating the immunoglobulin constant region segment of the antibody at particular regions necessary for FcγR interactions (see, e.g., Canfield, Stephen M., and Sherie L. Morrison. “The binding affinity of human IgG for its high affinity Fc receptor is determined by multiple amino acids in the CH2 domain and is modulated by the hinge region.” The Journal of experimental medicine 173.6 (1991): 1483-1491; and Lund, John, et al. “Human Fc gamma RI and Fc gamma RII interact with distinct but overlapping sites on human IgG.” The Journal of Immunology 147.8 (1991): 2657-2662.). FcγR binding may be enhanced, e.g. by afucosylation. Reducing FcγR binding may also reduce other effector functions which rely on FcγR interactions, such as opsonization, phagocytosis and antigen-dependent cellular cytotoxicity (“ADCC”).

Furthermore, addressing the interaction of Fc with FcRn allows to modulate the half-life of antibodies in vivo. Abrogating the interaction by e.g. introduction of mutation H435A leads to an extremely short half-life, since the antibody is no longer protected from lysosomal degradation by FcRn recycling. In some preferred embodiments according to all aspects, the antibody according to the current invention comprises mutation H435A or has otherwise been engineered for a reduced half-life.

In contrast, antibodies comprising “YTE” mutations (M252Y/S254T/T256E) and/or equivalent mutations such as “LS” mutations (M428L/N434S) have been shown to significantly extend the half-life by more efficient recycling from endosomes in both pre-clincal species as well as humans (Dall'Acqua, William F., et al. “Increasing the affinity of a human IgG1 for the neonatal Fc receptor: biological consequences.” The Journal of Immunology 169.9 (2002): 5171-5180.; Zalevsky, Jonathan, et al. “Enhanced antibody half-life improves in vivo activity.” Nature biotechnology 28.2 (2010): 157-159.). In some preferred embodiments according to all aspects, the antibody according to the current invention comprises YTE mutations (M252Y/S254T/T256E) and/or equivalent mutations such as LS (M428L/N434S) or has otherwise been engineered for an improved half-life. Suitable Fc engineering approaches for extension of half-life can be found in Haraya, Kenta, Tatsuhiko Tachibana, and Tomoyuki Igawa. “Improvement of pharmacokinetic properties of therapeutic antibodies by antibody engineering.” Drug metabolism and pharmacokinetics 34.1 (2019): 25-41., and/or Lee, Chang-Han, et al. “An engineered human Fc domain that behaves like a pH-toggle switch for ultra-long circulation persistence.” Nature communications 10.1 (2019): 1-11., both incorporated herein by reference.

“Afucosylated” antibodies are antibodies engineered such that the oligosaccharides in the Fc region of the antibody do not have any fucose sugar units. Glycosylation of an antibody can alter its function. For example, if glycosylation at N297 in the CH2 domain of an IgG is completely eliminated, binding to FcγRs is lost. However, modulation of the specific carbohydrate composition at N297 can have the opposite effect and enhance the ADCC activity of the antibody. In brief, the affinity of an antibody for the activating FcγRs depends on the composition of the N297 N-linked oligosaccharide. There are 32 different possible combinations of oligosaccharides that can occur at this site. Naturally occurring human IgG and those produced by hybridomas or other common expression systems are usually composed of N-acetylglucosamine (GlcNAc) and three mannose residues that form a core carbohydrate. This core is attached to two additional GlcNAc groups to form biantennary branches. The addition of galactose at each branch can occur as well as the terminal addition of sialic acid to these galactose molecules. Fucose is often part of the core GlcNAc. This fucose, through steric hindrance, obstructs the interaction of the antibody with the FcγRIIIA. Thus, elimination of this fucose molecule while maintaining other forms of glycosylation at this site increases the binding of the antibody to the activating FcγRs, enhancing its ability to elicit ADCC and/or ADCP (Almagro, Juan C., et al. “Progress and challenges in the design and clinical development of antibodies for cancer therapy.” Frontiers in immunology 8 (2018): 1751.). Methods of preparing fucose-less antibodies include growth in rat myeloma YB2/0 cells (ATCC CRL 1662). YB2/0 cells express low levels of FUT8 mRNA, which encodes α-1,6-fucosyltransferase, an enzyme necessary for fucosylation of polypeptides. Afucosylated antibodies are preferred for the current invention.

“Antibody-dependent cellular cytotoxicity” (“ADCC”), also referred to as “antibody-dependent cell-mediated cytotoxicity”, is a mechanism of cell-mediated immune defense whereby an immune cell actively lyses a target cell, whose membrane-surface antigens have been bound by specific antibodies. ADCC is mediated via interaction of the antibody or fragment with FcγRIIIa. In humans, FcγRIII exists in two different forms: FcγRIIIa (CD16a) and FcγRIIIb (CD16b). While FcγRIIIa is expressed on monocytes, neutrophils, mast cells, macrophages, and natural killer cells as a transmembrane receptor, FcγRIIIb is only expressed on neutrophils. These receptors bind to the Fc portion of IgG antibodies, which then activates antibody-dependent cell-mediated cytotoxicity (ADCC) mediated by the human effector cells.

Different assay systems to determine ADCC induction in human subjects have been described in the literature and are suitable for characterization of the subject matter disclosed herein. For example, Yao-Te Hsieh et al. have studied different ADCC assay systems, namely assays based on (i) natural killer cells from human donors (FcγRIIIA+primary NK), (ii) FcγRIIIA engineered NK-92 cells and (iii) FcγRIIIA/NFAT-RE/luc2 engineered Jurkat T cells (Hsieh, Yao-Te, et al. “Characterization of FcγRIIIA effector cells used in in vitro ADCC bioassay: comparison of primary NK cells with engineered NK-92 and Jurkat T cells.” Journal of Immunological Methods 441 (2017): 56-66, incorporated herein in entirety; in particular, reference is made to the method description for these assays). In brief, all three effector cell systems differentially express FcγRIIIA and provide dose-dependent ADCC pathway activity, yet only primary NK and engineered NK-92 cells are capable of inducing ADCC-mediated cell lysis. For functional assessment of ADCC activity, primary NK or NK-92 (V-158) cells thus better reflect the physiologically relevant ADCC mechanism of action. As an engineered cell line, NK-92 cells may behave more reproducibly than primary NK and is therefore the preferred assay system to determine ADCC response in human subjects, e.g. in case of doubt.

An antibody or antigen-binding fragment inducing ADCC is an antibody which may elicit a substantial amount of lysis of target cells in the presence of NK effector cells. Preferably, the ADCC induction results in the lysis of at least 2%, 5%, 10%, 15%, 20%, 25%, 30%, 35%, 40%, 45%, 50%, 55%, 60%, 65%, 70%, 75%, 80%, 85%, 90%, 95% or 99% of the target cells.

“Antibody-dependent cellular phagocytosis” (“ADCP”) is the mechanism by which antibody-opsonized target cells activate the FcγRs on the surface of macrophages to induce phagocytosis, resulting in internalization and degradation of the target cell. For ADCP, binding to macrophages as effector cells typically occurs via the interaction of the antibodies FC part with FcγRIIa (CD32a) expressed by macrophages.

An antibody or antigen-binding fragment inducing ADCP is an antibody which may elicit a substantial amount of phagocytosis of target cells in the presence of macrophages. Preferably, the ADCP induction results in the phagocytosis of at least 2%, 5%, 10%, 15%, 20%, 25%, 30%, 35%, 40%, 45%, 50%, 55%, 60%, 65%, 70%, 75%, 80%, 85%, 90%, 95% or 99% of the target cells.

“Complement-dependent cytotoxicity” (“CDC”) is an effector function of IgG and IgM antibodies. When they are bound to a surface antigen on a target cell (e.g. bacterial or viral infected cell), the classical complement pathway is triggered by bonding protein C1q to these antibodies, resulting in formation of a membrane attack complex (MAC) and target cell lysis. Complement system is efficiently activated by human IgG1, IgG3 and IgM antibodies, weakly by IgG2 antibodies and is not activated by IgG4 antibodies. It is one mechanism of action by which therapeutic antibodies—also specific embodiments of the antibodies according to the current invention—can achieve an antitumor effect. Several laboratory methods exist for determining the efficacy of CDC and are known in the art.

An antibody or antigen-binding fragment inducing CDC is an antibody which may elicit a substantial amount of formation of a membrane attack complex and lysis of target cells. Preferably, the CDC induction results in the lysis of at least 2%, 5%, 10%, 15%, 20%, 25%, 30%, 35%, 40%, 45%, 50%, 55%, 60%, 65%, 70%, 75%, 80%, 85%, 90%, 95% or 99% of the target cells.

Antibodies comprising an Fc region may or may not comprise a modification promoting the association of the first and the second subunit of the Fc domain.

Most preferably, the induction of ADCC and ADCP results in at least 50% Treg depletion.

A “fragment” of an antibody as used herein is required to substantially retain the desired affinity of the full-length antibody. As such, suitable fragments of an anti-human CCR8 antibody will retain the ability to bind to the target chemokine receptor, e.g. to bind to human CCR8 receptor. Fragments of an antibody comprise a portion of a full-length antibody, generally the antigen binding or variable region thereof. Examples of antibody fragments include, but are not limited to, Fab, Fab′, F(ab′)2, and Fv fragments, single-chain antibody molecules, diabodies and domain antibodies, see Holt, Lucy J., et al. “Domain antibodies: proteins for therapy.” Trends in biotechnology 21.11 (2003): 484-490.

A “Fab fragment” contains the constant domain of the light chain and the first constant domain (CH2) of the heavy chain.

“Fab′ fragments” differ from Fab fragments by the addition of a few residues at the carboxyl terminus of the heavy chain CH2 domain including one or more cysteines from the antibody hinge region.

“F(ab′) fragments” are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fc fragment of intact antibody, clear more rapidly from the circulation of animals, and may have less non-specific tissue binding than an intact antibody, see, e.g., Wahl, Richard L., Charles W. Parker, and Gordon W. Philpott. “Improved radioimaging and tumor localization with monoclonal F(ab′)2.” Journal of nuclear medicine: official publication, Society of Nuclear Medicine 24.4 (1983): 316-325.

An “Fv fragment” is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Often, the six CDRs confer antigen binding specificity upon the antibody. However, in some instances even a single variable domain (or half of an Fv comprising only three CDRs specific for a target) may have the ability to recognize and bind the antigen, although at a lower affinity than the entire binding site.

“Single-chain Fv” or “scFv” antibody fragments comprise the VH and VL domains of an antibody in a single polypeptide chain. Generally, the Fv polypeptide further comprises a polypeptide linker between the VH and VL domains which enables the scFv to form the desired structure for antigen binding.

“Single domain antibodies” are composed of single VH or VL domains which exhibit sufficient affinity to the target. In a specific embodiment, the single domain antibody is a camelized antibody, see, e.g., Riechmann, Lutz, and Serge Muyldermans. “Single domain antibodies: comparison of camel VH and camelised human VH domains.” Journal of immunological methods 231.1-2 (1999): 25-38.

A “minibody” is an antibody format that has a smaller molecular weight than a full-length antibody while maintaining the bivalent binding property against an antigen. For example, a minibody may be a bivalent homodimer with each monomer having a single-chain variable fragment (scFv) linked to the human IgG1 CH3 domain via modified IgG1 hinge sequence. Because of its smaller size, the minibody has a faster clearance from the system and enhanced penetration when targeting tumor tissue. With the ability for strong targeting combined with rapid clearance, the minibody is advantageous for diagnostic imaging and delivery of cytotoxic/radioactive payloads for which prolonged circulation times may result in adverse patient dosing or dosimetry.

A “Zr-89-labeled anti-CD8 minibody” is a minibody that binds specifically to CD8 and that is furthermore labeled with Zr-89. Preferably, the Zr-89-labeled anti-CD8 minibody binds human CD8 glycoprotein with an EC50 of <1 nM. For example, the minibody can be conjugated via desferrioxamine (Df) and radiolabeled with the positron emitting radionuclide “Zirconium-89” (89Zr; Tm 78.4 hours). According to an utmost preferred embodiment, the Zr-89-labeled anti-CD8 minibody is the Zr-89-labeled anti-CD8 minibody described in U.S. application Ser. No. 17/280,137.

“Bispecific antibodies” are monoclonal antibodies that have binding specificities for at least two different epitopes on the same or different antigens. In the present disclosure, one of the binding specificities can be directed towards the target chemokine receptor such as CCR8, the other can be for any other antigen, e.g., without limitation for a cell-surface protein, receptor, receptor subunit, tissue-specific antigen, virally derived protein, virally encoded envelope protein, bacterially derived protein, or bacterial surface protein. Bispecific antibody constructs according to the invention also encompass multispecific antibody constructs comprising multiple binding domains/binding sites, such as trispecific antibody constructs, where the construct comprises three binding domains.

“Derivatized antibodies” are typically modified by glycosylation, acetylation, pegylation, phosphorylation, sulfation, amidation, derivatization by known protecting/blocking groups, proteolytic cleavage, linkage to a cellular ligand or other protein. Any of numerous chemical modifications may be carried out by known techniques, including, but not limited to, specific chemical cleavage, acetylation, formylation, metabolic synthesis of tunicamycin, etc. Additionally, the derivative may contain one or more non-natural amino acids, e.g., using ambrx technology, see, e.g., Wolfson, Wendy. “Amber codon flashing ambrx augments proteins with unnatural amino acids.” Chemistry & biology 13.10 (2006): 1011-1012. Antibodies according to the current invention may be derivatized, e.g. glycosylated or sulfated.

“Monoclonal antibodies” are substantially homogenous populations of antibodies binding a particular antigen. Monoclonal immunoglobulins may be obtained by methods well known to those skilled in the art (see for example, Kohler, Georges, and Cesar Milstein. “Continuous cultures of fused cells secreting antibody of predefined specificity.” nature 256.5517 (1975): 495-497., and U.S. Pat. No. 4,376,110). An immunoglobulin or immunoglobulin fragment with specific binding affinity can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms. The antibodies according to the current invention are preferably monoclonal.

“Humanized antibodies” contain CDR regions derived from a non-human species, such as mouse, that have, for example, been engrafted, along with any necessary framework back-mutations, into human sequence-derived V regions. Thus, for the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues. Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally comprises at least a portion of an immunoglobulin constant region (Fc), typically that of a human immunoglobulin. For further details see Jones, Peter T., et al. “Replacing the complementarity-determining regions in a human antibody with those from a mouse.” Nature 321.6069 (1986): 522-525.; Riechmann, Lutz, et al. “Reshaping human antibodies for therapy.” Nature 332.6162 (1988): 323-327.; and Presta, Leonard G. “Antibody engineering.” Current Opinion in Structural Biology 2.4 (1992): 593-596., each incorporated herein by reference.

Fully human antibodies (human antibodies) comprise human derived CDRs, i.e. CDRs of human origin. Preferably, a fully human antibody according to the current invention is an antibody having at least 90%, 91%, 92%, 93%, 94%, 95%, 96%, 97%, 98%, 99%, 99.5% or 100% sequence identity with the closest human VH germline gene (e.g. sequence extracted from recommended list and analyzed in IMGT/Domain-gap-align).

As accepted by usual nomenclature systems such as the INN species subsystem in force until 2017, fully human antibodies may comprise a low number of germline deviations compared with the closest human germline reference determined based on the IMGT database (http://www.imgt.org, Nov. 29, 2019). For example, a fully human antibody according to the current invention may comprise up to 1, 2, 3, 4, 5, 6, 7, 8, 9, 10, 12, 13, 14 or 15 germline deviations in the CDRs compared with the closest human germline reference. Fully human antibodies can be developed from human derived B cells by cloning techniques in combination with a cell enrichment or immortalization step. The majority of fully human antibodies in clinical use, however, were isolated either from immunized mice transgenic for the human IgG locus or from sophisticated combinatorial libraries by phage display (Bruggemann, Marianne, et al. “Human antibody production in transgenic animals.” Archivum immunologiae et therapiae experimentalis 63.2 (2015): 101-108.; Carter, Paul J. “Potent antibody therapeutics by design.” Nature reviews immunology 6.5 (2006): 343-357.; Frenzel, Andre, Thomas Schirrmann, and Michael Hust. “Phage display-derived human antibodies in clinical development and therapy.” MAbs. Vol. 8. No. 7. Taylor & Francis, 2016.; Nelson, Aaron L., Eugen Dhimolea, and Janice M. Reichert. “Development trends for human monoclonal antibody therapeutics.” Nature reviews drug discovery 9.10 (2010): 767-774.).

Several techniques are available to generate fully human antibodies or to generate antibodies comprising human derived CDRs (cf. WO2008112640). Cambridge Antibody Technologies (CAT) and Dyax have obtained antibody cDNA sequences from peripheral B cells isolated from immunized humans and devised phage display libraries for the identification of human variable region sequences of a particular specificity. Briefly, the antibody variable region sequences are fused either with the Gene III or Gene VIII structure of the M13 bacteriophage. These antibody variable region sequences are expressed either as Fab or single chain Fv (scFv) structures at the tip of the phage carrying the respective sequences. Through rounds of a panning process using different levels of antigen binding conditions (stringencies), phages expressing Fab or scFv structures that are specific for the antigen of interest can be selected and isolated. The antibody variable region cDNA sequences of selected phages can then be elucidated using standard sequencing procedures. These sequences may then be used for the reconstruction of a full antibody having the desired isotype using established antibody engineering techniques. Antibodies constructed in accordance with this method are considered fully human antibodies (including the CDRs). In order to improve the immunoreactivity (antigen binding affinity and specificity) of the selected antibody, an in vitro maturation process can be introduced, including a combinatorial association of different heavy and light chains, deletion/addition/mutation at the CDR3 of the heavy and light chains (to mimic V-J, and V-D-J recombination), and random mutations (to mimic somatic hypermutation). An example of a “fully human” antibody generated by this method is the anti-tumor necrosis factor α antibody, Humira (adalimumab).

“Anti-drug-antibodies” (ADAs) are antibodies that bind a therapeutic antibody and result from an immune response of a subject or patient to a therapeutic antibody. ADAs can inactivate the therapeutic effects of the treatment and potentially induce adverse effects.

The term “quantifying” means estimating or measuring the amount of a molecule such as an antibody at least in a semi-quantitative way.

The term “polynucleotide” refers to a recombinantly or synthetically produced polymeric desoxyribonucleotide or analog thereof, or a modified polynucleotide. The term comprises double and single stranded DNA or RNA. The polynucleotide can be integrated e.g. into minicircles, plasmids, cosmids, minichromosomes, or artificial chromosomes. The polynucleotide can be isolated or integrated in another nucleic acid molecule, e.g. in an expression vector or chromosome of a eukaryotic host cell.

The term “vector”, as used herein, refers to a nucleic acid molecule capable of propagating a nucleic acid molecule to which it is linked. The term further comprises plasmids (non-viral) and viral vectors. Certain vectors are capable of directing the expression of nucleic acids or polynucleotides to which they are operatively linked. Such vectors are referred to herein as “expression vectors”. Expression vectors for eukaryotic use can be constructed by inserting a polynucleotide sequence encoding at least one protein of interest (POI) into a suitable vector backbone. The vector backbone can comprise the necessary elements to ensure maintenance of the vector and, if desirable, to provide amplification within the host. For viral vectors, e.g. lentiviral or retroviral vectors, further virus specific elements such as structural elements or other elements can be required and are well known in the art. These elements can be for instance provided in cis (on the same plasmid) or in trans (on a separate plasmid). Viral vectors may require helper viruses or packaging lines for large-scale transfection. Vectors may contain further elements such as e.g. enhancer elements (e.g. viral, eukaryotic), introns, and viral origins of plasmid replication for replication in mammalian cells. According to the current invention, expression vectors typically have a promoter sequence that drives expression of the POI. Expression of the POI and/or selective marker protein may be constitutive or regulated (e.g. inducible by addition or removal of small molecule inductors). Preferred regulatory sequences for mammalian host cell expression include viral elements that direct high levels of expression of a POI in mammalian cells, such as regulatory elements, promoters and/or enhancers derived from cytomegalovirus (CMV), Simian Virus 40 (SV40), adenovirus, (e.g., the adenovirus major late promoter Ad LP) or polyoma. For further description of viral regulatory elements, and sequences thereof, see e.g., U.S. Pat. Nos. 5,168,062, 4,510,245 and 4,968,615.

The term “linker” or “spacer” as used herein refers to any molecule enabling a direct topological connection between two moieties. A moiety may be inter alia a polypeptide, a protein, an antibody, an antibody fragment, a cytotoxic moiety, a binding moiety, a moiety for detection such as a fluorophore, a moiety for immobilization or retrieval such as beads or magnetic beads, a reactive moiety, or any other molecule. The two moieties may be of the same type or different. Linkers may be part of conjugates and may even contribute to their function. For instance, for a conjugate comprising a polypeptide and a biotin, the presence of a spacer of approximately 4 Å (˜5 atoms) between the carboxy group of the biotin and the 1st bulky amino acid of the peptide allows the biotin to reach the (strept)avidin binding pocket. Various linkers are known in the art and can be selected based on the moieties which shall be connected. The linker length typically ranges between 4 atoms and more than 200 atoms. Linkers exceeding 60 atoms in length generally comprise a population of compounds having an average length.

“Linkers for polypeptides” may be attached through an amide linkage or any other functional residue. Linkers for polypeptides may be attached N-terminal or C-terminal of the polypeptide or may be attached via a reactive functional group or amino acid side chain. Polypeptides may be coupled for example to biotin, proteins such as human serum albumin (HSA), carrier proteins such as keyhole limpet hemocyanin (KLH), ovalbumin (OVA) or bovine serum albumin (BSA), fluorescent dyes, short amino acid sequences such as Flag tag, HA tag, Myc tag or His tag, reactive tags such as maleimides, iodoacetamides, alkyl halides, 3-mercaptopropyl or 4-azidobutyric acid, or to various further suitable moieties. Non-limiting examples for suitable linkers, e.g. for conjugation of polypeptides, include beta-alanine, 4-aminobutyric acid (GABA), (2-aminoethoxy) acetic acid (AEA), 5-aminovaleric acid (Ava), 6-aminohexanoic acid (Ahx), PEG2 spacer (8-amino-3,6-dioxaoctanoic acid), PEG3 spacer (12-amino-4,7,10-trioxadodecanoic acid), PEG4 spacer (15-amino-4,7,10,13-tetraoxapenta-decanoic acid), and Ttds (Trioxatridecan-succinamic acid). In some cases, the linker may be derived from a reactive moiety, e.g. maleimides, iodoacetamides, alkyl halides, 3-mercaptopropyl or 4-azidobutyric acid. In some cases, the linker may comprise polyethylene glycol (PEG), polypropylene glycol, polyoxyalkylenes, or copolymers of polyethylene glycol or polypropylene glycol.

“Linkers for antibodies” are linkers establishing a covalent connection between different antibody portions and include peptide linker and non-proteinaceous polymers, including but not limited to polyethylene glycol (PEG), polypropylene glycol, polyoxyalkylenes, or copolymers of polyethylene glycol, polypropylene glycol.

“Treating” a disease in a subject or “treating” a subject having a disease refers to subjecting the subject to a pharmaceutical treatment, e.g., the administration of a drug, such that at least one symptom of the disease is decreased or prevented from worsening.

The terms “prevent”, “preventing”, “prevention” and the like refer to reducing the probability of developing a disease, disorder, or condition in a subject, who does not have, but is at risk of or susceptible to developing a disease, disorder, or condition.

The term “effective amount” or “therapeutically effective amount” are used interchangeably herein and refer to an amount sufficient to achieve a particular biological result or to modulate or ameliorate a symptom in a subject, or the time of onset of a symptom, typically by at least about 10%; usually by at least about 20%, preferably at least about 30%, or more preferably at least about 50%. Efficacy of the use of an antibody in cancer therapy can be assessed based on the change in tumor burden. Both tumor shrinkage (objective response) and time to the development of disease progression are important endpoints in cancer clinical trials. Standardized response criteria, known as RECIST (Response Evaluation Criteria in Solid Tumors), were published in 2000. An update (RECIST 1.1) was released in 2009. RECIST criteria are typically used in clinical trials where objective response is the primary study endpoint, as well as in trials where assessment of stable disease, tumor progression or time to progression analyses are undertaken because these outcome measures are based on an assessment of anatomical tumor burden and its change over the course of the trial. An effective amount for a particular subject may vary depending on factors such as the condition being treated, the overall health of the subject, the method, route, and dose of administration and the severity of side effects. When in combination, an effective amount is in ratio to a combination of components and the effect is not limited to individual components alone.

If not defined otherwise, “Complete Response” (CR) is defined as disappearance of all target lesions. Any pathological lymph nodes (whether target or non-target) must have reduction in short axis to <10 mm. For “Partial Response” (PR) at least a 30% decrease in the sum of diameters of target lesions has to be reached, taking as reference the baseline sum diameters. For “Progressive Disease” (PD) at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm.

In “Stable Disease” (SD) neither sufficient shrinkage to qualify for PR nor sufficient increase to qualify for PD is observed, taking as reference the smallest sum diameters while on study.

Secondary outcome measures that can be used to determine the therapeutic benefit of the inventive antibodies described herein include the following: “Objective Response Rate” (ORR) is defined as the proportion of subjects who achieve a complete response (CR) or partial response (PR). “Progression Free Survival” (PFS) is defined as the time from the first dose date of an antibody to either disease progression or death, whichever occurs first. “Overall Survival” (OS) is defined as the length of time from either the date of diagnosis or the start of treatment for a disease, that patients diagnosed with the disease are still alive. “Duration of Overall Response” (DOR) is defined as the time from the participant's initial CR or PR to the time of disease progression. “Depth of Response” (DpR) is defined as the percentage of tumor shrinkage observed at the maximal response point compared to baseline tumor load. Clinical endpoints for both ORR and PFS can be determined based on RECIST 1.1 criteria described above.

Where non-human subjects are analyzed, the aforementioned parameters to determine therapeutic efficacy and benefit have to be adapted.

Typical “subjects” according to the current invention include human and non-human subjects. Subjects can be mammals such as mice, rats, cats, dogs, primates and/or humans.

The term “patient” refers to a human subject having a medical condition.

“Pharmaceutical compositions” (also “therapeutic formulations”) of the antibody, fragment or conjugate can be prepared by mixing the antibody having the desired degree of purity with optional physiologically acceptable carriers, excipients or stabilizers, e.g. according to Remington's Pharmaceutical Sciences (18th ed.; Mack Pub. Co.: Eaton, Pa., 1990), e.g. in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween©, Pluronic© or polyethylene glycol (PEG).

A “host cell” is a cell that is used to receive, maintain, reproduce and amplify a vector. A host cell also can be used to express the polypeptide, e.g. an antibody or fragment thereof encoded by the vector. The nucleic acid contained in the vector is replicated when the host cell divides, thereby amplifying the nucleic acids. Preferred host cells are mammalian cells, such as CHO cells or HEK cells. Further preferred host cells are rat myeloma YB2/0 cell.

A “cell with endogenous target expression” is a cell which expresses a target protein at a level which is comparable to the physiological or diseased situation. Typically, cells which have been engineered for overexpression express a target protein at much higher levels.

A “lesion” as used herein refers to an area of abnormal tissue. A lesion may be benign or malignant (“cancer lesion”, also “tumor lesion”).

The term “intra-tumoral”, “intratumoral”, “tumor infiltrating” or “tumoral” in the context of cells, structures, proteins, antibodies, or markers refers to their localization within the tumor tissue.

Cells which are “positive” or “+” for a certain marker or protein are cells characterized by substantial expression of that marker or protein. Marker or protein expression can be determined and quantified as known in the art, e.g. to define different cell populations. For the characterization of (immune) cell populations, the marker expression can be determined by FACS or using any other technique described herein.

“Leukocytes” are immune cells expressing CD45.

“CD45+ cells”, as used herein, refer to all leukocytes. CD45 can be used as a marker to distinguish immune cells and non-immune cells.

The term “lymphocyte” refers to all immature, mature, undifferentiated, and differentiated white lymphocyte populations, including tissue specific and specialized varieties. It encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-I cells, B-2 cells, and anergic AN1/T3 cell populations.

“T cells” are immune cells expressing TCRαβ, CD3, and CD8 or CD4. As used herein, the term includes naive T cells, CD4+ T cells, CD8+ T cells, regulatory T cells, memory T cells, activated T cells, anergic T cells, tolerant T cells, chimeric B cells, and antigen-specific T cells and further T cell populations known in the art. In some embodiments, the presence of a T cell receptor (TCR) on the cell surface distinguishes T cells from other lymphocytes.

“CD8+ T cells” (also “cytotoxic T cell”, “TC”, “cytotoxic T lymphocyte”, “CTL”, “T-killer cell”, “cytolytic T cell”, “CD8+ T-cell” or “killer T cell”) are T cells expressing CD3, CD45 and CD8. CD8+ T cells can kill cancer cells, cells that are infected (particularly with viruses), or otherwise damaged cells.

“CD4+ T cells” (also “T helper cells”, “Th cells”) are immune cells expressing CD3, CD4 and CD45. There are several subsets of T helper cells, such as, without limitation, Th1, Th2, and Th17. CD4+ T cells help suppress or regulate immune responses. They are essential in B cell antibody class switching, in the activation and growth of cytotoxic T cells, and in maximizing bactericidal activity of phagocytes such as macrophages.

As used herein, the term “Treg cells” (also “Tregs”, “regulatory T cells”, “T regulatory cells”, “suppressor T cells”) refers to immune cells expressing CD3, CD4, CD45, and FoxP3, and furthermore expressing high levels of CD25 and low levels of CD127. Identification of Treg cells may be performed as described elsewhere herein. Treg cells typically also express high levels of CTLA-4, GITR, and LAG-3. In the literature, Tregs have furthermore been classified based on memory marker CD45RO.

Under physiological conditions, Treg cells maintain immunological tolerance. During an immune response, Treg cells stop T cell-mediated immunity and suppress auto-reactive T cells that have escaped negative selection within the thymus. Treg cells can also suppress other types of immune cells such as NK cells and B cells. Adaptive Treg cells (called Th3 or Tr1 cells) are thought to be generated during an immune response.

Treg cells furthermore play an important role in immune escape by suppressing antitumor immunity, thereby providing an environment of immune tolerance. T cells that recognize cancer cells are often present in large numbers in tumors, but their cytotoxic function is suppressed by nearby immune-suppressor cells. Tregs are abundant in many different cancers, are highly enriched in the tumor microenvironment, and are well known for their role in tumor progression.

“Activated Treg cells” express CD4, CD45, FoxP3, CD69 and CCR8, and furthermore have a high expression of CD25, and have a low expression of CD127. CD69 is a T cell activation marker.

“CCR8 positive regulatory T cells” or “CCR8+ regulatory T cells” are Tregs expressing CCR8.

“CD4conv cells” are conventional CD4+, CD25− T cells.

“Gamma delta T cells” are T cells that express a distinctive T-cell receptor, TCRγδ, on their surface. Gamma delta T cells also express CD3.

“B cells” are immune cells expressing CD19, and mature B cells express CD20 and CD22. B cells upon activation via CD40 undergo differentiation where somatic hypermutation and enhanced immunoglobulin class switch occur resulting in mature B cells or plasma cells (capable of secreting Abs). B cells are involved in humoral immunity of the adaptive immune system and are antigen presenting cells.

“Macrophages” are immune cells expressing low CD14, high CD16, CD11b, CD68, CD163, and CD206. Macrophages engulf and digest cellular debris, foreign substances, microbes or cancer cells by phagocytosis. Besides phagocytosis, macrophages play a critical role in innate immunity and also help initiate adaptive immunity by recruiting other immune cells. For example, macrophages are important as antigen presenters to T cells. Macrophages that encourage inflammation are called M1 macrophages, whereas those that decrease inflammation and encourage tissue repair are called M2 macrophages.

As used herein, “M1 macrophages” are a subset of macrophages expressing ACOD1. M1 macrophages have pro-inflammatory, bactericidal, and phagocytic functions.

As used herein, “M2 macrophages” are a subset of macrophages expressing MRC1 (CD206). M2 macrophages secrete anti-inflammatory interleukins, play a role in wound healing and are needed for revascularization and reepithelialization. Tumor-associated macrophages are mainly of the M2 phenotype and seem to actively promote tumor growth.

“Dendritic Cells” (DCs) are bone marrow derived leukocytes and are the most potent type of antigen-presenting cells. DCs are specialized to capture and process antigens, converting proteins to peptides that are presented on major histocompatibility complex (MHC) molecules recognized by T cells. As defined herein, DCs are characterized by expression of CD1c, CD14, CD16, CD141, CD11c and CD123. Different subpopulations of Dendritic cells exist. In human, DC1 are immunogenic while DC2 cells are tolerogenic. Mature DC express CD83, while plasmacytoid DC express CD123.

“NK cells” (also natural killer cells) are immune cells which express CD45, CD16, CD56, NKG2D, but are CD3 negative. NK cells do not require activation to kill cells that are missing “self” markers of MHC class 1. NCR1 (also referred to as CD335 or NKp46) is expressed on NK cells and on a subset of NKT cells.

“Natural killer T (NKT) cells” are a heterogeneous group of T cells that share properties of both T cells and natural killer cells.

“iNKT cells” (also “invariant natural killer T cells”) express invariant αβ TCR (Vα24-Jα18, CD24lo), CD44hi, NK1.1 (mouse), and NKG2D. The invariant TCR recognizes glycoplipid antigen presented by non-polymorphic MHC class I-like molecule, CD1d. These cells can influence an immune response by rapidly producing large amounts of cytokines, i.e. IFNγ.

As known in the art, “effector cells” are immune cells that actively support immune response after stimulation. As used herein, effector cells refer to immune cells expressing Fcγ receptors and are therefore able to mediate ADCC or ADCP. Non-limiting examples of effector cells are monocytes, neutrophils, mast cells, and, preferably, macrophages, and natural killer cells.

The term “chimeric antigen receptor” or “CAR” as used herein, refers to an artificial T cell surface receptor that is engineered to be expressed on an immune effector cell and specifically bind an antigen. CARs may be used as a therapy with adoptive cell transfer. Monocytes are removed from a patient (blood, tumor or ascites fluid) and modified so that they express the receptors specific to a particular form of antigen. In some embodiments, the CARs have been expressed with specificity to a tumor associated antigen. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting a monocyte/macrophage expressing the CAR specific for tumor associated antigens.

Dosing schemes are abbreviated as known in the art, e.g. every day (QD), every 2 days (Q2D), or every 3 days (Q3D). In accordance with this, “QW” means once every week, “Q2W” once every two weeks, “Q3W” once every three weeks, “Q4W” means once every four weeks, “Q5W” once every five weeks and, “Q6W” once every six weeks.

A “dosing cycle” or “treatment cycle” is a period of treatment followed by a period of rest (no treatment) that is repeated on a regular schedule. When this cycle is repeated multiple times on a regular schedule, it makes up a course of treatment.

In pharmacology, a “trough concentration”, abbreviated “Ctrough” is the concentration reached by a drug immediately before the next dose is administered.

“Cytokine release syndrome” (CRS), and when severe “cytokine storm”, is a supraphysiologic response that can occur in response to any immune therapy, as sequelae of the immune system activation associated with infusion reactions. Binding of a mAb results in the activation or engagement of endogenous or infused T cells and/or other immune effector cells that leads to rapid release of inflammatory cytokines from target immune cells into the circulation. CRS usually occurs within several hours to days after infusion of the monoclonal antibody. The incidence rate of CRS is relatively low for monoclonal antibodies, but high for chimeric antigen receptor (CAR)-T and T-cell engager varying between 17%-94%.

An intravenous line or “IV line” is a tube or cannula that can be used for an intravenous infusion.

A “PET scan” is a diagnostic technique that can be used to observe functions and metabolism of human organs and tissues at the molecular level. For a PET scan, a positron radioactive drug (e.g., 18F-FDG) can be injected into a human body. If FDG is used, because the metabolism of fluorodeoxyglucose (FDG) is similar to glucose, the FDG will gather in cells that digest the glucose. The uptake of the radioactive drug by rapidly growing tumor tissues is different. A positron emitted by the decay of 18F and an electron in tissues will undergo an annihilation reaction to generate two gamma-photons with the same energy in opposite directions. For a PET scan, a detector array surrounding the human body can detect the two photons using a coincidence measurement technique and determine position information of the positron. A tomography image of positrons in the human body can then be constructed by processing the position information using an image reconstruction software. In some situations, immuno-PET can be employed, where the label (e.g., 18F) is attached or associated with an antigen binding construct. In such embodiments, the distribution or abundance of the antigen binding construct can be monitored, which will depend upon the binding properties and distribution properties of the antigen binding construct. For example, if a CD8 directed minibody or Zr-89-labeled anti-CD8 minibody is used, then a PET scan can be used to monitor the distribution and/or abundance of the CD8 directed minibody or Zr-89-labeled anti-CD8 minibody, and therefore of the CD8 molecules through the subjects' system. PET systems are known in the art and include, for example U.S. Pat. Pub. No. 20170357015, 20170153337, 20150196266, 20150087974, 20120318988, and 20090159804, the entireties of each of which are incorporated by reference herein for their description regarding PET, PET scans and the use thereof.

The “standardized uptake value”, also “standard uptake value” or “SUV” is a term in the art in nuclear medicine and is used in positron emission tomography (PET) as well as in modern calibrated single photon emission tomography (SPECT) imaging for a semiquantitative analysis.

A computed tomography scan or “CT scan” (formerly called computed axial tomography scan or CAT scan) is a medical imaging technique used to obtain detailed internal images of the body. CT scanners use a rotating X-ray tube and a row of detectors placed in a gantry to measure X-ray attenuations by different tissues inside the body. The multiple X-ray measurements taken from different angles are then processed on a computer using tomographic reconstruction algorithms to produce tomographic (cross-sectional) images (virtual “slices”) of a body.

The term “distribution”, in the context of monitoring, detecting, comparing, or observing a distribution of a Zr-89-labeled anti-CD8 minibody which has been administered to a subject, means a visual or mathematical image of the biodistribution of that Zr-89-labeled anti-CD8 minibody in relation to a whole body or partial body scan of the subject, which image may be represented as a flat image (2-dimensional) or as computer assisted three-dimensional representation (including a hologram), and is in a format useful to the operator or clinician to observe distribution of the Zr-89-labeled anti-CD8 minibody at the individual tissue level, and the individual tumor level.

“Antihistamines” are a class of pharmaceutical compounds designed to alleviate various allergic reactions and symptoms by blocking the action of histamines in the human body. Examples include Diphenhydramine, Loratadine, Cetirizine, Fexofenadine, Desloratadine and Levocetirizine.

“Diphenhydramine” (DPH) is an antihistamine and sedative known in the art which is mainly used to treat allergies, insomnia, and symptoms of the common cold. It is also less commonly used for tremor in parkinsonism, and nausea.

“Paracetamol” (acetaminophen[a] or para-hydroxyacetanilide) is a non-opioid analgesic and antipyretic agent known in the art which is used to treat fever and mild to moderate pain. Common brand names include Tylenol and Panadol.

“Corticosteroids” are a class of synthetic or naturally occurring steroid hormones produced by the adrenal cortex, with potent anti-inflammatory, immunosuppressive, and metabolic effects. Examples include Prednisone, Dexamethasone or Methylprednisolone.

“Dexamethasone” is a glucocorticoid medication known in the art which is used to treat inter alia rheumatic problems, a number of skin diseases, severe allergies, asthma, chronic obstructive lung disease, croup, or brain swelling.

### EMBODIMENTS

### Aspect 1

### Dosing Regimen

According to a first aspect of the current invention there is provided an anti-human CCR8 antibody having ADCC activity and ADCP activity for use in a method of treatment, comprising administering intravenously to a patient in need thereof the anti-CCR8 antibody in a total amount of
a. 1 to 250 mg once every week, or
b. 16 to 1500 mg once every three weeks.

More specifically, and as described in detail e.g. in Example 17, there is provided an anti-human CCR8 antibody having ADCC activity and ADCP activity for use in a method of treatment, comprising administering intravenously to a patient in need thereof the anti-CCR8 antibody in a total amount of
a. Approximately 1, 2.5, 3, 10, 30, 50, 100, 125, or 250 mg once every week, or
b. Approximately 16, 450, 500, 750, 1000 or 1500 mg once every three weeks.

For example, there is provided an anti-human CCR8 antibody having ADCC activity and ADCP activity for use in a method of treatment comprising administering intravenously to a patient in need thereof the anti-CCR8 antibody in a total amount of approximately 1, 2.5, 3, 10, 30, 50, 100, 125, or 250 mg once every week, preferably 10, 30, 50, 100, 125, or 250 mg once every week. In one embodiment the total amount of the anti-CCR8 antibody is approximately 1 mg once every week. In another embodiment the total amount of the anti-CCR8 antibody is approximately 2.5 mg once every week. In another embodiment the total amount of the anti-CCR8 antibody is approximately 10 mg once every week. In a preferred embodiment the total amount of the anti-CCR8 antibody is approximately 30 mg once every week. In a further preferred embodiment the total amount of the anti-CCR8 antibody is approximately 50 mg once every week. In a further preferred embodiment the total amount of the anti-CCR8 antibody is approximately 100 mg once every week. In another preferred embodiment the total amount of the anti-CCR8 antibody is approximately 125 mg once every week. In a highly preferred embodiment the total amount of the anti-CCR8 antibody is approximately 250 mg once every week.

For example, there is provided an anti-human CCR8 antibody having ADCC activity and ADCP activity for use in a method of treatment comprising administering intravenously to a patient in need thereof the anti-CCR8 antibody in a total amount of approximately 500, 750, 1000 or 1500 mg once every three weeks.

In one highly preferred embodiment the total amount of the anti-CCR8 antibody is approximately 500 mg once every three weeks. In another highly preferred embodiment the total amount of the anti-CCR8 antibody is approximately 750 mg once every three weeks. In one highly preferred embodiment the total amount of the anti-CCR8 antibody is approximately 1000 mg once every three weeks. In another preferred embodiment the total amount of the anti-CCR8 antibody is approximately 1500 mg once every three weeks.

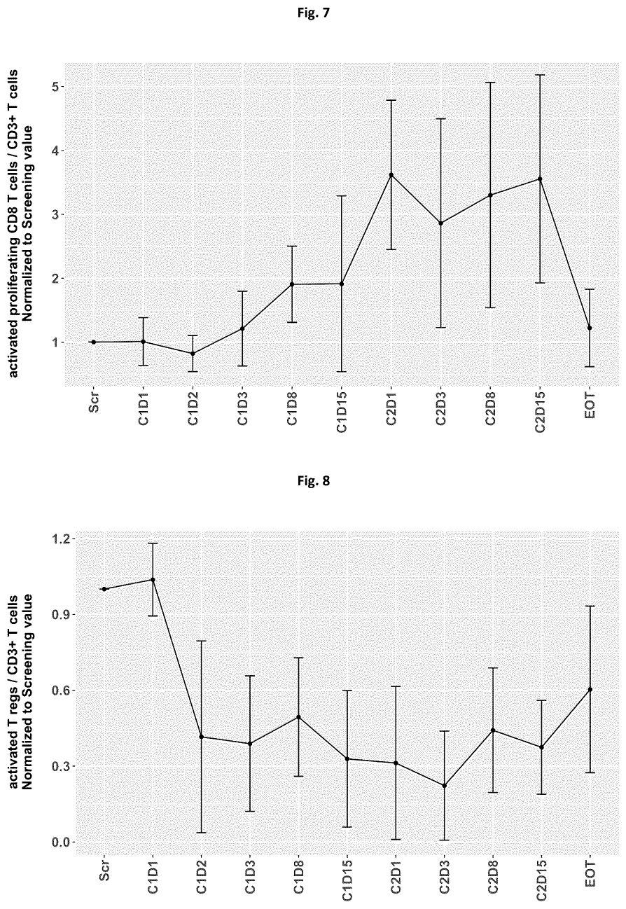

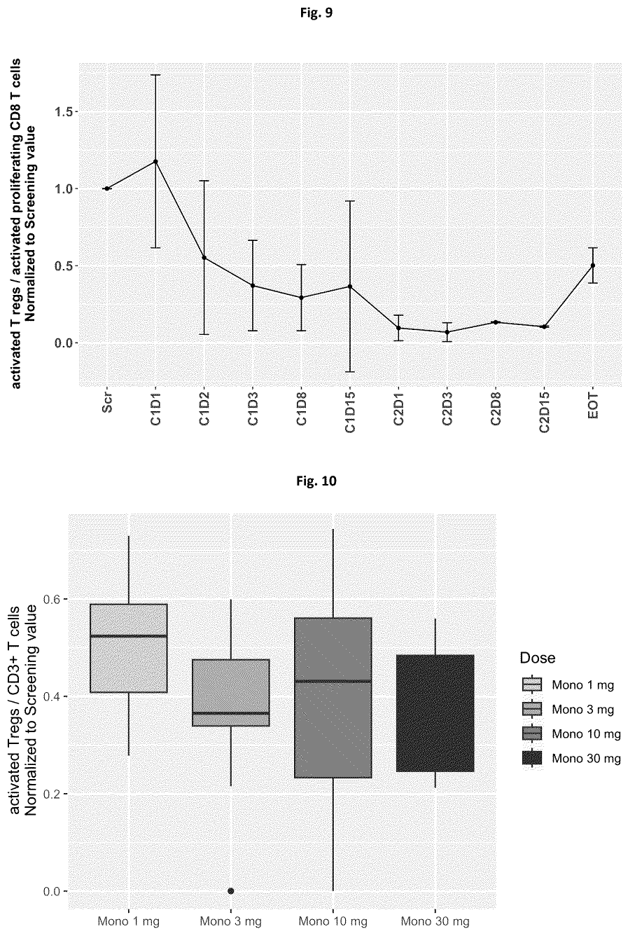

The pharmacologically active/efficacious dose range for the anti-CCR8 antibody TPP-23411, which induces substantial amounts of ADCC and ADCP but is characterized by a comparably low half-life, is 2.7 mg to 75 mg for a QW schedule and 16 mg to 450 mg for a Q3W schedule for a patient of 70 kg, see Example 16. Data substantiating the successful mode of action are shown e.g. in Example 24, see FIG. 7, FIG. 8, FIG. 9 and FIG. 10.

In accordance with these findings, in a most preferred embodiment there is provided an anti-human CCR8 antibody having ADCC activity and ADCP activity for use in a method of treatment comprising administering intravenously to a patient in need thereof the anti-CCR8 antibody in a total amount of 2.7 mg to 75 mg once every week.

The suggested medical use with a QW dosing schedule is superior because the anti-CCR8 antibody is provided with pharmacologically relevant plasma exposure levels, and also because the medical use allows for plasma Ctrough concentrations of the anti-CCR8 antibody above the estimated EC80 values for CCR8+ cell killing, which the inventors derived from in vitro studies.

In another most preferred embodiment there is provided an anti-human CCR8 antibody having ADCC activity and ADCP activity for use in a method of treatment comprising administering intravenously to a patient in need thereof the anti-CCR8 antibody in a total amount of 16 mg to 450 mg once every three weeks.

The medical use with a dosing schedule of Q3W comes with higher doses but is advantageous because these can be administered less frequently while still achieving the required plasma exposures during a dosing interval to produce the desired pharmacological response (CCR8+ Treg killing). The suggested Q3W dosing schedule also provides convenience of dosing and alignment with infusion of other drugs.

As understood by the skilled person, the total amount in the embodiments described herein is designed for a patient with an average weight of 70 kg and can be scaled based on the actual weight of the patient, i.e. by using the appropriate mg/kg.

Wherever reference is made to an anti-CCR8 antibody, this antibody is preferably TPP-23411, most preferably afucosylated TPP-23411.

In an utmost preferred example, the anti-CCR8 antibody having ADCC and ADCP activity for use in a method of treatment according to this aspect
a. comprises HCDR1, HCDR2, HCDR3, LCDR1, LCDR2 and LCDR3 sequences of SEQ ID numbers: 2, 3, 4, 6, 7 and 8, and/or
b. comprises a variable heavy chain sequence that has at least 98% or 100% sequence identity with the amino acid sequence set forth in SEQ ID NO:1 and/or a variable light chain sequence that has at least 98% or 100% sequence identity with the amino acid sequence set forth in SEQ ID NO:5, and/or
c. comprises a heavy chain sequence that has at least 98% or 100% sequence identity with the amino acid sequence set forth in SEQ ID NO:17 and/or a light chain sequence that has at least 98% or 100% sequence identity with the amino acid sequence set forth in SEQ ID NO:18.

However, the anti-CCR8 antibody can also be an alternative anti-human CCR8 antibody, e.g. an antibody described herein, inducing ADCC and ADCP and having a half-life that is comparable with the half-life of TPP-23411, i.e. an anti-human CCR8 antibody having a short half-life compared to other antibodies of the same IgG type. For example, the anti-human CCR8 antibody described herein may be characterized by a half-life of <14 days, preferably <12 days, more preferably <10 days, and most preferably <7 days, e.g. 0-120 h in human. Preferably, the anti-human CCR8 antibody is characterized by a half-life of <14 days, more preferably <10 days in human, most preferably <7 days in human.

Preferably, the anti-CCR8 antibody is a human IgG1 antibody.

Preferably, the anti-CCR8 antibody is (furthermore) a low internalizing or non-internalizing antibody.

In preferred embodiments, the anti-CCR8 antibody is characterized by a dissociation constant (KD) for binding CHO cells transfected with human CCR8 which is in the same order of magnitude as the KD of TPP-23411. As understood by the skilled person, an order-of-magnitude difference between two values is a factor of 10. In a most preferred embodiment according to the current aspect, the anti-CCR8 antibody is
a. characterized by a dissociation constant (KD) for binding CHO cells transfected with human CCR8 which is in the same order of magnitude as the KD of TPP-23411 for binding CHO cells transfected with human CCR8,
b. wherein the antibody induces ADCC and ADCP
   preferably wherein the antibody binds to human Fc gamma receptor IIIA variant V176 (CD16a) with a dissociation constant (KD) which is in the same order of magnitude as the KD of TPP-23411 for binding human Fc gamma receptor IIIA variant V176 (CD16a) and
   preferably wherein the antibody binds to human Fc gamma RIIA (CD32a) with a dissociation constant (KD) which is in the same order of magnitude as the KD of TPP-23411 for binding human Fc gamma RIIA (CD32a);
   preferably wherein the antibody is afucosylated; and
c. wherein the antibody is characterized by a half-life of <14 days, preferably <10 days, most preferably <7 days in human.

For the preparation of the intravenous infusions the required volume of the anti-CCR8 antibody solution can be withdrawn from (a) vial(s) and transferred into an intravenous (IV) bag containing 0.9% Sodium Chloride Injection, USP or 5% Dextrose Injection, USP. The diluted solution can be mixed by gentle inversion without shaking. The final concentration of the diluted solution can be for example between 1 mg/mL to 10 mg/mL.

Administration of the anti-CCR8 antibody (e.g. of the diluted solution) can occur intravenously over 15 to 120 minutes, preferably over 30 to 60 minutes, most preferably over 30, 45, 60 or 75 minutes.

Administration of the diluted solution can occur through an intravenous line, e.g. containing a sterile, non-pyrogenic, low-protein binding 0.2 micron to 5 micron in-line or add-on filter. Given the variability of infusion pumps from site to site, a window between −5 min and +10 min is permitted (i.e., infusion time is 30 min [−5 min/+10 min]).

The medical use according to the first aspect may further comprise administering intravenously to a patient in need thereof an anti-PD-(L)1 antibody in a total amount of
i. Approximately 200 mg once every three weeks, preferably wherein the anti-PD-(L)1 antibody is pembrolizumab, or
ii. Approximately 400 mg once every six weeks, preferably wherein the anti-PD-(L)1 antibody is pembrolizumab, or
iii. Approximately 240 mg once every two weeks, preferably wherein the anti-PD-(L)1 antibody is nivolumab, or
iv. Approximately 360 mg once every three weeks, preferably wherein the anti-PD-(L)1 antibody is nivolumab, or
v. Approximately 480 mg once every four weeks, preferably wherein the anti-PD-(L)1 antibody is nivolumab, or
vi. Approximately 840 mg every two weeks, preferably wherein the anti-PD-(L)1 antibody is atezolizumab, or
vii. Approximately 1200 mg every three weeks, preferably wherein the anti-PD-(L)1 antibody is atezolizumab, or
viii. Approximately 1680 mg every four weeks, preferably wherein the anti-PD-(L)1 antibody is atezolizumab, or
ix. Approximately 360 mg every three weeks, preferably wherein the anti-PD-(L)1 antibody is Zimberelimab, or
x. Approximately 3 mg/kg every two weeks, preferably wherein the anti-PD-(L)1 antibody is Toripalimab, or
xi. Approximately 10 mg/kg every two weeks, preferably wherein the anti-PD-(L)1 antibody is Durvalumab, or
xii. Approximately 1500 mg every 3 weeks, preferably wherein the anti-PD-(L)1 antibody is Durvalumab.

For example, preferably, the medical use according to the first aspect comprises administering intravenously to a patient in need thereof an anti-PD-(L)1 antibody in a total amount of approximately 200 mg once every three weeks, or approximately 400 mg once every six weeks, wherein the anti-PD-(L)1 antibody is pembrolizumab.

For example, preferably, the medical use according to the first aspect comprises administering intravenously to a patient in need thereof an anti-PD-(L)1 antibody in a total amount of approximately 240 mg once every two weeks, or approximately 360 mg once every three weeks, or approximately 480 mg once every four weeks, wherein the anti-PD-(L)1 antibody is nivolumab.

For example, preferably, the medical use according to the first aspect comprises administering intravenously to a patient in need thereof an anti-PD-(L)1 antibody in a total amount of approximately 840 mg every two weeks, approximately 1200 mg every three weeks, or approximately 1680 mg every four weeks, wherein the anti-PD-(L)1 antibody is atezolizumab.

In another example, the medical use according to the first aspect comprises administering intravenously to a patient in need thereof an anti-PD-(L)1 antibody in a total amount of approximately 360 mg every three weeks, wherein the anti-PD-(L)1 antibody is Zimberelimab.

In another example, the medical use according to the first aspect comprises administering intravenously to a patient in need thereof an anti-PD-(L)1 antibody in a total amount of approximately 3 mg/kg every two weeks, wherein the anti-PD-(L)1 antibody is Toripalimab.

In another example, the medical use according to the first aspect comprises administering intravenously to a patient in need thereof an anti-PD-(L)1 antibody in a total amount of approximately 10 mg/kg every two weeks, or approximately 1500 mg every 3 weeks, wherein the anti-PD-(L)1 antibody is Durvalumab.

In the alternative, the medical use according to the first aspect may comprise administering intravenously to a patient in need thereof an anti-PD-(L)1 antibody in a total amount of
a. Approximately 200 mg once every three weeks, or
b. Approximately 480 mg once every four weeks, or
c. Approximately 480 mg once every six weeks.

In this alternative, the anti-PD-(L)1 antibody is pembrolizumab, nivolumab, atezolizumab, avelumab, or durvalumab.

Using a fixed dose for the anti-PD-(L)1 antibody reduces dosing complexity and comes with a reduced potential for dosing errors. While the administration in a different order or even in a concomitant setup may be possible, the anti-PD-(L)1 antibody is preferably administered after the anti-CCR8 antibody.

In a most preferred embodiment of the current invention, there is provided an anti-human CCR8 antibody having ADCC activity and ADCP activity for use in a method of treatment
a. comprising administering intravenously to a patient in need thereof the anti-CCR8 antibody in a total amount of 2.7 mg to 75 mg once every week,
b. preferably further comprising administering intravenously to the patient an anti-PD-(L)1 antibody in a total amount of
   i. Approximately 200 mg once every three weeks, preferably wherein the anti-PD-(L)1 antibody is pembrolizumab, or
   ii. Approximately 400 mg once every six weeks, preferably wherein the anti-PD-(L)1 antibody is pembrolizumab, or
   iii. Approximately 240 mg once every two weeks, preferably wherein the anti-PD-(L)1 antibody is nivolumab, or
   iv. Approximately 360 mg once every three weeks, preferably wherein the anti-PD-(L)1 antibody is nivolumab, or
   v. Approximately 480 mg once every four weeks, preferably wherein the anti-PD-(L)1 antibody is nivolumab, or
   vi. Approximately 840 mg every two weeks, preferably wherein the anti-PD-(L)1 antibody is atezolizumab, or
   vii. Approximately 1200 mg every three weeks, preferably wherein the anti-PD-(L)1 antibody is atezolizumab, or
   viii. Approximately 1680 mg every four weeks, preferably wherein the anti-PD-(L)1 antibody is atezolizumab, or
   ix. Approximately 360 mg every three weeks, preferably wherein the anti-PD-(L)1 antibody is Zimberelimab, or
   x. Approximately 3 mg/kg every two weeks, preferably wherein the anti-PD-(L)1 antibody is Toripalimab, or
   xi. Approximately 10 mg/kg every two weeks, preferably wherein the anti-PD-(L)1 antibody is Durvalumab, or
   xii. Approximately 1500 mg every 3 weeks, preferably wherein the anti-PD-(L)1 antibody is Durvalumab.

In another most preferred embodiment there is provided an anti-human CCR8 antibody having ADCC activity and ADCP activity for use in a method of treatment
a. comprising administering intravenously to a patient in need thereof the anti-CCR8 antibody in a total amount of 16 mg to 450 mg once every three weeks,
b. preferably further comprising administering intravenously to the patient an anti-PD-(L)1 antibody in a total amount of
   i. Approximately 200 mg once every three weeks, preferably wherein the anti-PD-(L)1 antibody is pembrolizumab, or
   ii. Approximately 400 mg once every six weeks, preferably wherein the anti-PD-(L)1 antibody is pembrolizumab, or
   iii. Approximately 240 mg once every two weeks, preferably wherein the anti-PD-(L)1 antibody is nivolumab, or
   iv. Approximately 360 mg once every three weeks, preferably wherein the anti-PD-(L)1 antibody is nivolumab, or
   v. Approximately 480 mg once every four weeks, preferably wherein the anti-PD-(L)1 antibody is nivolumab, or
   vi. Approximately 840 mg every two weeks, preferably wherein the anti-PD-(L)1 antibody is atezolizumab, or
   vii. Approximately 1200 mg every three weeks, preferably wherein the anti-PD-(L)1 antibody is atezolizumab, or
   viii. Approximately 1680 mg every four weeks, preferably wherein the anti-PD-(L)1 antibody is atezolizumab, or
   ix. Approximately 360 mg every three weeks, preferably wherein the anti-PD-(L)1 antibody is Zimberelimab, or
   x. Approximately 3 mg/kg every two weeks, preferably wherein the anti-PD-(L)1 antibody is Toripalimab, or
   xi. Approximately 10 mg/kg every two weeks, preferably wherein the anti-PD-(L)1 antibody is Durvalumab, or
   xii. Approximately 1500 mg every 3 weeks, preferably wherein the anti-PD-(L)1 antibody is Durvalumab.

For the preparation of the intravenous infusions, the required volume of the anti-PD-(L)1 antibody solution can be withdrawn from the vial(s) and transferred into an intravenous (IV) bag containing 0.9% Sodium Chloride Injection, USP or 5% Dextrose Injection, USP. The diluted solution can be mixed by gentle inversion without shaking. The final concentration of the diluted solution can be for example between 1 mg/mL to 10 mg/mL.

For example, the intravenous administration of the anti-PD-(L)1 antibody may occur as a 15- to 60-minute intravenous infusion, and preferably as a 30-minute intravenous infusion.

Administration of the anti-PD-(L)1 antibody may occur through an intravenous line containing a sterile, non-pyrogenic, low-protein binding 0.2 micron to 5 micron in-line or add-on filter. Given the variability of infusion pumps from site to site, a window between −5 min and +10 min is permitted (i.e., infusion time is 30 min [−5 min/+10 min]).

For example, the intravenous administration of the anti-PD-(L)1 antibody may occur using the same IV line that was previously used for the intravenous administration of the anti-human CCR8 antibody. This setup is preferred and advantageous because the complexity of treatment administration is reduced and because this is highly convenient for both patients and medical professionals. Preferably the IV line is flushed with saline prior to the intravenous administration of second antibody, i.e. the anti-human PD-(L)1 antibody.

For example, the anti-human CCR8 antibody having ADCC and ADCP activity for use in a method of treatment according to this aspect may comprise at least one 21-day dosing cycle. Furthermore, the anti-CCR8 antibody and the anti-PD-(L)1 antibody may both be administered on day 1 of the 21-day dosing cycle.

If all previous infusions (e.g. in cycle 1, 2, 3, or 4) have been well tolerated by a patient, the PD-(L)1 antibody may be administered without substantial delay, i.e. without a pause of 15-60 minutes directly after the anti-CCR8 antibody. For example, where the medical use comprises at least two and preferably more dosing cycles, for the second, third, fourth, fifth or any subsequent dosing cycle the anti-CCR8 antibody and the anti-PD-(L)1 antibody can be administered without substantial delay directly after each other. This method is superior because it is more time efficient for the patients and the medical professionals.

In a preferred embodiment, the anti-PD-(L)1 antibody is pembrolizumab and is administered in a total amount of approximately 200 mg once every three weeks, or approximately 400 mg once every six weeks.

For example, in one embodiment the total amount of the anti-CCR8 antibody is approximately 10 mg once every week, and the anti-PD-(L)1 antibody is pembrolizumab and is administered in a total amount of approximately 200 mg once every three weeks, or approximately 400 mg once every six weeks.

For example, in one embodiment the total amount of the anti-CCR8 antibody is approximately 30 mg once every week, and the anti-PD-(L)1 antibody is pembrolizumab and is administered in a total amount of approximately 200 mg once every three weeks, or approximately 400 mg once every six weeks.

In a preferred example the total amount of the anti-CCR8 antibody is approximately 50 mg once every week and the anti-PD-(L)1 antibody is pembrolizumab and is administered in a total amount of approximately 200 mg once every three weeks, or approximately 400 mg once every six weeks.

For example, in one embodiment the total amount of the anti-CCR8 antibody is approximately 100 mg once every week, and the anti-PD-(L)1 antibody is pembrolizumab and is administered in a total amount of approximately 200 mg once every three weeks, or approximately 400 mg once every six weeks.

In another preferred example the total amount of the anti-CCR8 antibody is approximately 125 mg once every week and the anti-PD-(L)1 antibody is pembrolizumab and is administered in a total amount of approximately 200 mg once every three weeks, or approximately 400 mg once every six weeks.

In a highly preferred example the total amount of the anti-CCR8 antibody is approximately 250 mg once every week and the anti-PD-(L)1 antibody is pembrolizumab and is administered in a total amount of approximately 200 mg once every three weeks, or approximately 400 mg once every six weeks.

In a further highly preferred example the total amount of the anti-CCR8 antibody is approximately 500 mg once every three weeks and the anti-PD-(L)1 antibody is pembrolizumab and is administered in a total amount of approximately 200 mg once every three weeks, or approximately 400 mg once every six weeks.

In a highly preferred example the total amount of the anti-CCR8 antibody is approximately 750 mg once every three weeks and the anti-PD-(L)1 antibody is pembrolizumab and is administered in a total amount of approximately 200 mg once every three weeks, or approximately 400 mg once every six weeks.

In a highly preferred embodiment the total amount of the anti-CCR8 antibody is approximately 1000 mg once every three week and the anti-PD-(L)1 antibody is pembrolizumab and is administered in a total amount of approximately 200 mg once every three weeks, or approximately 400 mg once every six weeks.

In a preferred example the total amount of the anti-CCR8 antibody is approximately 1500 mg once every three week and the anti-PD-(L)1 antibody is pembrolizumab and is administered in a total amount of approximately 200 mg once every three weeks, or approximately 400 mg once every six weeks.

In another preferred embodiment, the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

For example, in one embodiment the total amount of the anti-CCR8 antibody is approximately 10 mg once every week, and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

For example, in one embodiment the total amount of the anti-CCR8 antibody is approximately 30 mg once every week, and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a preferred example the total amount of the anti-CCR8 antibody is approximately 50 mg once every week and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

For example, in one embodiment the total amount of the anti-CCR8 antibody is approximately 100 mg once every week, and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In another preferred example the total amount of the anti-CCR8 antibody is approximately 125 mg once every week and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a highly preferred example the total amount of the anti-CCR8 antibody is approximately 250 mg once every week and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a further highly preferred example the total amount of the anti-CCR8 antibody is approximately 500 mg once every three weeks and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a highly preferred example the total amount of the anti-CCR8 antibody is approximately 750 mg once every three weeks and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a highly preferred embodiment the total amount of the anti-CCR8 antibody is approximately 1000 mg once every three week and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a preferred example the total amount of the anti-CCR8 antibody is approximately 1500 mg once every three week and the anti-PD-(L)1 antibody is nivolumab and is administered in a total amount of approximately 240 mg once every two weeks, approximately 360 mg once every three weeks, approximately 480 mg once every four weeks, or approximately 480 mg once every six weeks.

In a preferred embodiment, the anti-PD-(L)1 antibody is atezolizumab and is administered in a total amount of approximately 840 mg once every two weeks, approximately 1200 mg once every three weeks, or approximately 1680 mg once every four weeks.

For example, in one embodiment the total amount of the anti-CCR8 antibody is approximately 10 mg once every week, and the anti-PD-(L)1 antibody is atezolizumab and is administered in a total amount of approximately 840 mg once every two weeks, approximately 1200 mg once every three weeks, or approximately 1680 mg once every four weeks.

For example, in one embodiment the total amount of the anti-CCR8 antibody is approximately 30 mg once every week, and the anti-PD-(L)1 antibody is atezolizumab and is administered in a total amount of approximately 840 mg once every two weeks, approximately 1200 mg once every three weeks, or approximately 1680 mg once every four weeks.

In a preferred example the total amount of the anti-CCR8 antibody is approximately 50 mg once every week and the anti-PD-(L)1 antibody is atezolizumab and is administered in a total amount of approximately 840 mg once every two weeks, approximately 1200 mg once every three weeks, or approximately 1680 mg once every four weeks.

For example, in one embodiment the total amount of the anti-CCR8 antibody is approximately 100 mg once every week, and the anti-PD-(L)1 antibody is atezolizumab and is administered in a total amount of approximately 840 mg once every two weeks, approximately 1200 mg once every three weeks, or approximately 1680 mg once every four weeks.

In another preferred example the total amount of the anti-CCR8 antibody is approximately 125 mg once every week and the anti-PD-(L)1 antibody is atezolizumab and is administered in a total amount of approximately 840 mg once every two weeks, approximately 1200 mg once every three weeks, or approximately 1680 mg once every four weeks.

In a highly preferred example the total amount of the anti-CCR8 antibody is approximately 250 mg once every week and the anti-PD-(L)1 antibody is atezolizumab and is administered in a total amount of approximately 840 mg once every two weeks, approximately 1200 mg once every three weeks, or approximately 1680 mg once every four weeks.

In a further highly preferred example the total amount of the anti-CCR8 antibody is approximately 500 mg once every three weeks and the anti-PD-(L)1 antibody is atezolizumab and is administered in a total amount of approximately 840 mg once every two weeks, approximately 1200 mg once every three weeks, or approximately 1680 mg once every four weeks.

In a highly preferred example the total amount of the anti-CCR8 antibody is approximately 750 mg once every three weeks and the anti-PD-(L)1 antibody is atezolizumab and is administered in a total amount of approximately 840 mg once every two weeks, approximately 1200 mg once every three weeks, or approximately 1680 mg once every four weeks.

In a highly preferred embodiment the total amount of the anti-CCR8 antibody is approximately 1000 mg once every three week and the anti-PD-(L)1 antibody is atezolizumab and is administered in a total amount of approximately 840 mg once every two weeks, approximately 1200 mg once every three weeks, or approximately 1680 mg once every four weeks.

In a preferred example the total amount of the anti-CCR8 antibody is approximately 1500 mg once every three week and the anti-PD-(L)1 antibody is atezolizumab and is administered in a total amount of approximately 840 mg once every two weeks, approximately 1200 mg once every three weeks, or approximately 1680 mg once every four weeks.

In a preferred embodiment, the anti-PD-(L)1 antibody is Zimberelimab and is administered in a total amount of approximately 360 mg once every three weeks.

For example, in one embodiment the total amount of the anti-CCR8 antibody is approximately 10 mg once every week, and the anti-PD-(L)1 antibody is Zimberelimab and is administered in a total amount of approximately 360 mg once every three weeks.

For example, in one embodiment the total amount of the anti-CCR8 antibody is approximately 30 mg once every week, and the anti-PD-(L)1 antibody is Zimberelimab and is administered in a total amount of approximately 360 mg once every three weeks.

In a preferred example the total amount of the anti-CCR8 antibody is approximately 50 mg once every week and the anti-PD-(L)1 antibody is Zimberelimab and is administered in a total amount of approximately 360 mg once every three weeks.

For example, in one embodiment the total amount of the anti-CCR8 antibody is approximately 100 mg once every week, and the anti-PD-(L)1 antibody is Zimberelimab and is administered in a total amount of approximately 360 mg once every three weeks.

In another preferred example the total amount of the anti-CCR8 antibody is approximately 125 mg once every week and the anti-PD-(L)1 antibody is Zimberelimab and is administered in a total amount of approximately 360 mg once every three weeks.

In a highly preferred example the total amount of the anti-CCR8 antibody is approximately 250 mg once every week and the anti-PD-(L)1 antibody is Zimberelimab and is administered in a total amount of approximately 360 mg once every three weeks.

In a further highly preferred example the total amount of the anti-CCR8 antibody is approximately 500 mg once every three weeks and the anti-PD-(L)1 antibody is Zimberelimab and is administered in a total amount of approximately 360 mg once every three weeks.

In a highly preferred example the total amount of the anti-CCR8 antibody is approximately 750 mg once every three weeks and the anti-PD-(L)1 antibody is Zimberelimab and is administered in a total amount of approximately 360 mg once every three weeks.

In a highly preferred embodiment the total amount of the anti-CCR8 antibody is approximately 1000 mg once every three week and the anti-PD-(L)1 antibody is Zimberelimab and is administered in a total amount of approximately 360 mg once every three weeks.

In a preferred example the total amount of the anti-CCR8 antibody is approximately 1500 mg once every three week and the anti-PD-(L)1 antibody is Zimberelimab and is administered in a total amount of approximately 360 mg once every three weeks.

In a preferred embodiment, the anti-PD-(L)1 antibody is Toripalimab and is administered in a total amount of approximately 3 mg/kg once every two weeks.

For example, in one embodiment the total amount of the anti-CCR8 antibody is approximately 10 mg once every week, and the anti-PD-(L)1 antibody is Toripalimab and is administered in a total amount of approximately 3 mg/kg once every two weeks.

For example, in one embodiment the total amount of the anti-CCR8 antibody is approximately 30 mg once every week, and the anti-PD-(L)1 antibody is Toripalimab and is administered in a total amount of approximately 3 mg/kg once every two weeks.

In a preferred example the total amount of the anti-CCR8 antibody is approximately 50 mg once every week and the anti-PD-(L)1 antibody is Toripalimab and is administered in a total amount of approximately 3 mg/kg once every two weeks.

For example, in one embodiment the total amount of the anti-CCR8 antibody is approximately 100 mg once every week, and the anti-PD-(L)1 antibody is Toripalimab and is administered in a total amount of approximately 3 mg/kg once every two weeks.

In another preferred example the total amount of the anti-CCR8 antibody is approximately 125 mg once every week and the anti-PD-(L)1 antibody is Toripalimab and is administered in a total amount of approximately 3 mg/kg once every two weeks.

In a highly preferred example the total amount of the anti-CCR8 antibody is approximately 250 mg once every week and the anti-PD-(L)1 antibody is Toripalimab and is administered in a total amount of approximately 3 mg/kg once every two weeks.

In a further highly preferred example the total amount of the anti-CCR8 antibody is approximately 500 mg once every three weeks and the anti-PD-(L)1 antibody is Toripalimab and is administered in a total amount of approximately 3 mg/kg once every two weeks.

In a highly preferred example the total amount of the anti-CCR8 antibody is approximately 750 mg once every three weeks and the anti-PD-(L)1 antibody is Toripalimab and is administered in a total amount of approximately 3 mg/kg once every two weeks.

In a highly preferred embodiment the total amount of the anti-CCR8 antibody is approximately 1000 mg once every three week and the anti-PD-(L)1 antibody is Toripalimab and is administered in a total amount of approximately 3 mg/kg once every two weeks.

In a preferred example the total amount of the anti-CCR8 antibody is approximately 1500 mg once every three week and the anti-PD-(L)1 antibody is Toripalimab and is administered in a total amount of approximately 3 mg/kg once every two weeks.

In a preferred embodiment, the anti-PD-(L)1 antibody is Durvalumab and is administered in a total amount of approximately 10 mg/kg every two weeks, or approximately 1500 mg every 3 weeks.

For example, in one embodiment the total amount of the anti-CCR8 antibody is approximately 10 mg once every week, and the anti-PD-(L)1 antibody is Durvalumab and is administered in a total amount of approximately 10 mg/kg every two weeks, or approximately 1500 mg every 3 weeks.

For example, in one embodiment the total amount of the anti-CCR8 antibody is approximately 30 mg once every week, and the anti-PD-(L)1 antibody is Durvalumab and is administered in a total amount of approximately 10 mg/kg every two weeks, or approximately 1500 mg every 3 weeks.

In a preferred example the total amount of the anti-CCR8 antibody is approximately 50 mg once every week and the anti-PD-(L)1 antibody is Durvalumab and is administered in a total amount of approximately 10 mg/kg every two weeks, or approximately 1500 mg every 3 weeks.

For example, in one embodiment the total amount of the anti-CCR8 antibody is approximately 100 mg once every week, and the anti-PD-(L)1 antibody is Durvalumab and is administered in a total amount of approximately 10 mg/kg every two weeks, or approximately 1500 mg every 3 weeks.

In another preferred example the total amount of the anti-CCR8 antibody is approximately 125 mg once every week and the anti-PD-(L)1 antibody is Durvalumab and is administered in a total amount of approximately 10 mg/kg every two weeks, or approximately 1500 mg every 3 weeks.

In a highly preferred example the total amount of the anti-CCR8 antibody is approximately 250 mg once every week and the anti-PD-(L)1 antibody is Durvalumab and is administered in a total amount of approximately 10 mg/kg every two weeks, or approximately 1500 mg every 3 weeks.

In a further highly preferred example the total amount of the anti-CCR8 antibody is approximately 500 mg once every three weeks and the anti-PD-(L)1 antibody is Durvalumab and is administered in a total amount of approximately 10 mg/kg every two weeks, or approximately 1500 mg every 3 weeks.

In a highly preferred example the total amount of the anti-CCR8 antibody is approximately 750 mg once every three weeks and the anti-PD-(L)1 antibody is Durvalumab and is administered in a total amount of approximately 10 mg/kg every two weeks, or approximately 1500 mg every 3 weeks.

In a highly preferred embodiment the total amount of the anti-CCR8 antibody is approximately 1000 mg once every three week and the anti-PD-(L)1 antibody is Durvalumab and is administered in a total amount of approximately 10 mg/kg every two weeks, or approximately 1500 mg every 3 weeks.

In a preferred example the total amount of the anti-CCR8 antibody is approximately 1500 mg once every three week and the anti-PD-(L)1 antibody is Durvalumab and is administered in a total amount of approximately 10 mg/kg every two weeks, or approximately 1500 mg every 3 weeks.

The medical use according to the first aspect preferably comprises at least one 21-day dosing cycle.

In some preferred of these embodiments, the anti-CCR8 antibody and the anti-PD-(L)1 antibody are both administered on day 1 of the 21-day dosing cycle.

The dosing schemes according to the current invention are particularly suited for the use of the anti-CCR8 antibody in a method of treating cancer such as non-small cell lung cancer (NSCLC), head and neck squamous cell carcinoma (HNSCC), triple-negative breast cancer (TNBC), melanoma, other types of skin cancer and cancer.

The dosing schemes according to the current invention are particularly suited for the use of the anti-CCR8 antibody in a method of treating a patient having a tumor or a disease characterized by chemokine receptor positive cells, preferably CCR8 positive cells, such as CCR8 positive regulatory T cells.

For example, the method of treatment is a method of treating cancer, preferably wherein the cancer is non-small cell lung cancer (NSCLC), triple-negative breast cancer (TNBC), head and neck squamous cell carcinoma (HNSCC), melanoma, or a skin cancer other than melanoma.

For example, the method of treatment is non-small cell lung cancer (NSCLC). For example, the method of treatment is triple-negative breast cancer (TNBC). For example, the method of treatment is head and neck squamous cell carcinoma (HNSCC). For example, the method of treatment is melanoma, or a skin cancer other than melanoma.

According to the current invention it was furthermore interestingly found, that certain side reactions to anti-CCR8 antibody administration could be prevented by administration of an effective dose of antihistamines, acetaminophen, corticosteroids or a combination thereof. In consequence, the method of treatment according to the current aspect may furthermore comprises administration of an effective dose of antihistamines, acetaminophen, corticosteroids or a combination thereof, preferably
a. At least 500 mg or at least 650 mg paracetamol prior to the administration of the anti-CCR8 antibody, and/or
b. At least 50 mg or at least 100 mg diphenhydramine prior to the administration of anti-CCR8 antibody and/or
c. At least 8 mg dexamethasone prior to the administration of anti-CCR8 antibody.

For example the acetaminophen may be administered orally. For example the diphenhydramine may be administered orally.

### Tumor Proportion Score/Combined Positive Score

Response rates to immune checkpoint inhibitors (ICIs) differ dramatically depending on cancer type, ranging from below 15% to over 60%. Significant proportions of ‘immunosensitive’ tumor types are either refractory to therapy from the start of ICI treatment (primary resistance) or will, after initial clinical benefit, eventually acquire resistance (secondary or acquired resistance). Therapeutic options after failure of frontline standard of care (SoC) with ICIs (including combinations with other anticancer agents) remain limited in most advanced tumor settings. There is therefore a high medical need in these “post ICI therapy” patients to define treatments that overcome the resistance to (combined) immunotherapy of their malignant diseases as alternative treatment options for these patients are limited.

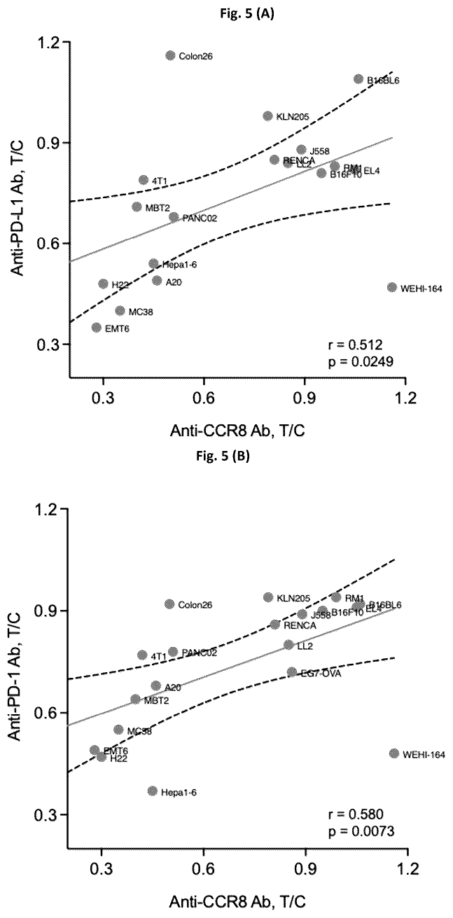

Association between gene expression and the in vivo efficacy of treatments with CCR8-depleting antibodies was studied by whole transcriptome sequencing of untreated syngeneic mouse tumor models. The inventors found that baseline expression of PD-L1 correlated well with the in vivo antitumor efficacy of CCR8 surrogate antibodies across 21 tumor models, see FIG. 5.

According to some preferred embodiments of the first aspect, the method of treatment is a method of treating cancer comprising the steps of
a. Analysing the Tumor Proportion Score or the Combined Positive Score as a measure for PD-(L)1 expression in a cancer tissue sample of the patient, and
b. Administering the anti-human CCR8 antibody to the patient if the patient has a Tumor Proportion Score of ≥50% or a Combined Positive Score of ≥10% or ≥1%.

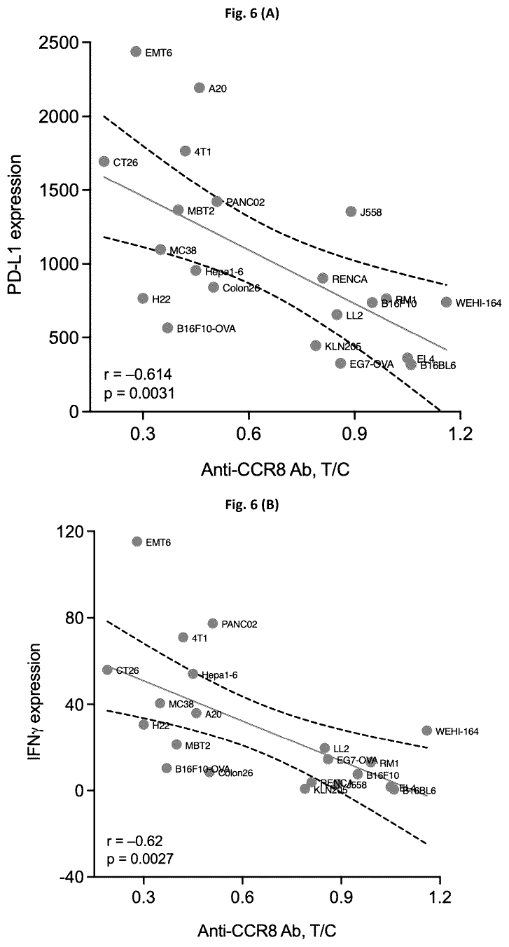

These stratification steps can be applied in order to determine the likelihood of the patient to profit from administration of the anti-human CCR8 antibody. In particular, PD-L1 expression can be used as stratification strategy for patient selection and as eligibility criterion to treat patients. For example, in the monotherapy-Mode of action (MoA) expansion arm (Arm 2A) of the clinical study, a historic PD-L1 score of tumor proportion score (TPS)≥50% is used as an eligibility criterion. PD-L1 expression seems to have general suitability as a predictive biomarker, see also FIG. 6.

According to some preferred of these embodiments,
a. the cancer is non-small cell lung cancer (NSCLC) and the Tumor Proportion Score is analysed as a measure for PD-(L)1 expression in a cancer tissue sample of the patient, or
b. the cancer is triple negative breast cancer and the Combined Positive Score is analysed as a measure for PD-(L)1 expression in a cancer tissue sample of the patient, or
c. the cancer is head and neck squamous cell carcinoma and the Combined Positive Score is analysed as a measure for PD-(L)1 expression in a cancer tissue sample of the patient.

Preferably, the Tumor Proportion Score is analysed using PD-L1-antibody 22C3 pharmDx assay. The PD-L1-antibody 22C3 pharmDx assay provides reliable results and has been approved by the FDA. PD-L1 IHC 22C3 pharmDx is a qualitative immunohistochemical assay using monoclonal mouse anti-PD-L1, Clone 22C3 and can be used in the detection of PD-L1 protein in formalin-fixed, paraffin-embedded (FFPE) cancer tissue, e.g. using EnVision FLEX visualization system on Autostainer Link 48. In the alternative, the VENTANA PD-L1 (SP142) Assay can be used. The VENTANA PD-L1 (SP142) Assay is another qualitative immunohistochemical assay using rabbit monoclonal anti-PD-L1 clone SP142 and can be used in FFPE tissues stained e.g. with OptiView DAB IHC Detection Kit and OptiView Amplification Kit on a BenchMark ULTRA instrument.

Reference is made to the PD-L1 IHC 22C3 pharmDx Interpretation Manual—NSCLC (Agilent Dako) for assessing the Tumor Proportion Score and reference is furthermore made to the PD-L1 IHC 22C3 pharmDx Interpretation Manual—Head and Neck Squamous Cell Carcinoma (Agilent Dako) for assessing the CPS.

For some cancer patients, a historic Tumor Proportion Score and/or a historic Combined Positive Score is available. According to the current invention, the historic Tumor Proportion Score or historic Combined Positive Score can be used to determine the likelihood of the patient to profit from administration of the anti-human CCR8 antibody. Using these historic scores, a sufficiently reliable, fast and highly convenient stratification decision can be made.

In particular, there is provided the anti-human CCR8 antibody having ADCC activity and ADCP activity for use in a method of treatment as described according to the first aspect, wherein the method of treatment is a method of treating cancer comprising administering the anti-human CCR8 antibody to the patient if the patient has a historic Tumor Proportion Score of ≥50% or a historic Combined Positive Score of ≥10% or ≥1%.

According to some preferred of these embodiments,
a. the cancer is non-small cell lung cancer (NSCLC) and the method of treatment comprises administering the anti-human CCR8 antibody to the patient if the patient has a historic Tumor Proportion Score of ≥50%, or
b. the cancer is triple negative breast cancer and the method of treatment comprises administering the anti-human CCR8 antibody to the patient if the patient has a historic Combined Positive Score of ≥10% or ≥1%, or
c. the cancer is head and neck squamous cell carcinoma and the method of treatment comprises administering the anti-human CCR8 antibody to the patient if the patient has a historic Combined Positive Score of ≥20%, or ≥1%.

According to some embodiments described in this section, the Tumor Proportion Score is analysed or was obtained using an FDA-approved PD-L1 assay such as PD-L1 IHC 22C3 pharmDx assay or VENTANA PD-L1 (SP263) assay.

### Cytokine Biomarker

Cytokine release was identified as an important biomarker in the context of anti-CCR8 antibodies. According to the current invention there is provided an anti-human CCR8 antibody having ADCC activity and ADCP activity for use in a method of treatment for a patient, e.g. without limitation according to any of the aspects or embodiments described herein, comprising the steps of:
a. Optionally analysing in a blood, plasma or serum screening sample of the patient the level of at least one and preferably at least 2, 3, 4, 5, 6, 7, 8, 9 or 10 inflammatory cytokines, selected from the group of IFN-γ, IL-1β, IL-2, IL-4, IL-6, IL-8, IL-10, IL 12p70, IL-13, and TNF-α,
b. Administering to the patient an effective dose of the anti-human CCR8 antibody,
c. Analysing in a blood, plasma or serum sample of the patient the level of the at least one and preferably at least 2, 3, 4, 5, 6, 7, 8, 9 or 10 inflammatory cytokines,
   wherein the blood, plasma or serum sample is drawn or collected after administering the effective dose of the anti-human CCR8 antibody according to step b),
d. Comparing the cytokine(s) level(s) obtained according to step c)
   i. either with the cytokine(s) level(s) obtained according to step a), or
   ii. with a reference value,
to identify safety-related events, or as a surrogate biomarker for Treg depletion, or as a biomarker for treatment success.

A screening sample is defined herein as a sample obtained from a patient before the actual dosing event, preferably from a patient that has not previously been treated with the anti-human CCR8 antibody having ADCC activity and ADCP activity.

To use the respective cytokine levels as a biomarker, the respective cytokine level can be compared for example to the cytokine level obtained before the first anti-CCR8 antibody administration for the same patient, or to a different reference value obtained for the same patient or a different group of subjects.

The skilled person is well aware that such a reference value can be obtained by calculating e.g. the average or median from cytokine levels obtained from multiple patients for a defined timepoint (e.g. without limitation before anti-CCR8 antibody administration, 1-12 hours after anti-CCR8 antibody administration, 1-3 days after anti-CCR8 antibody administration, at any of the time points measured according to Example 24 or at any other suitable time point thereafter. If the reference value is calculated using a time point after antibody administration, the skilled person is also aware that the antibody dose used for treating the subjects for the identification of the reference value should be well defined and could for example be for any of the dosages described herein for anti-CCR8 antibody administration. It is not expedient to define an exact pg/μl for each cytokine herein, because the skilled person knows how to calibrate the respective assay for biomarker evaluation. In any case, a substantial increase of the described biomarkers correlates with Treg depletion, and/or treatment success (compare FIG. 10 with FIGS. 11 to 16).

For example, a biomarker for treatment success can be a biomarker for monitoring, prediction or stratification. The term stratification refers to patient selection for treatment.

In a preferred embodiment, the at least one and preferably at least 2, 3, 4, 5, 6, 7, 8, 9 or 10 inflammatory cytokines, selected from the group of IFN-γ, IL-1β, IL-2, IL-4, IL-6, IL-8, IL-10, IL 12p70, IL-13, and TNF-α is IFN-γ or comprises IFN-γ.

In a preferred embodiment, the reference value is a value obtained from a sample of that same patient drawn/collected before the start of the treatment. For example, the blood, plasma or serum screening sample for analysing the cytokine level(s) may be drawn/collected 60-15 minutes and preferably ˜30 minutes before administering the effective dose of the anti-human CCR8 antibody.

### Cytokines as Safety Biomarkers for Anti-CCR8 Antibodies

In brief, cytokine release assays were conducted with human whole blood (with soluble antibody added) and human peripheral blood mononuclear cells (PBMCs) (with wet-coated antibody) to investigate the potential of anti-CCR8 antibodies alone or in combination with pembrolizumab to activate secretion of cytokines (IFN-γ, IL-1β, IL-2, IL-4, IL-6, IL-8, IL-10, and TNFα analyzed), see Example 14. Reference values for comparison of the cytokine(s) level(s) can be obtained for a specified anti-CCR8 antibody at a specific concentration as defined in this example. In the alternative, these reference values can be obtained based on an alternative antibody with a known safety profile, as also explained in this Example.

Indeed, the incubation of whole blood with soluble anti-human CCR8 antibody resulted in dose-dependent release of IFN-γ, IL-1β, IL-6, TNFα, and IL-8. When compared to rituximab, the anti-human CCR8 antibody induced higher IFN-γ, IL-6, TNFα, and IL-8 cytokine levels at 10 μg/mL dose but comparable levels at the lower antibody doses tested. IL-1 was not induced by rituximab but by the anti-human CCR8 antibody. IFN-γ was defined as lead cytokine due to its robust dose-response and the highest sensitivity.

Based on these findings, the inventors suggest the tested cytokine release panel as a safety biomarker for anti-CCR8 antibody cancer therapy to reduce the potential risks of infusion-related reactions due to the release of cytokines (including cytokine release syndrome) and to mitigate the risk of immune-related adverse events such as dermatologic toxicities (i.e., rash) observed with other Treg-depleting agents.

### Cytokines as Monitoring Biomarkers for Anti-CCR8 Antibodies

When testing fucosylated anti-CCR8 antibody variants or a ‘silenced’ Fc variant (with reduced binding to FcgRs) on two donors, those variants did not—or to a much lesser extent—induce IFN-γ, IL-1β, IL-6, TNFα, and IL-8 in the whole blood/soluble antibody cytokine release assay format, suggesting that the cytokine release indicates successful Treg depletion via ADCC and ADCP.

Because the anticipated mode of action of the afucosylated anti-CCR8 antibodies relies on ADCC and ADCP, the inventors concluded that cytokine release is useful as a surrogate biomarker for Treg depletion induced by an anti-CCR8 antibody and also as a predictive/stratification biomarker for treatment success. Indeed, FIG. 6 (B) shows a correlation between IFN-γ levels and treatment response for multiple mouse models, see also for human patients FIG. 10 in comparison with FIGS. 11 to 16.

In accordance with these findings, in some further embodiments the described anti-human CCR8 antibody having ADCC activity and ADCP activity for use in a method of treatment further comprises e. Administering to the patient at least one further effective dose of the anti-human CCR8 antibody, if the cytokine(s) level(s) obtained according to step c) are significantly increased
i. relative to the cytokine(s) level(s) obtained according to step a), or
ii. are increased relative to a reference value.

In the course of Example 13.2 and 13.3, cytokine levels were evaluated in cynomolgus monkeys. Example 14 and Example 25 discloses a cytokine release assay for human donors and increased cytokine release for increased anti-CCR8 antibody doses in human patients.

The analysis of the cytokine level(s) may occur e.g. using sandwich-based immunoassay techniques, e.g. the “Meso Scale Discovery” (MSD-ECL) platform. The MSD-ECL platform uses electrochemiluminescent labels that are conjugated to detection antibodies. These labels generate light when stimulated by electricity in the appropriate chemical environment, which can then be used to measure key proteins and molecules. The detection process is initiated at electrodes located in the bottom of the platform's microplates, and only labels near the electrode are excited and detected. Electricity is applied to the plate electrodes, leading to light emission by SULFO-TAG labels, which are electrochemiluminescent labels that allow for ultra-sensitive detection. Light intensity is then measured to quantify analytes in the sample.

In other words, in principle each well of the used microplate is pre-coated with capture antibody (specific for each of the cytokines to be detected) on spatially distinct spots. Plasma samples are added to the wells of the microplate and bound cytokines from the sample are detected with MSD SULFO-TAG labeled anti-cytokine detection antibodies. Electrochemiluminescence principle is used: For read out voltage is applied to the plate electrodes and the intensity of emitted light allows for quantitative measure of the respective cytokines present in the sample.

In an alternative example the analysis of the cytokine level(s) may also occur using “Single molecule array” (Simoa™). A Simoa is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not.

In some preferred embodiments, the level of at least one and preferably at least 2, 3, 4, 5, 6, 7, 8, 9 or 10 inflammatory cytokines, selected from the group of IFN-γ, IL-1β, IL-2, IL-4, IL-6, IL-8, IL-10, IL 12p70, IL-13, and TNF-α can be compared with baseline levels of the same cytokines obtained from the same patient at an earlier point in time or before the administration of the anti-CCR8 antibody.

In some preferred embodiments, the level of at least one and preferably at least 2, 3, 4, 5, 6, 7, 8, 9 or 10 inflammatory cytokines, selected from the group of IFN-γ, IL-1β, IL-2, IL-4, IL-6, IL-8, IL-10, IL 12p70, IL-13, and TNF-α can be compared to a reference value for each of the cytokines. This reference value can be determined by the skilled person and may deviate based on the concentration of the previously administered anti-CCR8 antibody, see Example 14. The reference value can be a general reference value or an individual patient specific reference value obtained from a sample drawn pre-treatment.

In some embodiments the blood, plasma or serum sample for analysing the cytokine level(s) is drawn on the same day after administering the effective dose of the anti-human CCR8 antibody, e.g. 1 hour, 2 hours, 3 hours, 4 hours, 5 hours or 6 hours after administering the effective dose of the anti-human CCR8 antibody. Drawing the blood sample on the same day may be convenient, in particular if the patient can be treated in an ambulatory setting.

In some embodiments the blood, plasma or serum sample for analysing the cytokine level(s) is drawn 1-24 hours or approx. 1, 2, 3, 4, 5, 6, 7, 8, 9, 10, 11, 12, 13, 14, 15, 16, 17, 18, 19, 20, or 21 days after administering the effective dose of the anti-human CCR8 antibody.

According to some preferred embodiments, the blood, plasma or serum sample for analysing the cytokine level(s) according to step c) is drawn 1-24 hours, 24-48 hours, 2-7 days, 7-14 days, 14-28 days, or more than 28 days after administering the effective dose of the anti-human CCR8 antibody according to step b).

According to some preferred embodiments, the blood, plasma or serum sample for analysing the cytokine level(s) according to step c) is drawn within the first three days after administering the effective dose of the anti-human CCR8 antibody. Drawing the sample within the first three days after administering the effective dose of the anti-human CCR8 antibody according to step b) is particularly suited to monitor safety relevant events.

According to some preferred embodiments, the blood, plasma or serum sample for analysing the cytokine level(s) according to step c) is drawn within 3-21 days after administering the effective dose of the anti-human CCR8 antibody. Drawing the sample 3-21 days after administering the effective dose of the anti-human CCR8 antibody according to step b) is particularly suited to monitor treatment success.

These embodiments are advantageous because they reduce the risks of infusion reactions and immune-related adverse events and/or improve the efficacy/treatment success by helping to identify those patient populations that would profit most from anti-CCR8 antibody treatment.

Preferably, fold-change in cytokine level(s) can be measured by an immune based assay in on-treatment compared to baseline serum samples as reference value.

### Responsiveness

Tregs are known to promote tumor growth by suppressing the function of cytotoxic T cells and contribute to an immunosuppressive tumor microenvironment (TME) via various mechanisms. Along this line, Tregs have also been identified as one of the key resistance mechanisms to ICIs across many tumor types. Moreover, established PD-1/PD-L1 inhibitors may not only induce the recovery of PD-1-positive dysfunctional cytotoxic T cells, but also enhance proliferation and suppressive activity of PD-1-positive Tregs. Consequently, higher abundance of PD-1+ Tregs with upregulated PD-1 expression were detected in patients whose tumors did not respond to ICIs. These observations support targeting Tregs as an attractive approach to enhance antitumor immune responses in monotherapy settings and in combination with ICIs.

This preclinical rationale also suggests that treatment with TPP-23411 might result in an innovative and effective treatment to counteract immunosuppressive pathways in tumors by overcoming resistance to PD-(L)1 inhibitors and thereby improving the efficacy of established PD-(L)1 inhibitors in a combination setting with anti-PD-(L)1 inhibitors in patients after ICI failure.

Non-responders are patients who have not received treatment with an anti-PD-(L)1 antibody for at least 6 months. This results from the fact, that the effect of the anti-PD-(L)1 antibody treatment is monitored and will be stopped in case no response is visible. As a consequence, responders are patients who have received treatment with an anti-PD-(L)1 antibody for at least 6 months.

According to a further aspect of the current invention there is provided an anti-human CCR8 antibody having ADCC activity and ADCP activity for use in a method of treatment, wherein the method of treatment is a method of treating cancer comprising
a. Stratifying a patient based on a previous treatment of the cancer for at least 6 months with an anti-PD-(L)1 antibody, and
b. Administering the anti-human CCR8 antibody to a patient only if the patient has previously been treated with an anti-PD-(L)1 antibody for at least 6 months.

In some embodiments, the anti-human CCR8 antibody is an anti-human CCR8 antibody for use in a method of treatment according to the first aspect.

### Aspect 2

### Minibody

It has been shown by the inventors that anti-mouse CCR8 surrogate antibodies deplete the tumor infiltrating CCR8+ Tregs via ADCC and ADCP mechanisms and induce robust in vivo single agent tumor growth inhibition of immunogenic murine tumor models. Furthermore, ex vivo analyses revealed that these antitumor efficacies were associated not only with efficient depletion of CCR8+ Tregs but also with a substantial increase of CD8+ T cells within the tumor micro-environment.

In U.S. application Ser. No. 17/358,841 filed on Jun. 25, 2021, PCT Appln No. PCT/EP2021/067504, PCT Appln. No. PCT/EP2021/067578, PCT Appln. No. PCT/EP2021/067574, PCT Appln. No. PCT/EP2021/067579 and PCT Appln. No. PCT/EP2021/067580 the inventors have therefore previously suggested a molecule binding a T cell marker for use in a method for diagnosing/stratifying a subject as having a tumor that is sensitive for treatment with an anti-CCR8 antibody, the method comprising
a. determining the level of the T cell marker expression in a tumor (sample),
b. comparing the level of the T cell marker expression with a reference sample or value, and
c. diagnosing/stratifying a subject as having a tumor that is sensitive for treatment with an anti-CCR8 antibody, if the level of the T cell marker is higher than or equal to a reference sample or value.

Because there is a strong interest to determine the level of a suitable T cell marker in a highly reliable way, in order to analyse the amount of T cells in a tumor biopsy sample, the inventors have now developed a method which uses PET technology rather than biopsy tissue for the evaluation, such that a more comprehensive, reliable and less error-prone picture is reached to evaluate treatment success or to decide on patient eligibility for treatment with (a further dose of) an anti-CCR8 antibody.

According to the current aspect there is provided an anti-human CCR8 antibody having ADCC activity and ADCP activity for use in a method of treating a cancer, comprising the steps of
a. Administering to a subject a Zr-89-labeled anti-CD8 minibody,
b. Performing at least one PET scan and optionally a CT scan to detect the Zr-89-labeled anti-CD8 minibody in the subject, to generate a first subject image,
c. Determining the abundance and/or distribution of the Zr-89-labeled anti-CD8 minibody in one or more cancer lesions of the subject based on the first subject image, and
d. Administering to the subject an effective dose of the anti-human CCR8 antibody, if the first subject image indicates an amount and/or a distribution of Zr-89-labeled anti-CD8 minibody in any of the one or more cancer lesions that indicates a substantial likelihood of the subject to profit from administration of the anti-human CCR8 antibody.

The minibody may be a bivalent homodimer with each monomer having a single-chain variable fragment (scFv) linked to the human IgG1 CH3 domain via modified IgG1 hinge sequence. The minibody preferably lacks Fc receptor interaction domains, and has a smaller size compared to intact monoclonal antibodies.

Preferably, the Zr-89-labeled anti-CD8 minibody binds human CD8 glycoprotein with an EC50 of <1 nM. For example, the minibody can be conjugated via desferrioxamine (Df) and radiolabeled with the positron emitting radionuclide Zirconium-89 (89Zr; Tm 78.4 hours). According to an utmost preferred embodiment, the Zr-89-labeled anti-CD8 minibody is the Zr-89-labeled anti-CD8 minibody described in U.S. application Ser. No. 17/280,137.

89Zr-Df-crefmirlimab uptake in tumor lesions correlates with CD8 expression in these lesions and can be used according to the current invention to monitor the influx of T cells into a tumor lesion after administration of a first dose of the anti-human CCR8 antibody. This influx documents the successful activation of the immune system by the anti-human CCR8 antibody and can be used e.g. as a surrogate biomarker for treatment success, as a monitoring biomarker, or as a predictive/stratification biomarker. More specifically, an increased abundance and/or an altered distribution of 89Zr-Df-crefmirlimab that indicates a substantially higher abundance or a substantial influx of T cells into at least one tumor lesion or into the tumor microenvironment, is indicative for a substantial likelihood of the subject to profit from administration of the anti-human CCR8 antibody.

The Zr-89-labeled anti-CD8 minibody or a pharmaceutical formulation thereof can be manufactured by conjugating a minibody to desferrioxamine to form a Df-minibody; radiolabeling the Df-minibody with 89Zr to form radiolabeled minibody; purifying the radiolabeled minibody; and mixing the radiolabeled minibody with a cold minibody to form a diagnostic composition, wherein the minibody and the cold minibody bind to a same epitope on CD8. A detailed description of the process can be found in U.S. application Ser. No. 17/280,137.

Preferably the abundance and/or distribution of the CD8 minibody in the subject is analyzed within 6 to 36 hours after administering the CD8 minibody to the patient, e.g. 24 h after administering the CD8 minibody to the patient.

In some embodiments, the method of positron emission tomography (“PET scan”) comprises
a. administering a Zr-89-labeled anti-CD8 minibody to a subject,
b. providing a scintillator,
c. using the scintillator to detect a pair of photons created by the Zr-89-labeled anti-CD8 minibody, and
d. using detection of the pair of photons to localize a source of the Zr-89-labeled anti-CD8 minibody via a list of coincidence events that are processed via a processor that is configured to take an output from the scintillator and convert it to the list of coincidence events,
e. wherein between about 300 and about 500 CD8 positive cells can be detected per mm³ of a tissue or cancer lesion within the subject.

In some embodiments, a MRI and/or CT scan can be performed to identify the location of the tumor and to get more accurate spatial information.

In some embodiments, the method comprises
a. providing an image of a distribution of the Zr-89-labeled anti-CD8 minibody,
b. providing an image of a distribution of a FDG marker via a second PET image of the subject,
c. creating a third PET image that comprises an overlay of the first PET image onto the second PET image.

For example, a method of determining a standard uptake value comprises
a. applying the Zr-89-labeled anti-CD8 minibody to a subject,
b. determining r, where r is the radioactivity concentration (kBq/ml) measured by a PET scanner within a region of interest of radiation from the Zr-89-labeled anti-CD8 minibody,
c. determining a′, wherein a′ is the decay-corrected amount of the injected radiolabelled tracer (kBq),
d. determining w, the weight of the subject, and
e. determining SUV as being the result of r(a′/W).

For example, an amount of Zr-89-labeled anti-CD8 minibody may be considered to indicate a substantial likelihood of the patient to profit from administration of the anti-human CCR8 antibody, if the standard uptake value (SUV) is >1, more preferably >2, >3 or >4, most preferably >5, >6 >7 or >8.

In some embodiments, a method of analyzing an image comprises
a. providing an image,
b. defining on the image a first region of interest (ROI) by marking the image,
c. determining a signal intensity for a data point within the first ROI,
d. determining a maximum signal intensity within the first ROI,
e. determining a mean value of the signal intensities within the first ROI,
f. summing together each signal intensity within the first ROI to obtain a first summed signal level for the first ROI,
   wherein the first ROI represents data for an amount of the Zr-89-labeled anti-CD8 minibody that has been administered to a subject.

In some preferred embodiments the amount and/or a distribution of Zr-89-labeled anti-CD8 minibody in any of the one or more cancer lesions is assessed
i. relative to the abundance and/or distribution of Zr-89-labeled anti-CD8 minibody in healthy tissue of the patient, or
ii. relative to one or more reference value(s) for the abundance and/or distribution of Zr-89-labeled anti-CD8 minibody.

According to some embodiments, there is provided an anti-human CCR8 antibody having ADCC activity and/or ADCP activity for use in a method of treating a cancer, comprising the steps of
a. Administering to a subject a first dose of a Zr-89-labeled anti-CD8 minibody,
b. Performing a first PET scan and optionally a CT scan to detect the Zr-89-labeled anti-CD8 minibody in the subject, to generate a first subject image,
c. Determining a first abundance and/or distribution of Zr-89-labeled anti-CD8 minibody in one or more cancer lesions in the subject based on the first subject image,
d. Administering to the subject an effective dose of the anti-human CCR8 antibody,
e. Administering to the subject a second dose of the Zr-89-labeled anti-CD8 minibody,
f. Performing a second PET scan and optionally a CT scan to detect the Zr-89-labeled anti-CD8 minibody in the subject, to generate a second subject image,
g. Determining a second abundance and/or distribution of Zr-89-labeled anti-CD8 minibody in one or more cancer lesions in the subject based on the second subject image,
h. Comparing the second subject image to the first subject image in order to evaluate if the abundance of Zr-89-labeled anti-CD8 minibody has substantially increased or if the distribution of Zr-89-labeled anti-CD8 minibody has substantially changed in one or more cancer lesions for monitoring disease progression or success of the anti-human CCR8 antibody treatment.

According to some of these embodiments, there is provided an anti-human CCR8 antibody having ADCC activity and ADCP activity for use in a method of treating a cancer, comprising the further step of administering to the patient at least one further effective dose of the anti-human CCR8 antibody if the abundance of Zr-89-labeled anti-CD8 minibody has substantially increased or if the distribution of Zr-89-labeled anti-CD8 minibody has substantially changed in one or more cancer lesions.

For example, the Zr-89-labeled anti-CD8 minibody may provide a radiation activity of about 0.5 to 3.6 mCi.

For example, the PET scan may be performed about 6 hours to 36 hours after administering the respective dose of the Zr-89-labeled anti-CD8 minibody.

In some highly preferred embodiments, the Zr-89-labeled anti-CD8 minibody is 89Zr-Df-crefmirlimab.

In some highly preferred embodiments, the anti-human CCR8 antibody is an anti-human CCR8 antibody for use in a method of treating a cancer according to another aspect disclosed herein.

In some highly preferred embodiments, the anti-human CCR8 antibody for use in a method of treating a cancer is any one of TPP-23411, TPP-29338, TPP-27454, TPP-31741, TPP-31742, TPP-31743, TPP-31744.

The anti-CCR8 antibody according to this aspect is preferably an isolated anti-CCR8 antibody or antigen-binding fragment thereof comprising six CDR sequences wherein each CDR sequence has at least 98% or 100% sequence identity with the HCDR1, HCDR2, HCDR3, LCDR1, LCDR2 and LCDR3 sequences of
a) SEQ ID NO:2, SEQ ID NO:3, SEQ ID NO:4, SEQ ID NO:6, SEQ ID NO:7 and SEQ ID NO:8,
b) SEQ ID NO:38, SEQ ID NO:39, SEQ ID NO:40, SEQ ID NO:42, SEQ ID NO:43 and SEQ ID NO:44,
c) SEQ ID NO:50, SEQ ID NO:51, SEQ ID NO:52, SEQ ID NO:54, SEQ ID NO:55, and SEQ ID NO:56,
d) SEQ ID NO:62, SEQ ID NO:63, SEQ ID NO:64, SEQ ID NO:66, SEQ ID NO:67 and SEQ ID NO:68,
e) SEQ ID NO:74, SEQ ID NO:75, SEQ ID NO:76, SEQ ID NO:78, SEQ ID NO:79 and SEQ ID NO:80,
f) SEQ ID NO:86, SEQ ID NO:87, SEQ ID NO:88, SEQ ID NO:90, SEQ ID NO:91 and SEQ ID NO:92.

Preferably, the antibody may further comprise
a. a variable heavy chain sequence that has at least 98% or 100% sequence identity with the amino acid sequence set forth in SEQ ID NO:1 and/or
   a variable light chain sequence that has at least 98% or 100% sequence identity with the amino acid sequence set forth in SEQ ID NO:5,
b. a variable heavy chain sequence that has at least 98% or 100% sequence identity with the amino acid sequence set forth in SEQ ID NO:37 and/or
   a variable light chain sequence that has at least 98% or 100% sequence identity with the amino acid sequence set forth in SEQ ID NO:41,
c. a variable heavy chain sequence that has at least 98% or 100% sequence identity with the amino acid sequence set forth in SEQ ID NO:49 and/or
   a variable light chain sequence that has at least 98% or 100% sequence identity with the amino acid sequence set forth in SEQ ID NO:53,
d. a variable heavy chain sequence that has at least 98% or 100% sequence identity with the amino acid sequence set forth in SEQ ID NO:61 and/or
   a variable light chain sequence that has at least 98% or 100% sequence identity with the amino acid sequence set forth in SEQ ID NO:65,
e. a variable heavy chain sequence that has at least 98% or 100% sequence identity with the amino acid sequence set forth in SEQ ID NO:73 and/or
   a variable light chain sequence that has at least 98% or 100% sequence identity with the amino acid sequence set forth in SEQ ID NO:77, or
f. a variable heavy chain sequence that has at least 98% or 100% sequence identity with the amino acid sequence set forth in SEQ ID NO:85 and/or
   a variable light chain sequence that has at least 98% or 100% sequence identity with the amino acid sequence set forth in SEQ ID NO:89.

Preferably, the antibody may further comprise
a. a heavy chain sequence that has at least 98% or 100% sequence identity with the amino acid sequence set forth in SEQ ID NO:17 and/or
   a light chain sequence that has at least 98% or 100% sequence identity with the amino acid sequence set forth in SEQ ID NO:18,
b. a heavy chain sequence that has at least 98% or 100% sequence identity with the amino acid sequence set forth in SEQ ID NO:47 and/or
   a light chain sequence that has at least 98% or 100% sequence identity with the amino acid sequence set forth in SEQ ID NO:48.
c. a heavy chain sequence that has at least 98% or 100% sequence identity with the amino acid sequence set forth in SEQ ID NO:59 and/or
   a light chain sequence that has at least 98% or 100% sequence identity with the amino acid sequence set forth in SEQ ID NO:60.
d. a heavy chain sequence that has at least 98% or 100% sequence identity with the amino acid sequence set forth in SEQ ID NO:71 and/or
   a light chain sequence that has at least 98% or 100% sequence identity with the amino acid sequence set forth in SEQ ID NO:72.
e. a heavy chain sequence that has at least 98% or 100% sequence identity with the amino acid sequence set forth in SEQ ID NO:83 and/or
   a light chain sequence that has at least 98% or 100% sequence identity with the amino acid sequence set forth in SEQ ID NO:84.
f. a heavy chain sequence that has at least 98% or 100% sequence identity with the amino acid sequence set forth in SEQ ID NO:95 and/or
   a light chain sequence that has at least 98% or 100% sequence identity with the amino acid sequence set forth in SEQ ID NO:96.

### Aspect 3

### Plasma Method

Described herein is a method to determine an anti-CCR8 antibody in cynomolgus plasma, the method comprising the steps of
a. Diluting a plasma or serum sample with buffer,
b. Capturing the anti-CCR8 antibody in the plasma with an immobilized biotinylated anti-human IgG-Fc antibody as capture molecule,
c. Detecting the abundance of the capture molecule using a fluorophore-labelled anti-human IgG antibody as detection reagent.

This method was used in the preclinical pharmacokinetic study in monkey and in the pivotal nonclinical safety studies. It was found to be suited for the reliable measurement of an anti-CCR8 antibody in plasma.

It was found that anti-CCR8 antibody can be determined in plasma using the Gyrolab Generic PK kit method assay with an appropriate detection range for cynomolgus subjects. In brief, samples (calibration standards, quality controls, qualification samples or unknown samples), antibodies and buffers are placed on a microplate. A biotinylated antibody is then used for capture, and an fluorophore tagged antibody is used for detection. The microplate can be loaded to the Gyrolab device, as well as the Gyrolab Bioaffy CD, and sufficient amount of buffer (Bioaffy Pump Liquid, Bioaffy Wash buffer 1 and 2). The generated fluorescence signal was found to be directly proportional to the anti-CCR8 antibody concentration present in the sample. The lower limit of quantification was 1.25 ng/mL. The working range is between 1.25 and 250 ng/mL at MRD (12.5 and 2500 ng/mL in 100% plasma). The fluorophore may be any fluorophore known in the art and is preferably an Alexa Dye such as AF647.

The immobilized biotinylated anti-human IgG-Fc antibody can be the CaptureSelect™ Human IgG-Fc PK Biotin Conjugate (Thermo Scientific, catalogue number 7103322100). CaptureSelect™ Human IgG-Fc PK Biotin Conjugate consists of a 13 kDa recombinant single domain antibody fragment (VHH affinity ligand) that specifically binds to the Fc part of all four human IgG subclasses without cross-binding to mouse, rat, rhesus, and cynomolgus monkey IgG. The affinity ligand is chemically conjugated to biotin via an appropriate spacer that retains the binding reactivity of the ligand when used in combination with streptavidin-based conjugates or streptavidin pre-coated surfaces.

### Aspect 4

### ADA Method

Biologic agents, including therapeutic antibodies, are known to have immunogenic potential, and administration to patients can induce an immune response leading to the formation of anti-drug antibodies (“ADAs”). Such ADAs may reduce the effectiveness of anti-CCR8 antibodies. For example, they may bind to or/and neutralize the anti-CCR8 antibody, resulting in changes of drug pharmacokinetics or pharmacodynamics that alters drug efficacy. ADAs may cause serious side effects, including allergic reactions, cross-reactivity against endogenous proteins by neutralizing antibodies (NAbs), and complement activation. There was therefore a need to develop a reliable assay to monitor the formation of anti-anti-CCR8 antibodies.

The inventors have now developed a method to reliably determine and (semi-)quantify anti-anti-CCR8 antibody formation in cynomolgus or human plasma or serum. This method was found to be superior compared with other tested approaches. In more detail, there is provided a method to determine and quantify anti-anti-CCR8 antibody formation in cynomolgus or human plasma or serum, the method comprising an anti-CCR8 antibody-based bridging ELISA method.

In brief, Anti-drug antibodies (ADA) against the anti-CCR8 antibody were found to be reliably detectable using a bridging ligand binding assay on the Meso Scale Discovery platform. Affinity Pure Goat Anti-Human IgG can be used as a positive control in cynomolgus plasma or serum. Positive and negative control samples as well as unknown samples can be prediluted (e.g. 1:8) with dilution buffer, mixed with dilution buffer and preincubated in a polypropylene plate, e.g. for 1 hour on an orbital shaker (e.g. RT, 600 rpm). To the sample mixture, master mix containing biotinylated anti-CCR8 antibody (e.g. 1 μg/mL) and SULFO-tagged anti-CCR8 antibody (e.g. 1 μg/mL) can be added and incubated for 2 hours (RT, 600 rpm). From the incubated samples 25 μL can be transferred in duplicates into wells of a blocked MSD Streptavidin Gold plate (150 μL Block buffer for a minimum of 30 minutes, 600 rpm) to which the biotinylated anti-CCR8 antibody can bind. If functional anti-drug antibodies are present they will bridge the biotinylated and SULFO-tagged anti-CCR8 antibody. The SULFO-tagged anti-CCR8 antibody produces an electrochemiluminescence (ECL) signal correlating to the amount of ADA in the well when voltage is applied. Plates can be read (e.g. using the Meso QuickPlex SQ 120) and data can be analyzed (e.g. with the MSD© Workbench™ software).

According to this aspect, a signal is generated if anti-anti-CCR8 antibody bridges a) biotinylated anti-CCR8 antibody and b) SULFO-tagged anti-CCR8 antibody. Methods to generate biotinylated antibodies are known in the art, and methods to generate SULFO-tagged antibodies are likewise accessible to the skilled person (e.g. based on NHS ester chemistry).

The antibody used to generated the SULFO-tagged anti-CCR8 antibody and the biotinylated anti-CCR8 antibody can be any one of TPP-23411, TPP-27454, TPP-31741, TPP-31742, TPP-31743, or TPP-31744.

The anti-CCR8 antibody according to this aspect may thus be an isolated anti-CCR8 antibody or antigen-binding fragment thereof comprising six CDR sequences wherein each CDR sequence has at least 98% or 100% sequence identity with the HCDR1, HCDR2, HCDR3, LCDR1, LCDR2 and LCDR3 sequences of
a) SEQ ID NO:2, SEQ ID NO:3, SEQ ID NO:4, SEQ ID NO:6, SEQ ID NO:7 and SEQ ID NO:8,
b) SEQ ID NO:38, SEQ ID NO:39, SEQ ID NO:40, SEQ ID NO:42, SEQ ID NO:43 and SEQ ID NO:44,
c) SEQ ID NO:50, SEQ ID NO:51, SEQ ID NO:52, SEQ ID NO:54, SEQ ID NO:55, and SEQ ID NO:56,
d) SEQ ID NO:62, SEQ ID NO:63, SEQ ID NO:64, SEQ ID NO:66, SEQ ID NO:67 and SEQ ID NO:68,
e) SEQ ID NO:74, SEQ ID NO:75, SEQ ID NO:76, SEQ ID NO:78, SEQ ID NO:79 and SEQ ID NO:80,
f) SEQ ID NO:86, SEQ ID NO:87, SEQ ID NO:88, SEQ ID NO:90, SEQ ID NO:91 and SEQ ID NO:92.

Preferably, the antibody may further comprise
a. a variable heavy chain sequence that has at least 98% or 100% sequence identity with the amino acid sequence set forth in SEQ ID NO:1 and/or
   a variable light chain sequence that has at least 98% or 100% sequence identity with the amino acid sequence set forth in SEQ ID NO:5,
b. a variable heavy chain sequence that has at least 98% or 100% sequence identity with the amino acid sequence set forth in SEQ ID NO:37 and/or
   a variable light chain sequence that has at least 98% or 100% sequence identity with the amino acid sequence set forth in SEQ ID NO:41,
c. a variable heavy chain sequence that has at least 98% or 100% sequence identity with the amino acid sequence set forth in SEQ ID NO:49 and/or
   a variable light chain sequence that has at least 98% or 100% sequence identity with the amino acid sequence set forth in SEQ ID NO:53,
d. a variable heavy chain sequence that has at least 98% or 100% sequence identity with the amino acid sequence set forth in SEQ ID NO:61 and/or
   a variable light chain sequence that has at least 98% or 100% sequence identity with the amino acid sequence set forth in SEQ ID NO:65,
e. a variable heavy chain sequence that has at least 98% or 100% sequence identity with the amino acid sequence set forth in SEQ ID NO:73 and/or
   a variable light chain sequence that has at least 98% or 100% sequence identity with the amino acid sequence set forth in SEQ ID NO:77, or
f. a variable heavy chain sequence that has at least 98% or 100% sequence identity with the amino acid sequence set forth in SEQ ID NO:85 and/or
   a variable light chain sequence that has at least 98% or 100% sequence identity with the amino acid sequence set forth in SEQ ID NO:89.

Preferably, the antibody may further comprise
a. a heavy chain sequence that has at least 98% or 100% sequence identity with the amino acid sequence set forth in SEQ ID NO:17 and/or
   a light chain sequence that has at least 98% or 100% sequence identity with the amino acid sequence set forth in SEQ ID NO:18,
b. a heavy chain sequence that has at least 98% or 100% sequence identity with the amino acid sequence set forth in SEQ ID NO:47 and/or
   a light chain sequence that has at least 98% or 100% sequence identity with the amino acid sequence set forth in SEQ ID NO:48.
c. a heavy chain sequence that has at least 98% or 100% sequence identity with the amino acid sequence set forth in SEQ ID NO:59 and/or
   a light chain sequence that has at least 98% or 100% sequence identity with the amino acid sequence set forth in SEQ ID NO:60.
d. a heavy chain sequence that has at least 98% or 100% sequence identity with the amino acid sequence set forth in SEQ ID NO:71 and/or
   a light chain sequence that has at least 98% or 100% sequence identity with the amino acid sequence set forth in SEQ ID NO:72.
e. a heavy chain sequence that has at least 98% or 100% sequence identity with the amino acid sequence set forth in SEQ ID NO:83 and/or
   a light chain sequence that has at least 98% or 100% sequence identity with the amino acid sequence set forth in SEQ ID NO:84.
f. a heavy chain sequence that has at least 98% or 100% sequence identity with the amino acid sequence set forth in SEQ ID NO:95 and/or
   a light chain sequence that has at least 98% or 100% sequence identity with the amino acid sequence set forth in SEQ ID NO:96.

### Aspect 5

### TPP-29338

When the inventors characterized the properties of clinical candidate TPP-23411, it was surprisingly found, that this antibody had an unexpected clearance behavior in cynomolgus monkey. While anti-mouse CCR8 surrogate antibodies like TPP-15285 have been created before to model the behavior of TPP-23411, these surrogate antibodies did not yet capture the unexpected clearance behavior of TPP-23411. Therefore, in order to derive a safe and effective dose scheme for the clinical candidate TPP-23411, in a first step, the inventors had to identify a murine surrogate antibody with a PK/PD behavior that closely resembles TPP-23411. Indeed, with TPP-29338 such a surrogate antibody could finally be obtained. The short half-life variant TPP-29338 was generated and was found to be suitable to model the high clearance of TPP-23411. TPP-29338 is an anti-mouse CCR8 antibody wherein the human VH/VL chain has been chimerized to mIgG2a with H310Q/H330N mutations. It induces both, ADCC and ADCP and has a short half-life, i.e. a half-life <10 days.

According to a further aspect there is thus provided an isolated anti-CCR8 antibody or antigen-binding fragment thereof comprising HCDR1, HCDR2, HCDR3, LCDR1, LCDR2 and LCDR3 sequences of SEQ ID numbers: 20, 21, 22, 24, 25, 26.

For example, the isolated anti-CCR8 antibody or antigen-binding fragment thereof further comprises
a. a variable heavy chain sequence that has at least 98% or 100% sequence identity with the amino acid sequence set forth in SEQ ID NO:19 and/or
b. a variable light chain sequence that has at least 98% or 100% sequence identity with the amino acid sequence set forth in SEQ ID NO:23.

For example, the isolated anti-CCR8 antibody or antigen-binding fragment thereof further comprises
a. a heavy chain sequence that has at least 98% or 100% sequence identity with the amino acid sequence set forth in SEQ ID NO:35 and/or
b. a light chain sequence that has at least 98% or 100% sequence identity with the amino acid sequence set forth in SEQ ID NO:36.

In some highly preferred embodiments, the antibody according to the further aspect is afucosylated. Furthermore, there is provided a polynucleotide encoding an antibody or antigen-binding fragment according to the second aspect. Also, there is provided a vector comprising that polynucleotide. Finally, there is provided an isolated host cell comprising the aforementioned polynucleotide.

### EXAMPLES

The following examples were performed to come up with a suitable dosing scheme for the (afucosylated) TPP-23411. In particular, a series of in vitro and in vivo pharmacodynamic (PD) and in vivo pharmacokinetic (PK) studies were performed with TPP-23411 and its murine surrogate antibodies.

Since TPP-23411 is cross-reactive only to cynomolgus monkey but not to the mouse CCR8 ortholog, murine surrogate antibodies were used for further in vitro and in vivo mode of action and efficacy assessments of CCR8+ Treg depletion in mice. Table 1.1 summarizes the antibodies used in in vitro and in vivo studies.

TABLE 1.1
Antibodies used in in vitro and in vivo studies.

| Antibody | Isotype | Description |
|---|---|---|
| TPP-5657 | hIgG1 | non-binding human IgG1 isotype control |
| TPP-9809 | hIgG1 | non-binding human IgG1 isotype control |
| | | (conventional glycosylation) |
| TPP-9809 (afucosylated) | hIgG1 (afuco) | non-binding human IgG1 isotype control |
| | | (afucosylated) |
| TPP-23411 (wild-type) | hIgG1 | anti-human CCR8 antibody hIgG1 isotype |
| | | (conventional glycosylation) |
| TPP-23411 (afucosylated) | hIgG1 (afuco) | anti-human CCR8 antibody hIgG1 isotype |
| | | (afucosylated) |
| TPP-10748 | mIgG2a | non-binding mouse IgG2a isotype control |
| TPP-15285 (normal half-life) | mIgG2a | anti-mouse CCR8 antibody, human VH/VL chimerized to mIgG2a |
| TPP-29338 (short half-life) | mIgG2a | anti-mouse CCR8 antibody, human VH/VL chimerized to mIgG2a |
| | | with H310Q/H330N mutations |
| TPP-14099 | hIgG1 | anti-mouse CCR8 antibody, hIgG1 isotype (conventional |
| | | glycosylation) |
| TPP-18208 (aglycosylated) | hIgG1 (aglyco) | anti-mouse CCR8 antibody, in aglycosylated hIgG1 format |
| | | (N297A) of TPP-14099 |
| TPP-15726 (aglycosylated) | hIgG1 (aglyco) | non-binding human IgG1 isotype control (aglycosylated) |
| Anti-PD-1 | rIgG2a | anti-mouse PD-1 antibody, rat IgG2a |
| BAY 1808806 | mIgG1 | non-binding mouse IgG1 isotype control |
| Anti-PD-L1 | mIgG1 | anti-mouse PD-L1 antibody, mouse IgG1 |

### Example 1: Generation of Murine Surrogate Antibody to Model the High Clearance of TPP-23411

When the inventors characterized the properties of clinical candidate TPP-23411, it was surprisingly found, that this antibody had an unexpected clearance behavior in cynomolgus monkey. While anti-mouse CCR8 surrogate antibodies like TPP-15285 have been created before to model the behavior of TPP-23411, these surrogate antibodies did not yet capture the unexpected clearance behavior of TPP-23411. Therefore, in order to derive a safe and effective dose scheme for the clinical candidate TPP-23411, in a first step, the inventors had to identify a murine surrogate antibody with a PK/PD behavior that closely resembles TPP-23411. Indeed, with TPP-29338 such a surrogate antibody could finally be obtained. The short half-life variant TPP-29338 was generated and was found to be suitable to model the high clearance of TPP-23411. TPP-29338 is an anti-mouse CCR8 antibody wherein the human VH/VL chain has been chimerized to mIgG2a with H310Q/H330N mutations.

### Example 2: Pharmacology Assessment for TPP-23411

A set of primary and secondary pharmacodynamics, safety pharmacology, and pharmacodynamic drug interaction studies was performed to characterize and assess the efficacy, specificity, and safety of TPP-23411. The noteworthy findings are presented in Table 2.1 (in vitro studies) and Table 2.2 (in vivo studies) and are further described below.

TABLE 2.1
Selected nonclinical pharmacology in vitro studies with TPP-23411. For further details on individual experiments, see PCT/EP2021/067504,
PCT/EP2021/067578, PCT/EP2021/067574, PCT/EP2021/067579 and PCT/EP2021/067580 which are incorporated herein by reference.

| | | | |
|---|---|---|---|
| | Test system | Dosing information | |
| Study title | (including species) | (concentration) | Noteworthy findings |

Primary pharmacodynamics

| | | | |
|---|---|---|---|
| Characterization of TPP-23411: | SPR: human and Cynomolgus monkey | 1.56 to 200 nM | TPP-23411 showed binding to human and Cynomolgus monkey CCR8, FcγR variants and |
| binding affinity | CCR8 N-terminal peptides and | (CCR8 peptide and fusion protein assays); | FcRn. |
| | fusion proteins, FcγR variants and | up to 25 μM | Afucosylation of TPP-23411 enhanced the affinity of the antibody to FcγRIII. TPP-23411 |
| | FcRn | (FcγR assay); | did not bind to mouse CCR8. |
| | | 15.6 to 2,000 nM | |
| | | (FcRn assay) | |
| Characterization of TPP-23411: | Flow cytometry and immuno- | 0.0032 to 50 μg/mL | TPP-23411 showed potent binding to human and Cynomolgus monkey CCR8 that was |
| cell binding | fluorescence assay (human and | (binding assay); | either ectopically expressed on HEK293 and CHO cells or endogenously expressed on |
| | Cynomolgus monkey CCR8- | 2 μg/mL | TALL-1 cells. |
| | expressing CHO and HEK293 cells, | (internalization assay) | TPP-23411 showed no internalization upon binding. |
| | and TALL-1 cells) | | |
| Characterization of TPP-23411: | Cell microarray, flow cytometry | 5 μg/mL | TPP-23411 confirmed the strong and dose-dependent binding to its target CCR8. |
| selectivity | using HEK293 cells | (microarray), and | EC₅₀ was 0.1289 μg/mL. |
| | | 0.0046 to 10 μg/mL | |
| | | (flow cytometry) | |
| Characterization of murine | SPR, mouse FcγR variants and FcRn | 1.5 to 25,000 nM | Mouse surrogate antibodies TPP-15285 and TPP-29338 exhibited similar binding |
| surrogate antibodies (for TPP-23411): | | (FcγR assay); | characteristics to mouse FcγR variants but only TPP-15285 showed binding to mouse |
| binding affinity | | 15.6 to 2,000 nM | FcRn. |
| | | (FcRn assay) | The short half-life variant TPP-29338 did not bind to mouse FcRn. |
| Characterization of murine | Flow cytometry, mouse CCR8- | 0.0009 to 70 or | Mouse surrogate antibodies TPP-15285, TPP-29338, TPP-14099, and TPP-18208 showed |
| surrogate antibodies (for TPP-23411): | expressing HEK293 cells | 1,744 nM | specific binding to mouse CCR8. |
| cell binding | | | Unexpectedly, TPP-29338 had a 10-fold higher affinity compared to TPP-15825. |
| CCR8 and FcγR densities on | Flow cytometry, human and mouse | N/A | Receptor densities of human and mouse CCR8 on target cells, and of human and mouse |
| cells used in in vitro studies | CCR8-expressing HEK293 and CHO | | FcγR variants on effector cells were determined. ADCC- and ADCP-relevant receptors are |
| | cells, human and mouse Tregs, | | present on the respective target cells and effector cells. |
| | mouse NK, human NK92V cells, | | |
| | human and mouse M2 | | |
| | macrophages | | |
| Characterization of TPP-23411: | ADCC, human CCR8-expressing | 0.0001-1 μg/mL | ADCC response to TPP-23411 using activated human Tregs with 85.0% CCR8 expression |
| ADCC assessment | HEK293 cells or human primary | (CytoTox-Glo assay); | was 52.7%, whereas, using Tregs with 31.4% CCR8 expression, ADCC response was 19.5%. |
| | Tregs as target cells, and NK92V as | up to 10 μg/mL | The corresponding EC₅₀ values were 28 pM and 56 pM, respectively. Afucosylation of TPP- |
| | effector cells: | (LDH-based assay) | 23411 enhanced the affinity of the antibody to FcγRIII and consequently clearly enhanced |
| | | | the ADCC potential of the therapeutic antibody. |
| Characterization of TPP-23411: | ADCP, human CCR8-expressing | 0.01-10 μg/mL | TPP-23411 induced dose dependent ADCP responses against human Tregs in the presence |
| ADCP assessment | HEK293 cells or human primary | (flow cytometry); | of M2c macrophages. The maximal response to TPP-23411 using human Tregs with 63% |
| | Tregs as target cells, and human | 0.00032-1 μg/mL | CCR8 expression was 49.9%, whereas ACP was 49.3% using Tregs with 40% CCR8 |
| | M2c macrophages as effector cells | (Incucyte) | expression. The corresponding EC₅₀ values were 143 pM and 440 pM, respectively. |
| Characterization of murine | ADCC, mouse CCR8-expressing | 0.0001-10 μg/mL | Murine surrogate antibodies TPP-15285, TPP-29338 and TPP-14099 induced potent and |
| surrogate antibodies (for TPP-23411): | HEK293 cells as target cells and | (Incucyte) | dose-dependent depletion of mouse CCR8-expressing target cells by mouse NK cells. |
| ADCC assessment | mouse primary NK cells as effector | | Maximum ADCC responses were between 16% and 32%, with the EC50 values between 9 |
| | cells | | and 584 pM. |
| | | | Aglycosylated surrogate antibody TPP-18208, despite good binding to mouse CCR8, |
| | | | showed no ADCC activity, demonstrating the relevance of the fully functional Fc part. |
| Characterization of murine | ADCP, mouse CCR8-expressing | 0.00064-10 μg/mL | Mouse surrogate antibodies TPP-15285, TPP-29338 and TPP-14099 induce potent and |
| surrogate antibodies (for TPP-23411): | HEK293 cells as target cells and | (Incucyte) | dose-dependent depletion of mouse CCR8-expressing target cells by mouse M2 |
| ADCP assessment | mouse M2 macrophages as effector | | macrophage. |
| | cells | | Maximum cumulative ADCP responses after 4 hours co-culture were between 67% and |
| | | | 75%, with the corresponding EC₅₀ values between 380 pM and 475 pM. |
| | | | Aglycosylated surrogate antibody TPP-18208, despite good binding to murine CCR8, |
| | | | showed no ADCP activity, thereby demonstrating the relevance of the fully functional Fc |
| | | | part. |

Secondary pharmacodynamics
SPR: Surface Plasmon Resonance; N/A: not applicable.

TABLE 2.2
Nonclinical pharmacology in vivo studies with TPP-23411.

| | | | | |
|---|---|---|---|---|
| | | Method of | Dosing | |
| | Test system | administration, | information | |
| Study title | (including species) | vehicle formulation | (dose, schedule) | Noteworthy findings |

Primary pharmacodynamics

| | | | | |
|---|---|---|---|---|
| Characterization of murine surrogate | Female | i.p., PBS | 0.01 mg/kg i.p. | T/C values of 1.12 |
| antibody TPP-15285: | BALB/cAnNCrl mice | | 0.1 mg/kg i.p. | T/C values of 0.90 |
| Strong monotherapeutic | inoculated with | | 1 mg/kg i.p. | T/C values of 0.45 |
| dose-dependent in vivo efficacy | EMT6 murine | | 10 mg/kg i.p. | T/C values of 0.33, showing dose-dependent anti-tumor efficacy. |
| | mammary | | TPP-15285, Q3/4Dx3 | TPP-15285 treatment resulted in dose-dependent intratumoral depletion of |
| | carcinoma cells | | | Tregs and in an increased abundance of CD8-positive T cells. |
| | | | | TPP-15285 at 10 mg/kg also increased IFN-y concentration in tumors and to a |
| | | | | lower extend in blood. |
| Characterization of murine surrogate | Female | i.p., PBS | TPP-15285 | TPP-15285: T/C = 0.02 |
| antibodies (for TPP-23411): | BALB/cAnNCrl mice | | TPP-14099 | TPP-14099: T/C = 0.11 |
| In vivo mode of action | inoculated with | | TPP-18208 | The TPP-18208 (aglycosylated form of TPP-14099) antibody, despite |
| in syngeneic CT26 murine carcinoma | CT26 murine colon | | All 10 mg/kg | high-affinity cellular binding to mouse CCR8, showed no antitumor efficacy and |
| model | carcinoma cells | | Efficacy study: Q3/4Dx4 | resulted in progressive disease in all animals. |
| | | | Satellite study: Q3/4Dx2 | Efficacy of mouse surrogate anti-CCR8 antibodies was thus mediated via the |
| | | | | fully functional Fc part inducing ADCC and ADCP. |
| Characterization of murine surrogate | Female | i.p., PBS | TPP-15285: 1 mg/kg | TPP-15285: T/C = 0.26 (1 mg/kg) |
| antibodies (for TPP-23411): | BALB/cOlaHsd mice | | Q3/4Dx5 | TPP-29338: T/C = 0.22 (3 mg/kg) |
| In vivo efficacy of | inoculated with | | TPP-29338: 0.3, 1, 3 mg/kg | Higher dose was required for the short half-life antibody TPP-29338 than for |
| normal half-life TPP-15285 and short | EMT6 murine | | Q3/4Dx5 | the normal half-life antibody TPP-15285 to achieve comparable in vivo efficacy. |
| half-life variant TPP-29338 in syngeneic | mammary | | | Both TPP-29338 at 3 mg/kg and TPP-15285 at 1 mg/kg depleted intratumoral |
| EMT6 murine carcinoma model | carcinoma cells | | | CCR8+ Tregs. |
| PK/PD relationship of normal half-life | EMT6 murine | i.p., PBS | TPP-15285: 0.25, 1, or 4 mg/kg | Direct correlation between the antibody plasma concentration and the extent |
| TPP-15285 and short half-life TPP-29338 | mammary | | TPP-29338: 0.25, 1, or 4 mg/kg | of intratumoral CCR8-positive Treg depletion was observed. |
| murine surrogate antibodies (for TPP- | carcinoma cells s.c. | | SD | Permanent coverage of a threshold antibody plasma concentration was |
| 23411), in the syngeneic EMT6 murine | inoculated into | | | needed to ensure sustained Treg depletion. |
| mammary carcinoma model | female | | | |
| | BALB/cAnNCrl mice | | | |
| Association between gene expression | 21 syngeneic | i.p., PBS | TPP-15285: 10 mg/kg, | CCR8 surrogate antibodies showed often superior efficacy compared to ICIs in |
| and response to anti-CCR8 antibody for | murine carcinoma | | Q3/4Dx6; | IO sensitive tumor models. |
| surrogate antibodies TPP-14099 and TPP- | models: MBT-2, 4T- | | TPP-14099: 10 mg/kg; | TPP-15285 was tested and showed efficacy in Colon-26, A20, MC38, Renca, |
| 15285, in syngeneic murine carcinoma | 1, EMT6, Colon-26, | | Q3/4Dx5 | MBT2 and Pan02 models. |
| models | CT26, MC38, WEHI- | | | TPP-14099 was tested and showed efficacy in H22, CT26, EMT6 and Hepa-16 |
| | 164, Renca, H22, | | | models |
| | Hepa1-6, LL/2, | | | Baseline expression of PD-L1 and IFN-γ in early untreated tumors correlated |
| | KLN205, A20, EL4, | | | with in vivo responses to mouse surrogate anti-CCR8 antibodies (mRNA). |
| | E.G7-OVA, B16F10, | | | Responses to anti-CCR8 antibody treatments correlated with response to anti- |
| | B16F10-OVA, | | | PD-L1 antibody and anti-PD 1 antibody treatments. |
| | B16BL6, J558, | | | |
| | Pan02, and RM-1 | | | |

Safety pharmacology (GLP studies)
Safety pharmacology endpoints (central nervous system [CNS], cardiovascular system including ECG, respiratory system) were included into the pivotal 4-week toxicity study in Cynomolgus
monkeys and remained unaffected by TPP-23411.
Pharmacodynamic drug interactions

| | | | | |
|---|---|---|---|---|
| Efficacy of TPP-15285, murine surrogate | bladder carcinoma | i.p., PBS | TPP-15285: 10 mg/kg | TPP-15285: T/C = 0.37 |
| antibody (for TPP-23411) in combination | Female C57BL/6N | | anti-PD-1 antibody: 10 mg/kg | Anti-PD-1 antibody: T/C = 0.63 |
| with anti-PD-1 antibody | Crl mice inoculated | | Both Q3/4Dx4 | Combination TPP-15285 and anti-PD-1 antibody: T/C = 0.16 |
| in the syngeneic MB49 murine carcinoma | with MB49 murine | | | Treatment with TPP-15285 as monotherapy and in combination with anti PD-1 |
| model | cells | | | antibody resulted in intratumoral reduction of Tregs. |
| | | | | Combination resulted in an additive antitumor effect. |
| Efficacy of TPP-15285, murine surrogate | Female C57BL/6N | i.p., PBS | TPP-15285: 10 mg/kg | TPP-15285: T/C = 0.17 |
| antibody (for TPP-23411) in combination | mice inoculated | | Anti-PD-L1 antibody: 3 mg/kg | Anti-PD-L1 antibody: T/C = 0.38 |
| with anti-PD-L1 antibody | with C38 murine | | Both Q3/4Dx5 | Combination TPP-15285 and anti-PD-L1 antibody: T/C = 0.02 |
| in the syngeneic C38 murine carcinoma | colon carcinoma | | | Treatment with TPP-15285 in monotherapy and in combination with anti PD- |
| model | cells | | | L1 antibody resulted in intratumoral reduction of Tregs. |
| | | | | Combination resulted in enhanced antitumor effect and in increased survival |
| | | | | compared to monotherapies. |

Q3/4D, twice weekly every three or four days; SD = single dose; T/C = Treatment/Control ratio, calculated from mean tumor volumes compared to isotype control.

### Example 3: Characterization of Murine Surrogate Antibodies: Binding Affinity

The binding of murine surrogate antibodies to mouse Fc receptor variants (mFcγR and mFcRn) was analyzed by SPR. The binding characteristics of TPP-15285 (mIgG2a surrogate antibody with a normal half-life of 141 hours), TPP-29338 (mIgG2a surrogate antibody with H310Q/H330N mutations interfering with interaction to murine FcRn resulting in short antibody half-life of 27 hours), and a mIgG2a isotype control TPP-10748 were assessed by SPR analysis. In this study, TPP-15285, TPP-29338, and TPP-10748, all exhibited similar binding characteristics to mFcγR variants (Table 3.1), but only TPP-15285 and TPP-10748 showed binding to mFcRn (Table 3.2).

TABLE 3.1
Binding of murine surrogate antibodies TPP-15285 and
TPP-29338 to mouse FcγR variants as determined by SPR.

| | K_{D} [nM] | | |
|---|---|---|---|
| Name | TPP-15285^{a} | TPP-29338^{a} | TPP-10748^{a} |
| Mouse FcγRI/CD64 | 240 | 320 | 99 |
| Mouse FcγRIIA/CD32a | 20,000 ^{b} | 12,000 ^{b} | 7,100 |
| (R167) | | | |
| Mouse FcγRIIIB/CD16b | >25,000 ^{c} | 19,000 ^{b} | 14,000 ^{b} |
| Mouse FcγRIV/CD16-2 | 1,100 | 1,400 | 480 |

^{a}TPP-15285, anti-mouse CCR8 antibody, a normal half-life murine surrogate antibody for anti-human CCR8 antibody TPP-23411; TPP-29338, anti-mouse CCR8 antibody, a short half-life murine surrogate antibody for anti-human CCR8 antibody TPP-23411; TPP-10748, mIgG2a isotype control.
^{b} Use K_{D} as approximation as saturation is not reached.
^{c} K_{D} >25,000 nM: fitted value outside of saturation curve.

TABLE 3.2
Binding of murine surrogate antibodies TPP-15285
and TPP-29338 to mouse FcRn as determined by SPR

| | K_{D} [nM] | | |
|---|---|---|---|
| Name | TPP-15285 | TPP-29938 | TPP-10748 |
| Mouse FcRn | 246 | No binding | 654 |

### Example 4: Characterization of Murine Surrogate Antibodies: Cell Binding

The binding properties of murine surrogate antibodies were assessed using flow cytometry on HEK293 cells ectopically expressing murine CCR8. The binding properties of the surrogate antibodies TPP-15285 (chimerized to mIgG2a), TPP-29338 (chimerized to mouse IgG2a, with H310Q/H330N mutations interfering with interaction to murine FcRn), TPP-14099 (human IgG1), and TPP-18208 (aglycosylated hIgG1 format of TPP-14099, where the N297A mutation entirely removes the N glycosylation site of the antibody) were compared with mIgG2a and hIgG1 non-binding isotype controls (TPP-10748 and TPP-9809, respectively).

TPP-15285, TPP-29338, TPP-14099, and TPP-18208 surrogate antibodies showed specific binding to murine CCR8. TPP-29338 had unexpectedly a 10-fold higher affinity compared to TPP-15825 (Table 4.1). In summary, these antibodies are suitable murine surrogates for the anti-human CCR8 antibody candidate TPP-23411 to be used in the in vivo efficacy and mode of action studies in mice.

TABLE 4.1
Binding affinity of tested antibodies on
murine CCR8-transfected HEK293 cells.

EC₅₀

| Antibody | nM | ng/mL |
|---|---|---|
| TPP-15285 | 12.48 | 1799 |
| TPP-29338 | 0.4946 | 71 |
| TPP-14099 | 5.577 | 805 |
| TPP-18208 | 2.469 | 357 |
| BioLegend | 1.109 | 159 |

EC₅₀: concentration required for 50% effect.

### Example 5: In Vitro Mode of Action Study: ADCC Assessment of Anti-Human CCR8 Antibody TPP-23411

ADCC mode of action assessment of the afucosylated TPP-23411 antibody using CCR8-expressing target cells and NK92V (an NK-like cell line) effector cells at effector-to-target cell ratio (E:T) 4:1 was evaluated in CytoTox-Glo assay. Additionally, the wild-type fucosylated variant of TPP-23411, both hIgG1, and hIgG1 isotype control antibodies with afucosylated and wild-type fucosylated forms were analyzed.

Maximal ADCC response was higher with higher CCR8 expression level on the target cells. Maximal ADCC response to TPP-23411 using activated human Tregs with 85.0% CCR8 expression was 52.7%, whereas using Tregs with 31.4% CCR8 expression it was 19.5%. Afucosylation of TPP-23411 enhanced the affinity of the antibody to FcγRIII and consequently clearly enhanced the ADCC potential of the therapeutic antibody. In summary, TPP-23411 triggered potent and dose-dependent depletion of both CCR8 expressing human Tregs and human-CCR8-HEK293 cells with EC50 values ranging from 55.9 pM to 12.8 pM.

TABLE 5.1
Summary of the human ADCC parameters of the tested antibodies.

| | | TPP- | TPP- | TPP- | TPP- |
|---|---|---|---|---|---|
| | | 23411 | 23411, | 9809, | 9809, |
| | | wild- | afucos- | wild- | afucos- |
| Target cells | Parameter | type | ylated | type | ylated |
| Tregs, | EC50 | N/A | 28.1 pM | N/A | N/A |
| donor 1100 ^{a} | Max response | 20.6% | 52.7% | 8.3% | 7.8% |
| Tregs, | EC50 | N/A | 55.9 pM | N/A | N/A |
| donor 1163 ^{b} | Max response | 7.8% | 19.5% | 3.0% | 2.9% |
| HEK-hCCR8 | EC50 | 70.2 pM | 12.8 pM | N/A | N/A |
| | Max response | 25.2% | 59.8% | 3.6% | 10.0% |

Afucosylated and wild-type TPP-23411, anti-CCR8 antibodies;
TPP-9809, isotype control;
N/A: not applicable;
EC₅₀: concentration required for 50% effect;
^{a} 85.0% CCR8 expression;
^{b} 31.4% CCR8 expression.

### Example 6: In Vitro Mode of Action Study: ADCP Assessment of the Anti-Human CCR8 Antibody TPP-23411

ADCP mode of action assessment of the afucosylated TPP-23411 was performed by flow cytometry-based assay using either activated human Tregs endogenously expressing CCR8 or HEK293 cells ectopically expressing human CCR8 as target cells and human M2c macrophages as effector cells at effector-to-target cell ratio (E:T) 4:1. Additionally, the wild-type fucosylated variant of TPP-23411 and hIgG1 isotype control antibodies with afucosylated and wild-type fucosylated forms were analyzed.

Neither antibody afucosylation nor the CCR8-expression level on the target cells did affect the ADCP activity. Both antibodies, wild-type and afucosylated TPP-23411, induced comparable dose-dependent ADCP responses against human Tregs in the presence of M2c macrophages. The maximal response to TPP-23411 using human Tregs with 63% CCR8 expression was 49.9%, whereas using Tregs with 40% CCR8 expression it was 49.3%. The corresponding EC50 values were 143 pM and 440 pM, respectively. In summary, TPP-23411 triggered potent and dose-dependent phagocytosis of both; CCR8 expressing human Tregs and human-CCR8-HEK293 cells with maximal ADCP responses ranging from 49.9% to 26.6% (Table 6.1).

TABLE 6.1
Summary of human ADCP parameters of the tested antibodies.

| | | TPP- | TPP- | TPP- | TPP- |
|---|---|---|---|---|---|
| | | 23411 | 23411, | 9809, | 9809, |
| | | wild- | afucos- | wild- | afucos- |
| Target cells | Parameter | type | ylated | type | ylated |
| Tregs, donor | EC₅₀ | 155.3 pM | 142.8 pM | N/A | N/A |
| 1100^{a} | Max response | 49.2% | 49.9% | 36.7% | 36.1% |
| Tregs, donor | EC₅₀ | 147.8 pM | 440.0 pM | N/A | N/A |
| 1163^{b} | Max response | 49.1% | 49.3% | 40.8% | 40.7% |
| Human- | EC₅₀ | N/A | N/A | N/A | N/A |
| CCR8- | Max response | 24.9% | 26.2% | 21.3% | 21.1% |
| HEK293 | | | | | |

TPP-9809, isotype control;
N/A: not applicable;
EC₅₀: concentration required for 50% effect;
^{a}63% CCR8 expression;
^{b}40% CCR8 expression.

### Example 7: In Vitro Mode of Action Study: ADCC Assessment of Murine Surrogate Antibodies for TPP-23411

ADCC mode of action assessment of the murine surrogate antibodies for TPP-23411 was evaluated using mouse CCR8-expressing HEK293 cells as target cells and mouse NK cells as effector cells at effector-to-target cell ratio (E:T) 10:1. Mouse-CCR8-HEK293 target cells were double-labeled with Cytolight Red Rapid Dye to label living cells and Caspase 3/7 Green Dye to label apoptotic cells, and cells were imaged in Incucyte for 24 hours. The normal half-life anti-mouse CCR8 antibody TPP-15285, an artificially modified short half-life variant of TPP-15285 named TPP-29338 (both mIgG2a), and mIgG2a derivative isotype control (TPP-10748) were evaluated in dose response. Additionally, the anti-mouse CCR8 antibodies with human IgG1 or aglycosylated human IgG1 formats (TPP-14099 and TPP-18208, respectively) were also assessed.

TPP-15285, TPP-29338, and TPP-14099 surrogate antibodies induced clear dose-dependent ADCC responses when compared to isotype control. TPP-15285 demonstrated an EC₅₀ of 227.38 pM and maximal ADCC response of 24.64%, while its short half-life variant TPP-29338 with a higher cellular affinity for mouse CCR8 was consequently more potent and had an EC₅₀ of 9.28 pM and a maximal ADCC response of 31.92%. TPP-14099 demonstrated an EC₅₀ of 584 pM and maximal ADCC response of 16.43%, whereas its N297A aglycosylated variant TPP-18208 showed no ADCC activity (Table 7.1). In summary, efficient ADCC usually requires a fully functional Fc part of the anti-mouse CCR8 surrogate antibodies TPP-15285, TPP-29338, and TPP-14099. These antibodies are suitable murine surrogates for the anti-human CCR8 antibody TPP-23411 to be used in the in vivo efficacy and mode of action studies in mice.

TABLE 7.1
Summary of the ADCC parameters for the surrogate
antibodies. ADCC measured at 20 hours.

| | TPP- | TPP- | TPP- | TPP- | TPP- |
|---|---|---|---|---|---|
| Parameter^{a} | 15285^{b} | 29338^{c} | 10748^{d} | 14099^{e} | 18208^{f} |
| EC₅₀ (ng/mL) | 32.60 | 1.33 | N/A | 87.64 | N/A |
| EC₅₀ (pM) | 227.38 | 9.28 | N/A | 584 | N/A |
| Max response (%) | 24.64 | 31.92 | N/A | 16.43 | N/A |

^{a}Calculated relative to no-antibody control.
^{b}Normal t_{1/2} mouse surrogate antibody.
^{c}Short t_{1/2} mouse surrogate antibody.
^{d}Isotype control.
^{e}Conventionally glycosylated mouse surrogate antibody.
^{f}Aglycosylated mouse surrogate antibody.
N/A, not applicable.

### Example 8: In Vitro Mode of Action Study: ADCP Assessment of Murine Surrogate Antibodies for TPP-23411

ADCP mode of action assessment of the murine surrogate antibodies for TPP-23411 was evaluated using mouse CCR8-expressing HEK293 cells as target cells and mouse M2 macrophages as effector cells at effector-to-target cell ratio (E:T) 4:1. Mouse-CCR8-HEK293 target cells were double-labeled with Cytolight Rapid Green Dye to label living cells and pHrodo Red Cell Labeling Dye to label phagocytic cells, and cells were imaged in Incucyte up to 12 hours. ADCP potential of the normal half-life anti-mouse CCR8 antibody TPP-15285, an artificially modified short half-life variant of TPP-15285 named TPP-29338 (both mIgG2a), and a mIgG2a derivative isotype control TPP-10748 was evaluated. Additionally, the anti-mouse CCR8 antibody with human IgG1 or aglycosylated human IgG1 format (TPP-14099 and TPP-18208, respectively), and a non-binding hIgG1 isotype control (TPP-9809) was also assessed for ADCP.

TPP-15285, TPP-29338, and TPP-14099 surrogate antibodies induced comparable dose-dependent ADCP responses. After 4 hours coculture, the surrogate antibodies produced maximal ADCP responses between 67% and 75%, while after 12 hours coculture, maximal ADCP responses were between 92% and 94%. TPP-15285 demonstrated an EC50 of 379.8 pM and 158.1 pM, while its short half-life variant TPP-29338 had an EC50 of 518.3 pM and 197.3 pM at 4 hours and 12 hours, respectively. TPP-14099 demonstrated an EC50 of 584 pM and maximal ADCC response of 16.43%, whereas its N297A aglycosylated variant TPP-18208 showed no ADCC activity (Table 8.1).

In summary, efficient ADCP usually requires a fully functional Fc part of the anti-mouse CCR8 surrogate antibodies TPP-15285, TPP-29338, and TPP-14099. These antibodies are suitable murine surrogates for the anti-human CCR8 antibody TPP-23411 to be used in the in vivo efficacy and mode of action studies in mice.

TABLE 8.1
Summary of the cumulative ADCP assessment of TPP-
15285, TPP-29338, TPP-14099 and TPP-18208.

| | |
|---|---|
| EC₅₀ | Maximal |

| | | | | |
|---|---|---|---|---|
| Timepoint | Antibody | pM | ng/mL | response^{a} |

| | | | | | |
|---|---|---|---|---|---|
| 4 | hours | TPP-15285^{b} | 379.8 | 54.5 | 75% |
| | | TPP-29338^{c} | 518.3 | 74.3 | 71% |
| | | TPP-14099^{d} | 474.7 | 68.6 | 67% |
| | | TPP-18208^{e} | N/A | N/A | 0% |
| 12 | hours | TPP-15285^{b} | 158.1 | 22.7 | 94% |
| | | TPP-29338^{c} | 197.3 | 28.3 | 93% |
| | | TPP-14099^{d} | 117.6 | 17.0 | 92% |
| | | TPP-18208^{e} | N/A | N/A | 0% |

^{a}Calculated relative to lowest concentration of the isotype control.
^{b}Normal half-life mouse surrogate antibody with mIgG2a format.
^{c}Short half-life mouse surrogate antibody with mIgG2a format.
^{d}Mouse surrogate antibody with hIgG1 format.
^{e}Mouse surrogate antibody with hIgG1 format, aglycosylated.
N/A: not applicable.

### Example 9: Primary Pharmacodynamics In Vivo

Since TPP-23411 is cross reactive only with Cynomolgus monkey but not with mouse CCR8 ortholog, murine surrogate antibodies were used for in vivo mode of action and efficacy assessments of CCR8+ Treg depletion in mice. The in vivo studies were performed either in monotherapy or in combination with ICIs such as anti-PD-1 and anti-PD-L1 antibodies.

### Example 9.1: In Vivo Efficacy of the Normal and Short Half-Life Murine Surrogate Antibodies in Syngeneic EMT6 Murine Carcinoma Model

Pharmacokinetics of two anti-mouse CCR8 surrogate antibodies, TPP-15285 (mIgG2a surrogate antibody for TPP-23411) and TPP-29338 (mIgG2a surrogate antibody with H310Q/H330N mutations interfering with interaction to murine FcRn), were investigated in mice after single i.v. administration at a dose of 5 mg/kg. For TPP-15825, the plasma clearance was 0.0012 L/(h*kg), the volume of distribution (Vss) 0.16 L/kg, and the plasma elimination half-life 141 hours. For TPP-29338, the plasma clearance was 0.0054 L/(h*kg), the volume of distribution (Vss) 0.14 L/kg, and the plasma elimination half-life 27 hours.

The in vivo antitumor efficacies of both anti-mouse CCR8 surrogate antibodies TPP15285 (mIgG2a surrogate antibody with normal half-life) and TPP-29338 (mIgG2a surrogate antibody with short half-life) were analyzed in monotherapy settings using the syngeneic EMT6 murine mammary carcinoma model. In addition, changes in intratumoral CCR8+ Tregs during the study were also determined. TPP-29338, the short half-life variant of TPP15285, was used to mimic the predicted short human half-life of the anti-human antibody TPP-23411 in mice.

Treatments with TPP-29338, the short half-life surrogate antibody, at 0.3, 1, or 3 mg/kg resulted in T/C values of 0.90, 0.72, and 0.22, respectively, showing dose-dependent antitumor efficacy. TPP-15285, the normal half-life surrogate antibody at 1 mg/kg resulted in a T/C value of 0.26. Both TPP-29338 at 3 mg/kg and TPP-15285 at 1 mg/kg depleted intratumoral CCR8+ Tregs. All treatments were well tolerated as indicated by increased mean body weights in all treatment groups.

In conclusion, both murine surrogate antibodies for TPP-23411 showed potent in vivo activities, with the short half-life surrogate anti-CCR8 antibody TPP-29338 requiring a higher dose than the normal half-life surrogate anti-CCR8 antibody TPP-15285 to achieve comparable exposure (Table 9.1.1) and to induce comparable intratumoral CCR8+ Treg depletion as well as in vivo antitumor efficacy.

TABLE 9.1.1
TPP-15285 and TPP-29338 plasma concentrations of EMT6
tumor-bearing mice treated with TPP-15285 or TPP-29338.

| | Plasma concentration [μg/L, GM ± GSD] | | | |
|---|---|---|---|---|
| Time- | TPP-15285 | TPP-29338 | TPP-29338 | TPP-29338 |
| point | 1 mg/kg | 0.3 mg/kg | 1.0 mg/kg | 3.0 mg/kg |
| 24 hours | 5,712.7 ± | 336.2 ± | 1,673.6 ± | 4,838.3 ± |
| | 1.1 | 1.3 | 1.5 | 1.2 |
| 48 hours | 3,745.4 ± | <LLQ | 440.2 ± | 1,310.3 ± |
| | 1.1 | | 1.3 | 1.3 |
| 72 hours | 2,004.0 ± | <LLQ | 144.2 ± | 335.9 ± |
| | 1.4 | | 1.0 ^{a} | 1.2 |

N = 3/timepoint, except for ^{a} n = 1. GM: geometric mean; GSD: geometric standard deviation; LLQ: lower limit of quantification; TPP-15285, a murine surrogate antibody for anti-human CCR8 hIgG1 antibody TPP-23411 with normal half-life (141 hours), TPP-29338, a murine surrogate antibody for the anti-human CCR8 hIgG1 antibody TPP-23411 with a short half-life (27 hours). Blood samples were collected 24, 48, and 72 hours after first treatment for pharmacokinetic analysis by ELISA.

### Example 9.2: PK/PD Relationship of Murine Surrogate Antibodies TPP-15285 and TPP-29338 in the Syngeneic EMT6 Murine Carcinoma Model

To evaluate the time course of Treg depletion and repopulation, and to better understand the PK/PD relationship of the TPP-15285 and TPP-29338 antibodies (both mIgG2a, surrogates for anti-human CCR8 antibody TPP-23411), further studies using the syngeneic EMT6 murine mammary carcinoma model were carried out. TPP-29338, the short half-life variant of TPP-15285, was used to mimic the predicted short human half-life of TPP-23411 in mice. Female BALB/cAnN mice from Charles River were randomized and the i.p. treatments of TPP-15285 (a single dose at 0.25, 1, and 4 mg/kg), TPP-29338 (a single dose at 0.25, 1, and 4 mg/kg), or mIgG2a isotype control TPP-10748 was administrated starting 8 days after the s.c. inoculation of EMT6 murine mammary carcinoma cells. Mice (n=5/group/time point) were sacrificed at time points 24, 48, 120, 192, and 336 hours after single-dose administration of the compounds or when the tumors reached the predetermined size of 225 mm². Tumors and blood were harvested for pharmacokinetic analyses and flow cytometric quantification of T cell populations.

Treatments with TPP-15285 and TPP-29338 resulted in intratumoral depletion of CCR8+ Treg cells when compared with the isotype control as determined by flow cytometry. CCR8+ Tregs repopulated the TME faster when mice were treated with the short half-life variant TPP-29338 in comparison to the normal half-life variant TPP-15285.

In conclusion, a direct correlation was observed between the antibody plasma concentration and the efficacy of intratumoral CCR8-positive Treg depletion for both murine surrogate antibodies TPP-15285 and TPP-29338 (Table 9.2.1, Table 9.2.2), suggesting that a permanent coverage of a threshold antibody concentration might be needed to ensure Treg depletion and downstream effects.

TABLE 9.2.1
TPP-15285 and TPP-29338 plasma concentrations in EMT6 tumor-bearing mice.

| | | Anti-mouse CCR8 antibody plasma concentration [μg/L, GM ± GSD] | | | | | |
|---|---|---|---|---|---|---|---|
| | | TPP-15285^{a} [mg/kg] | | | TPP-29338^{b} [mg/kg] | | |
| Time point | | 0.25 | 1 | 4 | 0.25 | 1 | 4 |
| 24 | hours | 276.9 ± 1.2 ^{c} | 1470.4 ± 1.4 | 7677.1 ± 1.2 | 113.2 ± 6.7 ^{c} | 1474.8 ± 1.7 | 6961.1 ± 1.1 ^{c} |
| 48 | hours | 273.4 ± 1.3 ^{c} | 1041.7 ± 1.4 ^{c} | 6032.3 ± 1.0 ^{d} | 157.0 ± 2.8 ^{e} | 672.1 ± 1.4 ^{c} | 1547.9 ± 1.5 ^{c} |
| 120 | hours | 84.5 ± 1.2 | 201.4 ± 4.0 ^{c} | 1439.1 ± 1.8 | 88.2 ± 1.2 ^{d} | 74.3 ± 1.0 ^{f} | 91.0 ± 2.2 ^{e} |
| 192 | hours | 70.3 ± 1.2 ^{e} | 54.4 ± 2.0 ^{c} | 88.3 ± 3.4 ^{c} | 99.0 ± 1.5 ^{c} | <LLQ | 88.3 ± 1.0 ^{f} |
| 336 | hours | 43.2 ± 1.7 ^{d} | 45.4 ± 1.5 ^{e} | 161.7 ± 1.0 ^{f} | <LLQ | 64.0 ± 2.6 ^{d} | 161.7 ± 1.3 ^{d} |

GM: geometric mean; GSD: geometric standard deviation; LLQ: lower limit of quantification.
^{a}A normal half-life (t_{1/2} = 141 hours) variant of murine surrogate antibody for anti-human CCR8 hIgG1 antibody TPP-23411.
^{b}A short half-life (t_{1/2} = 27 hours) variant of murine surrogate antibody for anti-human CCR8 hIgG1 antibody TPP-23411.
^{c} n = 4.
^{d} n = 2.
^{e} n = 3.
^{f} n = 1.
Note:
n = 5/timepoint unless indicated otherwise.

TABLE 9.2.2
Percentage of CCR8 positive cells of Tregs in tumors of EMT6
tumor-bearing mice at multiple timepoints and after treatment
with different amounts of TPP-15285 and TPP-29338.

| | | | | | | | TPP-15285 | | | | |
|---|---|---|---|---|---|---|---|---|---|---|---|
| | | Isotype mouse IgG2a | | | | | 0.25 mg/kg | | | | |
| 24 | h | 66.3 | 60.8 | 73.2 | 74.9 | 76.6 | 62.7 | 69.5 | 44.8 | 56.9 | 53.5 |
| 48 | h | 73.5 | 73.5 | 72.9 | 62.3 | 83.7 | 51.5 | 60.5 | 59.9 | 77.1 | 58.8 |
| 120 | h | 68.9 | 63.8 | 73.2 | 72.9 | 68.5 | 59.2 | 64.3 | 60.6 | 63.3 | 59.6 |
| 192 | h | 67.2 | 70.1 | 70.8 | 63.1 | 69.8 | 62.6 | 70.4 | 78.1 | 75.1 | 62.8 |
| 336 | h | 50.8 | 48.5 | 54.6 | 64 | 55 | 54.5 | 56.8 | 67.6 | 67.3 | 64.4 |

| | | TPP-15285 | | | | | TPP-15285 | | | | |
|---|---|---|---|---|---|---|---|---|---|---|---|
| | | 1 mg/kg | | | | | 4 mg/kg | | | | |
| 24 | h | 40.4 | 46.6 | 33.3 | 35.2 | 37.9 | 30.4 | 38.7 | 43.6 | 35 | 25.5 |
| 48 | h | 54.6 | 41.9 | 79.3 | 46.3 | 51.7 | 30.7 | 81.6 | 35.3 | 85 | 83 |
| 120 | h | 52.7 | 58.4 | 50.2 | 75 | | 37.2 | 41.7 | 36.7 | 43.9 | 33.9 |
| 192 | h | 68.6 | 71.3 | 69.9 | 71.6 | 73.6 | 72.1 | 75.3 | 67.7 | 64.5 | 65.3 |
| 336 | h | 61.9 | 61.9 | 69.9 | 63.2 | 62.5 | 56.1 | 57.1 | 74.2 | 63.9 | 60.3 |

| | | TPP-29338 | | | | | TPP-29338 | | | | |
|---|---|---|---|---|---|---|---|---|---|---|---|
| | | 0.25 mg/kg | | | | | 1 mg/kg | | | | |
| 24 | h | 70.9 | 62.1 | 73.8 | 58.5 | 72.9 | 62.3 | 50.9 | 62.7 | 57.8 | 54.4 |
| 48 | h | 69.6 | 75.1 | 77.5 | 78.5 | 82.8 | 82 | 68.9 | 68.9 | 73.2 | 64.2 |
| 120 | h | 75.7 | 61.6 | 66.2 | 74.6 | 80.1 | 68.2 | 65.2 | 74.1 | 80.2 | 81.3 |
| 192 | h | 62.8 | 78.9 | 72.4 | 70.9 | 76.2 | 73.3 | 71.6 | 77.8 | 73.4 | 75.4 |
| 336 | h | 58.1 | 64.7 | 67.3 | 55.8 | 53.9 | 64.9 | 77.8 | 76.8 | 67.6 | 67.4 |

| | | TPP-29338 | | | | |
|---|---|---|---|---|---|---|
| | | 4 mg/kg | | | | |
| 24 | h | 65.6 | 38.9 | 45.9 | 50.7 | 41.7 |
| 48 | h | 43.9 | 85.1 | 57.2 | 57 | 57.5 |
| 120 | h | 75.4 | 70.5 | 83.4 | 70 | 81.3 |
| 192 | h | 67.4 | 75.6 | 76.4 | 71.7 | 78.6 |
| 336 | h | 64.4 | 63.9 | 67.2 | 72.5 | 63.6 |

### Example 9.3: IFN-γ Concentrations Measured in Tumor or Blood of EMT6 Tumor-Bearing Mice

An ELISA analysis of IFN-γ concentrations was measured on day 19 at the study end in tumor (Table 9.3.1) and blood (Table 9.3.2) of EMT6 tumor-bearing mice tested with TPP-10748 isotype control or TPP-15285.

TABLE 9.3.1
IFN-γ concentration (pg/mL) in tumors of EMT6 tumor-bearing
mice treated with TPP-10748 or anti-mouse CCR8 antibody TPP-13285.

| TPP10748, | TPP15285, | TPP15285, | TPP15285, | TPP15285, |
|---|---|---|---|---|
| 10 mg/kg | 10 mg/kg | 1 mg/kg | 0.1 mg/kg | 0.01 mg/kg |
| 17.64271046 | 38.67835027 | 29.79365737 | 105.9723581 | 0.756231592 |
| 2.869074357 | 93.95588563 | 8.791223448 | 5.771133 | 5.26338557 |
| 1.29273685 | 47.25213496 | 25.67009096 | 5.768417254 | 3.007183809 |
| 3.757745297 | 48.70244183 | | 28.0077091 | 3.139902307 |
| 3.375602823 | 66.38236879 | 30.21765468 | 4.631030582 | 1.425002946 |

TABLE 9.3.2
IFN-γ concentration (pg/mL) in blood of EMT6 tumor-bearing
mice treated with TPP-10748 or anti-mouse CCR8 antibody TPP-13285.

| TPP10748, | TPP15285, | TPP15285, | TPP15285, | TPP15285, |
|---|---|---|---|---|
| 10 mg/kg | 10 mg/kg | 1 mg/kg | 0.1 mg/kg | 0.01 mg/kg |
| 0.411781013 | 1.254520663 | 1.253167548 | 6.034607588 | 0.441415921 |
| 0.554639402 | 2.748435225 | 0.828632408 | 0.40370028 | 0.723301203 |
| 0.395620219 | 2.155459212 | 1.326244397 | 1.1841669 | 0.701702426 |
| 0.467016395 | 1.71083374 | 0.823229383 | 1.976473053 | 0.920505596 |
| 0.661212261 | 2.871987891 | 1.643095384 | 0.798917619 | 0.379462172 |

### Example 10: Evaluation of Pharmacokinetic/Pharmacodynamic (PK/PD) Relationship and Efficacious Exposure

For the establishment of a PK/PD relationship, the concentration leading to maximum effect (EC80 for efficacious dose prediction) and the concentration leading to a minimal effect (EC20 for prediction of the minimal anticipated effect level) were derived from human in vitro ADCC and ADCP assays (see examples provided elsewhere herein).

These concentrations were corrected by an additional in vitro to in vivo correlation (IVIVC) factor informed from mice by in vitro ADCC and ADCP assays and the in vivo observed depletion of Tregs (see examples provided elsewhere herein).

Furthermore, an empirical correlation of Treg depletion to tumor shrinkage in mice in vivo was considered, demonstrating a T/C ratio <0.5, when Treg depletion was larger than 50% from baseline. Since only limited information was available regarding Treg dynamics in human, a permanent Treg depletion was assumed to be required for antitumor efficacy. As further exposure and Treg depletion were observed to be directly related in dedicated in vivo exposure-response studies in mice, permanent coverage of a threshold antibody concentration was assumed to ensure sustained Treg depletion required for antitumor efficacy. Therefore, trough concentration (Ctrough) at steady-state was assumed to be the driver for Treg depletion and any further effect on the anti-tumor immunity in humans.

In summary, for human efficacious dose prediction, the estimated EC80 associated with an expected ˜50% Treg depletion should be targeted as Ctrough. For prediction of a minimal effective dose level which is associated with ˜10% Treg depletion, the estimated EC20 should be targeted as Ctrough. Table 10.1 provides the estimated range of efficacious concentration in humans in addition to the estimated range of minimal effective concentrations.

TABLE 10.1
Extrapolation to efficacious and minimal biologically effective concentration
regarding Treg depletion of TPP-23411 in vivo in humans.

| | ADCC | | | ADCP | | |
|---|---|---|---|---|---|---|
| | | IVIVC | | | IVIVC | |
| | in vitro | factor | Pred. in vivo | in vitro | factor | Pred. in vivo |
| | concentration | from | concentration | concentration | from | concentration |
| | [μg/mL] | mouse | [μg/mL] | [μg/mL] | mouse | [μg/mL] |
| EC₈₀ | 0.0012-0.0052 | 42 | 0.050-0.22 | 0.077 | 9-18 | 0.69-1.4 |
| EC₂₀ | 7.5e−5-3.3e−4 | 42 | 0.0031-0.014 | 0.0048 | 9-18 | 0.043-0.088 |

### Example 11: Safety Pharmacology

Effects of TPP-23411 on vital organ function (central nervous system [CNS], cardiovascular system including ECG, respiratory system) were investigated according to the ICH guidelines S6 and S9, i.e., the respective endpoints were included into the pivotal 4 week toxicity study in Cynomolgus monkeys.

Safety pharmacology endpoints included detailed clinical observations, physical/neurological examinations (abdominal palpation, body temperature, heart and lung auscultation, general sensory aspects including cerebral [pupillary, orbicularis oculi] and spinal reflexes [patellar, anal], and foot grip reflex), respiration rate, arterial blood pressure (high definition oscillometry), and ECG (30 to 60 seconds recording). These investigations were done once during the pre dose phase, during week 1 and 4 of the dosing phase (pre dose and 1 to 2 hours post dose), and during week 2 of the recovery phase. Systemic exposure to TPP-23411 was also determined.

TPP-23411 was administered by i.v. slow bolus injection once/twice weekly at the doses of 2×0 (vehicle), 15, 50, and 2×40 mg/kg to 3 to 5 male/female Cynomolgus monkeys per group. None of the abovementioned parameters were significantly affected by treatment at mean plasma concentrations up to 1550 mg/L (50 mg/kg). These plasma concentrations are >500-fold higher than those currently anticipated for human therapeutic efficacy (Cmax range 0.7-20 mg/L at dose range 38-1100 μg/kg).

### Example 12: Nonclinical Pharmacokinetics and Drug Metabolism

Pharmacokinetics of TPP-23411 was studied in vivo in male Cynomolgus monkeys after single i.v. and s.c. administration of TPP-23411. TPP-23411 was measured in monkey plasma using an anti-human IgG generic assay (IgG-ELISA). Anti-TPP-23411 antibody formation was monitored with a validated TPP-23411-based bridging ELISA method (described elsewhere herein).

TABLE 12.1
Overview of single dose pharmacokinetics, in vivo study.

| | |
|---|---|
| Study type | Single dose pharmacokinetics, in vivo study |
| Test system | Monkey, 3 males/dose group, strain: Cynomolgus |
| Dosage | TPP-23411: 1 and 10 mg/kg iv and 3 and 10 mg/kg sc |
| Assay, matrix | IgG ELISA, plasma samples |

i.v.: intravenous; s.c.: subcutaneous; PK: pharmacokinetics; m: male.
Observation intervals were 336 hours for the low and 504 hours for the high dose.

Table 12.2 shows the pharmacokinetics of TPP-23411 after intravenous and subcutaneous administration, FIG. 2 shows dose-normalized plasma concentrations of TPP-23411 after intravenous and subcutaneous administration.

After single intravenous bolus administration of 1 and 10 mg/kg TPP-23411 to male cynomolgus monkeys, the exposure in terms of AUCnorm increased slightly more than dose-proportionally from 391 kg·h/L to 617 kg·h/L with no hints for target mediated drug disposition. The plasma elimination was bi-phasic and the plasma clearance was 2.55 mL/(h·kg) for the 1 mg/kg and 1.62 mL/(h·kg) (mean values) for the 10 mg/kg dose. The volume of distribution amounted to 0.154 and 0.110 L/kg (mean values) for the 1 and 10 mg/kg dose, respectively. The effective half-lives were short with 41.9 and 46.8 hours reflecting a relatively fast elimination of the antibody and the pharmacologically less relevant terminal elimination half-lives were 108 and 148 hours.

After single subcutaneous administration of 3 and 10 mg/kg TPP-23411 a dose-proportional increase of exposure in terms of AUCnorm from 331 kg·h/L to 403 kg·h/L and Cmax,norm from 2.22 kg/L to 2.95 kg/L was observed (mean values). Cmax was reached at 8 to 30 h hours (mean values) after administration. TPP-23411 was eliminated from plasma with terminal half-lives of 81 and 109 h (mean values) at dose levels of 3 and 10 mg/kg, respectively, with plasma concentrations running in parallel to the intravenous profile. Bioavailability in both dose groups was moderate to high and ranged from 54% to 103%.

TABLE 12.2
Pharmacokinetics of TPP-23411 after a single dose (non-rodent).

| | | | | | |
|---|---|---|---|---|---|
| Species/strain: | | Monkey/ | Monkey/ | Monkey/ | Monkey/ |
| | | Cynomolgus | Cynomolgus | Cynomolgus | Cynomolgus |
| Compound: | | TPP-23411 | TPP-23411 | TPP-23411 | TPP-23411 |
| Analyte: | | TPP-23411 | TPP-23411 | TPP-23411 | TPP-23411 |
| Route: | | iv | iv | sc | sc |
| Dose: | [mg/kg] | 1 | 10 | 3 | 10 |
| Gender/No. | | M/3 | M/3 | M/3 | M/3 |
| of animals: | | | | | |
| Duration: | [h] | 336 | 504 | 336 | 504 |
| Assay: | | ELISA | ELISA | ELISA | ELISA |

| | | Geometric | | Geometric | | Geometric | | Geometric | |
|---|---|---|---|---|---|---|---|---|---|
| Parameter | Unit | Mean | S.D. | Mean | S.D. | Mean | S.D. | Mean | S.D. |
| AUC | [μg · h/mL] | 391 | 1.16 | 6170 | 1.30 | 992 | 1.16 | 4030 | 1.49 |
| AUCₙₒᵣₘ | [kg · h/L] | 391 | 1.16 | 617 | 1.30 | 331 | 1.16 | 403 | 1.49 |
| AUC(0-tₙ) | [μg · h/mL] | 378 | 1.17 | 6020 | 1.29 | 930 | 1.15 | 3860 | 1.47 |
| tₙ | [h] | 336 | | 504 | | 336 | | 504 | |
| Cₘₐₓ | [μg/mL] | 20.6 | 1.07 | 236 | 1.08 | 6.67 | 1.28 | 29.5 | 1.29 |
| C_{max, norm} | [kg/L] | 20.6 | 1.07 | 23.6 | 1.08 | 2.22 | 1.28 | 2.95 | 1.29 |
| tₘₐₓ | [h] | 0.5 | n.c.^{b} | 1.00 | n.c.^{b} | 30.0 | n.c.^{b} | 8.00 | n.c.^{b} |
| t_{1/2 app} | [h] | 41.9 | 1.12 | 46.8 | 1.22 | | | | |
| t_{1/2} | [h] | 108 | 1.18 | 148 | 1.13 | 81.0 | 1.09 | 109 | 1.12 |
| Interval | [h] | 168-336 | | 168-504 | | 96-336 | | 96-336 | |
| CL | [mL/(kg · h)] | 2.55 | 1.16 | 1.62 | 1.30 | n.c.^{b} | n.c.^{b} | n.c.^{b} | n.c.^{b} |
| Vₛₛ | [mL/kg] | 154 | 1.29 | 110 | 1.12 | n.c.^{b} | n.c.^{b} | n.c.^{b} | n.c.^{b} |
| MRT | [h] | n.c.^{b} | n.c.^{b} | n.c.^{b} | n.c. ^{b} | 128 | 1.03 | 153 | 1.16 |
| F^{a} | [%] | (100) | | (100) | | 84.6 | | 65.8 | |

^{a}bioavailability, calculated with the IV AUCnorm value of 1 mg/kg b.w. in case of 3 mg/kg s.c. and calculated with the IV AUCnorm value of 10 mg/kg b.w. in case of 10 mg/kg s.c;
^{b}not calculated.
No significant anti-drug antibody formation was detected.

### Example 13: Toxicology

General objectives of the toxicity program were the identification of target organs of toxicity, assessment of reversibility, late onset of findings and the determination of exposure-response relationship.

TPP-23411 is a human monoclonal antibody with no cross-reactivity in animal species other than non-human primates. Therefore, the in vivo toxicology program included a comprehensive evaluation in the Cynomolgus monkey only. The amino acid sequence of CCR8 is 94% identical between humans and Cynomolgus monkeys, and TPP-23411 binds to human and Cynomolgus monkey CCR8 with a similar affinity. In addition, monkeys are considered the most human-like species concerning immune function.

The toxicology program in monkeys consisted of a high-dose toxicokinetic study, a 4-week pilot study with repeat administration and a pivotal 4-week repeated dose toxicity study under immunostimulatory conditions (KLH immunization) including a wash out/recovery phase. The in vivo toxicity studies included several non-routine immunological parameters such as immune phenotyping, cytokine levels, and inflammatory markers, and mRNA sequence evaluations on CCR8 mRNA expression was assessed for selected tissues.

In addition, a comprehensive tissue cross-reactivity investigation and in vitro cytokine release assay with human full blood and human PBMCs were performed.

The pivotal studies were conducted in accordance with the OECD principles on GLP at the test facility of Charles River Laboratories Evreux, France and at the test facility of Labcorp Early Development Services GmbH (formerly Covance Preclinical Services GmbH) Munster, Germany. A detailed overview of the toxicological studies conducted with TPP-23411 is given in Table 13.1 below.

TABLE 13.1
Toxicology program.

| | | | | |
|---|---|---|---|---|
| Type of study/ | Species and | Method of | Compound administered | |
| duration | strain | administration | [mg/kg/week] | GLP |

Single-/repeat-dose, TK

| | | | | |
|---|---|---|---|---|
| High dose TK^{a} | Monkey, | i.v., s.c. | i.v.: 2 × 17, 2 × 50 | No |
| | Cynomolgus | | s.c.: 1 × 50 | |

Repeat-dose toxicity

| | | | | |
|---|---|---|---|---|
| 4-week, TK | Monkey, | i.v. | 0; 6; 17; 50 | No |
| | Cynomolgus | | | |
| 4-week, KLH, TK, | Monkey, | i.v. | 2 × 0; 15; 50; 2 × 40 | Yes |
| 2-week recovery | Cynomolgus | | | |

Tissue cross-reactivity

| | | | | |
|---|---|---|---|---|
| Species comparative TCR | Human, monkey | in vitro | 3 and 9 μg/mL | No |
| Species comparative TCR | Human, monkey | in vitro | 3 and 10 μg/mL | Yes |

Cytokine release assay

| | | | | |
|---|---|---|---|---|
| CRA | Human whole | in vitro | Test 1: 2, 10, and 50 μg/mL | No |
| | blood and PBMC | | alone/in combination with | |
| | | | pembrolizumab 2 μg/mL | |
| | | | Test 2: 0.000128 to 10 μg/mL | |

GLP: Good Laboratory Practice; TK: toxicokinetics; KLH: keyhole limpet hemocyanin; PBMC: peripheral blood mononuclear cell; TCR: tissue cross-reactivity; CRA: cytokine release assay; i.v.: intravenous; s.c.: subcutaneous.
^{a}Twice weekly i.v. administration of 17 or 50 mg/kg, single s.c. administration of 50 mg/kg.

Local tolerability was evaluated within the repeat-dose toxicity studies in monkeys. No signs of local intolerance reactions have been observed by histopathological evaluation of the injection sites after the administration of the TPP-23411 formulation.

### Example 13.1: Repeat-Dose Toxicity Studies in Monkeys: Toxicokinetic Study in Cynomolgus Monkeys with Once Weekly s.c. And Twice Weekly i.v. Administration

A preliminary high-dose toxicokinetic study was conducted to evaluate the toxicokinetic profile of TPP-23411 in plasma after one s.c. and two i.v. administrations in Cynomolgus monkeys. The animals received a single s.c. administration of 50 mg/kg or twice weekly 17 mg/kg or 50 mg/kg of TPP-23411 on Days 1 and 4.

TPP-23411 was systemically and locally well tolerated up to the highest dose. Reduced food consumption was observed in all animals of all dose groups during the morning feeding. The second feeding (overnight) was normal. Reduced food intake, associated with slight body weight loss (1% to 5%) was not attributed to the test material but to the treatments with repeated blood sampling. No necropsy and histopathology were performed in this study. Mean plasma concentrations and pharmacokinetic parameters of TPP-23411 are compiled in Table 13.1.1 and Table 13.1.2 below.

TABLE 13.1.1
Exposure of TPP-23411 in the high-dose pharmacokinetic
study in monkeys on Day 1

| | | Sex | | |
|---|---|---|---|---|
| | | F | F | F |
| Dose [mg/kg] and route | | 50 s.c. | 17 i.v. | 50 i.v. |
| AUC₍₀₋ₜₗₐₛₜ₎ | [μg · h/mL] | 22000 | 12400 | 46700 |
| AUC₍₀₋ₜₗₐₛₜ₎ₙₒᵣₘ | [kg · h/L] | 440 | 728 | 934 |
| tₗₐₛₜ | [h] | 168 | 72.0 | 72.0 |
| AUC_{(0-72 h)} | [μg · h/mL] | 9140 | 12400 | 46700 |
| AUC_{(0-72 h)norm} | [kg · h/L] | 183 | 728 | 934 |
| Cₘₐₓ | [μg/mL] | 192 | 439 | 1490 |
| C_{max, norm} | [kg/L] | 3.83 | 25.8 | 29.9 |
| tₘₐₓ | [h] | 96.0 | 0.500 | 0.250 |
| C₍ₜᵢₙₜ₎/Cₘₐₓ | [%] | 42.3 | 12.1 | 21.5 |
| T₍ᵢₙₜ₎ | [h] | 168 | 72.0 | 72.0 |

TABLE 13.1.2
Exposure of TPP-23411 in the high-dose pharmacokinetic
study in monkeys on Day 4.

Sex

| | F | F |
|---|---|---|
| Dose [mg/kg] and route | 17 i.v. | 50 i.v. |

| | | | |
|---|---|---|---|
| AUC₍₀₋ₜₗₐₛₜ₎ | [μg · h/mL] | 19500 | 88400 |
| AUC₍₀₋ₜₗₐₛₜ₎ₙₒᵣₘ | [kg · h/L] | 1150 | 1770 |
| tₗₐₛₜ | [h] | 168 | 168 |
| AUC_{(0-72 h)} | [μg · h/mL] | 14900 | 58800 |
| AUC_{(0-72 h)norm} | [kg · h/L] | 877 | 1180 |
| Cₘₐₓ | [μg/mL] | 528 | 1710 |
| C_{max, norm} | [kg/L] | 31.1 | 34.3 |
| tₘₐₓ | [h] | 0.500 | 0.250 |
| C₍ₜᵢₙₜ₎/Cₘₐₓ | [%] | 4.5 | 12.4 |
| T₍ᵢₙₜ₎ | [h] | 168 | 168 |
| RA-AUC₍₀₋ₜₗₐₛₜ₎ | [%] | 158 | 189 |
| RA-Cₘₐₓ | [%] | 120 | 115 |

RA-AUC = Accumulation ratio (AUC(0-last)_{Day 4}/AUC(0-last)_{Day 1}); RA-Cₘₐₓ = Accumulation ratio (C_{max, Day 4}/C_{max, Day 1})

### Example 13.2: Repeat-Dose Toxicity Studies in Monkeys: Four-Week Repeat-Dose Toxicology Study in Cynomolgus Monkeys with Once-Weekly i.v. Administration

TABLE 13.2.1
Key data of the pilot repeat-dose
(4-week) toxicity study in monkeys.

| | |
|---|---|
| Test item | TPP-23411 (preformulated); Purity: 98.69% (monomer |
| | content) |
| Animals | Female Cynomolgus monkeys (Macaca fascicularis) at |
| | start of predose phase approximately 2 years old |
| | with body weight of 2.4 kg to 4.5 kg; two animals |
| | per group |
| Dose | 0 (vehicle control); 6 mg TPP-23411/kg/week; |
| | 17 mg TPP-23411/kg/week; 50 mg TPP-23411/kg/week |
| Administration | One intravenous (bolus) administration per week |
| | over a period of 4 weeks. |
| Vehicle | 5% dextrose solution |
| General | Clinical observations including functional observation |
| investigations | battery (FOB), food consumption, body weights, |
| | ophthalmology, cardiovascular investigations |
| | (electrocardiogram, blood pressure measurements, and |
| | respiratory rate), hematology, coagulation, clinical |
| | pathology (blood, urine), physical and neurological |
| | examinations including body temperature, gross |
| | pathology at necropsy, organ weights, histopathology. |
| Special | Deep mRNA analysis, C-reactive protein (CRP) and |
| investigations | cytokines/chemokines levels, immunophenotyping (IPT) |
| | on blood. |
| Toxicokinetics | Analysis of plasma concentrations. |

Four groups of 2 female Cynomolgus monkeys received TPP-23411 by i.v. administration on Days 1, 8, 15, and 22 at 0, 6, 17, or 50 mg/kg/injection at a constant dose volume of 2 mL/kg/administration. The assessment of toxicity was based on survival, clinical signs, body weight, food consumption, electrocardiography examinations, blood pressure recording, ophthalmological examinations, body temperature recording, the functional observation battery (FOB), hematology, coagulation and blood biochemistry analyses, urinalysis, immunophenotyping on blood (IPT), determination of C-reactive protein (CRP) and cytokine/chemokine levels, organ weights, and macroscopic and microscopic examinations. Blood samples were collected for toxicokinetic and immunogenicity evaluation. In addition, samples of blood and selected tissues were collected for deep RNA sequencing.

### Clinical Observations

There were no unscheduled deaths during the study. No test item-related clinical signs were observed. Body weight, food consumption, and electrocardiographic quantitative and qualitative parameters were unaffected at any dose level during the study. No ophthalmological findings were observed at the end of the treatment period.

### Clinical Pathology

There were no test item-related changes in any hematology, coagulation, blood biochemistry, and urinary parameters or on CRP levels throughout the study. There were no effects of the test item on the level of any cytokine determined during the study (IFN-γ, TNF-α, IL-1β, IL-2, IL-4, IL-10, IL-6, and IL-8).

### Immunotoxicity

Immunophenotyping (IPT) analyses of T lymphocytes, cytotoxic T lymphocytes (conventional, activated, and regulatory features), helper T lymphocytes (conventional, activated, and regulatory features), CD4−/CD8− T lymphocytes (conventional, activated, and regulatory features), natural killer T lymphocytes, B lymphocytes, and natural killer cells did not reveal evidence of immunotoxicity. The cell populations studied were within a normal range, except for a trend (not statistically significant) of slight reduction in natural killer cells after administration of TPP-23411.

### Cross Pathology, Histopathology

TPP-23411 administration at ≥6 mg/kg/injection induced non-adverse minimally or slightly decreased lymphoid cellularity in the iliac and axillary lymph nodes, particularly in the germinal centers, i.e., secondary lymphoid follicles, in all treated females, however, without any dose-relationship. In the thymus, lymphoid cellularity was minimally decreased in 1 out of 2 females from each group at ≥6 mg/kg/injection, correlating with lower thymus weights. It remained unclear whether this finding was related to TPP-23411 or was due to physiological thymic involution. In addition, a minimal increase of tingible body macrophages was noted in 1 out of 2 females at 17 mg/kg/injection and in both females at 50 mg/kg/injection. No TPP-23411-related changes were observed at the injection sites, showing that the test item administration was locally well tolerated.

### Deep RNA Sequencing Analysis

An investigation on whether anti-CCR8 treatment causes significant depletion of Tregs or changes in the expression of immune cell type specific markers, RNA sequencing on different tissue samples from Cynomolgus monkeys was performed. The samples were collected from the following tissues: intestines (ileum, jejunum, cecum, and colon), lymph nodes (mandibular, mesenteric, axillar, and iliac), liver, spleen, lungs, skin, thymus, and tonsils. In line with expectation, CCR8 mRNA was preferentially detected in thymus tissue as well as different lymph node samples. With the exception of the 2 thymus samples from the highest dose treatment group, no relevant decrease in CCR8 mRNA levels was detected in any of the tissue. None of the changes were significant after multiple testing correction or showed consistent dose dependency.

Evaluation of the expression of pro-inflammation markers CXCL9/10 and IFN-γ, cytotoxic T cell markers CD8A/B, NK cell markers NCR1/SH2D1B, B cell markers CD19/CD20/CD22, M2 macrophage markers CSF1R/MRC1/CD163, and FCg receptors FCGR2A/2B did not point to significant changes in any of these markers in any of the evaluated tissues.

TABLE 13.2.3
Noteworthy findings of the pilot repeat-dose (4-week) toxicity
study in male (M) and female (F) Cynomolgus monkeys.

| | | | Dose |
|---|---|---|---|
| Investigation | Findings | Sex | [mg/kg] |
| Clinical | No mortality | M + F | all |
| observations | No relevant effect on | M + F | all |
| | neurobehavior, body weight | | |
| | or body weight gain or | | |
| | food consumption | | |
| Ophthalmology | No relevant effects | M + F | all |
| ECG etc. | No relevant effects on ECG, | M + F | all |
| | blood pressure, body | | |
| | temperature | | |
| Hematology, | No relevant effects | M + F | all |
| coagulation | | | |
| Clinical Chemistry | | | |
| blood, urine | No relevant effects | M + F | all |
| Immunotoxicology | No relevant effects on C- | M + F | all |
| | reactive protein or cytokine | | |
| | levels | | |
| | No relevant effects in | M + F | all |
| | immunophenotyping (IPT) | | |
| | analysis | | |
| Post-mortem | | | |
| cross pathology | No relevant effects | M + F | all |
| organ weights | No relevant effects | M + F | all |
| histopathology | No relevant effects on organs | M + F | all |
| | and tissues; no local | | |
| | intolerance | | |

### Toxicokinetic Evaluation

Results of the determinations are summarized in Table 13.2.2. The low and the medium dose group show clear dose linearity at steady state. Based on AUC(0-tlast)norm, the highest dose group showed over-proportional exposure on Day 22. Cmax,norm is increasing dose proportionally on Day 22. In all dose groups, no relevant accumulation was seen based on Cmax,norm. Based on AUC(0-tlast)norm a slight increase was seen in all dose groups. The factor for AUC(0-tlast)norm was most prominent in the highest dose group (factor 1.5-fold). At the end of the observation interval of 168 hours post dose, no relevant concentrations were remaining.

TABLE 13.2.2
Exposure of TPP-23411 in the pilot 4-week
toxicology study in monkeys on Day 22.

| | | Sex | | |
|---|---|---|---|---|
| | | F | F | F |
| Dose [mg/kg] and route | | 6 i.v. | 17 i.v. | 50 i.v. |
| AUC₍₀₋ₜₗₐₛₜ₎ | [μg · h/mL] | 4780 | 14000 | 62800 |
| AUC₍₀₋ₜₗₐₛₜ₎ₙₒᵣₘ | [kg · h/L] | 797 | 822 | 1260 |
| tₗₐₛₜ | [h] | 168 | 168 | 168 |
| Cₘₐₓ | [μg/mL] | 160 | 480 | 1510 |
| C_{max, norm} | [kg/L] | 26.7 | 28.2 | 30.2 |
| tₘₐₓ | [h] | 0.250 | 0.250 | 0.500 |
| C₍ₜᵢₙₜ₎/Cₘₐₓ | [%] | 3.3 | 2.9 | 6.2 |
| T₍ᵢₙₜ₎ | [h] | 168 | 168 | 168 |
| RA-AUC₍₀₋ₜₗₐₛₜ₎ | [%] | 133 | 126 | 147 |
| RA-Cₘₐₓ | [%] | 110 | 120 | 112 |

RA-AUC = Accumulation ratio (AUC(0-last)Day 22/AUC(0-last)Day 1); RA-Cmax = Accumulation ratio (Cmax, Day 22/Cmax, Day 1).

### Example 13.3: Four-Week Repeat-Dose Toxicology Study in Cynomolgus Monkeys with Once- or Twice-Weekly i.v. Administration, KLH Immunization and 2-Week Treatment-Free Period

A pivotal GLP repeat-dose study with a treatment period of 4 weeks was conducted in male and female Cynomolgus monkeys. TPP-23411 was administered intravenously once weekly at doses of 15 mg/kg and 50 mg/kg and twice weekly at doses of 0 mg/kg (vehicle control) and 40 mg/kg. The dose levels were chosen based on the pilot toxicokinetic and repeat-dose toxicology studies which were complemented by a simulation model to compensate for the short half-life of TPP-23411 in Cynomolgus monkeys.

TABLE 13.3.1
Treatment groups.

| | Dose | | Dose | Number of | |
|---|---|---|---|---|---|
| | Level^{b, c} | | Concentration^{b, c} | Animals ^{d} | |
| Group^{a} | (mg/kg) | | (mg/mL) | Males | Females |
| 1 (Control) | 0 | 2qw | 0 2qw | 5 | 5 |
| 2 (Low) | 15 | | 7.5 | 3 | 3 |
| 3 (Intermediate) | 50 | | 25 | 3 | 3 |
| 4 (High) | 40 | 2qw^{e} | 20 2qw | 5 | 5 |

2qw = twice weekly.
^{a}Group 1 were administered vehicle control only;
^{b}Animals were dosed at a volume of 2 mL/kg;
^{c}Groups 1 and 4: twice weekly (2qw) dosing on Days 1, 4, 8, 11, 15, 18, 22, 25, and 29. Groups 2 and 3: once weekly dosing on Days 1, 8, 15, 22, and 29;
^{d} Three animals/sex/group were designated as terminal animals, and 2 animals/sex/Group 1 and 4 were designated as recovery animals (based on survival);
^{e}80 mg/kg/week in total.

The objectives of this pivotal study were to determine the potential toxicity including the highest non-severely toxic dose of TPP-23411 formulated in a 5% dextrose solution for the treatment of cancer when given once or twice weekly intravenously for 4 weeks to the Cynomolgus monkey and to evaluate the potential reversibility of any findings during a 2-week recovery period. The evaluation of toxicity was based on survival, clinical signs, body weight, food consumption, electrocardiography examinations, blood pressure recording, ophthalmological examinations, body temperature recording, the FOB, respiratory rate, hematology, coagulation and blood biochemistry analyses, urinalysis, organ weights, and macroscopic and microscopic examinations. In addition, an extensive panel of immunotoxicological endpoints such as IPT on blood, cytokines/chemokines levels (IL-1β, IL-2, IL-4, IL 5, IL-6, IL-8, IL-10, MCP-1, IFN-γ, and TNF-α), and KLH immunization was included. Blood samples were collected for toxicokinetic and immunogenicity (anti-drug antibodies [ADA]) evaluation. In addition, samples of blood and selected tissues (mesenteric lymph nodes, thymus, and tonsils) were collected for deep RNA sequencing.

TABLE 13.3.2
Key data of the pivotal repeat-dose
(4-week) toxicity study in monkeys

| | |
|---|---|
| Test item | TPP-23411 (preformulated) |
| GLP compliance | Yes |
| Animals | A total of 16 male and 16 female Cynomolgus |
| | monkeys (Macaca fascicularis), at start of |
| | predose phase approximately 2 years old with |
| | body weight of 2.4 to 4.5 kg. |
| Dose | 0 (vehicle control); 15 mg/kg/week; 50 mg/kg/ |
| | week; 2 × 40 (80) mg/kg/week. |
| Administration | Intravenous (bolus) administration over a |
| | period of 4 weeks. |
| Vehicle | 5% dextrose solution |
| General | Clinical observations, food consumption, body |
| investigations | weights, ophthalmology, cardiovascular investigations |
| | (electrocardiogram, blood pressure measurements, and |
| | respiratory rate), hematology, coagulation, clinical |
| | pathology (blood, urine), physical and neurological |
| | examinations including body temperature, gross |
| | pathology at necropsy, organ weights, histopathology. |
| Safety | Effects on vital organs function: Detailed clinical |
| pharmacology | observations, physical/neurological examinations |
| endpoints | (abdominal palpation, body temperature, heart, and |
| | lung auscultation; general sensory aspects, cerebral |
| | (pupillary, orbicularis oculi) and spinal reflexes |
| | (patellar, anal), foot grip reflex), respiration rate, |
| | arterial blood pressure (high definition oscillometry), |
| | and ECG (30-60 s recording). |
| Special | Deep mRNA analysis, C-reactive protein (CRP) and |
| investigations | cytokines/chemokines levels, immunophenotyping |
| | (IPT), T cell dependent antibody response (TDAR) |
| | analysis, anti-drug antibody (ADA) evaluations. |
| Reversibility | Investigation of adverse effects during a post- |
| | treatment period of two weeks. |
| Toxicokinetics | Analysis of plasma concentrations. |

### Clinical Observations

Mortality was unaffected by dosing with TPP-23411. No signs of overt toxicity and no effects on food consumption or body weight were observed during the study.

### Clinical Pathology

No relevant impact on hematology, coagulation, clinical chemistry, ECG or ophthalmoscopy was found.

### Immunotoxicological Parameters

There were no adverse effects of the test item on the level of any cytokine determined during the study. Levels for IL-2, IL-4, IL-5, IL-6, and TNFα were generally low throughout the study and were considered within normal physiological variation. For minor elevations in IFN-γ at the high dose noted for a few animals, a potential relation to TPP-23411 cannot be excluded. Elevated levels for IL-1β in 1 high-dose animal and for IL-10 in 2 high-dose animals were noted, which exceeded the individual pre-treatment or control group.

The assessment of the humoral immune response upon antigen stimulation with KLH revealed no signs of alterations based on test item administration.

Treatment with TPP-23411 between 15 and 50 mg/kg once weekly or 40 mg/kg twice weekly had no adverse effect on any leucocyte subpopulation measured by flow cytometry during this study. Reduced absolute CD16+ NK cell counts were noted at ≥15 mg/kg throughout the dosing phase for females on Days 15 and 29 and for males on Days 8 and 15. The mean NK cell counts were decreased by 74% and 90% for males and females, respectively, at the dose of 40 mg/kg twice weekly on Day 29 when compared to second pre-dose group mean. This effect was not reversible until the end of the 2-week recovery period.

### Cross Pathology, Histopathology

Moderate increases in spleen weights were observed during necropsy at all dose levels in males and at 40 mg/kg twice weekly or 50 mg/kg once weekly in females. It corresponded with a higher incidence of macroscopic enlargement of the organ. In the absence of microscopic correlate, this finding was considered incidental.

All histopathological changes matched to the spectrum of spontaneous pathology commonly noted in Cynomolgus monkeys of this age and origin.

TPP-23411 was well tolerated at the injection site. Procedure-related changes, such as fibrosis, edema, hemorrhage or inflammatory cell infiltrates, were observed with a similar incidence and severity in controls and TPP-23411-administered animals.

Minimal ADA formation was observed in all dose groups, including the control group. The only moderate ADA response was observed in one animal from control group 1, which indicates the presence of pre-existing antibodies. In all other animals with ADA formation, only low responses were detected at Day 1 at 0 hours, Day 22 at 0 hours and 168 hours and Day 29 at 336 hours. No effect on the plasma concentrations of TPP-23411 in the affected animals could be detected. Therefore, the sporadic occurrence of ADA in all the study groups was considered incidental without any biological relevance.

With respect to toxicokinetics, no relevant gender difference in the systemic exposure of TPP-23411 was observed. Cₘₐₓ levels were achieved after 0.25 hours on Day 1 for all dosing levels. However, at Day 22, Cₘₐₓ levels were mainly achieved after 1 hour. On Day 22, both AUC₀₋₁₆₈ and Cₘₐₓ increased in an approximately dose-proportional manner when increasing the dose of TPP-23411 from 15.0 to 50.0 mg/kg in male and female monkeys. For comparison of all groups, AUC₀₋₁₄₄ was calculated for 15.0 and 50.0 mg/kg group and estimated for group 4 by combining AUC₀₋₇₂ on Day 1 with Day 4 or Day 22 with Day 25.

On Day 22, AUC₀₋₁₄₄ of 40 mg/kg dose group was significantly increased compared to the 15.0 mg/kg dose group and moderately increased compared to the 50 mg/kg dose group due to the twice weekly vs. once weekly dosing.

On Day 22 and 25, Cₘₐₓ increased in the 40.0 mg/kg dose group in an approximately dose-proportional manner compared to Day 22 of the 15.0 and 50.0 mg/kg dose groups.

Repeated dosing did not influence both AUC₀₋₁₆₈ and Cₘₐₓ levels, if animals were dosed once weekly (Group 2 and 3). However, combined AUC levels were slightly upregulated (compared to Day 1/4 levels), if TPP-23411 was administered twice weekly slightly accumulating with each dose. Cₘₐₓ levels were slightly upregulated compared to Day 1.

TABLE 13.3.3
Summary on exposure in the repeat-dose (4-week) toxicity
study in male (M) and female (F) monkeys on Day 22.

| | | 15 | 50 | 2 × 40 |
|---|---|---|---|---|
| | | Sex | | |
| Dose | [mg/kg] | M + F | M + F | M + F |
| Cₘₐₓ | [μg/mL] | 418 | 1490 | 1400 |
| tₘₐₓ | [h] | 1.00 | 0.625 | 1.00 |
| C_{max, norm} | [kg/L] | 27.9 | 29.8 | 17.5 |
| C₍ₜᵢₙₜ₎-Cₘₐₓ | [%] | 2.65 | 4.99 | 18.4 |
| AUC₍₀₋₁₄₄₎ | [μg · h/mL] | 11400 | 41300 | 78800 ^{a} |
| AUC₍₀₋₁₄₄₎ₙₒᵣₘ | [kg · h/L] | 761 | 825 | 985 ^{a} |
| AUCₐₗₗ | [μg · h/L] | 11700 | 43300 | 78800 |
| AUC_{all, norm} | [kg · h/L] | 781 | 865 | 985 |
| RA-AUCₐₗₗ | [%] | 101 | 87.0 | 144 |
| RA-Cₘₐₓ | [%] | 89.6 | 86.8 | 128 |

^{a} extrapolated values.
AUC: area under the plasma concentration vs. time curve; AUC₍₀₋₁₄₄₎: AUC from 0 to 144 hours; AUC₍₀₋₁₄₄₎ₙₒᵣₘ: AUC₍₀₋₁₄₄₎ normalized to dose and body weight; Cₘₐₓ: maximum drug concentration in plasma; C_{max, norm}: Cₘₐₓ normalized to dose and body weight; tₘₐₓ: time to reach maximum drug concentration in plasma; C(tint): concentration at 168 hours; RA_{Auc}: accumulation ratio calculated from AUCτ after multiple dosing and AUCτ after single dosing; RA_{Cmax}: relative maximum drug concentration in plasma; RA_{AUC(0-168)}: RA_{Auc} from 0 to 168 hours.

Deep mRNA Analysis

RNA-sequencing study was performed to assess whether the described treatment with TPP-23411 has any general effects on gene expression in specific tissues of Cynomolgus monkeys, such as thymus, tonsils, and mesenteric lymph nodes. The gene CCR8 showed elevated baseline expression in thymus, when compared to tonsils and mesenteric lymph nodes, but no significant changes in gene expression upon treatment with TPP-23411. Other genes of interest showed only minor, statistically not significant changes in expression upon treatment across all three tissues but considerable inter-animal variability of expression values.

TABLE 13.3.4
Noteworthy findings of the pivotal repeat-dose
(4-week) toxicity study in Cynomolgus monkeys.

| | | | Dose |
|---|---|---|---|
| Investigation | Findings | Sex | [mg/kg] |
| Clinical | No mortality | M + F | all |
| observations | No relevant effects on body | M + F | all |
| | weight or body weight gain | | |
| | No relevant effects on food | M + F | all |
| | consumption | | |
| Safety | No relevant effect on vital | M + F | all |
| pharmacology | organ functions | | |
| Ophthalmology | No relevant effects | M + F | all |
| ECG | No relevant effects on ECG, | M + F | all |
| | blood pressure, respiratory | | |
| | rate | | |
| Hematology, | No relevant effects | M + F | all |
| coagulation | | | |
| Clinical Chemistry | | M + F | all |
| blood | No relevant effects | M + F | all |
| urine | No relevant effects | M + F | all |
| Immunotoxicology | No relevant effects regarding | M + F | all |
| | cytokine analysis, T cell | | |
| | dependent antibody response | | |
| | (TDAR) analysis, anti-drug | | |
| | antibody (ADA) evaluations | | |
| | Immunophenotyping: isolated | M + F | ≥15 |
| | reduction of NK cells | | |
| Post-mortem | | M + F | all |
| cross pathology | No relevant effects | M + F | all |
| organ weights | No relevant effects | M + F | all |
| Histopathology | No relevant effects on organs | M + F | all |
| | and tissues; no local | | |
| | intolerance | | |
| Recovery | No relevant effects on organ | M + F | all |
| | weights and no relevant | | |
| | macroscopic or microscopic | | |
| | alterations after 2-week | | |
| | post treatment period | | |

### Example 14: Cytokine Release Assay (CRA)

Cytokine release assays were conducted with human whole blood (with soluble antibody added) and human PBMCs (with wet-coated antibody) to investigate the potential of TPP-23411 alone or in combination with pembrolizumab to activate secretion of cytokines (e.g. IFN-γ, IL-1β, IL-2, IL-4, IL-6, IL-8, IL-10, and TNFα analyzed).

### Example 14.1: Cytokine Release Assay—Study 1

Material (heparinized human peripheral blood) from 10 healthy donors was incubated for 24 hours with either PBS (negative control, NC), LPS/PHA (positive control, PC), commercially available control antibodies (anti CD3 antibody OKT3 or anti CD28 antibody ANC28.1, anti-CD20 antibody rituximab (MabThera), anti-EGFR antibody cetuximab (Erbitux)), TPP-23411 or corresponding isotype control antibody TPP-9809. All antibodies were tested at 3 different concentrations (50, 10, and 2 μg/mL). In addition, TPP-23411 and isotype control antibody TPP-9809 were tested in combination with 2 μg/mL pembrolizumab. The concentration of the cytokines in the supernatant was determined by a multiplex electro-chemiluminescence method.

The incubation of whole blood with soluble TPP-23411 antibody resulted in a clear dose-dependent release of IFN-γ to median concentration levels (over all donors) higher than those observed for control antibody MabThera and lower than those observed for the control antibody ANC28.1. IL-1β, IL-6, and TNFα were induced to levels comparable or slightly higher to those observed for the control antibody MabThera but clearly lower compared to those observed for the control antibody ANC28.1. The incubation of PBMCs with wet-coated TPP-23411 antibody induced a clear release of all analyzed cytokines (IFN-γ, IL-1β, IL-2, IL-4, IL-6, IL-8, IL-10, and TNFα) to levels higher than those obtained with control antibody MabThera, but lower or comparable to those observed for the control antibody OKT3.

The combination of pembrolizumab with TPP-23411 in the two assay formats led to comparable cytokine pattern, released cytokine concentration levels and responder frequencies as the assays with TPP-23411 alone.

TABLE 14.1.1
CRA with PBMC/wet-coated antibodies. Median Cytokine Concentration (pg/ml) after stimulation
with the indicated antibodies and concentrations (50 μg/ml, 10 μg/ml, 2 μg/ml or
50 μg/ml, 10 μg/ml, 2 μg/ml of TPP-23411 in combination with 2 μg/ml Pembrolizumab(Pembro)).

| Antibody - μg/mL | IFN-γ | IL-10 | IL1β | IL-2 | IL-4 | IL-6 | IL-8 | TNF-α |
|---|---|---|---|---|---|---|---|---|
| MabThera - 50 | 5.97 | 0.25 | 4.83 | 1.68 | 0.02 | 41.20 | 4082.61 | 74.88 |
| MabThera - 10 | 1.83 | 0.47 | 3.57 | 1.86 | 0.07 | 25.17 | 2984.65 | 32.88 |
| MabThera - 2 | 2.46 | 0.33 | 3.42 | 1.32 | 0.01 | 24.90 | 2520.92 | 78.33 |
| TPP-23411 - 50 | 42.44 | 0.87 | 30.75 | 5.59 | 0.18 | 203.47 | 7196.68 | 1150.85 |
| TPP-23411 - 10 | 34.33 | 2.06 | 26.28 | 5.54 | 0.61 | 192.56 | 7149.24 | 927.01 |
| TPP-23411 - 2 | 83.15 | 0.49 | 14.94 | 3.21 | 0.09 | 121.05 | 7045.00 | 557.71 |
| IgG1 Iso Ctrl - 50 | 0.69 | 0.37 | 2.85 | 2.32 | 0.12 | 30.32 | 3369.87 | 49.70 |
| IgG1 Iso Ctrl - 10 | 2.05 | 0.19 | 0.64 | 0.85 | 0.00 | 8.21 | 550.54 | 8.58 |
| IgG1 Iso Ctrl - 2 | 0.20 | 0.30 | 0.35 | 0.86 | 0.00 | 3.11 | 231.68 | 2.84 |
| Pembrolizumab - 2 | 6.15 | 0.00 | 0.45 | 1.69 | 0.05 | 3.75 | 159.03 | 2.38 |
| TPP-23411/Pembro - 50/2 | 40.19 | 0.92 | 33.88 | 4.44 | 0.11 | 263.86 | 7157.22 | 1184.49 |
| TPP-23411/Pembro - 10/2 | 33.33 | 1.90 | 32.93 | 4.61 | 0.53 | 267.41 | 6676.28 | 950.20 |
| TPP-23411/Pembro - 2/2 | 64.01 | 0.67 | 15.25 | 3.47 | 0.06 | 148.86 | 6938.65 | 621.80 |
| IgG1 Iso Ctrl/Pembro - 50/2 | 0.61 | 0.40 | 2.83 | 1.43 | 0.09 | 26.21 | 3682.52 | 53.16 |
| IgG1 Iso Ctrl/Pembro - 10/2 | 2.13 | 0.25 | 0.57 | 0.76 | 0.00 | 7.91 | 641.38 | 7.53 |
| IgG1 Iso Ctrl/Pembro - 2/2 | 0.23 | 0.24 | 0.32 | 0.61 | 0.00 | 4.29 | 214.28 | 3.69 |
| Erbitux - 50 | 2.46 | 0.38 | 0.24 | 1.11 | 0.00 | 4.93 | 294.85 | 5.06 |
| Erbitux - 10 | 0.90 | 0.37 | 0.15 | 1.24 | 0.00 | 2.60 | 134.02 | 2.63 |
| Erbitux - 2 | 3.96 | 0.00 | 0.15 | 1.61 | 0.00 | 3.55 | 105.31 | 6.05 |
| OKT3 - 50 | 991.36 | 14.92 | 78.09 | 27.43 | 1.13 | 594.06 | 8564.64 | 2600.41 |
| OKT3 - 10 | 2750.49 | 19.67 | 67.38 | 70.32 | 1.70 | 433.96 | 8828.46 | 2785.61 |
| OKT3 - 2 | 8772.88 | 50.32 | 67.00 | 117.26 | 2.13 | 366.19 | 8756.87 | 3430.74 |
| NC | 0.41 | 0.11 | 0.42 | 0.72 | 0.03 | 4.19 | 184.41 | 2.80 |
| PC | 1943.69 | 507.23 | 2188.60 | 292.45 | 11.30 | 5716.38 | 8918.73 | 3068.80 |

TABLE 14.1.2
95^{th} Percentile of Mean Cytokine Concentration (pg/ml)
after stimulation of PBMCs with wet-coated Erbitux antibody
(concentrations 50 μg/ml, 10 μg/ml, 2 μg/ml); N = 8

| | IFN- | IL- | IL- | | | | | TNF- |
|---|---|---|---|---|---|---|---|---|
| | γ | 10 | 1β | IL-2 | IL-4 | IL-6 | IL-8 | α |
| Erbitux - 50 | 14.16 | 0.49 | 6.44 | 1.76 | 0.02 | 35.76 | 5364.55 | 58.78 |
| Erbitux - 10 | 25.50 | 0.50 | 5.68 | 2.13 | 0.09 | 32.41 | 5058.72 | 41.62 |
| Erbitux - 2 | 16.98 | 0.34 | 5.93 | 2.63 | 0.07 | 29.22 | 4751.53 | 53.78 |

TABLE 14.1.3
CRA with PBMC/wet-coated antibodies. Frequency of positive response
after stimulation with the indicated antibodies and concentrations
(50 μg/ml, 10 μg/ml, 2 μg/ml or 50 μg/ml, 10 μg/ml,
2 μg/ml of TPP-23411 in combination with 2 μg/ml Pembrolizumab(Pembro)); N = 8

| Antibody - μg/mL | IFN-γ | IL-10 | IL-1β | IL-2 | IL-4 | IL-6 | IL-8 | TNF-α |
|---|---|---|---|---|---|---|---|---|
| MabThera - 50 | 25 | 13 | 50 | 50 | 50 | 63 | 38 | 63 |
| MabThera - 10 | 13 | 50 | 25 | 38 | 38 | 38 | 38 | 38 |
| MabThera - 2 | 13 | 38 | 25 | 0 | 25 | 38 | 38 | 50 |
| TPP-23411 - 50 | 88 | 75 | 75 | 88 | 75 | 75 | 75 | 100 |
| TPP-23411 - 10 | 50 | 88 | 75 | 88 | 75 | 75 | 75 | 88 |
| TPP-23411 - 2 | 100 | 75 | 75 | 63 | 63 | 75 | 75 | 88 |
| IgG1 Iso Ctrl - 50 | 13 | 38 | 38 | 63 | 63 | 50 | 38 | 50 |
| IgG1 Iso Ctrl - 10 | 0 | 0 | 25 | 25 | 13 | 25 | 25 | 25 |
| IgG1 Iso Ctrl - 2 | 0 | 38 | 25 | 13 | 25 | 25 | 25 | 13 |
| Pembrolizumab - 2 | 0 | 0 | 14 | 14 | 29 | 14 | 14 | 0 |
| TPP-23411/Pembro - 50/2 | 88 | 63 | 75 | 88 | 75 | 75 | 75 | 88 |
| TPP-23411/Pembro - 10/2 | 50 | 63 | 75 | 88 | 75 | 75 | 75 | 88 |
| TPP-23411/Pembro - 2/2 | 100 | 88 | 75 | 75 | 50 | 75 | 75 | 75 |
| IgG1 Iso Ctrl/Pembro - 50/2 | 0 | 50 | 38 | 38 | 75 | 50 | 38 | 50 |
| IgG1 Iso Ctrl/Pembro - 10/2 | 0 | 13 | 25 | 13 | 0 | 25 | 25 | 25 |
| IgG1 Iso Ctrl/Pembro - 2/2 | 0 | 25 | 25 | 13 | 25 | 25 | 25 | 25 |
| Erbitux - 50 | 14 | 14 | 14 | 14 | 14 | 14 | 14 | 14 |
| Erbitux - 10 | 14 | 14 | 14 | 14 | 14 | 14 | 14 | 14 |
| Erbitux - 2 | 14 | 14 | 14 | 14 | 14 | 14 | 14 | 14 |
| OKT3 - 50 | 100 | 100 | 100 | 100 | 100 | 75 | 100 | 100 |
| OKT3 - 10 | 100 | 100 | 100 | 100 | 100 | 88 | 100 | 100 |
| OKT3 - 2 | 100 | 100 | 100 | 100 | 100 | 100 | 100 | 100 |

TABLE 14.1.4
CRA with whole blood/soluble antibodies. Median Cytokine Concentration (pg/ml)
after stimulation with the indicated antibodies and concentrations (50 μg/ml,
10 μg/ml, 2 μg/ml or 50 μg/ml, 10 μg/ml, 2 μg/ml of TPP-23411
in combination with 2 μg/ml Pembrolizumab(Pembro)); N = 10.

| Antibody - μg/mL | IFN-γ | IL-10 | IL1β | IL-2 | IL-4 | IL-6 | IL-8 | TNF-α |
|---|---|---|---|---|---|---|---|---|
| MabThera - 50 | 11.18 | 0.24 | 0.74 | 0.24 | 0.03 | 2.23 | 137.89 | 1.45 |
| MabThera - 10 | 9.21 | 0.24 | 1.22 | 0.18 | 0.06 | 2.43 | 279.62 | 1.68 |
| MabThera - 2 | 17.50 | 0.28 | 1.12 | 0.19 | 0.01 | 4.18 | 267.48 | 3.09 |
| TPP-23411 - 50 | 395.86 | 0.42 | 3.02 | 0.40 | 0.00 | 9.25 | 323.43 | 6.49 |
| TPP-23411 - 10 | 217.35 | 0.25 | 1.35 | 0.28 | 0.00 | 5.20 | 146.16 | 3.46 |
| TPP-23411 - 2 | 32.16 | 0.13 | 0.42 | 0.25 | 0.00 | 1.85 | 87.46 | 1.70 |
| IgG1 Iso Ctrl - 50 | 3.82 | 0.32 | 0.71 | 0.24 | 0.00 | 1.06 | 119.21 | 1.21 |
| IgG1 Iso Ctrl - 10 | 4.73 | 0.29 | 0.40 | 0.25 | 0.00 | 0.91 | 92.23 | 1.19 |
| IgG1 Iso Ctrl - 2 | 3.87 | 0.27 | 0.34 | 0.20 | 0.00 | 0.84 | 71.12 | 0.89 |
| Pembrolizumab - 2 | 12.54 | 0.00 | 0.57 | 1.41 | 0.18 | 1.81 | 118.07 | 0.74 |
| TPP-23411-Pembro - 50/2 | 346.06 | 0.50 | 2.82 | 0.58 | 0.00 | 8.57 | 328.84 | 5.28 |
| TPP-23411-Pembro - 10/2 | 214.64 | 0.29 | 1.51 | 0.33 | 0.00 | 4.25 | 165.83 | 3.36 |
| TPP-23411-Pembro - 2/2 | 33.33 | 0.20 | 0.47 | 0.25 | 0.00 | 2.07 | 116.83 | 1.50 |
| IgG1 Iso Ctrl/Pembro - 50/2 | 3.90 | 0.41 | 0.93 | 0.15 | 0.00 | 1.21 | 210.04 | 0.89 |
| IgG1 Iso Ctrl/Pembro - 10/2 | 5.52 | 0.34 | 0.71 | 0.17 | 0.00 | 0.77 | 217.76 | 1.02 |
| IgG1 Iso Ctrl/Pembro - 2/2 | 4.92 | 0.22 | 0.38 | 0.18 | 0.01 | 0.51 | 88.71 | 0.62 |
| Erbitux - 50 | 4.75 | 0.15 | 0.52 | 0.18 | 0.00 | 0.63 | 80.77 | 0.61 |
| Erbitux - 10 | 3.58 | 0.19 | 0.46 | 0.24 | 0.00 | 0.49 | 70.57 | 0.75 |
| Erbitux - 2 | 6.12 | 0.00 | 0.27 | 0.57 | 0.00 | 0.97 | 77.96 | 0.79 |
| ANC28.1 - 50 | 557.89 | 50.04 | 4.43 | 23.23 | 4.26 | 63.97 | 3489.95 | 13.12 |
| ANC28.1 - 10 | 464.63 | 36.41 | 2.87 | 27.22 | 3.83 | 54.69 | 890.76 | 9.11 |
| ANC28.1 - 2 | 336.53 | 19.64 | 1.06 | 21.65 | 1.03 | 7.74 | 236.21 | 4.32 |
| NC | 5.07 | 0.23 | 0.35 | 0.11 | 0.06 | 0.76 | 59.79 | 0.77 |
| PC | 4341.39 | 684.18 | 2679.40 | 14.91 | 10.76 | 6262.81 | 5524.70 | 4169.81 |

TABLE 14.1.5
95^{th} Percentile of Mean Cytokine Concentration (pg/ml) after
stimulation of whole blood with soluble Erbitux antibody
(concentrations 50 μg/ml, 10 μg/ml, 2 μg/ml); N = 10.

| Antibody - | IFN- | IL- | IL- | | | | | TNF- |
|---|---|---|---|---|---|---|---|---|
| μg/mL | γ | 10 | 1β | IL-2 | IL-4 | IL-6 | IL-8 | α |
| Erbitux - 50 | 17.10 | 0.50 | 0.84 | 0.53 | 0.11 | 1.68 | 388.40 | 1.57 |
| Erbitux - 10 | 14.19 | 0.57 | 0.82 | 0.66 | 0.02 | 1.15 | 330.38 | 1.89 |
| Erbitux - 2 | 18.12 | 0.08 | 2.18 | 1.32 | 0.03 | 3.77 | 300.83 | 1.95 |

TABLE 14.1.6
Frequency of positive response after stimulation with the indicated
antibodies and concentrations (50 μg/ml, 10 μg/ml, 2 μg/ml
or 50 μg/ml, 10 μg/ml, 2 μg/ml of TPP-23411 in combination
with 2 μg/ml Pembrolizumab(Pembro)); N = 10.

| Antibody - μg/mL | IFN-γ | IL-10 | IL-1β | IL-2 | IL-4 | IL-6 | IL-8 | TNF-α |
|---|---|---|---|---|---|---|---|---|
| MabThera - 50 | 40 | 0 | 30 | 20 | 10 | 70 | 10 | 40 |
| MabThera - 10 | 40 | 0 | 90 | 0 | 70 | 100 | 50 | 40 |
| MabThera - 2 | 50 | 90 | 30 | 10 | 40 | 50 | 40 | 80 |
| TPP-23411 - 50 | 100 | 40 | 90 | 20 | 0 | 100 | 30 | 100 |
| TPP-23411 - 10 | 100 | 20 | 100 | 0 | 20 | 100 | 10 | 80 |
| TPP-23411 - 2 | 80 | 70 | 0 | 0 | 10 | 0 | 10 | 30 |
| IgG1 Iso Ctrl - 50 | 20 | 30 | 50 | 20 | 10 | 40 | 20 | 30 |
| IgG1 Iso Ctrl - 10 | 20 | 10 | 20 | 20 | 30 | 40 | 20 | 30 |
| IgG1 Iso Ctrl - 2 | 10 | 80 | 0 | 20 | 40 | 10 | 10 | 10 |
| Pembrolizumab - 2 | 30 | 0 | 30 | 100 | 70 | 60 | 10 | 20 |
| TPP-23411-Pembro - 50/2 | 100 | 50 | 90 | 60 | 20 | 100 | 20 | 100 |
| TPP-23411-Pembro - 10/2 | 100 | 40 | 70 | 30 | 10 | 100 | 10 | 90 |
| TPP-23411-Pembro - 2/2 | 90 | 80 | 0 | 10 | 20 | 10 | 0 | 40 |
| IgG1 Iso Ctrl/Pembro - 50/2 | 20 | 30 | 60 | 10 | 0 | 30 | 30 | 20 |
| IgG1 Iso Ctrl/Pembro - 10/2 | 30 | 10 | 50 | 20 | 20 | 30 | 40 | 20 |
| IgG1 Iso Ctrl/Pembro - 2/2 | 10 | 80 | 10 | 0 | 30 | 0 | 20 | 10 |
| Erbitux - 50 | 0 | 0 | 0 | 0 | 0 | 0 | 0 | 0 |
| Erbitux - 10 | 0 | 0 | 0 | 0 | 0 | 0 | 0 | 0 |
| Erbitux - 2 | 0 | 0 | 0 | 0 | 0 | 0 | 0 | 0 |
| ANC28.1 - 50 | 100 | 100 | 100 | 100 | 100 | 100 | 100 | 100 |
| ANC28.1 - 10 | 90 | 90 | 90 | 90 | 90 | 90 | 80 | 90 |
| ANC28.1 - 2 | 100 | 100 | 30 | 90 | 80 | 80 | 50 | 80 |

### Example 14.2: In Vitro Cytokine Release Assay—Study 2

As follow-up investigation, cytokine release assays with lower concentrations of TPP-23411 ranging from 10 μg/mL to 0.128 ng/mL were conducted under the same assay conditions.

Material from 8 healthy donors was incubated for 24 hours with either PBS (negative control, NC), LPS/PHA (positive control, PC), or commercially available control antibodies (anti CD3 antibody OKT3 or anti CD28 antibody ANC28.1, anti-CD20 antibody rituximab (MabThera), anti-EGFR antibody cetuximab (Erbitux), anti-CD52 antibody alemtuzumab (Lemtrada), and anti-CCR4 antibody mogamulizumab (Poteligeo)), TPP-23411 or corresponding isotype control antibody TPP-9809.

TPP-23411, its isotype control and Poteligeo were tested at 8 different concentrations (10/2/0.4/0.08/0.016/0.0032/0.00064/0.000128 μg/mL). All other commercially available antibodies were tested at 4 or 5 different concentrations (10/2/0.4/0.08/0.016 μg/mL).

The concentration of the cytokines in the supernatant was determined. In addition, flow cytometric determination of cell numbers (monocytes, T cells, NK cells, granulocytes and lymphocytes) in the cellular fraction of this cytokine release assay set up after the 24 hour incubation from two of the donors (stimulated with TPP-23411, isotype control antibody, Lemtrada and Poteligeo only) was conducted.

The incubation of whole blood with soluble TPP-23411 antibody resulted in dose-dependent release of IFN-γ, IL-1β, IL-6, TNFα, and IL-8. Cytokines were induced to median concentration levels (over all donors) lower than those observed with alemtuzumab, ANC28.1 and mogamulizumab (except for IL-1 β levels that were comparable to mogamulizumab and a higher concentration of IL-8 at 10 μg/mL when compared to mogamulizumab). When compared to rituximab, TPP-23411 induced higher IFN-γ, IL-6, TNFα, and IL-8 cytokine levels at 10 μg/mL dose but comparable levels at the lower antibody doses tested. IL-1 β was not induced by rituximab but by TPP-23411. IFN-γ was defined as lead cytokine due to its robust dose-response and the highest sensitivity. Slight effects have been observed at concentrations starting at 0.4 μg/mL.

An analysis of the cell pellet of this cytokine release assay set up (with test items TPP-23411, isotype control antibody, alemtuzumab, and mogamulizumab only) by flow cytometry of 2 donors demonstrated a dose-dependent decrease of monocytes, T cells, NK cells, and lymphocytes by Lemtrada, as expected. None of other test items lead to a reduction of any of those cell types. Only one of the 2 donors tested showed a reduction of monocytes below 50% at the highest concentration of 10 μg/mL when treated with TPP-23411.

The incubation of PBMCs with wet-coated TPP-23411 antibody induced a clear dose-dependent release of IFN-γ, IL-1@, IL-6, IL-8, IL-10, and TNFα, very low amounts of IL-2 and no induction of IL-4.

The comparator antibodies mogamulizumab and alemtuzumab did show cytokine induction but no clear dose-response relationship. In comparison to the more dose-dependent inducer OKT3, the IFN-γ, IL-1β, IL-6, IL-10, TNFα, and IL-2 median cytokine levels (over all donors) obtained with TPP-23411 were much lower. For IL-6 the median cytokine level obtained with 10 μg/mL antibody was comparable between OKT3 and TPP-23411. For all lower doses tested (from 2 μ/mL to 0.128 ng/mL), IL-6 levels were lower to those induced by OKT3.

Intracellular staining and subsequent flow cytometry analysis after a 5 hours incubation of whole blood with TPP-23411 or its isotype control antibody demonstrated IFN-γ production (and degranulation as detected by CD107a marker) by CD3-CD16+CD56+ NK cells (and not CD3+ T cells) in this initial phase.

Precautions will be taken during administration to patients to reduce the potential risks of infusion-related reactions due to the release of cytokines (including cytokine release syndrome) and to mitigate the risk of immune-related adverse events such as dermatologic toxicities (i.e., rash) observed with other Treg-depleting agents.

When testing fucosylated anti-CCR8 antibody variants or a ‘silenced’ Fc variant (with reduced binding to FcgRs) on two donors, those variants did not—or to a much lesser extent—induce IFN-γ, IL-1β, IL-6, TNFα, and IL-8 in the whole blood/soluble antibody cytokine release assay format. Data with the PBMC/wet coated format were less clear and showed high donor dependency. Data on commercially available anti-CCR8 antibodies 433H or L263G8 could not be interpreted due to high background with respective isotype control antibody.

TABLE 14.2.1
CRA with PBMC/wet-coated antibodies. Median Cytokine Concentration (pg/ml) after stimulation with
the indicated antibodies and concentrations (with largest range of 10 μg/ml, 2 μg/ml, 0.4
μg/ml, 0.08 μg/ml, 0.016 μg/ml, 0.0032 μg/ml, 0.00064 μg/ml, 0.000128 μg/ml for TPP-23411); N = 8

| Antibody - μg/mL | IFN-γ | IL-10 | IL-1β | IL-2 | IL-4 | IL-6 | IL-8 | TNF-α |
|---|---|---|---|---|---|---|---|---|
| TPP-23411 - 10 | 48.03 | 0.21 | 10.62 | 2.54 | 0.00 | 22.02 | 6229.57 | 131.78 |
| TPP-23411 - 2 | 74.84 | 0.49 | 6.41 | 3.37 | 0.01 | 15.91 | 5113.90 | 98.79 |
| TPP-23411 - 0.4 | 38.04 | 0.29 | 1.76 | 1.37 | 0.00 | 8.48 | 1243.62 | 34.80 |
| TPP-23411 - 0.08 | 7.69 | 0.10 | 0.14 | 1.29 | 0.00 | 0.93 | 194.36 | 3.50 |
| TPP-23411 - 0.016 | 0.91 | 0.13 | 0.00 | 0.26 | 0.00 | 0.00 | 78.42 | 1.16 |
| TPP-23411 - 0.0032 | 0.03 | 0.10 | 0.03 | 0.18 | 0.00 | 0.23 | 60.13 | 0.75 |
| TPP-23411 - 0.00064 | 0.42 | 0.05 | 0.01 | 0.23 | 0.01 | 0.45 | 71.21 | 0.88 |
| TPP-23411 - 0.000128 | 0.45 | 0.05 | 0.08 | 0.21 | 0.02 | 1.22 | 81.07 | 0.85 |
| IgG1 Iso Ctrl, afuco - 10 | 25.62 | 0.32 | 0.54 | 0.84 | 0.00 | 3.85 | 599.56 | 9.69 |
| IgG1 Iso Ctrl, afuco - 2 | 0.93 | 0.14 | 0.01 | 0.68 | 0.00 | 0.00 | 117.53 | 2.25 |
| IgG1 Iso Ctrl, afuco - 0.4 | 0.00 | 0.17 | 0.03 | 0.36 | 0.00 | 0.00 | 64.11 | 1.01 |
| IgG1 Iso Ctrl, afuco - 0.08 | 0.08 | 0.12 | 0.00 | 0.34 | 0.00 | 0.00 | 47.32 | 0.69 |
| IgG1 Iso Ctrl, afuco - 0.016 | 0.22 | 0.15 | 0.01 | 0.23 | 0.00 | 0.04 | 59.98 | 0.63 |
| IgG1 Iso Ctrl, afuco - 0.0032 | 0.15 | 0.07 | 0.00 | 0.19 | 0.00 | 0.11 | 43.96 | 0.51 |
| IgG1 Iso Ctrl, afuco - 0.00064 | 0.00 | 0.06 | 0.02 | 0.16 | 0.00 | 0.24 | 63.46 | 0.60 |
| IgG1 Iso Ctrl, afuco - 0.000128 | 0.17 | 0.07 | 0.03 | 0.28 | 0.00 | 1.31 | 105.14 | 1.26 |
| IgG1 Iso Ctrl - 10 | 0.00 | 0.21 | 0.06 | 0.44 | 0.00 | 0.00 | 226.32 | 1.95 |
| Lemtrada - 10 | 9.31 | 0.33 | 0.25 | 1.35 | 0.00 | 0.27 | 247.87 | 4.27 |
| Lemtrada - 2 | 0.00 | 0.15 | 0.01 | 0.43 | 0.00 | 0.01 | 104.75 | 1.18 |
| Lemtrada - 0.4 | 0.00 | 0.09 | 0.00 | 0.28 | 0.00 | 0.00 | 61.83 | 0.87 |
| Lemtrada - 0.08 | 0.10 | 0.17 | 0.06 | 0.60 | 0.00 | 0.05 | 69.54 | 0.82 |
| Lemtrada - 0.016 | 0.00 | 0.07 | 0.05 | 0.37 | 0.00 | 0.05 | 62.77 | 0.75 |
| Mogamulizumab - 10 | 4.18 | 0.20 | 0.13 | 0.87 | 0.00 | 0.00 | 192.35 | 2.71 |
| Mogamulizumab - 2 | 0.82 | 0.31 | 0.05 | 0.78 | 0.00 | 0.00 | 133.39 | 1.72 |
| Mogamulizumab - 0.4 | 0.29 | 0.30 | 0.19 | 0.57 | 0.00 | 1.27 | 266.14 | 2.02 |
| Mogamulizumab - 0.08 | 0.49 | 0.30 | 0.37 | 1.06 | 0.01 | 1.73 | 249.08 | 1.45 |
| Mogamulizumab - 0.016 | 2.89 | 0.88 | 10.47 | 3.53 | 0.03 | 144.82 | 3547.39 | 11.64 |
| Mogamulizumab - 0.0032 | 2.83 | 1.87 | 15.22 | 4.49 | 0.22 | 244.50 | 3553.80 | 11.57 |
| Mogamulizumab - 0.00064 | 10.95 | 8.41 | 95.29 | 5.83 | 0.39 | 970.36 | 5781.14 | 93.36 |
| Mogamulizumab - 0.000128 | 24.95 | 22.95 | 537.84 | 9.19 | 1.96 | 2346.23 | 6813.28 | 343.56 |
| Erbitux - 10 | 1.07 | 0.33 | 0.07 | 0.58 | 0.00 | 0.17 | 184.74 | 2.65 |
| Erbitux - 2 | 0.00 | 0.40 | 0.26 | 0.58 | 0.00 | 0.62 | 264.04 | 2.54 |
| Erbitux - 0.4 | 0.02 | 0.17 | 0.18 | 0.32 | 0.00 | 0.99 | 179.81 | 1.28 |
| Erbitux - 0.08 | 0.00 | 0.35 | 0.36 | 1.23 | 0.01 | 2.58 | 380.29 | 1.51 |
| MabThera - 10 | 1.46 | 0.63 | 10.84 | 2.17 | 0.00 | 170.94 | 2572.71 | 9.26 |
| MabThera - 2 | 4.20 | 2.61 | 21.44 | 5.01 | 0.23 | 332.18 | 5126.43 | 17.50 |
| MabThera - 0.4 | 7.10 | 6.17 | 82.02 | 3.41 | 0.23 | 856.35 | 6839.74 | 69.48 |
| MabThera - 0.08 | 39.51 | 19.45 | 386.05 | 8.66 | 1.71 | 2008.57 | 6996.02 | 259.75 |
| OKT3 - 10 | 2940.50 | 39.30 | 98.49 | 297.20 | 1.30 | 220.17 | 7427.41 | 2050.14 |
| OKT3 - 2 | 4933.67 | 73.11 | 115.45 | 399.06 | 1.96 | 309.51 | 6852.12 | 1158.73 |
| OKT3 - 0.4 | 1953.99 | 33.01 | 45.06 | 95.11 | 0.77 | 254.44 | 6298.42 | 309.81 |
| OKT3 - 0.08 | 44.73 | 4.08 | 10.44 | 13.58 | 0.10 | 92.32 | 3260.90 | 32.00 |
| OKT3 - 0.016 | 8.58 | 0.77 | 7.92 | 3.97 | 0.00 | 113.14 | 2820.99 | 7.87 |
| TPP-23411 - 2 | 75.13 | 0.90 | 22.04 | 4.45 | 0.12 | 116.42 | 6267.56 | 123.79 |
| NC | 6.15 | 1.86 | 50.56 | 2.68 | 0.16 | 322.08 | 3335.87 | 27.22 |
| PC | 3391.69 | 373.69 | 1358.31 | 167.08 | 9.06 | 4550.41 | 6645.47 | 837.85 |

TABLE 14.2.2
95^{th} Percentile of Mean Cytokine Concentration (pg/ml) stimulation
of PBMC with wet coated Erbitux antibody (concentrations
10 μg/ml, 2 μg/ml, 0.4 μg/ml, 0.08 μg/ml); N = 8

| Antibody - μg/mL | IFN-γ | IL-10 | IL-1β | IL-2 | IL-4 | IL-6 | IL-8 | TNF-α |
|---|---|---|---|---|---|---|---|---|
| Erbitux 10 | 16.73 | 1.00 | 17.72 | 5.53 | 0.00 | 208.67 | 4441.76 | 20.41 |
| Erbitux 2 | 6.37 | 1.79 | 24.86 | 2.78 | 7.86 | 350.50 | 4902.00 | 26.78 |
| Erbitux 0.4 | 10.14 | 5.12 | 60.31 | 3.20 | 0.28 | 1042.14 | 6126.36 | 47.63 |
| Erbitux 0.08 | 18.61 | 9.38 | 93.52 | 6.47 | 0.87 | 1358.85 | 7146.00 | 68.82 |

TABLE 14.2.3
95^{th} Percentile of Mean Cytokine Concentration (pg/ml) stimulation of PBMC with wet
coated IgG1 Iso Ctrl afuco antibody (concentrations 10 μg/ml, 2 μg/ml, 0.4
μg/ml, 0.08 μg/ml, 0.016 μg/ml, 0.0032 μg/ml, 0.00064 μg/ml, 0.000128 μg/ml); N = 8.

| Antibody - μg/mL | IFN-γ | IL-10 | IL-1β | IL-2 | IL-4 | IL-6 | IL-8 | TNF-α |
|---|---|---|---|---|---|---|---|---|
| IgG1 Iso Ctrl, afuco - 10 | 93.17 | 0.64 | 0.77 | 6.14 | 0.00 | 8.43 | 1369.71 | 26.08 |
| IgG1 Iso Ctrl, afuco - 2 | 13.44 | 0.31 | 1.06 | 2.93 | 0.00 | 2.63 | 265.54 | 4.03 |
| IgG1 Iso Ctrl, afuco - 0.4 | 5.12 | 0.40 | 0.09 | 1.08 | 0.00 | 0.32 | 159.18 | 1.55 |
| IgG1 Iso Ctrl, afuco - 0.08 | 5.23 | 0.31 | 0.08 | 1.85 | 0.01 | 1.17 | 176.70 | 1.20 |
| IgG1 Iso Ctrl, afuco - 0.016 | 3.61 | 0.22 | 0.44 | 1.17 | 0.02 | 0.55 | 116.88 | 1.19 |
| IgG1 Iso Ctrl, afuco - 0.0032 | 7.52 | 0.29 | 0.22 | 2.31 | 0.02 | 0.56 | 107.82 | 1.25 |
| IgG1 Iso Ctrl, afuco - 0.00064 | 7.86 | 0.24 | 0.10 | 1.89 | 0.02 | 1.10 | 147.03 | 1.33 |
| IgG1 Iso Ctrl, afuco - 0.000128 | 6.05 | 1.74 | 14.86 | 2.76 | 0.05 | 2.66 | 190.41 | 6.70 |

TABLE 14.2.4
Frequency of positive response after stimulation with the indicated antibodies and
concentrations *(with largest range of 10 μg/ml, 2 μg/ml, 0.4 μg/ml, 0.08
μg/ml, 0.016 μg/ml, 0.0032 μg/ml, 0.00064 μg/ml, 0.000128 μg/ml for TPP-23411); N = 8.

| | IFN-γ | IL-10 | IL-1β | IL-2 | IL-4 | IL-6 | IL-8 | TNF-α |
|---|---|---|---|---|---|---|---|---|
| TPP-23411 - 10 | 25 | 13 | 100 | 0 | 38 | 100 | 100 | 100 |
| TPP-23411 - 2 | 100 | 75 | 100 | 50 | 50 | 100 | 100 | 100 |
| TPP-23411 - 0.4 | 100 | 25 | 100 | 63 | 13 | 100 | 100 | 100 |
| TPP-23411 - 0.08 | 50 | 13 | 63 | 25 | 25 | 50 | 63 | 88 |
| TPP-23411 - 0.016 | 25 | 13 | 0 | 13 | 13 | 25 | 38 | 50 |
| TPP-23411 - 0.0032 | 13 | 13 | 0 | 13 | 25 | 38 | 25 | 13 |
| TPP-23411 - 0.00064 | 0 | 13 | 25 | 13 | 38 | 25 | 25 | 13 |
| TPP-23411 - 0.000128 | 13 | 0 | 0 | 0 | 25 | 0 | 13 | 0 |
| IgG1 Iso Ctrl, afuco - 10 | 13 | 13 | 13 | 13 | 13 | 13 | 13 | 13 |
| IgG1 Iso Ctrl, afuco - 2 | 13 | 13 | 13 | 13 | 0 | 13 | 13 | 13 |
| IgG1 Iso Ctrl, afuco - 0.4 | 13 | 13 | 13 | 13 | 0 | 13 | 13 | 13 |
| IgG1 Iso Ctrl, afuco - 0.08 | 13 | 13 | 13 | 13 | 13 | 13 | 13 | 13 |
| IgG1 Iso Ctrl, afuco - 0.016 | 13 | 13 | 13 | 13 | 13 | 13 | 13 | 13 |
| IgG1 Iso Ctrl, afuco - 0.0032 | 13 | 13 | 13 | 13 | 13 | 13 | 13 | 13 |
| IgG1 Iso Ctrl, afuco - 0.00064 | 13 | 13 | 13 | 13 | 13 | 13 | 13 | 13 |
| IgG1 Iso Ctrl, afuco - 0.000128 | 13 | 13 | 13 | 13 | 13 | 13 | 13 | 13 |
| IgG1 Iso Ctrl - 10 | 0 | 0 | 0 | 0 | 0 | 0 | 0 | 0 |
| Lemtrada - 10 | 25 | 0 | 0 | 13 | 13 | 0 | 0 | 0 |
| Lemtrada - 2 | 13 | 0 | 0 | 25 | 0 | 0 | 0 | 0 |
| Lemtrada - 0.4 | 0 | 0 | 0 | 0 | 0 | 0 | 0 | 0 |
| Lemtrada - 0.08 | 0 | 0 | 0 | 0 | 0 | 0 | 0 | 0 |
| Lemtrada - 0.016 | 0 | 0 | 0 | 0 | 0 | 0 | 0 | 0 |
| Mogamulizumab - 10 | 0 | 13 | 38 | 13 | 0 | 25 | 25 | 13 |
| Mogamulizumab - 2 | 0 | 50 | 25 | 13 | 25 | 25 | 25 | 25 |
| Mogamulizumab - 0.4 | 25 | 38 | 50 | 38 | 25 | 63 | 63 | 63 |
| Mogamulizumab - 0.08 | 25 | 50 | 63 | 38 | 50 | 50 | 50 | 50 |
| Mogamulizumab - 0.016 | 50 | 63 | 63 | 63 | 50 | 63 | 63 | 63 |
| Mogamulizumab - 0.0032 | 25 | 63 | 75 | 63 | 75 | 75 | 75 | 75 |
| Mogamulizumab - 0.00064 | 50 | 63 | 75 | 75 | 63 | 75 | 75 | 75 |
| Mogamulizumab - 0.000128 | 75 | 75 | 75 | 75 | 75 | 75 | 75 | 75 |
| Erbitux - 10 | 13 | 13 | 13 | 13 | 0 | 13 | 13 | 13 |
| Erbitux - 2 | 13 | 13 | 13 | 13 | 13 | 13 | 13 | 13 |
| Erbitux - 0.4 | 13 | 13 | 13 | 13 | 13 | 13 | 13 | 13 |
| Erbitux - 0.08 | 13 | 13 | 13 | 13 | 13 | 13 | 13 | 13 |
| MabThera - 10 | 25 | 50 | 50 | 0 | 38 | 50 | 50 | 38 |
| MabThera - 2 | 38 | 50 | 50 | 63 | 0 | 50 | 50 | 38 |
| MabThera - 0.4 | 38 | 50 | 50 | 50 | 38 | 38 | 63 | 50 |
| MabThera - 0.08 | 63 | 75 | 75 | 75 | 75 | 50 | 50 | 75 |
| OKT3 - 10 | 100 | 100 | 88 | 100 | 100 | 50 | 88 | 100 |
| OKT3 - 2 | 100 | 100 | 75 | 100 | 25 | 50 | 75 | 100 |
| OKT3 - 0.4 | 100 | 75 | 38 | 100 | 63 | 13 | 63 | 88 |
| OKT3 - 0.08 | 50 | 25 | 13 | 75 | 13 | 13 | 25 | 13 |
| OKT3 - 0.016 | 25 | 0 | 25 | 0 | 0 | 25 | 25 | 38 |
| TPP-23411 - 2 | 100 | 88 | 100 | 63 | 88 | 100 | 100 | 100 |

TABLE 14.2.5
CRA with whole blood/soluble antibodies. Median Cytokine Concentration (pg/ml) after stimulation
with the indicated antibodies and concentrations (with largest range of 10 μg/ml, 2 μg/ml,
0.4 μg/ml, 0.08 μg/ml, 0.016 μg/ml, 0.0032 μg/ml, 0.00064 μg/ml, 0.000128 μg/ml for TPP-23411); N = 8.

| Antibody - μg/mL | IFN-γ | IL-10 | IL-1β | IL-2 | IL-4 | IL-6 | IL-8 | TNF-α |
|---|---|---|---|---|---|---|---|---|
| TPP-23411 - 10 | 400.16 | 0.45 | 4.60 | 0.64 | 0.00 | 12.27 | 544.94 | 5.38 |
| TPP-23411 - 2 | 122.36 | 0.33 | 1.93 | 0.59 | 0.02 | 2.54 | 118.70 | 2.62 |
| TPP-23411 - 0.4 | 19.44 | 0.15 | 0.28 | 0.10 | 0.04 | 1.15 | 58.25 | 0.65 |
| TPP-23411 - 0.08 | 7.04 | 0.32 | 0.24 | 0.20 | 0.02 | 0.39 | 42.91 | 0.67 |
| TPP-23411 - 0.016 | 6.81 | 0.18 | 0.17 | 0.10 | 0.04 | 0.35 | 39.16 | 0.39 |
| TPP-23411 - 0.0032 | 5.86 | 0.20 | 0.22 | 0.08 | 0.04 | 0.11 | 40.82 | 0.51 |
| TPP-23411 - 0.00064 | 6.97 | 0.16 | 0.20 | 0.22 | 0.06 | 0.91 | 39.58 | 0.47 |
| TPP-23411 - 0.000128 | 6.96 | 0.15 | 0.32 | 0.23 | 0.13 | 1.01 | 67.56 | 0.58 |
| IgG1 Iso Ctrl, afuco - 10 | 41.50 | 0.28 | 0.57 | 0.04 | 0.00 | 0.80 | 137.67 | 0.99 |
| IgG1 Iso Ctrl, afuco - 2 | 13.26 | 0.35 | 0.27 | 0.26 | 0.03 | 0.87 | 52.43 | 0.78 |
| IgG1 Iso Ctrl, afuco - 0.4 | 7.61 | 0.18 | 0.31 | 0.03 | 0.05 | 0.68 | 47.56 | 0.47 |
| IgG1 Iso Ctrl, afuco - 0.08 | 6.16 | 0.18 | 0.26 | 0.21 | 0.04 | 1.27 | 42.00 | 0.42 |
| IgG1 Iso Ctrl, afuco - 0.016 | 4.18 | 0.12 | 0.14 | 0.00 | 0.03 | 0.79 | 39.19 | 0.35 |
| IgG1 Iso Ctrl, afuco - 0.0032 | 5.82 | 0.13 | 0.18 | 0.07 | 0.05 | 0.38 | 40.42 | 0.42 |
| IgG1 Iso Ctrl, afuco - 0.00064 | 6.38 | 0.15 | 0.17 | 0.07 | 0.11 | 0.51 | 43.71 | 0.40 |
| IgG1 Iso Ctrl, afuco - 0.000128 | 7.50 | 0.19 | 0.33 | 0.10 | 0.15 | 1.68 | 44.22 | 0.39 |
| IgG1 Iso Ctrl - 10 | 8.46 | 0.00 | 0.13 | 0.50 | 0.01 | 0.27 | 50.29 | 0.68 |
| Lemtrada - 10 | 7772.03 | 2.21 | 18.00 | 4.87 | 0.40 | 121.19 | 2538.26 | 58.69 |
| Lemtrada - 2 | 11005.28 | 0.89 | 13.51 | 1.43 | 0.32 | 159.32 | 1622.31 | 87.81 |
| Lemtrada - 0.4 | 9807.07 | 1.86 | 11.46 | 8.11 | 0.31 | 139.57 | 1335.94 | 96.62 |
| Lemtrada - 0.08 | 1471.51 | 0.50 | 1.77 | 0.85 | 0.22 | 25.81 | 419.54 | 13.97 |
| Lemtrada - 0.016 | 59.32 | 0.07 | 0.59 | 0.25 | 0.06 | 1.98 | 86.72 | 1.13 |
| Mogamulizumab - 10 | 1178.28 | 0.35 | 1.42 | 0.43 | 0.03 | 14.58 | 179.14 | 9.90 |
| Mogamulizumab - 2 | 841.18 | 0.53 | 1.27 | 0.87 | 0.06 | 12.13 | 140.04 | 6.58 |
| Mogamulizumab - 0.4 | 495.92 | 0.19 | 0.73 | 0.30 | 0.07 | 7.22 | 151.87 | 3.68 |
| Mogamulizumab - 0.08 | 139.68 | 0.29 | 0.54 | 0.33 | 0.06 | 2.57 | 103.16 | 2.06 |
| Mogamulizumab - 0.016 | 31.63 | 0.21 | 0.27 | 0.31 | 0.07 | 1.18 | 92.84 | 0.74 |
| Mogamulizumab - 0.0032 | 10.54 | 0.28 | 0.25 | 0.41 | 0.07 | 0.51 | 72.08 | 0.69 |
| Mogamulizumab - 0.00064 | 8.31 | 0.22 | 0.41 | 0.15 | 0.13 | 1.18 | 50.81 | 0.55 |
| Mogamulizumab - 0.000128 | 8.49 | 0.14 | 0.38 | 0.36 | 0.13 | 1.36 | 54.93 | 0.58 |
| Erbitux - 10 | 6.26 | 0.27 | 0.26 | 0.27 | 0.04 | 0.69 | 79.15 | 0.77 |
| Erbitux - 2 | 5.51 | 0.35 | 0.26 | 0.41 | 0.05 | 0.54 | 81.18 | 0.70 |
| Erbitux - 0.4 | 5.71 | 0.16 | 0.20 | 0.09 | 0.09 | 0.34 | 54.64 | 0.46 |
| Erbitux - 0.08 | 4.79 | 0.35 | 0.40 | 0.67 | 0.07 | 0.88 | 46.07 | 0.53 |
| MabThera - 10 | 86.26 | 0.26 | 0.45 | 0.41 | 0.08 | 2.20 | 83.08 | 1.31 |
| MabThera - 2 | 171.29 | 0.52 | 0.51 | 0.32 | 0.12 | 3.55 | 79.87 | 1.87 |
| MabThera - 0.4 | 160.43 | 0.30 | 0.40 | 0.33 | 0.05 | 3.57 | 80.72 | 1.71 |
| MabThera - 0.08 | 60.04 | 0.19 | 0.36 | 0.25 | 0.09 | 1.71 | 73.22 | 1.12 |
| ANC28.1 - 10 | 1351.46 | 62.76 | 12.97 | 96.88 | 14.60 | 254.75 | 6333.23 | 16.33 |
| ANC28.1 - 2 | 186.90 | 14.99 | 2.25 | 26.99 | 1.56 | 12.75 | 519.36 | 3.33 |
| ANC28.1 - 0.4 | 8.75 | 0.45 | 0.29 | 1.32 | 0.07 | 0.65 | 68.83 | 0.46 |
| ANC28.1 - 0.08 | 5.91 | 0.29 | 0.35 | 0.37 | 0.09 | 0.91 | 44.76 | 0.50 |
| ANC28.1 - 0.016 | 9.16 | 0.57 | 0.32 | 0.85 | 0.29 | 1.82 | 44.78 | 0.79 |
| TPP-23411 - 2 | 130.85 | 0.15 | 2.68 | 0.90 | 0.11 | 5.47 | 171.62 | 2.06 |
| NC | 15.02 | 0.00 | 0.55 | 1.99 | 0.12 | 2.41 | 53.86 | 0.60 |
| PC | 9759.55 | 348.91 | 1125.00 | 247.22 | 10.92 | 7444.52 | 6632.76 | 2929.30 |

TABLE 14.2.6
95^{th} Percentile of Mean Cytokine Concentration (pg/ml) stimulation
of whole blood with soluble Erbitux antibody (concentrations
10 μg/ml, 2 μg/ml, 0.4 μg/ml, 0.08 μg/ml); N = 8

| | IFN-γ | IL-10 | IL-1β | IL-2 | IL-4 | IL-6 | IL-8 | TNF-α |
|---|---|---|---|---|---|---|---|---|
| Erbitux - 10 | 20.18 | 1.36 | 0.39 | 0.81 | 0.17 | 1.80 | 109.48 | 1.26 |
| Erbitux - 2 | 21.85 | 4.72 | 0.96 | 2.00 | 0.11 | 3.10 | 178.83 | 2.31 |
| Erbitux - 0.4 | 19.50 | 1.19 | 0.42 | 0.54 | 0.17 | 1.66 | 110.68 | 0.93 |
| Erbitux - 0.08 | 18.62 | 2.96 | 0.81 | 1.70 | 0.14 | 2.30 | 106.90 | 0.96 |

TABLE 14.2.7
95^{th} Percentile of Mean Cytokine Concentration (pg/ml) stimulation
of whole blood with soluble IgG1 Iso Ctrl, Afuco antibody (concentrations
10 μg/ml, 2 μg/ml, 0.4 μg/ml, 0.08 μg/ml); N = 8.

| Antibody - μg/mL | IFN-γ | IL-10 | IL-1β | IL-2 | IL-4 | IL-6 | IL-8 | TNF-α |
|---|---|---|---|---|---|---|---|---|
| IgG1 Iso Ctrl, afuco - 10 | 87.69 | 1.17 | 1.02 | 0.56 | 0.04 | 2.54 | 547.36 | 1.93 |
| IgG1 Iso Ctrl, afuco - 2 | 32.75 | 2.11 | 5.52 | 3.40 | 0.22 | 42.78 | 1702.04 | 8.39 |
| IgG1 Iso Ctrl, afuco - 0.4 | 25.54 | 0.96 | 0.68 | 0.51 | 0.11 | 3.55 | 124.62 | 0.90 |
| IgG1 Iso Ctrl, afuco - 0.08 | 21.77 | 0.99 | 0.60 | 0.57 | 0.09 | 1.96 | 112.84 | 0.55 |
| IgG1 Iso Ctrl, afuco - 0.016 | 23.00 | 0.90 | 0.53 | 0.41 | 0.14 | 1.87 | 103.49 | 0.52 |
| IgG1 Iso Ctrl, afuco - 0.032 | 20.91 | 0.96 | 0.42 | 0.58 | 0.14 | 2.06 | 105.06 | 0.83 |
| IgG1 Iso Ctrl, afuco - 0.0064 | 25.57 | 0.88 | 0.47 | 0.35 | 0.15 | 1.97 | 105.71 | 0.55 |
| IgG1 Iso Ctrl, afuco - 0.000128 | 23.10 | 1.01 | 0.61 | 0.46 | 0.24 | 2.56 | 106.43 | 0.56 |

TABLE 14.2.8
Frequency of positive response after stimulation with the indicated antibodies and
concentrations (with largest range of 10 μg/ml, 2 μg/ml, 0.4 μg/ml, 0.08
μg/ml, 0.016 μg/ml, 0.0032 μg/ml, 0.00064 μg/ml, 0.000128 μg/ml for TPP-23411); N = 8.

| | IFN-γ | IL-10 | IL-1β | IL-2 | IL-4 | IL-6 | IL-8 | TNF-α |
|---|---|---|---|---|---|---|---|---|
| TPP-23411 - 10 | 88 | 13 | 88 | 63 | 13 | 88 | 50 | 88 |
| TPP-23411 - 2 | 88 | 0 | 0 | 0 | 0 | 0 | 0 | 0 |
| TPP-23411 - 0.4 | 38 | 13 | 0 | 13 | 0 | 13 | 0 | 38 |
| TPP-23411 - 0.08 | 25 | 13 | 0 | 38 | 13 | 13 | 0 | 63 |
| TPP-23411 - 0.016 | 13 | 13 | 0 | 13 | 0 | 13 | 13 | 13 |
| TPP-23411 - 0.0032 | 13 | 13 | 25 | 38 | 0 | 25 | 13 | 13 |
| TPP-23411 - 0.00064 | 13 | 13 | 0 | 13 | 13 | 0 | 25 | 38 |
| TPP-23411 - 0.000128 | 13 | 13 | 25 | 38 | 0 | 25 | 25 | 63 |
| IgG1 Iso Ctrl, afuco - 10 | 13 | 13 | 13 | 13 | 13 | 13 | 13 | 13 |
| IgG1 Iso Ctrl, afuco - 2 | 13 | 13 | 13 | 13 | 13 | 13 | 13 | 13 |
| IgG1 Iso Ctrl, afuco - 0.4 | 13 | 13 | 13 | 13 | 13 | 13 | 13 | 13 |
| IgG1 Iso Ctrl, afuco - 0.08 | 13 | 13 | 13 | 13 | 13 | 13 | 13 | 13 |
| IgG1 Iso Ctrl, afuco - 0.016 | 13 | 13 | 13 | 13 | 13 | 13 | 13 | 13 |
| IgG1 Iso Ctrl, afuco - 0.0032 | 13 | 13 | 13 | 13 | 13 | 13 | 13 | 13 |
| IgG1 Iso Ctrl, afuco - 0.00064 | 13 | 13 | 13 | 13 | 13 | 13 | 13 | 13 |
| IgG1 Iso Ctrl, afuco - 0.000128 | 13 | 13 | 13 | 13 | 13 | 13 | 13 | 13 |
| IgG1 Iso Ctrl - 10 | 0 | 13 | 13 | 50 | 25 | 13 | 13 | 0 |
| Lemtrada - 10 | 100 | 75 | 100 | 75 | 88 | 100 | 100 | 100 |
| Lemtrada - 2 | 100 | 0 | 100 | 38 | 100 | 100 | 100 | 100 |
| Lemtrada - 0.4 | 100 | 88 | 100 | 75 | 75 | 100 | 100 | 100 |
| Lemtrada - 0.08 | 100 | 0 | 63 | 0 | 88 | 100 | 88 | 100 |
| Lemtrada - 0.016 | 75 | 0 | 25 | 0 | 0 | 38 | 25 | 50 |
| Mogamulizumab - 10 | 100 | 13 | 63 | 38 | 50 | 100 | 38 | 100 |
| Mogamulizumab - 2 | 100 | 0 | 0 | 13 | 0 | 13 | 0 | 50 |
| Mogamulizumab - 0.4 | 100 | 13 | 50 | 38 | 38 | 75 | 63 | 100 |
| Mogamulizumab - 0.08 | 88 | 13 | 38 | 38 | 38 | 63 | 50 | 100 |
| Mogamulizumab - 0.016 | 75 | 13 | 0 | 25 | 13 | 0 | 38 | 63 |
| Mogamulizumab - 0.0032 | 25 | 13 | 25 | 25 | 25 | 13 | 13 | 25 |
| Mogamulizumab - 0.00064 | 13 | 13 | 50 | 38 | 38 | 13 | 13 | 50 |
| Mogamulizumab - 0.000128 | 13 | 13 | 13 | 13 | 0 | 25 | 13 | 50 |
| Erbitux - 10 | 13 | 13 | 13 | 13 | 13 | 13 | 13 | 13 |
| Erbitux - 2 | 13 | 13 | 13 | 13 | 13 | 13 | 13 | 13 |
| Erbitux - 0.4 | 13 | 13 | 13 | 13 | 13 | 13 | 13 | 13 |
| Erbitux - 0.08 | 13 | 13 | 13 | 13 | 13 | 13 | 13 | 13 |
| MabThera - 10 | 75 | 13 | 63 | 25 | 13 | 75 | 13 | 50 |
| MabThera - 2 | 88 | 0 | 25 | 13 | 63 | 50 | 13 | 38 |
| MabThera - 0.4 | 100 | 13 | 38 | 38 | 25 | 75 | 25 | 75 |
| MabThera - 0.08 | 75 | 0 | 0 | 0 | 25 | 25 | 0 | 50 |
| ANC28.1 - 10 | 100 | 100 | 100 | 100 | 100 | 100 | 100 | 100 |
| ANC28.1 - 2 | 88 | 88 | 75 | 100 | 88 | 75 | 75 | 75 |
| ANC28.1 - 0.4 | 25 | 13 | 50 | 63 | 0 | 13 | 25 | 25 |
| ANC28.1 - 0.08 | 13 | 0 | 13 | 0 | 25 | 25 | 25 | 25 |
| ANC28.1 - 0.016 | 25 | 0 | 0 | 0 | 100 | 38 | 0 | 25 |
| TPP-23411 - 2 | 75 | 0 | 0 | 13 | 0 | 0 | 0 | 0 |

### CONCLUSION

Based on the results of the CRAs, precautions in the clinical trial were recommended to reduce the potential risks of infusion reactions, see also Example 23.

### Example 15: Estimation of Human Pharmacokinetic Parameters

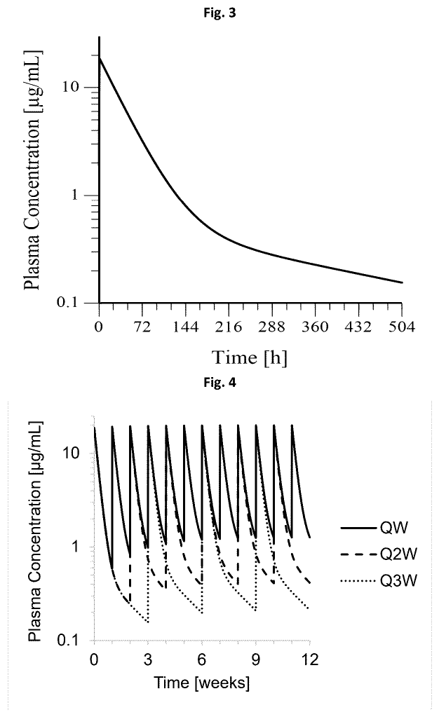

The assessment of human PK parameters was based on the data obtained from in vivo experiments after the administration of TPP-23411 to Cynomolgus monkeys. To simulate human pharmacokinetic parameters and concentration time (c/t) profiles, plasma concentrations after intravenous administration were fitted to a 2-compartment model using Phoenix 8.0. Fitted single parameters for total plasma clearance (CL), distribution clearance (CLD), volume of central compartment (Vc), and volume of tissue compartment (Vt) were allometrically scaled to humans by assuming an average body weight of 70 kg and using the fixed exponent approach with an exponent of 1 for scaling of volumes and 0.8 for scaling of clearances. The obtained human PK parameters are summarized in Table 15.1 and were used to model the c/t profile in human following a single intravenous infusion of 1 mg/kg over one hour, see FIG. 3.

For human, the obtained CL of TPP-23411 is moderately high with 1.1 mL/h/kg and the obtained volume of distribution at steady state (Vss) is low with 133 mL/kg. This results in a rather short effective half-life (t½, effective) of 84 h, whereas the obtained terminal half-life (t½,terminal) is long with 284 h, see Table 15.1.

TABLE 15.1
Estimated human key pharmacokinetic parameters of TPP-23411.

| Parameter | Unit | Predicted Value |
|---|---|---|
| CL | [mL/h/kg] | 1.1 |
| CLD | [mL/h/kg] | 0.27 |
| Vc | [mL/kg] | 53 |
| Vt | [mL/kg] | 81 |
| Vₛₛ | [mL/kg] | 133 |
| t_{1/2, effective} | [hours] | 84 |
| t_{1/2, terminal} | [hours] | 284 |

CL: total plasma clearance; CLD: distribution clearance; Vc: volume of central compartment; Vt: volume of tissue compartment; Vss: volume of distribution; t_{1/2, effective}: effective half-life; t_{1/2, terminal}: half-life in the terminal phase.

The obtained human PK parameters were used to furthermore obtain corresponding concentration time profiles after single and multiple i.v. infusion of 1 mg/kg TPP-23411 over 1 hour for QW, Q2W and Q3W administration. Results are summarized in FIG. 4. After three to four consecutive infusions, steady state is well approximated in all evaluated dosing regimens (see FIG. 4) with less accumulation observed for longer dosing intervals. For QW administration, e.g., a minor increase in terms of AUC (accumulation ratio of 120%) and a 2-fold increase in trough concentrations (accumulation ratio of 224%) is anticipated (see Table 15.2). For Q3W administration, both accumulation ratios decrease, to no significant accumulation anticipated in case of AUC (accumulation ratio of 107%) and to only a minor accumulation predicted in case of Ctrough (accumulation ratio of 131%).

Table 15.2 lists the relevant human PK parameters as estimated.

TABLE 15.2
Estimated human PK parameters after single and
multiple i.v. infusion of 1 mg/kg TPP-23411 over
1 hour for QW, Q2W and Q3W administration.

| Parameter | Unit | QW | Q2W | Q3W |
|---|---|---|---|---|
| AUC_{τ}^{§} | [μg*h/mL] | 758 | 817 | 850 |
| AUC_{τ, ss}^{§} | [μg*h/mL] | 913 | 913 | 913 |
| Cₘₐₓ | [μg/mL] | 19 | 19 | 19 |
| C_{max, ss} | [μg/mL] | 20 | 19 | 19 |
| C_{trough} | [μg/mL] | 0.58 | 0.24 | 0.16 |
| C_{trough, ss} | [μg/mL] | 1.3 | 0.41 | 0.21 |

^{§}AUCτ refers to the AUC between the first and second dose with dosing interval τ, AUCτ, ss refers to the AUC at steady-state between 2 consecutive doses with dosing interval τ, the dosing interval τ is equal to 168 hours for QW, and 504 hours for Q3W administration, respectively.

### Example 16: Estimation of Human Doses and Exposure

### Pharmacologically Active Dose and Human Efficacious Dose

Considering the human PK prediction based on translation from monkey data as described elsewhere herein, human efficacious dose ranges were estimated for different dosing schedules, as provided in Table 16.1. Predicted doses were estimated to keep C_{trough} at steady-state above predicted EC₈₀ concentrations regarding Treg depletion of TPP-23411 in vivo in humans, as provided in Table 10.1. Considering the human PK prediction based on monkey data, human dose is suggested to range from 38 μg/kg (2.7 mg/70 kg) to 1100 μg/kg (75 mg/70 kg) for a once weekly (QW) administration and for every three-week schedule (Q3W) from 230 (16 mg/70 kg) to 6400 μg/kg (450 mg/70 kg).

TABLE 16.1
Estimated efficacious dose range.

| Estimated efficacious doses | | | | | | |
|---|---|---|---|---|---|---|
| based on EC₈₀ from | | | ADCC | | ADCP | |
| QW | Dose | μg/kg | 38 | 170 | 530 | 1100 |
| schedule | | mg/70 kg | 2.7 | 12 | 37 | 75 |
| | Cₘₐₓ | μg/mL | 0.76 | 3.4 | 11 | 22 |
| | AUC_{τ, ss} | μg*h/mL | 35 | 160 | 480 | 1000 |
| Q3W | Dose | μg/kg | 230 | 1000 | 3100 | 6400 |
| schedule | | mg/70 kg | 16 | 70 | 220 | 450 |
| | Cₘₐₓ | μg/mL | 4.4 | 19 | 59 | 120 |
| | AUC_{τ, ss} | μg*h/mL | 210 | 910 | 2800 | 5800 |

For estimation of a minimal effective dose, the predicted EC₂₀ concentrations (¼×EC₅₀) regarding Treg depletion of TPP-23411 in vivo in humans were considered, see Table 10.1. In addition, a receptor occupancy (RO) based approach was taken into consideration: The RO (3.8%) at EC₂₀ of Treg depletion in the in vivo experiments in mice was estimated and re-projected to a corresponding EC₂₀ in humans using the in vitro measured binding affinities of TPP-23411 in mice and human cells. This resulted in an RO-based estimated EC₂₀ concentration of 0.019 pg/mL in vivo in human.

Coverage of the estimated EC₂₀ concentrations at C_{trough} at steady-state was considered for estimation of minimal effective doses and is presented in Table 16.2 for the QW schedule.

TABLE 16.2
Estimated minimal effective dose range. ADCC: antibody-dependent
cellular/cell-mediated cytotoxicity; ADCP: antibody-dependent
cellular phagocytosis; RO: receptor occupancy.

| Predicted minimal effective doses | | | | | | | RO | |
|---|---|---|---|---|---|---|---|---|
| based on EC₂₀ from | | | ADCC | | ADCP | | based | Median |
| QW | Dose | μg/kg | 2.4 | 11 | 33 | 67 | 14 | 14 |
| schedule | | mg/70 kg | 0.17 | 0.74 | 2.3 | 4.7 | 1.0 | 1.0 |
| | C_{max, ss} | μg/mL | 0.048 | 0.21 | 0.66 | 1.3 | 0.28 | 0.28 |
| | AUC_{τ, ss} | μg*h/mL | 2.2 | 10 | 30 | 61 | 13 | 13 |

### Example 17: First-In-Human Dose-Escalation and Expansion Study to Evaluate the Safety, Tolerability, and Pharmacokinetics of the Anti-CCR8 Antibody TPP-23411 as Monotherapy and in Combination with Pembrolizumab in Participants with Selected Advanced Solid Tumors

The study is an open-label, multicenter, non-randomized Phase 1 first-in-human (FiH) study to determine the maximum tolerated dose (MTD)/maximum administered dose (MAD), recommended dose for expansion (RDE), and the recommended Phase 2 dose (RP2D) of TPP-23411 in combination with the PD-(L)1-targeting monoclonal antibody (mAb) pembrolizumab.

The maximum tolerated dose (MTD) is defined as the maximum dose at which it is expected that ≤30% of participants experience a dose-limiting toxicity (DLT) during the DLT observation period. The DLT observation period will be 21 days after the first administration of TPP-23411 (Cycle 1). If MTD is not reached, MAD is the highest administered dose. The maximum administered dose (MAD) is the highest dose administered.

### Study Objectives

The study evaluates TPP-23411 as a new immunotherapeutic agent in advanced solid tumor settings of high medical need by assessing the safety, tolerability, pharmacokinetics, pharmacodynamics, and preliminary anti-tumor activity of TPP-23411, both as monotherapy and in combination with pembrolizumab.

### Primary Objectives of the Study are Inter Alia

a. determination of the safety and tolerability of TPP-23411 in participants with advanced solid tumors, when administered as monotherapy and in combination with pembrolizumab,
b. determination of the MTD/MAD or RDE of TPP-23411, when administered as monotherapy and in combination with pembrolizumab and
c. characterization of the single and multiple dose PK of TPP-23411, when administered as monotherapy and in combination with pembrolizumab.

### Secondary Objectives of the Study are Inter Alia

d. a preliminary estimation of antitumor activity of TPP-23411 when administered as monotherapy and in combination with pembrolizumab and
e. evaluation of target engagement and pharmacodynamic effects of TPP-23411 by predefined blood and tumor biomarkers using backfill cohorts and
f. determination of the RP2D of TPP-23411 in combination with pembrolizumab.

### Study Schema

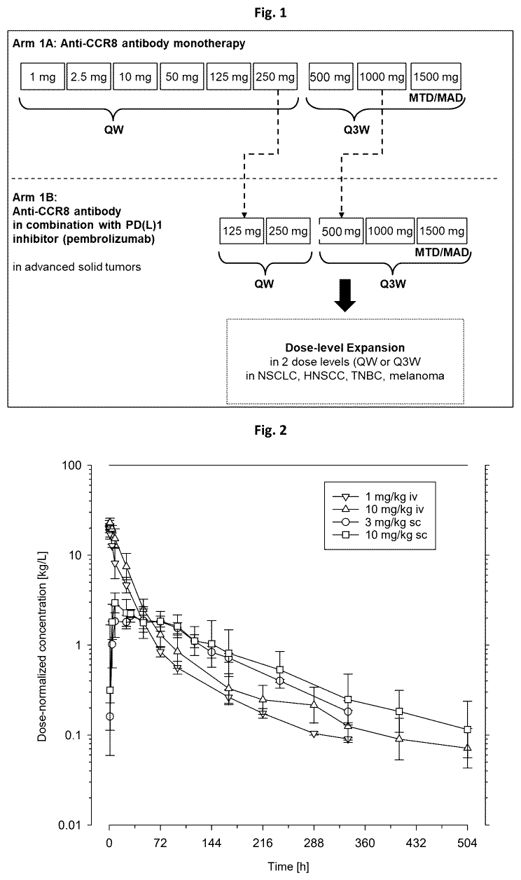

The study comprises 2 parts, a dose-escalation and an expansion part. Dose escalation is conducted for TPP-23411 both as monotherapy and in combination with pembrolizumab. To characterize the safety and preliminary antitumor activity of TPP-23411 as a single agent, a monotherapy dose escalation is conducted and a dedicated TPP-23411 monotherapy-MoA expansion is recruited. To offer participants a potentially higher therapeutic benefit, TPP-23411 is also administrated in combination with a fixed dose and schedule of pembrolizumab (e.g. 200 mg Q3W) in a combination dose escalation and in a disease-specific combination expansion. For an overview of the dose escalation part of the study schema, see FIG. 1 or Table 17.1.

The recommended starting dose was selected using the predicted minimal effective doses based on the median EC20 of the pharmacological ADCC/ADCP and receptor occupancy readout, supplemented by the results of the in vitro cytokine release assay in human whole blood and human PBMCs. The minimal effective doses based on the median EC20 considerations of TPP-23411 are approximately 0.14 mg to 0.66 mg for ADCC, 1.98 mg to 4.02 mg for ADCP, 0.84 mg for receptor occupancy (RO), and 1.2 mg for CRA. The starting dose was thus selected to balance between safety aspects and minimize exposure of patients to subtherapeutic doses.

The dose-escalation part of the study starts with TPP-23411 monotherapy escalation Arm 1A. For TPP-23411 QW dosing, combination with pembrolizumab (Arm 1B) is initiated either at dose level 100 mg or 125 mg QW after dose level 250 mg has been declared safe in Arm 1A, or at 1 dose level below the MTD after MTD has been defined in Arm 1A as <250 mg. For TPP-23411 Q3W dosing, combination with pembrolizumab (Arm 1B) is initiated at dose level 500 mg Q3W after dose level 1000 mg has been declared safe in Arm 1A.

The study enrolls participants with advanced stages of solid, preferably ICI-sensitive tumor types in the dose-escalation part and ICI relapsed/refractory tumor indications including NSCLC, HNSCC, TNBC and melanoma in the expansion part. During the FiH study, TPP-23411 is administered as an 1 hour intravenous (IV) infusion either as a QW or Q3W schedule during a 21-day treatment cycle.

The dose-escalation part contains 2 arms. In both arms, dose-escalation starts with QW dosing schedule and later switches to Q3W dosing schedule.
Arm 1A: Dose escalation of TPP-23411 as monotherapy to characterize the safety, tolerability, PK and PD profiles, and to determine the maximum tolerated dose (MTD)/maximum administered dose (MAD) and recommended dose for expansion (RDE) of TPP-23411.
Arm 1B: Dose escalation of TPP-23411 in combination with pembrolizumab to characterize the safety, tolerability, PK and PD profiles, and to determine the MTD/MAD or RDE of TPP-23411 in combination with a fixed, approved dose and schedule of pembrolizumab (e.g. 200 mg Q3W).

TABLE 17.1
Dose levels for monotherapy dose escalation scheme in Arm 1A.

| Dose Level | TPP-23411 | | Dosing schedule |
|---|---|---|---|
| 1 | 1 | mg (starting dose) | QW |
| 2 | 2.5 mg or 3 mg | | |
| 3 | 10 | mg | |
| 4 | 50 mg or 30 mg | | |
| 5 | 125 mg or 100 mg | | |
| 6 | 250 | mg | |
| 7 | 500 | mg | Q3W |
| 8 | 1000 | mg | |
| 9 | 1500 | mg | |

QW = Once a week; Q3W = Once every 3 weeks.

TABLE 17.2
Dose levels for dose escalation scheme
of combination therapy in Arm 1B.

| | TPP-23411 dose | | Pembrolizumab dose |
|---|---|---|---|
| Dose Level | and dosing schedule | | and dosing schedule |
| 1 | 125 mg QW or 100 mg QW | | 200 mg Q3W |
| 2 | 250 | mg QW | 200 mg Q3W |
| 3 | 500 | mg Q3W | 200 mg Q3W |
| 4 | 1000 | mg Q3W | 200 mg Q3W |
| 5 | 1500 | mg Q3W | 200 mg Q3W |

QW = Once a week; Q3W = Once every 3 weeks.

For Arm 1B the IV line is flushed with saline immediately after completion of infusion for TPP-23411 to prepare the IV line for pembrolizumab infusion. After 60 min, pembrolizumab can then be infused using the same IV line at a planned dose of 200 mg administered once every 3 weeks.

Preferably, on days when both anti-CCR8 antibody and PD-(L)1 inhibitor are to be administered (e.g. day 1 of each cycle, for both QW and Q3W dosing schedules), the dose of PD-(L)1 inhibitor is administered after a pause of 60 minutes following completion of anti-CCR8 antibody infusion to allow for monitoring of safety events (infusion-related reactions). PD-(L)1 inhibitor can be administered via the same IV line, following completion of the IV infusion for anti-CCR8 antibody. It is highly recommended that the IV line has been flushed with saline before infusing PD-(L)1 inhibition. If a dose lower than 250 mg QW is declared as the MTD for the QW dosing schedule, then this dose level is explored in the Q3W dosing schedule, provided this dose level falls within the predicted efficacious dose range for the Q3W dosing schedule (17 mg to 450 mg TPP-23411). However, if the QW MTD is below the predicted lowest efficacious dose for Q3W dosing schedule (i.e., <17 mg TPP-23411), then Q3W dosing schedule may not be investigated.

If 500 mg TPP-23411 Q3W as monotherapy is not tolerated, then the next lower dose level (250 mg) may still be explored in combination with pembrolizumab in the Q3W dosing schedule provided that 250 mg QW is demonstrably safe and well tolerated as monotherapy.

Conducting separate dose escalations for TPP-23411 monotherapy and for TPP-23411 in combination with pembrolizumab helps to understand and differentiate between the TPP-23411 monotherapy safety, tolerability, and pharmacodynamic effects and effects of the combination with pembrolizumab and helps in identifying separate monotherapy and combination treatment MTD/MAD and RDE (a dose level selected following review of all available pharmacokinetic, pharmacodynamic, and safety data).

After the 250 mg dose level (or alternate MTD) of the TPP-23411 monotherapy escalation (Arm 1A) in the QW dosing schedule has been declared safe in at least 3 evaluable participants, dose escalation of TPP-23411 in combination with pembrolizumab (fixed dose of 200 mg Q3W) begins at a TPP-23411 dose level that is 1 level lower (i.e., at 100 mg or 125 mg TPP-23411 QW). If the MTD of TPP-23411 monotherapy (as defined in Arm 1A) is lower than 250 mg QW, TPP-23411 dose escalation in Arm 1B begins at 1 dose level below the MTD.

Similarly, once the 1000 mg dose of TPP-23411 monotherapy (Arm 1A) in the Q3W dosing schedule has been declared safe in at least 3 evaluable participants, dose escalation of TPP-23411 Q3W in combination with pembrolizumab (200 mg Q3W) begins at a TPP-23411 dose level that is one level lower (i.e., at 500 mg TPP-23411 Q3W).

The expansion part contains 2 arms.
Arm 2A: TPP-23411 monotherapy-mode-of-action (monotherapy-MoA) expansion in NSCLC participants (PD-L1 tumor proportion score [TPS]≥50%) with primary (ICI-refractory) or secondary (ICI-relapsed) resistance to prior ICI-therapy to provide a preliminary estimation of TPP-23411 antitumor activity and to evaluate target engagement and pharmacodynamic effects of TPP-23411 by predefined blood and tumor biomarkers.
Arm 2B: Disease-specific combination expansion with separate cohorts in 4 ICI-relapsed tumor types (NSCLC, TNBC, HNSCC, and melanoma) to obtain a preliminary efficacy evaluation, to provide further safety and pharmacokinetic/pharmacodynamic (PK/PD) data, and to confirm the RP2D for TPP-23411 in combination with a fixed, approved dose and schedule of pembrolizumab (e.g. 200 mg Q3W).

Based on the results obtained during the dose-escalation part, 1 of these 2 schedules (QW or Q3W) with a fixed dose of TPP-23411 is chosen for the TPP-23411 monotherapy-MoA expansion and for the disease-specific combination expansion (in combination with pembrolizumab 200 mg Q3W).

### Treatment Period

The start of the treatment period or first treatment cycle is defined by the first administration of study treatment (i.e., intravenous [IV] infusion of TPP-23411 as monotherapy or in combination with a fixed, approved IV dose of pembrolizumab [e.g. 200 mg Q3W]).

The cycle length is 21 days, with administration of study treatment either on Days 1, 8, and 15 (dose levels with QW schedule) or on Day 1 (Q3W schedule) during the dose-escalation part.

Participants receive study treatment until the occurrence of disease progression or unacceptable toxicity, or until another specified withdrawal criterion is met.

The active follow-up (FU) period starts after completion of the end of treatment (EOT) visit and includes a safety FU visit/contact and (if applicable) efficacy FU visits. A safety FU visit/contact takes place 90 days (±7 days) after the last administration of study treatment; efficacy FU visits takes place every 12 weeks (±14 days) from the last dose for participants who permanently discontinue study treatment for other reasons than disease progression and did not start a new anticancer treatment (ACT). After completion of the active FU period, participants enter the long-term FU period during which all participants are contacted to determine the survival status and subsequent systemic ACT every 6 months (±14 days) for up to 24 months after the last participant's EOT, or until the end of the study (whichever occurs first).

### Monitoring & Biomarker

The TPP-23411 monotherapy-MoA expansion includes evaluation of paired biopsies for TME analysis. Furthermore, to characterize the CD8 immune cell status of tumor lesions at baseline and changes induced by treatment with TPP-23411 89-ZR-anti-CD8 minibody PET/CT with 89Zr-Df-crefmirlimab is used as a biomarker in TPP-23411 monotherapy-MoA expansion for quantitative imaging of CD8 T cells (Arm 2A). The Zr89 anti-CD8 PET/CT scan will expose participants to modest additional radiation exposure. Baseline quantification and distribution of tracer uptake in tumor lesions to classify lesions as hot, immune-excluded, or cold by central review is performed. Change in tracer uptake/distribution between baseline and end of Cycle 2 by central review and comparison of these changes with BOR and DOR based on RECIST 1.1 criteria for individual participants is performed.

To closely monitor study participants for potential CRS occurrence, blood collection for assessment of inflammatory cytokines (e.g., IFN-γ, IL-1β, IL-2, IL-4, IL-6, IL-8, IL-10, IL 12p70, IL-13, and TNF-α) will be performed at least pre-dose, at 4 h and 24 h after start of every TPP-23411 infusion in Cycle 1 in the first 3 dose levels.

T cells, B cells, NK cells, and T cell subpopulations will be quantified and activation status of T cell populations will be determined by flow cytometry in peripheral blood.

Fold change in peripheral IFN-γ is measured by an immune based assay in on-treatment compared to baseline serum samples as reference. Fold change in intratumor CD8+ T cell/Treg ratio is measured by IHC in on-treatment compared to baseline biopsies.

Biomarker samples will be collected from participants, and include:
Archival tumor tissue and/or fresh pretreatment biopsies
Paired on-study tumor tissue biopsies
Blood samples before and during study treatment for:
   Cytokines and chemokines
   Flow-cytometric analyses
   RNA and ctDNA preparation

Pharmacodynamic biomarkers will be evaluated in samples collected before and during study treatment to investigate the impact of TPP-23411 on these biomarkers.

Baseline candidate biomarkers are evaluated for correlation with the response to study treatment (to further validate predictive biomarkers). Samples from tumor biopsies and blood will retrospectively be analyzed. Candidate predictive biomarkers include (but are not limited to):
Tumor immune infiltration (e.g., immune gene expression profiles; presence, phenotype, and activation status of tumor-infiltrating leukocytes with special focus on CCR8 positive Treg cells, CD8 T cells, NK cells, and macrophages)
PD-L1 expression level as a surrogate of immune sensitivity
Tumor mutational status (microsatellite instability [MSI], tumor mutations, tumor mutational burden [TMB])
Imaging-based biomarkers for tumor-infiltrating CD8 T cells

### PD-L1 Expression for Patient Selection

Since CD8 T cells are essential for 10-related antitumor effect, the functional relevance of Treg depletion is assumed to be highest in tumors with already high T-cell infiltrate. In addition, macrophages and NK cells are essential for anti-CCR8-mediated Treg depletion via ADCC or ADCP. Based on mRNA expression, PD-L1 positivity correlates with the presence of immune cells including Tregs, macrophages, and CD8 Teffs in the TME across all indications from The Cancer Genome Atlas (TCGA) project. The relevance of PD-L1 expression is further supported by the observation that PD-L1 mRNA expression is a significant positive predictive biomarker for anti-CCR8 efficacy across mouse tumor models. Patients with PD-L1-positive tumors from immunosensitive indications are therefore expected to best respond to TPP-23411 as monotherapy.

As a result, in the monotherapy-MoA expansion arm (Arm 2A), which enrolls NSCLC patients, PD-L1 expression levels will be used as an inclusion criterion. Patients therefore must have an historic PD-L1 score of TPS ≥50% (as previously determined by a locally approved assay).

Alternatively, Arm 2A comprises anti-CCR8 antibody monotherapy-MoA expansion
in NSCLC participants (with PD-L1 TPS ≥50%) OR
TNBC (with PD-L1 CPS ≥10%) OR
HNSCC (with PD-L1 CPS ≥1%) OR
HNSCC (with PD-L1 CPS ≥20%) OR
melanoma (no PD-L1 cut-off), respectively.

### PET/CT Imaging of CD8 Cells

Zirconium-89 (89Zr) anti-CD8 minibody PET/CT are performed in the monotherapy-MoA expansion (Arm 2A) and utilize 89Zr-Df-crefmirlimab, a minibody labeled with 89Zr which targets CD8 cells. The CD8 tracer uptake has been shown to correlate with CD8 expression by IHC and to correlate positively with tumor response assessments by RECIST 1.1.

In this study, the exploratory outputs are the following:
At baseline/screening, tumor uptake, and biodistribution of the 89Zr CD8 tracer using whole-body PET/CT allows categorization of tumor lesions throughout the body as hot, immune-excluded, or cold with respect to CD8 cell infiltration.
89Zr anti-CD8 minibody PET/CT early during treatment (end of Cycle 2) are used to assess the change in CD8 cell quantification and distribution pattern in tumor lesions/TME induced by TPP-23411. CD8 patterns are correlated for each participant in the monotherapy-MoA expansion (Arm 2A) with best overall response (BOR) and time to progression data from each participant's tumor response imaging assessed using RECIST 1.1.

At each time point, the infusion of the 89Zr-Df-crefmirlimab is followed by a whole-body PET/CT scan (skull vertex to mid-thigh) approximately 24 h±3 h later.

### Radiomics Analysis

Radiomics is an area of imaging biomarker research defined as the use of automated or semiautomated analytical methods to extract quantitative metrics (often referred to as radiomic features) from medical images (Mayerhoefer et al. 2020). These metrics capture tissue and lesion characteristics such as shape and heterogeneity and may, alone or in combination with histologic, genetic, or proteomic data, be used to help better understand the clinical activity of experimental medicines. In this study, radiomic analysis is an exploratory objective and may be performed on all image data, including images collected for tumor response assessments. CT images from tumor response assessments will be centrally collected in DICOM format with a reconstructed slice thickness of 1.0 mm. Depletion of CCR8-expressing Tregs by TPP-23411 might lead to an increase in tumor-infiltrating CD8 lymphocytes. A change in CD8 radiomics metrics from baseline may be used as an exploratory endpoint to assess the pharmacodynamic effect of TPP-23411. The CD8 radiomic metric will also be correlated to paired tumor biopsy results (e.g., CD8 IHC) when possible. Similarly, radiomic metrics derived from study images (e.g., tumor assessment) will be compared with other study biomarkers and endpoints.

### Example 18: Biomarker: Association Between Gene Expression and Response to Murine Surrogate Antibodies TPP-14099 and TPP-15285 in Syngeneic Murine Carcinoma Models

The association between gene expression and the efficacy of treatments with CCR8-depleting antibodies was studied by whole transcriptome sequencing of early untreated tumors of 21 syngeneic murine carcinoma models. The associations between anti-CCR8 treatment and treatments inhibiting the immune checkpoints PD-1 and PD-L1 were also assessed.

The studied anti-mouse CCR8 antibodies TPP-14099 (hIgG1) and TPP-15285 (mIgG2a) were efficacious in immune checkpoint sensitive models. The anti-mouse CCR8 antibodies showed almost always superior efficacy compared to anti-PD-1 antibody or anti-PD-L1 antibody treatments. The efficacy of the anti-mouse CCR8 antibodies did not depend on mouse strain or correlate with mutational burden of the respective carcinoma cell line.

FIG. 5 shows that responses to anti-mouse CCR8 antibody treatments correlated with response to anti-PD-L1 antibody and anti-PD-1 antibody treatments.

FIG. 6 shows that the baseline mRNA expression of PD-L1 and inflammation markers such as IFN-γ correlated with responses to anti-mouse CCR8 antibody treatment in early untreated tumors.

### Example 19: Determination of Unchanged Compound in Plasma

In the preclinical pharmacokinetic study in monkey and the pivotal nonclinical safety studies TPP-23411 was determined from plasma using an anti-human IgG generic assay format (IgG-ELISA).

In this protocol, TPP-23411 was measured in plasma after dilution with buffer by a Gyrolab method with fluorescence readout, using an immobilized biotinylated anti-human IgG-Fc antibody as capture molecule and an Alexa fluorophore-labelled anti-human IgG antibody as detection reagent. Method specific parameters are listed in Table 19.1.

TABLE 19.1
Method specification for monkey plasma. Accuracy and
precision were calculated from validation quality
control samples (including LLOQ and ULOQ results).

| | LLOQ | Precision | | Accuracy | | |
|---|---|---|---|---|---|---|
| Species | [μg/L] | [%] | | [%] | | |
| Cynomolgus monkey | 12.5 | ≤ | 16.4 | −0.4 | to | 3.5 |
| (IgG ELISA) | | | | | | |

TPP-23411 proved to be stable as indicated under the conditions listed above, that are relevant for sample handling, see Table 19.2.

TABLE 19.2
Stability data.

| Stability type | Species/Matrix | Temperature | Period | | Result |
|---|---|---|---|---|---|
| Short term^{a} | Cynomolgus monkey/ | RT | 24 | h | acceptable |
| | Plasma | | | | |
| Freeze/thaw^{a} | Cynomolgus monkey/ | −20 to RT | 10 | cycles | acceptable |
| | Plasma | | | | |
| Freezer^{a} | Cynomolgus monkey/ | −75 +/− 15 | 35 | days | acceptable |
| | Plasma | | | | |
| Benchtop^{a} | Calibration solution | RT | 6 | h | acceptable |

### Example 20: Determination of Anti-Anti-CCR8 Antibody Antibodies in Plasma

Binding antibodies against TPP-23411 (anti-drug antibodies) were determined in monkey plasma with a Meso Scale Discovery (MSD)-based bridging assay using biotinylated TPP-23411 as antigen. Anti-TPP-23411 antibodies present in positive controls or study-specific plasma samples bound to the biotinylated TPP-23411 and were captured on the streptavidin plate, while other plasma components were washed away. Bound antibodies were then detected using SULFO-tagged TPP-23411 and ECL readout (see U.S. Pat. Nos. 7,855,287 and 7,803,573 for ECL reagent).

In more detail, positive and negative control samples (monkey serum with and without positive control, respectively) as well as unknown samples were prediluted (1:8) with dilution buffer, mixed with dilution buffer and preincubated in a polypropylene plate for 1 hour on an orbital shaker (RT, 600 rpm). To the sample mixture, master mix containing biotinylated TPP-23411 (1 μg/mL) and SULFO-tagged TPP-23411 (1 μg/mL) were added and incubated for 2 hours (RT, 600 rpm). From the incubated samples 25 μL were then transferred in duplicates into wells of a blocked MSD Streptavidin Gold plate (150 μL Block buffer for a minimum of 30 minutes, 600 rpm) to which biotinylated TPP-23411 can bind. Functional anti-drug antibodies bridge the biotinylated and SULFO-tagged TPP-23411. Sulfonated TPP-23411 produces an electrochemiluminescence (ECL) signal correlating to the amount of ADA in the well when voltage is applied. Plates were read using the Meso QuickPlex SQ 120 and data was analyzed with the MSD© Workbench™ software. All samples were determined in duplicates.

The positive control antibody was an affinity pure goat anti-human IgG antibody. The sensitivity of this positive control in the assay was 4.79 μg/L (assay cut-off concentration).

Method validation and the analysis of study samples was conducted in accordance with internal SOPs and the pertinent guidelines on “Assay Development for Immunogenicity Testing of Therapeutic Protein Products” (FDA, 2019) and “Guideline on Immunogenicity Assessment of Therapeutic Proteins” (EMA, 2017). Bioanalytical methods applied to pivotal nonclinical safety studies were fully validated and samples from these studies were analyzed in compliance with Good Laboratory Practice (GLP).

TABLE 20.1
Method specification for anti-TPP-23411 antibody methods in monkey plasma. Precision
was calculated from low, medium, and high-quality control samples.

| | Sensitivity | Precision | Cut point | | Drug tolerance |
|---|---|---|---|---|---|
| Species | [μg/L] | [%] | factor | MRD | μg/mL |
| Cynomolgus | 4.79 | <14.8 | NC 1.179 | 32 | 327 (at 1 μg/mL PC); |
| monkey | | | (floating) | | 134 (at 0.2 μg/mLPC) |

Stability of the positive control antibody mimicking the study sample in plasma under different storage conditions covering the different steps of sample processing during assay performance and the storage intervals pertinent to actual study samples was investigated. TPP-23411 (positive control antibody) was stable under all conditions relevant for sample handling.

TABLE 20.2
Selected plasma stability data of polyclonal goat
anti-human IgG (positive control antibody)

| Stability type | Species | Temperature | Period | | Stable |
|---|---|---|---|---|---|
| Short term | Cynomolgus monkey | RT | 24 | hours | Yes |
| Freeze-thaw | Cynomolgus monkey | −75° C./−20° C. to RT | 5 | cycles | Yes |

TABLE 20.3
Selected plasma stability data of polyclonal goat
anti-human IgG (positive control antibody)

| Stability type | Species | Temperature | Period | | Stable |
|---|---|---|---|---|---|
| Short term | Cynomolgus monkey | RT | 24 | hours | Yes |
| Freeze-thaw | Cynomolgus monkey | −75° C./−20° C. to RT | 5 | cycles | Yes |

### Example 21: Tissue Cross-Reactivity

A preliminary tissue cross-reactivity (TCR) study was conducted to validate the immunohistochemical (ICH) method for detecting TPP-23411-FITC bound to CCR8. This was a non-GLP study but conducted according to current scientific and regulatory standards.

The objective of this preliminary TCR study was to evaluate the potential cross-reactivity of the fluorescein isothiocyanate (FITC)-conjugated form of TPP-23411, using a panel of frozen tissues and blood smears from three human and three Cynomolgus monkey donors (per tissue), using immunohistochemistry (IHC) techniques. The IHC method for detecting TPP-23411-FITC bound to CCR8 was successfully validated in terms of specificity, sensitivity, range, linearity, precision (repeatability), and reproducibility.

TPP-23411-FITC yielded positive, membranous/cytoplasmic staining of monkey CCR8-positive cells from 0.1 to 9 μg/mL and did not elicit any staining in the negative cells. No staining was observed in any positive and negative cells incubated with IgG1-FITC at any concentrations.

The full FDA tissue list was investigated in human and Cynomolgus monkey species and the following results were achieved at 3 and 9 μg/mL:

### Human Tissues

### TPP-23411-FITC Yielded:

positive membranous, variably cytoplasmic staining of mononuclear cells within some germinal centers of the GALT in ileum at 3 and 9 μg/mL (suggestive of dendritic cells and/or macrophages; other cell types cannot be excluded).
membranous, variably cytoplasmic staining in subepithelial cells in the jejunum villi. These cells were considered to represent myoepithelial cells or pericytes (other cell types cannot be excluded).
staining in the peritubular interstitial tissues (i.e., fibroblasts and/or lamina propria of the tubules/blood vessels) of the kidney.
minimal to moderate staining in histological glandular compartments with secretory function, such as the mammary gland (breast), prostate, parotid gland, gall bladder, and stomach. TPP-23411-FITC also produced minimal staining of umbrella cells in the ureter.

### Cynomolgus Monkey

### TPP-23411-FITC Elicited Staining:

in interstitial spindle cells of the pancreas (suggestive of endothelial cells)
in scattered large cells in the parathyroid
in umbrella cells (luminal aspect) of the urothelium (urinary bladder)
in the adrenal medulla (plasmatic proteins).

### Example 22: Tissue Cross-Reactivity (GLP Study)

The optimized IHC assay was validated in the preliminary non-GLP study using automated techniques and a DAB Map kit from Ventana as the detection system. In the GLP-compliant study two concentrations of the test item TPP-23411-FITC were investigated, 3 and 10 μg/mL. The tissues investigated are shown in Table 22.1.

TABLE 22.1
Tissue cross-reactivity - GLP study.
Tissue type

Adrenal
Bladder (urinary)
Blood Cells ^{a}
Blood Vessels (endothelium) ^{b}
Bone Marrow
Brain - cerebellum
Brain - cerebral cortex
Breast (mammary gland)
Eye
Fallopian Tube (oviduct)
Gastrointestinal (GI) Tract ^{c}
Heart
Kidney (glomerulus, tubule)
Liver
Lung
Lymph Node
Ovary
Pancreas
Parathyroid
Peripheral Nerve
Pituitary
Placenta
Prostate
Parotid Gland
Skin
Spinal Cord
Spleen
Striated Muscle (skeletal)
Testis
Thymus
Thyroid
Tonsil
Ureter
Uterus - Cervix
Uterus - endometrium
^{a} evaluated from peripheral blood smears.
^{b} evaluated from all tissues where present.
^{c} included esophagus, large intestine/colon, small intestine (duodenum or jejunum), and stomach (including underlying smooth muscle).

TPP-23411-FITC elicited membranous, variably cytoplasmic staining of human and Cynomolgus monkey CCR8-positive cells while no staining was produced in the negative cells at 3 and 10 μg/mL. Negative control item antibody TPP-9809-FITC did not produce any staining in human and Cynomolgus monkey CCR8-positive cells or CCR8-negative cells at 10 μg/mL.

Tissue integrity was shown to be adequate. All tissues were considered acceptable for microscopic evaluation in terms of morphology and inclusion of tissue elements.

In human tissues, TPP-23411-FITC investigated at 3 and/or 10 μg/mL produced:
Membranous staining in platelets (rare to frequent, 3/3 donors)
Staining of interstitial cells in the liver, membranous/cytoplasmic (2/3 donors), suggestive of Kupffer cells
Staining of mixed inflammatory cells (macrophages and granulocytes/neutrophils) in the lumen of the oviduct (2/3 donors)
Staining of occasional glial cells in the spinal cord (1/3 donors)
Membranous, variably cytoplasmic staining of mononuclear cells, suggestive of macrophages, in the serosa of the ureter and adjacent tissues (1/3 donors)
Staining of stromal spindle cells in the small intestine (villi, 2/3 donors), suggestive of smooth cells and/or fibroblasts
Diffuse staining in the fibrous and/or fibrovascular tissue (mainly extracellular matrix) of several organs (adrenal (1/3 donors), heart (1/3 donors), kidney (3/3 donors), ovary (3/3 donors), stomach (1/3 donors), and tonsil (1/3 donors))

In Cynomolgus monkeys, TPP-23411-FITC investigated at 3 and/or 10 μg/mL produced:
Diffuse staining in the fibrous and/or fibrovascular tissue (mainly extracellular matrix) of several organs (breast (2/3 donors), corneal stroma (2/3 donors), kidney (3/3 donors), ovary (3/3 donors), parotid glands (3/3 donors), more prominently around some ducts, including possibly some myoepithelial cells), pituitary gland (3/3 donors), prostate (3/3 donors), stomach (3/3 donors), testis (3/3 donors), thyroid (3/3 donors), urinary bladder (3/3 donors), uterus cervix and endometrium (3/3 donors))
Staining in occasional scattered cells in parathyroid glands (2/3 donors)
Staining of cells within the red pulp in the spleen (3/3 donors, most cells, a mixture of mononuclear cells, granulocytes, and mesenchymal cells) Based on immunohistochemistry, TPP-23411-FITC tested at 3 and/or 10 μg/mL, produced staining in the fibro-vascular tissues (mainly extracellular matrix) in both human and monkey tissues. In addition, some staining was observed in inflammatory cells (granulocytes and/or mononuclear cells suggestive of macrophages) in both species as well (more prominent in humans, limited to the spleen in monkeys). Staining that was seen in humans and not in monkeys involved the platelets and the spinal cord (nervous tissue).

A comprehensive histopathological investigation in the monkey toxicology studies did not reveal any morphological alterations of tissues with positive staining in the TCR assay. Cytoplasmic staining (e.g., in the spleen, spinal cord) was judged to be of little to no safety concern as the monoclonal antibody access to the cytoplasmic compartment is considered to be limited in vivo.

### Example 23: Administration of Antihistamines, Acetaminophen, Corticosteroids for Side Effect Management

If infusion related reactions occurred upon administration of the anti-CCR8 antibody, participants received additional medication, optionally with prolonged study treatment infusion time for the subsequent administrations. Additional medication included, e. g. antihistamines, acetaminophen or corticosteroids.

After an infusion reaction occurred upon administration of the anti-CCR8 antibody, between 650 mg and 1000 mg paracetamol was administered before the subsequent infusion. With this premedication, no infusion reaction was observed for the subsequent administration of anti-CCR8 antibody.

Based on these data it is understood that a dosis of 500 mg to 1000 mg paracetamol could be administered, either before the first dose of the anti-CCR8 antibody or before a subsequent dose of the anti-CCR8 antibody to prevent or mitigate side reactions.

After an infusion reaction occurred upon first administration of the anti-CCR8 antibody, 650 mg paracetamol and 100 mg diphenhydramine were administered before the subsequent infusion. With this premedication, no infusion reaction was observed for the subsequent administration of anti-CCR8 antibody.

In the alternative, dexamethasone can be administered before administration of a dose of the anti-CCR8 antibody. Suitable doses were e.g. 2 doses of 8 mg dexamethasone, e.g. 3 h pre and 4 h post administration of anti-CCR8 antibody.

Based on the severity of the adverse reaction, pembrolizumab may be withheld and corticosteroids may be administered. Upon improvement to Grade 1, corticosteroid taper can be initiated and continued over at least 1 month.

### Example 24: Demonstration of Treg Depletion & Mode of Action for Suggested Dosing Regimen

Expression of CD45, CD45RA, CD3, CD4, CD8, CD25 (IL2RA), CD127 (IL7RA), CD69, CCR7, GZMB, Ki67, PDL1, OX40 and CD137 (4-1BB=activation marker) was analyzed by flow cytometry to monitor inter alia the percentage of activated proliferating CD8+ T cells and the percentage of Treg cells. Based on the postulated mode of action an increase of the percentage of activated proliferating CD8+ T cells and a decrease of the percentage of residual Treg cells are a prerequisite for the treatment success and could be demonstrated for the suggested dosing scheme:

Activated proliferating CD8+ T cells were defined by expression of proliferation marker Ki67, activation marker 4-1BB (CD137), CD8 and CD3.

Activated Tregs were defined by expression of CD137 (4-1BB), CD25, CD127low, CD4, and CD3. 4-1BB was used instead of CCR8 to allow for evaluation of Treg levels and Treg depletion, because the binding of the treatment antibody might compete with binding of a flow cytometry marker antibody directed to CCR8.

FIG. 7 shows an increase of activated proliferating CD8+ T cells relative to the total number of CD3+ T cells starting ˜3 days after the first administration of 10 mg of anti-CCR8 antibody. A continuous increase was observed in particular starting from the second week of treatment, presumably by induction of immune activation in the tumor. End of treatment is ˜3 weeks after the last infusion.

The ratio of the two cell types at the timepoint of screening was set to 1.

The abbreviations listed on the x-axis of FIG. 7, FIG. 8 and FIG. 9 are:
Scr: Screening value (during screening of patient, i.e. before the first anti-CCR8 antibody administration);
C1D1: Cycle 1, day 1, 0 h (immediately before start of anti-CCR8 antibody administration);
C1D2: Cycle 1, day 2, 0 h (˜1 day after first anti-CCR8 antibody administration);
C1D3: Cycle 1, day 3, 0 h (˜2 days after first anti-CCR8 antibody administration);
C1D8: Cycle 1, day 8, 0 h (˜7 days after first anti-CCR8 antibody administration, immediately before second anti-CCR8 antibody administration);
C1D15: Cycle 1, day 15, 0 h (˜7 days after second anti-CCR8 antibody administration, immediately before third anti-CCR8 antibody administration);
C2D1: Cycle 2, day 1, 0 h (˜7 days after third anti-CCR8 antibody administration, immediately before fourth anti-CCR8 antibody administration);
C2D3: Cycle 2, day 3, 0 h (˜1 day after fourth anti-CCR8 antibody administration);
C2D8: Cycle 2, day 8, 0 h (˜7 day after fourth anti-CCR8 antibody administration, immediately before fifth anti-CCR8 antibody administration);
C2D15: Cycle 2, day 15, 0 h (˜7 day after fifth anti-CCR8 antibody administration, immediately before sixth anti-CCR8 antibody administration);
EOT: End of treatment (see protocol, e.g. 30 days after last infusion).

FIG. 8 shows a substantial decrease of the ratio of activated Tregs relative to the total number of CD3+ T cells starting ˜1 day after the first anti-CCR8 antibody administration (dosis of 10 mg). The ratio of the two cell types at the timepoint of screening was set to 1.

FIG. 9 shows the ratio of activated Tregs relative to the number of activated proliferating CD8+ T cells. The ratio of the two cell types at the timepoint of screening was set to 1.

Other tested doses showed a similar trend.

FIG. 10 shows the reduction of Tregs in patient blood samples upon treatment with 1 mg, 3 mg, 10 mg or 30 mg of anti-CCR8 antibody. For each patient the ratios (activated Tregs/total CD3+ T cells) obtained for the timepoints C1D2 to C2D15 were divided by the ratios (activated Tregs/total CD3+ T cells) obtained upon screening (Scr) for the same patient, i.e. before administration of anti-CCR8 antibody. To obtain the data for one box, all patients treated at the indicated dose were grouped together. Even the lowest 1 mg cohort shows a median reduction of the activated Tregs to ˜53% of the screening value.

### Example 25: Biomarkers for Detecting Immune Cell Activation in Blood

Human blood/serum samples were collected from patients as known in the art and as described elsewhere herein. TNF-alpha and cytokine levels were subsequently analyzed using the commercially available V-plex assay (V-PLEX Proinflammatory Panel 1 Human Kit, MSD; see https://www.messoscale.com/en/productss/v-plex-proinflammatory-panel-1-human-kit-k15049d/).

FIG. 11 shows the boxplots for TNF-alpha levels measured in pg/μl in blood or serum samples that were collected 4 hours after administration of 1 mg, 3 mg, 10 mg or 30 mg anti-CCR8 antibody to human patients. The levels of the biomarker increased with increased anti-CCR8 antibody dosing.

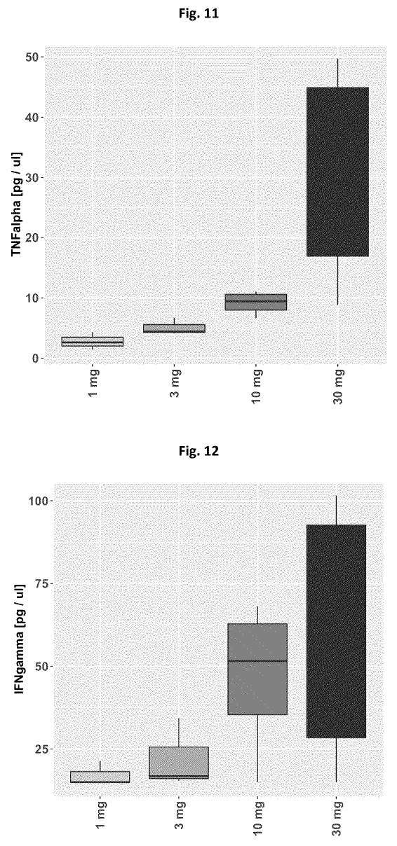

FIG. 12 shows the boxplots for IFN-gamma levels measured in pg/μl in blood or serum samples that were collected 4 hours after administration of 1 mg, 3 mg, 10 mg or 30 mg anti-CCR8 antibody from human patients. The levels of the biomarker increased with increased anti-CCR8 antibody dosing.

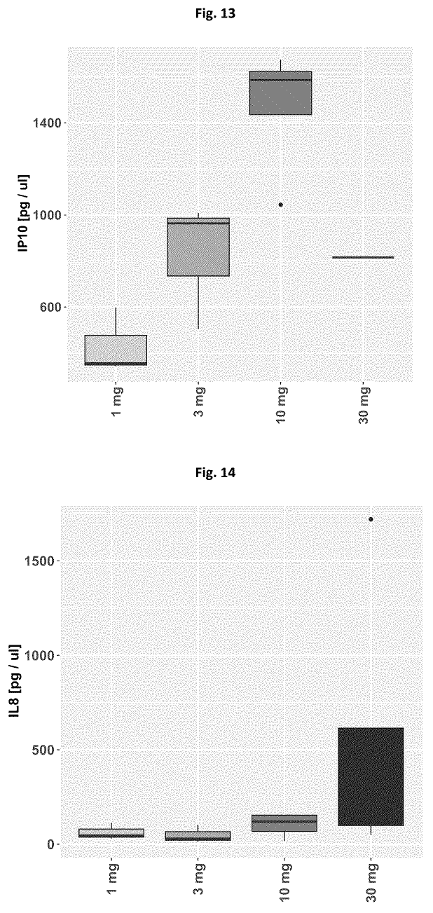

FIG. 13 shows the boxplots for IP10 levels measured in pg/μl in blood or serum samples that were collected 4 hours after administration of 1 mg, 3 mg, 10 mg or 30 mg anti-CCR8 antibody to human patients.

FIG. 14 shows the boxplots for IL8 levels measured in pg/μl in blood or serum samples that were collected 4 hours after administration of 1 mg, 3 mg, 10 mg or 30 mg anti-CCR8 antibody to human patients.

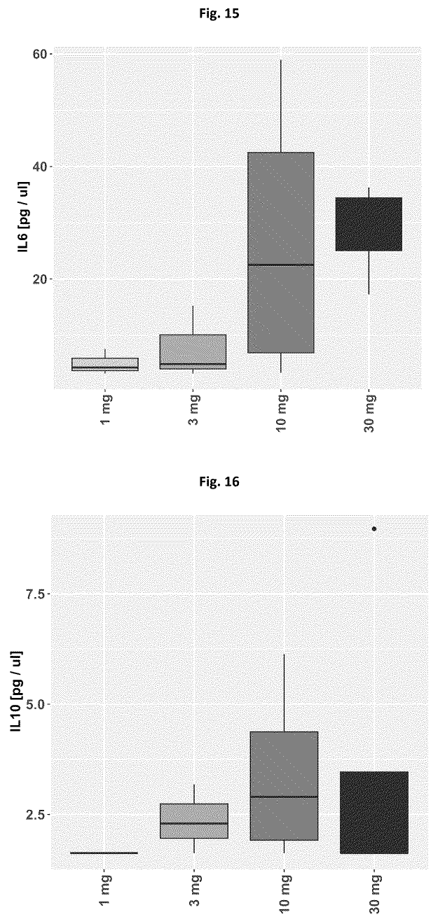

FIG. 15 shows the boxplots for IL6 levels measured in pg/μl in blood or serum samples that were collected 4 hours after administration of 1 mg, 3 mg, 10 mg or 30 mg anti-CCR8 antibody to human patients.

FIG. 16 shows the boxplots for IL10 levels measured in pg/μl in blood or serum samples that were collected 4 hours after administration of 1 mg, 3 mg, 10 mg or 30 mg anti-CCR8 antibody to human patients.

SEQUENCES

| SEQ ID | |
|---|---|
| NO | Sequence |
| 1 | EVQLLESGGGLVQPGGSLRLSCAASGFTFSSYGMHWVRQAPGKGLEWVSAINWNGGSTGYADSVKG |
| | RFTISRDNSKNTLYLQMNSLRAEDTAVYYCARGHHSGYDGRFFDYWGQGTLVTVSS |
| 2 | SYGMH |
| 3 | AINWNGGSTGYADSVKG |
| 4 | GHHSGYDGRFFDY |
| 5 | QSVLTQPPSVSGAPGQRVTISCTGSSSNIGAGYNVHWYQQLPGTAPKLLIYTNNRRPSGVPDRFSG |
| | SKSGTSASLAISGLRSEDEADYYCAAWDASLSGWVFGGGTKLTVL |
| 6 | TGSSSNIGAGYNVH |
| 7 | TNNRRPS |
| 8 | AAWDASLSGWV |
| 9 | GAGGTGCAGCTGCTGGAATCTGGCGGAGGATTGGTTCAGCCTGGCGGCTCTCTGAGACTGTCTTGT |
| | GCCGCTTCCGGCTTCACCTTCTCCAGCTACGGAATGCACTGGGTCCGACAGGCCCCTGGCAAAGGA |
| | TTGGAATGGGTGTCCGCCATCAACTGGAACGGCGGCTCTACCGGCTACGCCGATTCTGTGAAGGGC |
| | AGATTCACCATCAGCCGGGACAACTCCAAGAACACCCTGTACCTGCAGATGAACTCCCTGAGAGCC |
| | GAGGACACCGCCGTGTACTATTGTGCTAGAGGCCACCACTCTGGCTACGACGGCAGATTCTTCGAC |
| | TATTGGGGCCAGGGCACCCTGGTCACAGTTTCTTCA |
| 10 | AGCTACGGAATGCAC |
| 11 | GCCATCAACTGGAACGGCGGCTCTACCGGCTACGCCGATTCTGTGAAGGGC |
| 12 | GGCCACCACTCTGGCTACGACGGCAGATTCTTCGACTAT |
| 13 | CAGTCTGTTCTGACACAGCCTCCATCTGTGTCTGGCGCCCCTGGACAGAGAGTGACCATCAGCTGT |
| | ACAGGCAGCAGCTCCAATATCGGAGCCGGCTACAACGTGCACTGGTATCAGCAGCTGCCTGGCACA |
| | GCCCCTAAACTGCTGATCTACACCAACAACAGACGGCCCAGCGGCGTGCCCGATAGATTTTCTGGC |
| | AGCAAGAGCGGCACAAGCGCCAGCCTGGCTATCTCTGGACTGAGATCTGAGGACGAGGCCGACTAC |
| | TATTGCGCCGCCTGGGATGCTTCTCTGAGCGGATGGGTTTTCGGCGGAGGCACCAAACTGACAGTG |
| | CTA |
| 14 | ACAGGCAGCAGCTCCAATATCGGAGCCGGCTACAACGTGCAC |
| 15 | ACCAACAACAGACGGCCCAGC |
| 16 | GCCGCCTGGGATGCTTCTCTGAGCGGATGGGTT |
| 17 | EVQLLESGGGLVQPGGSLRLSCAASGFTFSSYGMHWVRQAPGKGLEWVSAINWNGGSTGYADSVKG |
| | RFTISRDNSKNTLYLQMNSLRAEDTAVYYCARGHHSGYDGRFFDYWGQGTLVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| 18 | QSVLTQPPSVSGAPGQRVTISCTGSSSNIGAGYNVHWYQQLPGTAPKLLIYTNNRRPSGVPDRFSG |
| | SKSGTSASLAISGLRSEDEADYYCAAWDASLSGWVFGGGTKLTVLGQPKAAPSVTLFPPSSEELQA |
| | NKATLVCLISDFYPGAVTVAWKADSSPVKAGVETTTPSKQSNNKYAASSYLSLTPEQWKSHRSYSC |
| | QVTHEGSTVEKTVAPTECS |
| 19 | EVQLLESGGGLVQPGGSLRLSCAASGFTFSDYGVHWVRQAPGKGLEWVSGVSWNGSRTHYADSVKG |
| | RFTISRDNSKNTLYLQMNSLRAEDTAVYYCVTRGAWGQGTLVTVSS |
| 20 | DYGVH |
| 21 | GVSWNGSRTHYADSVKG |
| 22 | RGA |
| 23 | QSVLTQPPSASGTPGQRVTISCSGSSFNIGSHFVYWYQQLPGTAPKLLIYKNNQRPSGVPDRFSGS |
| | KSGTSASLAISGLRSEDEADYYCAAWDDSLNGPVFGGGTKLTVL |
| 24 | SGSSFNIGSHFVY |
| 25 | KNNQRPS |
| 26 | AAWDDSLNGPV |
| 27 | GAAGTTCAGCTGCTGGAATCTGGCGGCGGACTGGTTCAACCTGGCGGATCTCTGAGACTGAGCTGT |
| | GCCGCCAGCGGCTTCACCTTTAGCGATTATGGCGTGCACTGGGTCCGACAGGCCCCTGGAAAAGGA |
| | CTGGAATGGGTTTCAGGCGTGTCCTGGAACGGCAGCAGAACCCACTATGCCGACAGCGTGAAGGGC |
| | AGATTCACCATCAGCCGGGACAACAGCAAGAACACCCTGTACCTGCAGATGAACAGCCTGAGAGCC |
| | GAGGACACCGCCGTGTACTACTGTGTGACAAGAGGCGCTTGGGGCCAGGGCACACTGGTCACAGTT |
| | TCTTCA |
| 28 | GATTATGGCGTGCAC |
| 29 | GGCGTGTCCTGGAACGGCAGCAGAACCCACTATGCCGACAGCGTGAAGGGC |
| 30 | AGAGGCGCT |
| 31 | CAGTCTGTTCTGACACAGCCTCCTAGCGCCTCTGGCACACCTGGACAGAGAGTGACCATCAGCTGT |
| | AGCGGCAGCAGCTTCAACATCGGCAGCCACTTCGTGTACTGGTATCAGCAGCTGCCTGGCACAGCC |
| | CCTAAACTGCTGATCTACAAGAACAACCAGCGGCCTAGCGGCGTGCCCGATAGATTTTCTGGCAGC |
| | AAGAGCGGCACAAGCGCCAGCCTGGCTATCTCTGGACTGAGATCTGAGGACGAGGCCGACTACTAT |
| | TGCGCCGCCTGGGACGATTCTCTGAACGGCCCTGTTTTTGGCGGAGGCACCAAGCTGACAGTGCTA |
| 32 | AGCGGCAGCAGCTTCAACATCGGCAGCCACTTCGTGTAC |
| 33 | AAGAACAACCAGCGGCCTAGC |
| 34 | GCCGCCTGGGACGATTCTCTGAACGGCCCTGTT |
| 35 | EVQLLESGGGLVQPGGSLRLSCAASGFTFSDYGVHWVRQAPGKGLEWVSGVSWNGSRTHYADSVKG |
| | RFTISRDNSKNTLYLQMNSLRAEDTAVYYCVTRGAWGQGTLVTVSSAKTTAPSVYPLAPVCGDTTG |
| | SSVTLGCLVKGYFPEPVTLTWNSGSLSSGVHTFPAVLQSDLYTLSSSVTVTSSTWPSQSITCNVAH |
| | PASSTKVDKKIEPRGPTIKPCPPCKCPAPNLLGGPSVFIFPPKIKDVLMISLSPIVTCVVVDVSED |
| | DPDVQISWFVNNVEVHTAQTQTHREDYNSTLRVVSALPIQANDWMSGKEFKCKVNNKDLPAPIERT |
| | ISKPKGSVRAPQVYVLPPPEEEMTKKQVTLTCMVTDFMPEDIYVEWTNNGKTELNYKNTEPVLDSD |
| | GSYFMYSKLRVEKKNWVERNSYSCSVVHEGLAQHHTTKSFSRTPGK |
| 36 | QSVLTQPPSASGTPGQRVTISCSGSSFNIGSHFVYWYQQLPGTAPKLLIYKNNQRPSGVPDRFSGS |
| | KSGTSASLAISGLRSEDEADYYCAAWDDSLNGPVFGGGTKLTVLGQPKSSPSVTLFPPSSEELETN |
| | KATLVCTITDFYPGVVTVDWKVDGTPVTQGMETTQPSKQSNNKYMASSYLTLTARAWERHSSYSCQ |
| | VTHEGHTVEKSLSRADCS |
| 37 | EVQLVESGGALVKPGGSLRLSCAASGFTFSTYALYWVRQAPGKGLEWVGRIRSKSNNYATYYADSV |
| | KDRFTISRDDSKNTLYLQMNSLKTEDTAVYYCTRARFYYSDYGYAMDYWGQGTLVTVSS |
| 38 | TYALY |
| 39 | RIRSKSNNYATYYADSVKD |
| 40 | ARFYYSDYGYAMDY |
| 41 | DIVMTQSPDSLAVSLGERATINCRSSKSLLHSNRNTYLYWYQQKPGQPPKLLIYRMSQLASGVPDR |
| | FSGSGSGTDFTLTISSLQAEDVAVYYCMQHLEYPLTFGQGTKLEIK |
| 42 | RSSKSLLHSNRNTYLY |
| 43 | RMSQLAS |
| 44 | MQHLEYPLT |
| 45 | GAAGTGCAGCTGGTGGAATCTGGCGGAGCCCTTGTGAAACCTGGCGGCTCTCTGAGACTGAGCTGT |
| | GCCGCTTCCGGCTTCACCTTCAGCACATACGCCCTGTACTGGGTCCGACAGGCCCCTGGAAAAGGC |
| | CTGGAATGGGTCGGACGGATCAGAAGCAAGAGCAACAACTACGCCACCTACTACGCCGACAGCGTG |
| | AAGGACAGATTCACCATCAGCCGGGACGACAGCAAGAACACCCTGTACCTGCAGATGAACAGCCTG |
| | AAAACCGAGGACACCGCCGTGTACTACTGCACCAGAGCCAGATTCTACTACAGCGACTACGGCTAC |
| | GCCATGGACTATTGGGGCCAGGGCACACTGGTTACCGTTAGCTCA |
| 46 | GACATCGTGATGACACAGAGCCCTGACAGCCTGGCCGTGTCTCTGGGAGAAAGAGCCACCATCAAC |
| | TGCAGAAGCAGCAAGTCCCTGCTGCACAGCAACCGGAACACCTACCTGTACTGGTATCAGCAGAAG |
| | CCCGGCCAGCCTCCTAAGCTGCTGATCTACAGAATGTCCCAGCTGGCCTCCGGCGTGCCCGATAGA |
| | TTTTCTGGCTCTGGCAGCGGCACCGACTTCACCCTGACAATTTCTAGCCTGCAAGCCGAGGACGTG |
| | GCCGTGTACTACTGTATGCAGCACCTCGAGTACCCTCTGACCTTTGGCCAGGGCACCAAGCTGGAA |
| | ATCAAA |
| 47 | EVQLVESGGALVKPGGSLRLSCAASGFTFSTYALYWVRQAPGKGLEWVGRIRSKSNNYATYYADSV |
| | KDRFTISRDDSKNTLYLQMNSLKTEDTAVYYCTRARFYYSDYGYAMDYWGQGTLVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 48 | DIVMTQSPDSLAVSLGERATINCRSSKSLLHSNRNTYLYWYQQKPGQPPKLLIYRMSQLASGVPDR |
| | FSGSGSGTDFTLTISSLQAEDVAVYYCMQHLEYPLTFGQGTKLEIKRTVAAPSVFIFPPSDEQLKS |
| | GTASVVCLLNNFYPREAKVQWKVDNALQSGNSQESVTEQDSKDSTYSLSSTLTLSKADYEKHKVYA |
| | CEVTHQGLSSPVTKSFNRGEC |
| 49 | QVQLVQSGAEVKKPGASVKVSCKASGYTFTSYYMHWVRQAPGQGLEWMGIINPSGGSTSYAQKFQG |
| | RVTMTRDTSTSTVYMELSSLRSEDTAVYYCARAVRNRFRFDYWGQGTLVTVSS |
| 50 | SYYMH |
| 51 | IINPSGGSTSYAQKFQG |
| 52 | AVRNRFRFDY |
| 53 | QSALTQPASVSGSPGQSITISCTGTSSDVGSYNLVSWYQQHPGKAPKLMIYEVSKRPSGVSNRFSG |
| | SKSGNTASLTISGLQAEDEADYYCSSYAGSSTFVVFGGGTKLTVL |
| 54 | TGTSSDVGSYNLVS |
| 55 | EVSKRPS |
| 56 | SSYAGSSTFVV |
| 57 | CAGGTTCAGCTGGTTCAGTCTGGCGCCGAAGTGAAGAAACCTGGCGCCTCTGTGAAGGTGTCCTGC |
| | AAGGCCAGCGGCTACACCTTTACCAGCTACTACATGCACTGGGTCCGACAGGCCCCTGGACAAGGA |
| | CTTGAGTGGATGGGCATCATCAACCCTAGCGGCGGCAGCACAAGCTACGCCCAGAAATTCCAGGGC |
| | AGAGTGACCATGACCAGAGACACCAGCACCTCCACCGTGTACATGGAACTGAGCAGCCTGAGAAGC |
| | GAGGACACCGCCGTGTACTATTGTGCCAGAGCCGTGCGGAACAGATTCAGATTCGACTACTGGGGC |
| | CAGGGCACCCTGGTTACAGTTTCTTCA |
| 58 | CAGTCTGCTCTTACACAGCCTGCCTCTGTGTCTGGCTCTCCTGGCCAGAGCATCACCATCAGCTGT |
| | ACCGGCACCAGCTCTGACGTGGGCAGCTACAATCTGGTGTCCTGGTATCAGCAGCACCCCGGCAAA |
| | GCCCCTAAGCTGATGATCTACGAGGTGTCCAAGAGGCCCAGCGGCGTGTCCAATAGATTCAGCGGC |
| | AGCAAGAGCGGCAACACCGCCAGCCTGACAATTAGCGGACTGCAGGCCGAGGACGAGGCCGATTAC |
| | TACTGTAGCAGCTACGCCGGCAGCTCCACCTTCGTGGTTTTTGGCGGAGGCACCAAGCTGACCGTT |
| | CTA |
| 59 | QVQLVQSGAEVKKPGASVKVSCKASGYTFTSYYMHWVRQAPGQGLEWMGIINPSGGSTSYAQKFQG |
| | RVTMTRDTSTSTVYMELSSLRSEDTAVYYCARAVRNRFRFDYWGQGTLVTVSSASTKGPSVFPLAP |
| | SSKSTSGGTAALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |
| 60 | QSALTQPASVSGSPGQSITISCTGTSSDVGSYNLVSWYQQHPGKAPKLMIYEVSKRPSGVSNRFSG |
| | SKSGNTASLTISGLQAEDEADYYCSSYAGSSTFVVFGGGTKLTVLGQPKAAPSVTLFPPSSEELQA |
| | NKATLVCLISDFYPGAVTVAWKADSSPVKAGVETTTPSKQSNNKYAASSYLSLTPEQWKSHRSYSC |
| | QVTHEGSTVEKTVAPTECS |
| 61 | EVQLLESGGGLVQPGGSLRLSCAAGGFTFSAYTMNWVRQAPGKGLEWVSAISASGGRTYYADSVKG |
| | RFTISRDNSKNTLYLQMNSLRAEDTAVYYCARRFARGWFDPWGQGTLVTVSS |
| 62 | AYTMN |
| 63 | AISASGGRTYYADSVKG |
| 64 | RFARGWFDP |
| 65 | QSVLTQPPSVSAAPGQKVTISCSGSSSNIGNNYVSWYQQLPGTAPKLLIYDNNKRPSGIPDRFSGS |
| | KSGTSATLGITGLQTGDEADYYCGTWDSSLSAWVFGGGTKLTVL |
| 66 | SGSSSNIGNNYVS |
| 67 | DNNKRPS |
| 68 | GTWDSSLSAWV |
| 69 | GAAGTTCAGCTGCTGGAATCTGGCGGCGGACTGGTTCAACCTGGCGGATCTCTGAGACTGTCTTGT |
| | GCCGCTGGCGGCTTCACCTTTAGCGCCTACACCATGAACTGGGTCCGACAGGCCCCTGGCAAAGGC |
| | CTTGAATGGGTGTCAGCCATCTCTGCCTCTGGCGGCAGAACCTACTACGCCGATTCTGTGAAGGGC |
| | AGATTCACCATCAGCCGGGACAACAGCAAGAACACCCTGTACCTGCAGATGAACAGCCTGAGAGCC |
| | GAGGACACCGCCGTGTACTACTGCGCCAGACGCTTTGCCAGAGGATGGTTCGATCCTTGGGGCCAG |
| | GGAACCCTGGTTACAGTCTCTTCA |
| 70 | CAGTCTGTTCTGACACAGCCTCCATCCGTGTCTGCTGCCCCTGGCCAGAAAGTGACCATCAGCTGT |
| | AGCGGCAGCAGCTCCAACATCGGCAACAACTACGTGTCCTGGTATCAGCAGCTGCCCGGCACAGCT |
| | CCCAAACTGCTGATCTACGACAACAACAAGCGGCCCAGCGGCATCCCCGATAGATTTTCTGGCAGC |
| | AAGAGCGGCACCAGCGCCACACTGGGAATTACAGGACTGCAGACAGGCGACGAGGCCGACTACTAT |
| | TGTGGCACCTGGGATTCTAGCCTGAGCGCCTGGGTTTTCGGCGGAGGCACAAAACTGACAGTGCTA |
| 71 | EVQLLESGGGLVQPGGSLRLSCAAGGFTFSAYTMNWVRQAPGKGLEWVSAISASGGRTYYADSVKG |
| | RFTISRDNSKNTLYLQMNSLRAEDTAVYYCARRFARGWFDPWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTP |
| | PVLDSDGSFFLYSKLTVDKSRWQQGNVFSCSVMHEALHNHYTQKSLSLSPGK |
| 72 | QSVLTQPPSVSAAPGQKVTISCSGSSSNIGNNYVSWYQQLPGTAPKLLIYDNNKRPSGIPDRFSGS |
| | KSGTSATLGITGLQTGDEADYYCGTWDSSLSAWVFGGGTKLTVLGQPKAAPSVTLFPPSSEELQAN |
| | KATLVCLISDFYPGAVTVAWKADSSPVKAGVETTTPSKQSNNKYAASSYLSLTPEQWKSHRSYSCQ |
| | VTHEGSTVEKTVAPTECS |
| 73 | QVQLVQSGAEVKKPGASVKVSCKASGYTFTDSEMHWVRQATGQGLEWMGAIQPETGGTAYNQKFKA |
| | RVTMTRDTSISTAYMELSSLRSEDTAVYYCARRRRNFDYWGQGTLVTVSS |
| 74 | DSEMH |
| 75 | AIQPETGGTAYNQKFKA |
| 76 | RRRNFDY |
| 77 | DIVMTQTPLSLSVTPGQPASISCRSSQSLFHSSGNTYLHWYLQKPGQPPQLLIYKVSNRFSGVPDR |
| | FSGSGSGTDFTLKISRVEAEDVGVYYCSQSTHVPFTFGQGTKLEIK |
| 78 | RSSQSLFHSSGNTYLH |
| 79 | KVSNRFS |
| 80 | SQSTHVPFT |
| 81 | CAGGTTCAGCTGGTTCAGTCTGGCGCCGAAGTGAAGAAACCTGGCGCCTCTGTGAAGGTGTCCTGC |
| | AAGGCCAGCGGCTACACCTTTACCGACAGCGAGATGCACTGGGTCCGACAGGCTACAGGACAGGGA |
| | CTCGAATGGATGGGAGCCATCCAGCCTGAAACAGGCGGCACCGCCTACAACCAGAAATTCAAGGCC |
| | AGAGTGACCATGACCAGAGACACCAGCATCAGCACAGCCTACATGGAACTGAGCAGCCTGAGAAGC |
| | GAGGACACCGCCGTGTACTACTGCGCCCGCAGAAGAAGAAACTTCGACTACTGGGGCCAGGGCACC |
| | CTGGTTACAGTTTCTTCA |
| 82 | GACATCGTGATGACCCAGACACCTCTGAGCCTGAGCGTGACACCTGGACAGCCTGCCAGCATCAGC |
| | TGTAGAAGCAGCCAGAGCCTGTTCCACAGCTCCGGCAATACCTACCTGCACTGGTATCTGCAGAAG |
| | CCCGGACAGCCTCCTCAGCTGCTGATCTACAAGGTGTCCAACCGGTTCAGCGGCGTGCCCGATAGA |
| | TTTTCTGGCAGCGGCTCTGGCACCGACTTCACCCTGAAGATCTCCAGAGTGGAAGCCGAGGACGTG |
| | GGCGTGTACTACTGTAGCCAGAGCACCCACGTGCCATTCACCTTTGGCCAGGGCACCAAGCTGGAA |
| | ATCAAA |
| 83 | QVQLVQSGAEVKKPGASVKVSCKASGYTFTDSEMHWVRQATGQGLEWMGAIQPETGGTAYNQKFKA |
| | RVTMTRDTSISTAYMELSSLRSEDTAVYYCARRRRNFDYWGQGTLVTVSSASTKGPSVFPLAPSSK |
| | STSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| 84 | DIVMTQTPLSLSVTPGQPASISCRSSQSLFHSSGNTYLHWYLQKPGQPPQLLIYKVSNRFSGVPDR |
| | FSGSGSGTDFTLKISRVEAEDVGVYYCSQSTHVPFTFGQGTKLEIKRTVAAPSVFIFPPSDEQLKS |
| | GTASVVCLLNNFYPREAKVQWKVDNALQSGNSQESVTEQDSKDSTYSLSSTLTLSKADYEKHKVYA |
| | CEVTHQGLSSPVTKSFNRGEC |
| 85 | EVQLVESGGGLVQPGGSLKLSCAASGFTFNTYAMNWVRQASGKGLEWVGRIRSKSNNYATYYADSV |
| | KDRFTISRDDSKNTAYLQMNSLKTEDTAVYYCVRGLLRYRFFDVWGQGTTVTVSS |
| 86 | TYAMN |
| 87 | RIRSKSNNYATYYADSVKD |
| 88 | GLLRYRFFDV |
| 89 | DIVMTQSPLSLPVTPGEPASISCRSSKSLLHSNGNTYLYWFLQKPGQSPQLLIYRMSNLASGVPDR |
| | FSGSGSGTDFTLKISRVEAEDVGVYYCMQHLEYPFTFGGGTKVEIK |
| 90 | RSSKSLLHSNGNTYLY |
| 91 | RMSNLAS |
| 92 | MQHLEYPFT |
| 93 | GAAGTGCAGCTGGTGGAATCTGGCGGAGGACTGGTTCAACCTGGCGGCTCTCTGAAGCTGTCTTGT |
| | GCCGCCAGCGGCTTCACCTTCAACACCTACGCCATGAACTGGGTCCGACAGGCCTCTGGCAAAGGC |
| | CTGGAATGGGTCGGACGGATCAGAAGCAAGAGCAACAATTACGCCACCTACTACGCCGACAGCGTG |
| | AAGGACAGATTCACCATCAGCCGGGACGACAGCAAGAACACCGCCTACCTGCAGATGAACAGCCTG |
| | AAAACCGAGGACACCGCCGTGTACTATTGTGTGCGGGGCCTGCTGCGGTACAGATTCTTTGATGTG |
| | TGGGGCCAGGGCACCACCGTGACAGTTTCTTCA |
| 94 | GACATCGTGATGACACAGAGCCCTCTGAGCCTGCCTGTGACACCTGGCGAACCTGCCAGCATCAGC |
| | TGCAGAAGCAGCAAGTCTCTGCTGCACAGCAACGGCAATACCTACCTGTACTGGTTCCTGCAGAAA |
| | CCCGGCCAGTCTCCTCAGCTGCTGATCTACAGAATGAGCAACCTGGCCAGCGGCGTGCCCGATAGA |
| | TTTTCTGGCTCTGGCAGCGGCACCGACTTCACCCTGAAGATCTCTAGAGTGGAAGCCGAGGACGTG |
| | GGCGTGTACTACTGTATGCAGCACCTCGAGTACCCCTTCACCTTTGGCGGCGGAACAAAGGTGGAA |
| | ATCAAA |
| 95 | EVQLVESGGGLVQPGGSLKLSCAASGFTFNTYAMNWVRQASGKGLEWVGRIRSKSNNYATYYADSV |
| | KDRFTISRDDSKNTAYLQMNSLKTEDTAVYYCVRGLLRYRFFDVWGQGTTVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 96 | DIVMTQSPLSLPVTPGEPASISCRSSKSLLHSNGNTYLYWFLQKPGQSPQLLIYRMSNLASGVPDR |
| | FSGSGSGTDFTLKISRVEAEDVGVYYCMQHLEYPFTFGGGTKVEIKRTVAAPSVFIFPPSDEQLKS |
| | GTASVVCLLNNFYPREAKVQWKVDNALQSGNSQESVTEQDSKDSTYSLSSTLTLSKADYEKHKVYA |
| | CEVTHQGLSSPVTKSFNRGEC |

1. A method for treating a patient in need thereof, the method comprising administering intravenously to the patient an anti-human CCR8 antibody having ADCC activity and ADCP activity in a total amount of
a) approximately 1 to 250 mg once every week, preferably 3, 10, 30, 50, 100, 125, or 250 mg once every week, or
b) approximately 16 to 1500 mg once every three weeks, preferably 16, 450, 500, 750, 1000 or 1500 mg once every three weeks.

2. The method of claim 1, comprising administering the anti-CCR8 antibody in a total amount of 2.7 mg to 75 mg once every week, or a total amount of 16 mg to 450 mg once every three weeks.

3. (canceled)

4. The method of claim 1, further comprising administering intravenously to the patient in need thereof an anti-PD-(L)1 antibody in a total amount of
a) approximately 200 mg once every three weeks, preferably wherein the anti-PD-(L)1 antibody is pembrolizumab, or
b) approximately 400 mg once every six weeks, preferably wherein the anti-PD-(L)1 antibody is pembrolizumab, or
c) approximately 240 mg once every two weeks, preferably wherein the anti-PD-(L)1 antibody is nivolumab, or
d) approximately 360 mg once every three weeks, preferably wherein the anti-PD-(L)1 antibody is nivolumab, or
e) approximately 480 mg once every four weeks, preferably wherein the anti-PD-(L)1 antibody is nivolumab, or
f) approximately 840 mg once every two weeks, preferably wherein the anti-PD-(L)1 antibody is atezolizumab, or
g) approximately 1200 mg once every three weeks, preferably wherein the anti-PD-(L)1 antibody is atezolizumab, or
h) approximately 1680 mg once every four weeks, preferably wherein the anti-PD-(L)1 antibody is atezolizumab, or
i) approximately 360 mg once every three weeks, preferably wherein the anti-PD-(L)1 antibody is Zimberelimab, or
j) approximately 3 mg/kg once every two weeks, preferably wherein the anti-PD-(L)1 antibody is Toripalimab, or
k) approximately 10 mg/kg once every two weeks, preferably wherein the anti-PD-(L)1 antibody is Durvalumab, or
l) approximately 1500 mg once every 3 weeks, preferably wherein the anti-PD-(L)1 antibody is Durvalumab.

5. (canceled)

6. The method of claim 4, wherein the anti-PD-(L)1 antibody is administered after the anti-CCR8 antibody.

7. The method of claim 1, wherein the intravenous administration of the anti-human CCR8 antibody occurs as a 15- to 120-minute intravenous infusion.

8. The method of claim 7, wherein the intravenous administration of the anti-PD-(L)1 antibody occurs as a 15- to 60-minute intravenous infusion.

9. (canceled)

10. (canceled)

11. The method of claim 4, wherein the method comprises at least one 21-day dosing cycle, and preferably wherein the anti-CCR8 antibody and the anti-PD-(L)1 antibody are both administered on day 1 of the 21-day dosing cycle.

12. The method of claim 4, wherein the method comprises at least two and preferably more dosing cycles, and wherein for the second, third, fourth, fifth or any subsequent dosing cycle the anti-human CCR8 antibody and the anti-PD-(L)1 antibody are administered without substantial delay directly after each other.

13. The method of claim 1, wherein the anti-CCR8 antibody is a human IgG1 antibody.

14. The method of claim 1, wherein the anti-CCR8 antibody is a low internalizing or non-internalizing antibody.

15. (canceled)

16. The method of claim 4, wherein the anti-PD-(L)1 antibody is pembrolizumab, nivolumab, atezolizumab, avelumab, Zimberelimab, Toripalimab or Durvalumab.

17. The method of claim 4, wherein the anti-CCR8 antibody comprises at least one of:
a) HCDR1, HCDR2, HCDR3, LCDR1, LCDR2 and LCDR3 sequences of SEQ ID NOs: 2, 3, 4, 6, 7 and 8,
b) at least one of a variable heavy chain sequence that has at least 98% or 100% sequence identity with the amino acid sequence set forth in SEQ ID NO:1 and a variable light chain sequence that has at least 98% or 100% sequence identity with the amino acid sequence set forth in SEQ ID NO:5, and
c) at least one of a heavy chain sequence that has at least 98% or 100% sequence identity with the amino acid sequence set forth in SEQ ID NO:17 and a light chain sequence that has at least 98% or 100% sequence identity with the amino acid sequence set forth in SEQ ID NO:18.

18. The method of claim 1, wherein the method treats a cancer.

19. The method of claim 1, wherein prior to the administration of the anti-CCR8 antibody, the method further comprises administering: an effective dose of antihistamines, acetaminophen, corticosteroids or a combination thereof, preferably:
a) at least 500 mg or at least 650 mg paracetamol,
b) at least 50 mg or at least 100 mg diphenhydramine, and
c) at least 8 mg dexamethasone.

20. The method of claim 1, wherein the method is for treating cancer, and wherein the method further comprises the steps of:
a) analysing the Tumor Proportion Score or the Combined Positive Score as a measure for PD-(L)1 expression in a cancer tissue sample of the patient, and
b) administering the anti-human CCR8 antibody to the patient if the patient has a Tumor Proportion Score of ≥50% or a Combined Positive Score of ≥10% or ≥1%.

21.-24. (canceled)

25. The method of claim 1, wherein the method is for treating cancer, and wherein the method further comprises the steps of:
a) optionally analysing in a blood, plasma or serum screening sample of the patient the level of at least one and preferably at least 2, 3, 4, 5, 6, 7, 8, 9 or 10 inflammatory cytokines, selected from the group of IFN-γ, IL-1β, IL-2, IL-4, IL-6, IL-8, IL-10, IL 12p70, IL-13, and TNF-α,
b) administering to the patient an effective dose of the anti-human CCR8 antibody,
c) analysing in a blood, plasma or serum sample of the patient the level of the at least one and preferably at least 2, 3, 4, 5, 6, 7, 8, 9 or 10 inflammatory cytokines,
wherein the blood, plasma or serum sample is drawn after administering the effective dose of the anti-human CCR8 antibody according to step b),
d) comparing the cytokine(s) level(s) obtained according to step c)
i. either with the cytokine(s) level(s) obtained according to step a), or
ii. with a reference value,
to identify safety-related events, or as a surrogate biomarker for Treg depletion, or as a biomarker for treatment success.

26. The method of claim 25, further comprising
a. administering to the patient at least one further effective dose of the anti-human CCR8 antibody, if the cytokine(s) level(s) obtained according to step c) are increased
i. relative to the cytokine(s) level(s) obtained according to step a), or
relative to a reference value.

27.-28. (canceled)

29. A method for treating cancer in a patient in need thereof, the method comprising:
a) administering to the patient an anti-human CCR8 antibody having ADCC activity and ADCP activity:
b) stratifying the patient based on a previous treatment of the cancer with an anti-PD-(L)1 antibody for at least 6 months, and
c) administering the anti-human CCR8 antibody to the patient only if the patient has previously been treated with an anti-PD-(L)1 antibody for at least 6 months.

30. (canceled)

31. A method for treating cancer in a subject in need thereof, the method comprising the steps of:
a) administering to the subject a Zr-89-labeled anti-CD8 minibody,
b) performing at least one PET scan and optionally a CT scan to detect the Zr-89-labeled anti-CD8 minibody in the subject to generate a first subject image,
c) determining at least one of an abundance and a distribution of Zr-89-labeled anti-CD8 minibody in one or more cancer lesions of the subject based on the first subject image, and
d) administering to the subject an effective dose of an anti-human CCR8 antibody having ADCC activity and ADCP activity if the first subject image indicates the abundance and/or the distribution of Zr-89-labeled anti-CD8 minibody in any of the one or more cancer lesions that indicates a substantial likelihood of the subject to profit from administration of the anti-human CCR8 antibody.

32. (canceled)

33. A method for treating cancer in a subject in need thereof, the method comprising the steps of:
a) administering to the subject a first dose of a Zr-89-labeled anti-CD8 minibody,
b) performing a first PET scan and optionally a CT scan to detect the Zr-89-labeled anti-CD8 minibody in the subject, to generate a first subject image,
c) determining a first abundance and/or distribution of Zr-89-labeled anti-CD8 minibody in one or more cancer lesions in the subject based on the first subject image,
d) administering to the subject an effective dose of an anti-human CCR8 antibody having ADCC activity and ADCP activity,
e) administering to the subject a second dose of the Zr-89-labeled anti-CD8 minibody,
f) performing a second PET scan and optionally a CT scan to detect the Zr-89-labeled anti-CD8 minibody in the subject, to generate a second subject image,
g) determining a second abundance and/or distribution of Zr-89-labeled anti-CD8 minibody in one or more cancer lesions in the subject based on the second subject image,
h) comparing the second subject image to the first subject image in order to evaluate if the abundance of Zr-89-labeled anti-CD8 minibody has substantially increased or if the distribution of Zr-89-labeled anti-CD8 minibody has substantially changed in one or more cancer lesions for monitoring disease progression or success of the anti-human CCR8 antibody treatment, and optionally
i) administering to the patient at least one further effective dose of the anti-human CCR8 antibody if the abundance of Zr-89-labeled anti-CD8 minibody has substantially increased or if the distribution of Zr-89-labeled anti-CD8 minibody has substantially changed in one or more cancer lesions.

34.-38. (canceled)

39. A method for determining and quantifying anti-anti-CCR8 antibody formation in cynomolgus or human plasma, the method comprising a bridging ELISA method, optionally wherein a signal is generated if the anti-anti-CCR8 antibody bridges a) biotinylated anti-CCR8 antibody and b) SULFO-tagged anti-CCR8 antibody.

40. (canceled)

41. An isolated anti-CCR8 antibody or antigen-binding fragment thereof comprising HCDR1, HCDR2, HCDR3, LCDR1, LCDR2 and LCDR3 sequences of SEQ ID NOs: 20, 21, 22, 24, 25 and 26.

42. The isolated anti-CCR8 antibody or antigen-binding fragment thereof of claim 41, further comprising at least one of:
a) a variable heavy chain sequence that has at least 98% or 100% sequence identity with the amino acid sequence set forth in SEQ ID NO:19, and
b) a variable light chain sequence that has at least 98% or 100% sequence identity with the amino acid sequence set forth in SEQ ID NO:23.

43. The isolated anti-CCR8 antibody or antigen-binding fragment thereof of claim 41, further comprising at least one of:
a) a heavy chain sequence that has at least 98% or 100% sequence identity with the amino acid sequence set forth in SEQ ID NO:35 and
b) a light chain sequence that has at least 98% or 100% sequence identity with the amino acid sequence set forth in SEQ ID NO:36.


SEQUENCE LISTING (96 sequences)

SEQ ID NO: 1 - protein, 122 aa, synthetic construct
EVQLLESGGGLVQPGGSLRLSCAASGFTFSSYGMHWVRQAPGKGLEWVSAINWNGGSTGYADSVKGRFTISRDNSKNTLYLQMNSLRAEDTAVYYCARGHHSGYDGRFFDYWGQGTLVTVSS

SEQ ID NO: 2 - protein, 5 aa, synthetic construct
SYGMH

SEQ ID NO: 3 - protein, 17 aa, synthetic construct
AINWNGGSTGYADSVKG

SEQ ID NO: 4 - protein, 13 aa, synthetic construct
GHHSGYDGRFFDY

SEQ ID NO: 5 - protein, 111 aa, synthetic construct
QSVLTQPPSVSGAPGQRVTISCTGSSSNIGAGYNVHWYQQLPGTAPKLLIYTNNRRPSGVPDRFSGSKSGTSASLAISGLRSEDEADYYCAAWDASLSGWVFGGGTKLTVL

SEQ ID NO: 6 - protein, 14 aa, synthetic construct
TGSSSNIGAGYNVH

SEQ ID NO: 7 - protein, 7 aa, synthetic construct
TNNRRPS

SEQ ID NO: 8 - protein, 11 aa, synthetic construct
AAWDASLSGWV

SEQ ID NO: 9 - DNA, 366 bp, synthetic construct
  source 1..366: mol_type = other DNA
gaggtgcagctgctggaatctggcggaggattggttcagcctggcggctctctgagactgtcttgtgccgcttccggcttcaccttctccagctacggaatgcactgggtccgacaggcccctggcaaaggattggaatgggtgtccgccatcaactggaacggcggctctaccggctacgccgattctgtgaagggcagattcaccatcagccgggacaactccaagaacaccctgtacctgcagatgaactccctgagagccgaggacaccgccgtgtactattgtgctagaggccaccactctggctacgacggcagattcttcgactattggggccagggcaccctggtcacagtttcttca

SEQ ID NO: 10 - DNA, 15 bp, synthetic construct
  source 1..15: mol_type = other DNA
agctacggaatgcac

SEQ ID NO: 11 - DNA, 51 bp, synthetic construct
  source 1..51: mol_type = other DNA
gccatcaactggaacggcggctctaccggctacgccgattctgtgaagggc

SEQ ID NO: 12 - DNA, 39 bp, synthetic construct
  source 1..39: mol_type = other DNA
ggccaccactctggctacgacggcagattcttcgactat

SEQ ID NO: 13 - DNA, 333 bp, synthetic construct
  source 1..333: mol_type = other DNA
cagtctgttctgacacagcctccatctgtgtctggcgcccctggacagagagtgaccatcagctgtacaggcagcagctccaatatcggagccggctacaacgtgcactggtatcagcagctgcctggcacagcccctaaactgctgatctacaccaacaacagacggcccagcggcgtgcccgatagattttctggcagcaagagcggcacaagcgccagcctggctatctctggactgagatctgaggacgaggccgactactattgcgccgcctgggatgcttctctgagcggatgggttttcggcggaggcaccaaactgacagtgcta

SEQ ID NO: 14 - DNA, 42 bp, synthetic construct
  source 1..42: mol_type = other DNA
acaggcagcagctccaatatcggagccggctacaacgtgcac

SEQ ID NO: 15 - DNA, 21 bp, synthetic construct
  source 1..21: mol_type = other DNA
accaacaacagacggcccagc

SEQ ID NO: 16 - DNA, 33 bp, synthetic construct
  source 1..33: mol_type = other DNA
gccgcctgggatgcttctctgagcggatgggtt

SEQ ID NO: 17 - protein, 452 aa, synthetic construct
EVQLLESGGGLVQPGGSLRLSCAASGFTFSSYGMHWVRQAPGKGLEWVSAINWNGGSTGYADSVKGRFTISRDNSKNTLYLQMNSLRAEDTAVYYCARGHHSGYDGRFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 217 aa, synthetic construct
QSVLTQPPSVSGAPGQRVTISCTGSSSNIGAGYNVHWYQQLPGTAPKLLIYTNNRRPSGVPDRFSGSKSGTSASLAISGLRSEDEADYYCAAWDASLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 112 aa, synthetic construct
EVQLLESGGGLVQPGGSLRLSCAASGFTFSDYGVHWVRQAPGKGLEWVSGVSWNGSRTHYADSVKGRFTISRDNSKNTLYLQMNSLRAEDTAVYYCVTRGAWGQGTLVTVSS

SEQ ID NO: 20 - protein, 5 aa, synthetic construct
DYGVH

SEQ ID NO: 21 - protein, 17 aa, synthetic construct
GVSWNGSRTHYADSVKG

SEQ ID NO: 22 - skipped

SEQ ID NO: 23 - protein, 110 aa, synthetic construct
QSVLTQPPSASGTPGQRVTISCSGSSFNIGSHFVYWYQQLPGTAPKLLIYKNNQRPSGVPDRFSGSKSGTSASLAISGLRSEDEADYYCAAWDDSLNGPVFGGGTKLTVL

SEQ ID NO: 24 - protein, 13 aa, synthetic construct
SGSSFNIGSHFVY

SEQ ID NO: 25 - protein, 7 aa, synthetic construct
KNNQRPS

SEQ ID NO: 26 - protein, 11 aa, synthetic construct
AAWDDSLNGPV

SEQ ID NO: 27 - DNA, 336 bp, synthetic construct
  source 1..336: mol_type = other DNA
gaagttcagctgctggaatctggcggcggactggttcaacctggcggatctctgagactgagctgtgccgccagcggcttcacctttagcgattatggcgtgcactgggtccgacaggcccctggaaaaggactggaatgggtttcaggcgtgtcctggaacggcagcagaacccactatgccgacagcgtgaagggcagattcaccatcagccgggacaacagcaagaacaccctgtacctgcagatgaacagcctgagagccgaggacaccgccgtgtactactgtgtgacaagaggcgcttggggccagggcacactggtcacagtttcttca

SEQ ID NO: 28 - DNA, 15 bp, synthetic construct
  source 1..15: mol_type = other DNA
gattatggcgtgcac

SEQ ID NO: 29 - DNA, 51 bp, synthetic construct
  source 1..51: mol_type = other DNA
ggcgtgtcctggaacggcagcagaacccactatgccgacagcgtgaagggc

SEQ ID NO: 30 - skipped

SEQ ID NO: 31 - DNA, 330 bp, synthetic construct
  source 1..330: mol_type = other DNA
cagtctgttctgacacagcctcctagcgcctctggcacacctggacagagagtgaccatcagctgtagcggcagcagcttcaacatcggcagccacttcgtgtactggtatcagcagctgcctggcacagcccctaaactgctgatctacaagaacaaccagcggcctagcggcgtgcccgatagattttctggcagcaagagcggcacaagcgccagcctggctatctctggactgagatctgaggacgaggccgactactattgcgccgcctgggacgattctctgaacggccctgtttttggcggaggcaccaagctgacagtgcta

SEQ ID NO: 32 - DNA, 39 bp, synthetic construct
  source 1..39: mol_type = other DNA
agcggcagcagcttcaacatcggcagccacttcgtgtac

SEQ ID NO: 33 - DNA, 21 bp, synthetic construct
  source 1..21: mol_type = other DNA
aagaacaaccagcggcctagc

SEQ ID NO: 34 - DNA, 33 bp, synthetic construct
  source 1..33: mol_type = other DNA
gccgcctgggacgattctctgaacggccctgtt

SEQ ID NO: 35 - protein, 442 aa, synthetic construct
EVQLLESGGGLVQPGGSLRLSCAASGFTFSDYGVHWVRQAPGKGLEWVSGVSWNGSRTHYADSVKGRFTISRDNSKNTLYLQMNSLRAEDTAVYYCVTRGAWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQANDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLAQHHTTKSFSRTPGK

SEQ ID NO: 36 - protein, 216 aa, synthetic construct
QSVLTQPPSASGTPGQRVTISCSGSSFNIGSHFVYWYQQLPGTAPKLLIYKNNQRPSGVPDRFSGSKSGTSASLAISGLRSEDEADYYCAAWDDSLNGPVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 37 - protein, 125 aa, synthetic construct
EVQLVESGGALVKPGGSLRLSCAASGFTFSTYALYWVRQAPGKGLEWVGRIRSKSNNYATYYADSVKDRFTISRDDSKNTLYLQMNSLKTEDTAVYYCTRARFYYSDYGYAMDYWGQGTLVTVSS

SEQ ID NO: 38 - protein, 5 aa, synthetic construct
TYALY

SEQ ID NO: 39 - protein, 19 aa, synthetic construct
RIRSKSNNYATYYADSVKD

SEQ ID NO: 40 - protein, 14 aa, synthetic construct
ARFYYSDYGYAMDY

SEQ ID NO: 41 - protein, 112 aa, synthetic construct
DIVMTQSPDSLAVSLGERATINCRSSKSLLHSNRNTYLYWYQQKPGQPPKLLIYRMSQLASGVPDRFSGSGSGTDFTLTISSLQAEDVAVYYCMQHLEYPLTFGQGTKLEIK

SEQ ID NO: 42 - protein, 16 aa, synthetic construct
RSSKSLLHSNRNTYLY

SEQ ID NO: 43 - protein, 7 aa, synthetic construct
RMSQLAS

SEQ ID NO: 44 - protein, 9 aa, synthetic construct
MQHLEYPLT

SEQ ID NO: 45 - DNA, 375 bp, synthetic construct
  source 1..375: mol_type = other DNA
gaagtgcagctggtggaatctggcggagcccttgtgaaacctggcggctctctgagactgagctgtgccgcttccggcttcaccttcagcacatacgccctgtactgggtccgacaggcccctggaaaaggcctggaatgggtcggacggatcagaagcaagagcaacaactacgccacctactacgccgacagcgtgaaggacagattcaccatcagccgggacgacagcaagaacaccctgtacctgcagatgaacagcctgaaaaccgaggacaccgccgtgtactactgcaccagagccagattctactacagcgactacggctacgccatggactattggggccagggcacactggttaccgttagctca

SEQ ID NO: 46 - DNA, 336 bp, synthetic construct
  source 1..336: mol_type = other DNA
gacatcgtgatgacacagagccctgacagcctggccgtgtctctgggagaaagagccaccatcaactgcagaagcagcaagtccctgctgcacagcaaccggaacacctacctgtactggtatcagcagaagcccggccagcctcctaagctgctgatctacagaatgtcccagctggcctccggcgtgcccgatagattttctggctctggcagcggcaccgacttcaccctgacaatttctagcctgcaagccgaggacgtggccgtgtactactgtatgcagcacctcgagtaccctctgacctttggccagggcaccaagctggaaatcaaa

SEQ ID NO: 47 - protein, 455 aa, synthetic construct
EVQLVESGGALVKPGGSLRLSCAASGFTFSTYALYWVRQAPGKGLEWVGRIRSKSNNYATYYADSVKDRFTISRDDSKNTLYLQMNSLKTEDTAVYYCTRARFYYSDYGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 219 aa, synthetic construct
DIVMTQSPDSLAVSLGERATINCRSSKSLLHSNRNTYLYWYQQKPGQPPKLLIYRMSQLASGVPDRFSGSGSGTDFTLTISSLQAEDVAVYYCMQHLEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 119 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAVRNRFRFDYWGQGTLVTVSS

SEQ ID NO: 50 - protein, 5 aa, synthetic construct
SYYMH

SEQ ID NO: 51 - protein, 17 aa, synthetic construct
IINPSGGSTSYAQKFQG

SEQ ID NO: 52 - protein, 10 aa, synthetic construct
AVRNRFRFDY

SEQ ID NO: 53 - protein, 111 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSSTFVVFGGGTKLTVL

SEQ ID NO: 54 - protein, 14 aa, synthetic construct
TGTSSDVGSYNLVS

SEQ ID NO: 55 - protein, 7 aa, synthetic construct
EVSKRPS

SEQ ID NO: 56 - protein, 11 aa, synthetic construct
SSYAGSSTFVV

SEQ ID NO: 57 - DNA, 357 bp, synthetic construct
  source 1..357: mol_type = other DNA
caggttcagctggttcagtctggcgccgaagtgaagaaacctggcgcctctgtgaaggtgtcctgcaaggccagcggctacacctttaccagctactacatgcactgggtccgacaggcccctggacaaggacttgagtggatgggcatcatcaaccctagcggcggcagcacaagctacgcccagaaattccagggcagagtgaccatgaccagagacaccagcacctccaccgtgtacatggaactgagcagcctgagaagcgaggacaccgccgtgtactattgtgccagagccgtgcggaacagattcagattcgactactggggccagggcaccctggttacagtttcttca

SEQ ID NO: 58 - DNA, 333 bp, synthetic construct
  source 1..333: mol_type = other DNA
cagtctgctcttacacagcctgcctctgtgtctggctctcctggccagagcatcaccatcagctgtaccggcaccagctctgacgtgggcagctacaatctggtgtcctggtatcagcagcaccccggcaaagcccctaagctgatgatctacgaggtgtccaagaggcccagcggcgtgtccaatagattcagcggcagcaagagcggcaacaccgccagcctgacaattagcggactgcaggccgaggacgaggccgattactactgtagcagctacgccggcagctccaccttcgtggtttttggcggaggcaccaagctgaccgttcta

SEQ ID NO: 59 - protein, 449 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAVRNRFRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 217 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSSTFVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 61 - protein, 118 aa, synthetic construct
EVQLLESGGGLVQPGGSLRLSCAAGGFTFSAYTMNWVRQAPGKGLEWVSAISASGGRTYYADSVKGRFTISRDNSKNTLYLQMNSLRAEDTAVYYCARRFARGWFDPWGQGTLVTVSS

SEQ ID NO: 62 - protein, 5 aa, synthetic construct
AYTMN

SEQ ID NO: 63 - protein, 17 aa, synthetic construct
AISASGGRTYYADSVKG

SEQ ID NO: 64 - protein, 9 aa, synthetic construct
RFARGWFDP

SEQ ID NO: 65 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 66 - protein, 13 aa, synthetic construct
SGSSSNIGNNYVS

SEQ ID NO: 67 - protein, 7 aa, synthetic construct
DNNKRPS

SEQ ID NO: 68 - protein, 11 aa, synthetic construct
GTWDSSLSAWV

SEQ ID NO: 69 - DNA, 354 bp, synthetic construct
  source 1..354: mol_type = other DNA
gaagttcagctgctggaatctggcggcggactggttcaacctggcggatctctgagactgtcttgtgccgctggcggcttcacctttagcgcctacaccatgaactgggtccgacaggcccctggcaaaggccttgaatgggtgtcagccatctctgcctctggcggcagaacctactacgccgattctgtgaagggcagattcaccatcagccgggacaacagcaagaacaccctgtacctgcagatgaacagcctgagagccgaggacaccgccgtgtactactgcgccagacgctttgccagaggatggttcgatccttggggccagggaaccctggttacagtctcttca

SEQ ID NO: 70 - DNA, 330 bp, synthetic construct
  source 1..330: mol_type = other DNA
cagtctgttctgacacagcctccatccgtgtctgctgcccctggccagaaagtgaccatcagctgtagcggcagcagctccaacatcggcaacaactacgtgtcctggtatcagcagctgcccggcacagctcccaaactgctgatctacgacaacaacaagcggcccagcggcatccccgatagattttctggcagcaagagcggcaccagcgccacactgggaattacaggactgcagacaggcgacgaggccgactactattgtggcacctgggattctagcctgagcgcctgggttttcggcggaggcacaaaactgacagtgcta

SEQ ID NO: 71 - protein, 448 aa, synthetic construct
EVQLLESGGGLVQPGGSLRLSCAAGGFTFSAYTMNWVRQAPGKGLEWVSAISASGGRTYYADSVKGRFTISRDNSKNTLYLQMNSLRAEDTAVYYCARRFARGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 216 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - protein, 116 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSS

SEQ ID NO: 74 - protein, 5 aa, synthetic construct
DSEMH

SEQ ID NO: 75 - protein, 17 aa, synthetic construct
AIQPETGGTAYNQKFKA

SEQ ID NO: 76 - protein, 7 aa, synthetic construct
RRRNFDY

SEQ ID NO: 77 - protein, 112 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIK

SEQ ID NO: 78 - protein, 16 aa, synthetic construct
RSSQSLFHSSGNTYLH

SEQ ID NO: 79 - protein, 7 aa, synthetic construct
KVSNRFS

SEQ ID NO: 80 - protein, 9 aa, synthetic construct
SQSTHVPFT

SEQ ID NO: 81 - DNA, 348 bp, synthetic construct
  source 1..348: mol_type = other DNA
caggttcagctggttcagtctggcgccgaagtgaagaaacctggcgcctctgtgaaggtgtcctgcaaggccagcggctacacctttaccgacagcgagatgcactgggtccgacaggctacaggacagggactcgaatggatgggagccatccagcctgaaacaggcggcaccgcctacaaccagaaattcaaggccagagtgaccatgaccagagacaccagcatcagcacagcctacatggaactgagcagcctgagaagcgaggacaccgccgtgtactactgcgcccgcagaagaagaaacttcgactactggggccagggcaccctggttacagtttcttca

SEQ ID NO: 82 - DNA, 336 bp, synthetic construct
  source 1..336: mol_type = other DNA
gacatcgtgatgacccagacacctctgagcctgagcgtgacacctggacagcctgccagcatcagctgtagaagcagccagagcctgttccacagctccggcaatacctacctgcactggtatctgcagaagcccggacagcctcctcagctgctgatctacaaggtgtccaaccggttcagcggcgtgcccgatagattttctggcagcggctctggcaccgacttcaccctgaagatctccagagtggaagccgaggacgtgggcgtgtactactgtagccagagcacccacgtgccattcacctttggccagggcaccaagctggaaatcaaa

SEQ ID NO: 83 - protein, 446 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 219 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 121 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSS

SEQ ID NO: 86 - protein, 5 aa, synthetic construct
TYAMN

SEQ ID NO: 87 - protein, 19 aa, synthetic construct
RIRSKSNNYATYYADSVKD

SEQ ID NO: 88 - protein, 10 aa, synthetic construct
GLLRYRFFDV

SEQ ID NO: 89 - protein, 112 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 90 - protein, 16 aa, synthetic construct
RSSKSLLHSNGNTYLY

SEQ ID NO: 91 - protein, 7 aa, synthetic construct
RMSNLAS

SEQ ID NO: 92 - protein, 9 aa, synthetic construct
MQHLEYPFT

SEQ ID NO: 93 - DNA, 363 bp, synthetic construct
  source 1..363: mol_type = other DNA
gaagtgcagctggtggaatctggcggaggactggttcaacctggcggctctctgaagctgtcttgtgccgccagcggcttcaccttcaacacctacgccatgaactgggtccgacaggcctctggcaaaggcctggaatgggtcggacggatcagaagcaagagcaacaattacgccacctactacgccgacagcgtgaaggacagattcaccatcagccgggacgacagcaagaacaccgcctacctgcagatgaacagcctgaaaaccgaggacaccgccgtgtactattgtgtgcggggcctgctgcggtacagattctttgatgtgtggggccagggcaccaccgtgacagtttcttca

SEQ ID NO: 94 - DNA, 336 bp, synthetic construct
  source 1..336: mol_type = other DNA
gacatcgtgatgacacagagccctctgagcctgcctgtgacacctggcgaacctgccagcatcagctgcagaagcagcaagtctctgctgcacagcaacggcaatacctacctgtactggttcctgcagaaacccggccagtctcctcagctgctgatctacagaatgagcaacctggccagcggcgtgcccgatagattttctggctctggcagcggcaccgacttcaccctgaagatctctagagtggaagccgaggacgtgggcgtgtactactgtatgcagcacctcgagtaccccttcacctttggcggcggaacaaaggtggaaatcaaa

SEQ ID NO: 95 - protein, 451 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 219 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC